SEQUENCE LISTING (435 sequences)

SEQ ID NO: 1 - RNA, 36 nt, Artificial Sequence
  Description of Artificial Sequence Synthetic oligonucleotide
  modified_base (1)..(20): a, c, u, g, unknown or other
nnnnnnnnnnnnnnnnnnnnguuuuagagcuaugcu

SEQ ID NO: 2 - RNA, 67 nt, Artificial Sequence
  Description of Artificial Sequence Synthetic oligonucleotide
agcauagcaaguuaaaauaaggcuaguccguuaucaacuugaaaaaguggcaccgagucggugcuuu

SEQ ID NO: 3 - RNA, 36 nt, Artificial Sequence
  Description of Artificial Sequence Synthetic oligonucleotide
  modified_base (1)..(20): a, c, u, g, unknown or other
nnnnnnnnnnnnnnnnnnnnguuuuagagcgagcgc

SEQ ID NO: 4 - RNA, 67 nt, Artificial Sequence
  Description of Artificial Sequence Synthetic oligonucleotide
gcgcucgcaaguuaaaauaaggcuaguccguuaucaacuugaaaaaguggcaccgagucggugcuuu

SEQ ID NO: 5 - DNA, 36 bp, Artificial Sequence
  Description of Artificial Sequence Synthetic oligonucleotide
  Description of Combined DNA/RNA Molecule Synthetic oligonucleotide
  modified_base (1)..(20): a, c, t, g, unknown or other
nnnnnnnnnnnnnnnnnnnnguuuuagagcuaugcu

SEQ ID NO: 6 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 7 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 8 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 9 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 10 - RNA, 67 nt, Artificial Sequence; identical to SEQ ID NO: 2
  Description of Artificial Sequence Synthetic oligonucleotide

SEQ ID NO: 11 - RNA, 67 nt, Artificial Sequence; identical to SEQ ID NO: 2
  Description of Artificial Sequence Synthetic oligonucleotide

SEQ ID NO: 12 - RNA, 67 nt, Artificial Sequence; identical to SEQ ID NO: 2
  Description of Artificial Sequence Synthetic oligonucleotide

SEQ ID NO: 13 - RNA, 67 nt, Artificial Sequence; identical to SEQ ID NO: 2
  Description of Artificial Sequence Synthetic oligonucleotide

SEQ ID NO: 14 - RNA, 67 nt, Artificial Sequence; identical to SEQ ID NO: 2
  Description of Artificial Sequence Synthetic oligonucleotide

SEQ ID NO: 15 - RNA, 67 nt, Artificial Sequence; identical to SEQ ID NO: 2
  Description of Artificial Sequence Synthetic oligonucleotide

SEQ ID NO: 16 - RNA, 67 nt, Artificial Sequence; identical to SEQ ID NO: 2
  Description of Artificial Sequence Synthetic oligonucleotide

SEQ ID NO: 17 - RNA, 67 nt, Artificial Sequence; identical to SEQ ID NO: 2
  Description of Artificial Sequence Synthetic oligonucleotide

SEQ ID NO: 18 - RNA, 67 nt, Artificial Sequence; identical to SEQ ID NO: 2
  Description of Artificial Sequence Synthetic oligonucleotide

SEQ ID NO: 19 - RNA, 67 nt, Artificial Sequence; identical to SEQ ID NO: 2
  Description of Artificial Sequence Synthetic oligonucleotide

SEQ ID NO: 20 - RNA, 67 nt, Artificial Sequence; identical to SEQ ID NO: 2
  Description of Artificial Sequence Synthetic oligonucleotide

SEQ ID NO: 21 - RNA, 67 nt, Artificial Sequence; identical to SEQ ID NO: 2
  Description of Artificial Sequence Synthetic oligonucleotide

SEQ ID NO: 22 - RNA, 67 nt, Artificial Sequence; identical to SEQ ID NO: 2
  Description of Artificial Sequence Synthetic oligonucleotide

SEQ ID NO: 23 - RNA, 67 nt, Artificial Sequence; identical to SEQ ID NO: 2
  Description of Artificial Sequence Synthetic oligonucleotide

SEQ ID NO: 24 - RNA, 67 nt, Artificial Sequence; identical to SEQ ID NO: 2
  Description of Artificial Sequence Synthetic oligonucleotide

SEQ ID NO: 25 - RNA, 67 nt, Artificial Sequence; identical to SEQ ID NO: 2
  Description of Artificial Sequence Synthetic oligonucleotide

SEQ ID NO: 26 - RNA, 67 nt, Artificial Sequence; identical to SEQ ID NO: 2
  Description of Artificial Sequence Synthetic oligonucleotide

SEQ ID NO: 27 - RNA, 67 nt, Artificial Sequence; identical to SEQ ID NO: 2
  Description of Artificial Sequence Synthetic oligonucleotide

SEQ ID NO: 28 - RNA, 67 nt, Artificial Sequence; identical to SEQ ID NO: 2
  Description of Artificial Sequence Synthetic oligonucleotide

SEQ ID NO: 29 - RNA, 67 nt, Artificial Sequence; identical to SEQ ID NO: 2
  Description of Artificial Sequence Synthetic oligonucleotide

SEQ ID NO: 30 - RNA, 67 nt, Artificial Sequence; identical to SEQ ID NO: 2
  Description of Artificial Sequence Synthetic oligonucleotide

SEQ ID NO: 31 - RNA, 67 nt, Artificial Sequence; identical to SEQ ID NO: 2
  Description of Artificial Sequence Synthetic oligonucleotide

SEQ ID NO: 32 - RNA, 67 nt, Artificial Sequence; identical to SEQ ID NO: 2
  Description of Artificial Sequence Synthetic oligonucleotide

SEQ ID NO: 33 - RNA, 67 nt, Artificial Sequence; identical to SEQ ID NO: 2
  Description of Artificial Sequence Synthetic oligonucleotide

SEQ ID NO: 34 - RNA, 67 nt, Artificial Sequence; identical to SEQ ID NO: 2
  Description of Artificial Sequence Synthetic oligonucleotide

SEQ ID NO: 35 - RNA, 67 nt, Artificial Sequence; identical to SEQ ID NO: 2
  Description of Artificial Sequence Synthetic oligonucleotide

SEQ ID NO: 36 - RNA, 67 nt, Artificial Sequence; identical to SEQ ID NO: 2
  Description of Artificial Sequence Synthetic oligonucleotide

SEQ ID NO: 37 - RNA, 67 nt, Artificial Sequence; identical to SEQ ID NO: 2
  Description of Artificial Sequence Synthetic oligonucleotide

SEQ ID NO: 38 - RNA, 67 nt, Artificial Sequence; identical to SEQ ID NO: 2
  Description of Artificial Sequence Synthetic oligonucleotide

SEQ ID NO: 39 - RNA, 67 nt, Artificial Sequence; identical to SEQ ID NO: 2
  Description of Artificial Sequence Synthetic oligonucleotide

SEQ ID NO: 40 - RNA, 67 nt, Artificial Sequence; identical to SEQ ID NO: 2
  Description of Artificial Sequence Synthetic oligonucleotide

SEQ ID NO: 41 - RNA, 67 nt, Artificial Sequence; identical to SEQ ID NO: 2
  Description of Artificial Sequence Synthetic oligonucleotide

SEQ ID NO: 42 - RNA, 67 nt, Artificial Sequence; identical to SEQ ID NO: 2
  Description of Artificial Sequence Synthetic oligonucleotide

SEQ ID NO: 43 - RNA, 67 nt, Artificial Sequence; identical to SEQ ID NO: 2
  Description of Artificial Sequence Synthetic oligonucleotide

SEQ ID NO: 44 - RNA, 67 nt, Artificial Sequence; identical to SEQ ID NO: 2
  Description of Artificial Sequence Synthetic oligonucleotide

SEQ ID NO: 45 - RNA, 67 nt, Artificial Sequence; identical to SEQ ID NO: 2
  Description of Artificial Sequence Synthetic oligonucleotide

SEQ ID NO: 46 - RNA, 67 nt, Artificial Sequence; identical to SEQ ID NO: 2
  Description of Artificial Sequence Synthetic oligonucleotide

SEQ ID NO: 47 - RNA, 67 nt, Artificial Sequence; identical to SEQ ID NO: 2
  Description of Artificial Sequence Synthetic oligonucleotide

SEQ ID NO: 48 - RNA, 67 nt, Artificial Sequence; identical to SEQ ID NO: 2
  Description of Artificial Sequence Synthetic oligonucleotide

SEQ ID NO: 49 - RNA, 67 nt, Artificial Sequence; identical to SEQ ID NO: 2
  Description of Artificial Sequence Synthetic oligonucleotide

SEQ ID NO: 50 - RNA, 67 nt, Artificial Sequence; identical to SEQ ID NO: 2
  Description of Artificial Sequence Synthetic oligonucleotide

SEQ ID NO: 51 - RNA, 67 nt, Artificial Sequence; identical to SEQ ID NO: 2
  Description of Artificial Sequence Synthetic oligonucleotide

SEQ ID NO: 52 - RNA, 67 nt, Artificial Sequence; identical to SEQ ID NO: 2
  Description of Artificial Sequence Synthetic oligonucleotide

SEQ ID NO: 53 - RNA, 67 nt, Artificial Sequence; identical to SEQ ID NO: 2
  Description of Artificial Sequence Synthetic oligonucleotide

SEQ ID NO: 54 - RNA, 67 nt, Artificial Sequence; identical to SEQ ID NO: 2
  Description of Artificial Sequence Synthetic oligonucleotide

SEQ ID NO: 55 - RNA, 67 nt, Artificial Sequence; identical to SEQ ID NO: 2
  Description of Artificial Sequence Synthetic oligonucleotide

SEQ ID NO: 56 - RNA, 67 nt, Artificial Sequence; identical to SEQ ID NO: 2
  Description of Artificial Sequence Synthetic oligonucleotide

SEQ ID NO: 57 - RNA, 67 nt, Artificial Sequence; identical to SEQ ID NO: 2
  Description of Artificial Sequence Synthetic oligonucleotide

SEQ ID NO: 58 - RNA, 67 nt, Artificial Sequence; identical to SEQ ID NO: 2
  Description of Artificial Sequence Synthetic oligonucleotide

SEQ ID NO: 59 - RNA, 67 nt, Artificial Sequence; identical to SEQ ID NO: 2
  Description of Artificial Sequence Synthetic oligonucleotide

SEQ ID NO: 60 - RNA, 67 nt, Artificial Sequence; identical to SEQ ID NO: 2
  Description of Artificial Sequence Synthetic oligonucleotide

SEQ ID NO: 61 - RNA, 67 nt, Artificial Sequence; identical to SEQ ID NO: 2
  Description of Artificial Sequence Synthetic oligonucleotide

SEQ ID NO: 62 - RNA, 67 nt, Artificial Sequence; identical to SEQ ID NO: 2
  Description of Artificial Sequence Synthetic oligonucleotide

SEQ ID NO: 63 - RNA, 67 nt, Artificial Sequence; identical to SEQ ID NO: 2
  Description of Artificial Sequence Synthetic oligonucleotide

SEQ ID NO: 64 - RNA, 67 nt, Artificial Sequence; identical to SEQ ID NO: 2
  Description of Artificial Sequence Synthetic oligonucleotide

SEQ ID NO: 65 - RNA, 67 nt, Artificial Sequence; identical to SEQ ID NO: 2
  Description of Artificial Sequence Synthetic oligonucleotide

SEQ ID NO: 66 - RNA, 67 nt, Artificial Sequence; identical to SEQ ID NO: 2
  Description of Artificial Sequence Synthetic oligonucleotide

SEQ ID NO: 67 - RNA, 67 nt, Artificial Sequence; identical to SEQ ID NO: 2
  Description of Artificial Sequence Synthetic oligonucleotide

SEQ ID NO: 68 - RNA, 67 nt, Artificial Sequence; identical to SEQ ID NO: 2
  Description of Artificial Sequence Synthetic oligonucleotide

SEQ ID NO: 69 - RNA, 67 nt, Artificial Sequence; identical to SEQ ID NO: 2
  Description of Artificial Sequence Synthetic oligonucleotide

SEQ ID NO: 70 - RNA, 67 nt, Artificial Sequence; identical to SEQ ID NO: 2
  Description of Artificial Sequence Synthetic oligonucleotide

SEQ ID NO: 71 - RNA, 67 nt, Artificial Sequence; identical to SEQ ID NO: 2
  Description of Artificial Sequence Synthetic oligonucleotide

SEQ ID NO: 72 - RNA, 67 nt, Artificial Sequence; identical to SEQ ID NO: 2
  Description of Artificial Sequence Synthetic oligonucleotide

SEQ ID NO: 73 - RNA, 67 nt, Artificial Sequence; identical to SEQ ID NO: 2
  Description of Artificial Sequence Synthetic oligonucleotide

SEQ ID NO: 74 - RNA, 67 nt, Artificial Sequence; identical to SEQ ID NO: 2
  Description of Artificial Sequence Synthetic oligonucleotide

SEQ ID NO: 75 - RNA, 67 nt, Artificial Sequence; identical to SEQ ID NO: 2
  Description of Artificial Sequence Synthetic oligonucleotide

SEQ ID NO: 76 - RNA, 67 nt, Artificial Sequence; identical to SEQ ID NO: 2
  Description of Artificial Sequence Synthetic oligonucleotide

SEQ ID NO: 77 - RNA, 67 nt, Artificial Sequence; identical to SEQ ID NO: 2
  Description of Artificial Sequence Synthetic oligonucleotide

SEQ ID NO: 78 - RNA, 67 nt, Artificial Sequence; identical to SEQ ID NO: 2
  Description of Artificial Sequence Synthetic oligonucleotide

SEQ ID NO: 79 - RNA, 67 nt, Artificial Sequence; identical to SEQ ID NO: 2
  Description of Artificial Sequence Synthetic oligonucleotide

SEQ ID NO: 80 - DNA, 67 bp, Artificial Sequence
  Description of Artificial Sequence Synthetic oligonucleotide
  Description of Combined DNA/RNA Molecule Synthetic oligonucleotide
agcauagcaaguuaaaauaaggcuaguccguuaucaacuugaaaaaguggcaccgagucggugcuuu

SEQ ID NO: 81 - DNA, 67 bp, Artificial Sequence; identical to SEQ ID NO: 80
  Description of Artificial Sequence Synthetic oligonucleotide
  Description of Combined DNA/RNA Molecule Synthetic oligonucleotide

SEQ ID NO: 82 - DNA, 67 bp, Artificial Sequence; identical to SEQ ID NO: 80
  Description of Artificial Sequence Synthetic oligonucleotide
  Description of Combined DNA/RNA Molecule Synthetic oligonucleotide

SEQ ID NO: 83 - DNA, 67 bp, Artificial Sequence; identical to SEQ ID NO: 80
  Description of Artificial Sequence Synthetic oligonucleotide
  Description of Combined DNA/RNA Molecule Synthetic oligonucleotide

SEQ ID NO: 84 - DNA, 67 bp, Artificial Sequence; identical to SEQ ID NO: 80
  Description of Artificial Sequence Synthetic oligonucleotide
  Description of Combined DNA/RNA Molecule Synthetic oligonucleotide

SEQ ID NO: 85 - DNA, 67 bp, Artificial Sequence; identical to SEQ ID NO: 80
  Description of Artificial Sequence Synthetic oligonucleotide
  Description of Combined DNA/RNA Molecule Synthetic oligonucleotide

SEQ ID NO: 86 - DNA, 67 bp, Artificial Sequence; identical to SEQ ID NO: 80
  Description of Artificial Sequence Synthetic oligonucleotide
  Description of Combined DNA/RNA Molecule Synthetic oligonucleotide

SEQ ID NO: 87 - DNA, 67 bp, Artificial Sequence; identical to SEQ ID NO: 80
  Description of Artificial Sequence Synthetic oligonucleotide
  Description of Combined DNA/RNA Molecule Synthetic oligonucleotide

SEQ ID NO: 88 - DNA, 67 bp, Artificial Sequence; identical to SEQ ID NO: 80
  Description of Artificial Sequence Synthetic oligonucleotide
  Description of Combined DNA/RNA Molecule Synthetic oligonucleotide

SEQ ID NO: 89 - DNA, 67 bp, Artificial Sequence; identical to SEQ ID NO: 80
  Description of Artificial Sequence Synthetic oligonucleotide
  Description of Combined DNA/RNA Molecule Synthetic oligonucleotide

SEQ ID NO: 90 - DNA, 67 bp, Artificial Sequence; identical to SEQ ID NO: 80
  Description of Artificial Sequence Synthetic oligonucleotide
  Description of Combined DNA/RNA Molecule Synthetic oligonucleotide

SEQ ID NO: 91 - DNA, 67 bp, Artificial Sequence; identical to SEQ ID NO: 80
  Description of Artificial Sequence Synthetic oligonucleotide
  Description of Combined DNA/RNA Molecule Synthetic oligonucleotide

SEQ ID NO: 92 - DNA, 67 bp, Artificial Sequence; identical to SEQ ID NO: 80
  Description of Artificial Sequence Synthetic oligonucleotide
  Description of Combined DNA/RNA Molecule Synthetic oligonucleotide

SEQ ID NO: 93 - DNA, 67 bp, Artificial Sequence; identical to SEQ ID NO: 80
  Description of Artificial Sequence Synthetic oligonucleotide
  Description of Combined DNA/RNA Molecule Synthetic oligonucleotide

SEQ ID NO: 94 - RNA, 67 nt, Artificial Sequence; identical to SEQ ID NO: 2
  Description of Artificial Sequence Synthetic oligonucleotide

SEQ ID NO: 95 - RNA, 67 nt, Artificial Sequence; identical to SEQ ID NO: 2
  Description of Artificial Sequence Synthetic oligonucleotide

SEQ ID NO: 96 - RNA, 67 nt, Artificial Sequence; identical to SEQ ID NO: 2
  Description of Artificial Sequence Synthetic oligonucleotide

SEQ ID NO: 97 - RNA, 67 nt, Artificial Sequence; identical to SEQ ID NO: 2
  Description of Artificial Sequence Synthetic oligonucleotide

SEQ ID NO: 98 - RNA, 67 nt, Artificial Sequence; identical to SEQ ID NO: 2
  Description of Artificial Sequence Synthetic oligonucleotide

SEQ ID NO: 99 - RNA, 67 nt, Artificial Sequence; identical to SEQ ID NO: 2
  Description of Artificial Sequence Synthetic oligonucleotide

SEQ ID NO: 100 - RNA, 67 nt, Artificial Sequence; identical to SEQ ID NO: 2
  Description of Artificial Sequence Synthetic oligonucleotide

SEQ ID NO: 101 - RNA, 67 nt, Artificial Sequence; identical to SEQ ID NO: 2
  Description of Artificial Sequence Synthetic oligonucleotide

SEQ ID NO: 102 - RNA, 67 nt, Artificial Sequence; identical to SEQ ID NO: 2
  Description of Artificial Sequence Synthetic oligonucleotide

SEQ ID NO: 103 - RNA, 67 nt, Artificial Sequence; identical to SEQ ID NO: 2
  Description of Artificial Sequence Synthetic oligonucleotide

SEQ ID NO: 104 - RNA, 67 nt, Artificial Sequence; identical to SEQ ID NO: 2
  Description of Artificial Sequence Synthetic oligonucleotide

SEQ ID NO: 105 - RNA, 67 nt, Artificial Sequence; identical to SEQ ID NO: 2
  Description of Artificial Sequence Synthetic oligonucleotide

SEQ ID NO: 106 - RNA, 67 nt, Artificial Sequence; identical to SEQ ID NO: 2
  Description of Artificial Sequence Synthetic oligonucleotide

SEQ ID NO: 107 - RNA, 67 nt, Artificial Sequence; identical to SEQ ID NO: 2
  Description of Artificial Sequence Synthetic oligonucleotide

SEQ ID NO: 108 - DNA, 67 bp, Artificial Sequence; identical to SEQ ID NO: 80
  Description of Artificial Sequence Synthetic oligonucleotide
  Description of Combined DNA/RNA Molecule Synthetic oligonucleotide

SEQ ID NO: 109 - DNA, 67 bp, Artificial Sequence; identical to SEQ ID NO: 80
  Description of Artificial Sequence Synthetic oligonucleotide
  Description of Combined DNA/RNA Molecule Synthetic oligonucleotide

SEQ ID NO: 110 - DNA, 67 bp, Artificial Sequence; identical to SEQ ID NO: 80
  Description of Artificial Sequence Synthetic oligonucleotide
  Description of Combined DNA/RNA Molecule Synthetic oligonucleotide

SEQ ID NO: 111 - RNA, 67 nt, Artificial Sequence; identical to SEQ ID NO: 2
  Description of Artificial Sequence Synthetic oligonucleotide

SEQ ID NO: 112 - RNA, 67 nt, Artificial Sequence; identical to SEQ ID NO: 2
  Description of Artificial Sequence Synthetic oligonucleotide

SEQ ID NO: 113 - RNA, 67 nt, Artificial Sequence; identical to SEQ ID NO: 2
  Description of Artificial Sequence Synthetic oligonucleotide

SEQ ID NO: 114 - RNA, 67 nt, Artificial Sequence; identical to SEQ ID NO: 2
  Description of Artificial Sequence Synthetic oligonucleotide

SEQ ID NO: 115 - RNA, 67 nt, Artificial Sequence; identical to SEQ ID NO: 2
  Description of Artificial Sequence Synthetic oligonucleotide

SEQ ID NO: 116 - RNA, 67 nt, Artificial Sequence; identical to SEQ ID NO: 2
  Description of Artificial Sequence Synthetic oligonucleotide

SEQ ID NO: 117 - RNA, 67 nt, Artificial Sequence; identical to SEQ ID NO: 2
  Description of Artificial Sequence Synthetic oligonucleotide

SEQ ID NO: 118 - RNA, 67 nt, Artificial Sequence; identical to SEQ ID NO: 2
  Description of Artificial Sequence Synthetic oligonucleotide

SEQ ID NO: 119 - RNA, 67 nt, Artificial Sequence; identical to SEQ ID NO: 2
  Description of Artificial Sequence Synthetic oligonucleotide

SEQ ID NO: 120 - RNA, 67 nt, Artificial Sequence; identical to SEQ ID NO: 2
  Description of Artificial Sequence Synthetic oligonucleotide

SEQ ID NO: 121 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 122 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 123 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 124 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 125 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 126 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 127 - RNA, 67 nt, Artificial Sequence; identical to SEQ ID NO: 2
  Description of Artificial Sequence Synthetic oligonucleotide

SEQ ID NO: 128 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 129 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 130 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 131 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 132 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 133 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 134 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 135 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 136 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 137 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 138 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 139 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 140 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 141 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 142 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 143 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 144 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 145 - RNA, 36 nt, Artificial Sequence
  Description of Artificial Sequence Synthetic oligonucleotide
ggugagcucuuauuugcguaguuuuagagcuaugcu

SEQ ID NO: 146 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 147 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 148 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 149 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 150 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 151 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 152 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 153 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 154 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 155 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 156 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 157 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 158 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 159 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 160 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 161 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 162 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 163 - RNA, 37 nt, Artificial Sequence
  Description of Artificial Sequence Synthetic oligonucleotide
  modified_base (1)..(20): a, c, u, g, unknown or other
nnnnnnnnnnnnnnnnnnnnaguuuuagagcuaugcu

SEQ ID NO: 164 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 165 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 166 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 167 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 168 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 169 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 170 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 171 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 172 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 173 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 174 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 175 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 176 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 177 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 178 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 179 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 180 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 181 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 182 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 183 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 184 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 185 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 186 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 187 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 188 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 189 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 190 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 191 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 192 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 193 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 194 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 195 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 196 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 197 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 198 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 199 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 200 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 201 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 202 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 203 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 204 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 205 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 206 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 207 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 208 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 209 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 210 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 211 - RNA, 37 nt, Artificial Sequence
  Description of Artificial Sequence Synthetic oligonucleotide
  modified_base (1)..(19): a, c, u, g, unknown or other
  modified_base (21)..(21): a, c, u, g, unknown or other
nnnnnnnnnnnnnnnnnnngnguuuuagagcuaugcu

SEQ ID NO: 212 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 213 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 214 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 215 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 216 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 217 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 218 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 219 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 220 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 221 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 222 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 223 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 224 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 225 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 226 - DNA, 36 bp, Artificial Sequence; identical to SEQ ID NO: 5
  Description of Artificial Sequence Synthetic oligonucleotide
  Description of Combined DNA/RNA Molecule Synthetic oligonucleotide
  modified_base (1)..(20): a, c, t, g, unknown or other

SEQ ID NO: 227 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 228 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 229 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 230 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 231 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 232 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 233 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 234 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 235 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 236 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 237 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 238 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 239 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 240 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 241 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 242 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 243 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 244 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 245 - RNA, 36 nt, Artificial Sequence; identical to SEQ ID NO: 1
  Description of Artificial Sequence Synthetic oligonucleotide
  modified_base (1)..(20): a, c, u, g, unknown or other

SEQ ID NO: 246 - RNA, 36 nt, Artificial Sequence; identical to SEQ ID NO: 3
  Description of Artificial Sequence Synthetic oligonucleotide
  modified_base (1)..(20): a, c, u, g, unknown or other

SEQ ID NO: 247 - RNA, 67 nt, Artificial Sequence; identical to SEQ ID NO: 4
  Description of Artificial Sequence Synthetic oligonucleotide

SEQ ID NO: 248 - RNA, 36 nt, Artificial Sequence
  Description of Artificial Sequence Synthetic oligonucleotide
gagacaaaucaccugccucgguuuuagagcuaugcu

SEQ ID NO: 249 - RNA, 36 nt, Artificial Sequence; identical to SEQ ID NO: 248
  Description of Artificial Sequence Synthetic oligonucleotide

SEQ ID NO: 250 - DNA, 36 bp, Artificial Sequence
  Description of Artificial Sequence Synthetic oligonucleotide
  Description of Combined DNA/RNA Molecule Synthetic oligonucleotide
gagacaaaucaccugccucgguuuuagagcuaugcu

SEQ ID NO: 251 - RNA, 36 nt, Artificial Sequence; identical to SEQ ID NO: 248
  Description of Artificial Sequence Synthetic oligonucleotide

SEQ ID NO: 252 - RNA, 36 nt, Artificial Sequence
  Description of Artificial Sequence Synthetic oligonucleotide
cccauaccuuggagcaacggguuuuagagcuaugcu

SEQ ID NO: 253 - RNA, 36 nt, Artificial Sequence; identical to SEQ ID NO: 252
  Description of Artificial Sequence Synthetic oligonucleotide

SEQ ID NO: 254 - RNA, 36 nt, Artificial Sequence; identical to SEQ ID NO: 252
  Description of Artificial Sequence Synthetic oligonucleotide

SEQ ID NO: 255 - DNA, 36 bp, Artificial Sequence
  Description of Artificial Sequence Synthetic oligonucleotide
  Description of Combined DNA/RNA Molecule Synthetic oligonucleotide
cccauaccuuggagcaacggguuuuagagcuaugcu

SEQ ID NO: 256 - DNA, 36 bp, Artificial Sequence; identical to SEQ ID NO: 255
  Description of Artificial Sequence Synthetic oligonucleotide
  Description of Combined DNA/RNA Molecule Synthetic oligonucleotide

SEQ ID NO: 257 - RNA, 36 nt, Artificial Sequence; identical to SEQ ID NO: 248
  Description of Artificial Sequence Synthetic oligonucleotide

SEQ ID NO: 258 - RNA, 36 nt, Artificial Sequence; identical to SEQ ID NO: 248
  Description of Artificial Sequence Synthetic oligonucleotide

SEQ ID NO: 259 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 260 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 261 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 262 - DNA, 38 bp, Artificial Sequence
  Description of Artificial Sequence Synthetic oligonucleotide
  Description of Combined DNA/RNA Molecule Synthetic oligonucleotide
gagacaaaucaccugccucgguuuuagagcuaugcutt

SEQ ID NO: 263 - DNA, 38 bp, Artificial Sequence; identical to SEQ ID NO: 262
  Description of Artificial Sequence Synthetic oligonucleotide
  Description of Combined DNA/RNA Molecule Synthetic oligonucleotide

SEQ ID NO: 264 - DNA, 38 bp, Artificial Sequence; identical to SEQ ID NO: 262
  Description of Artificial Sequence Synthetic oligonucleotide
  Description of Combined DNA/RNA Molecule Synthetic oligonucleotide

SEQ ID NO: 265 - DNA, 38 bp, Artificial Sequence; identical to SEQ ID NO: 262
  Description of Artificial Sequence Synthetic oligonucleotide
  Description of Combined DNA/RNA Molecule Synthetic oligonucleotide

SEQ ID NO: 266 - DNA, 38 bp, Artificial Sequence; identical to SEQ ID NO: 262
  Description of Artificial Sequence Synthetic oligonucleotide
  Description of Combined DNA/RNA Molecule Synthetic oligonucleotide

SEQ ID NO: 267 - RNA, 36 nt, Artificial Sequence; identical to SEQ ID NO: 145
  Description of Artificial Sequence Synthetic oligonucleotide

SEQ ID NO: 268 - RNA, 36 nt, Artificial Sequence; identical to SEQ ID NO: 145
  Description of Artificial Sequence Synthetic oligonucleotide

SEQ ID NO: 269 - RNA, 36 nt, Artificial Sequence; identical to SEQ ID NO: 145
  Description of Artificial Sequence Synthetic oligonucleotide

SEQ ID NO: 270 - RNA, 36 nt, Artificial Sequence; identical to SEQ ID NO: 145
  Description of Artificial Sequence Synthetic oligonucleotide

SEQ ID NO: 271 - RNA, 36 nt, Artificial Sequence; identical to SEQ ID NO: 145
  Description of Artificial Sequence Synthetic oligonucleotide

SEQ ID NO: 272 - RNA, 36 nt, Artificial Sequence; identical to SEQ ID NO: 145
  Description of Artificial Sequence Synthetic oligonucleotide

SEQ ID NO: 273 - RNA, 36 nt, Artificial Sequence; identical to SEQ ID NO: 145
  Description of Artificial Sequence Synthetic oligonucleotide

SEQ ID NO: 274 - RNA, 36 nt, Artificial Sequence; identical to SEQ ID NO: 145
  Description of Artificial Sequence Synthetic oligonucleotide

SEQ ID NO: 275 - RNA, 36 nt, Artificial Sequence; identical to SEQ ID NO: 145
  Description of Artificial Sequence Synthetic oligonucleotide

SEQ ID NO: 276 - RNA, 36 nt, Artificial Sequence; identical to SEQ ID NO: 145
  Description of Artificial Sequence Synthetic oligonucleotide

SEQ ID NO: 277 - RNA, 36 nt, Artificial Sequence; identical to SEQ ID NO: 145
  Description of Artificial Sequence Synthetic oligonucleotide

SEQ ID NO: 278 - RNA, 36 nt, Artificial Sequence; identical to SEQ ID NO: 145
  Description of Artificial Sequence Synthetic oligonucleotide

SEQ ID NO: 279 - RNA, 36 nt, Artificial Sequence; identical to SEQ ID NO: 145
  Description of Artificial Sequence Synthetic oligonucleotide

SEQ ID NO: 280 - RNA, 36 nt, Artificial Sequence; identical to SEQ ID NO: 145
  Description of Artificial Sequence Synthetic oligonucleotide

SEQ ID NO: 281 - RNA, 36 nt, Artificial Sequence; identical to SEQ ID NO: 145
  Description of Artificial Sequence Synthetic oligonucleotide

SEQ ID NO: 282 - RNA, 36 nt, Artificial Sequence; identical to SEQ ID NO: 145
  Description of Artificial Sequence Synthetic oligonucleotide

SEQ ID NO: 283 - RNA, 36 nt, Artificial Sequence; identical to SEQ ID NO: 145
  Description of Artificial Sequence Synthetic oligonucleotide

SEQ ID NO: 284 - RNA, 36 nt, Artificial Sequence; identical to SEQ ID NO: 145
  Description of Artificial Sequence Synthetic oligonucleotide

SEQ ID NO: 285 - RNA, 36 nt, Artificial Sequence; identical to SEQ ID NO: 145
  Description of Artificial Sequence Synthetic oligonucleotide

SEQ ID NO: 286 - RNA, 36 nt, Artificial Sequence; identical to SEQ ID NO: 145
  Description of Artificial Sequence Synthetic oligonucleotide

SEQ ID NO: 287 - RNA, 36 nt, Artificial Sequence; identical to SEQ ID NO: 145
  Description of Artificial Sequence Synthetic oligonucleotide

SEQ ID NO: 288 - RNA, 36 nt, Artificial Sequence; identical to SEQ ID NO: 145
  Description of Artificial Sequence Synthetic oligonucleotide

SEQ ID NO: 289 - RNA, 36 nt, Artificial Sequence; identical to SEQ ID NO: 145
  Description of Artificial Sequence Synthetic oligonucleotide

SEQ ID NO: 290 - RNA, 36 nt, Artificial Sequence; identical to SEQ ID NO: 145
  Description of Artificial Sequence Synthetic oligonucleotide

SEQ ID NO: 291 - RNA, 36 nt, Artificial Sequence; identical to SEQ ID NO: 145
  Description of Artificial Sequence Synthetic oligonucleotide

SEQ ID NO: 292 - RNA, 36 nt, Artificial Sequence; identical to SEQ ID NO: 145
  Description of Artificial Sequence Synthetic oligonucleotide

SEQ ID NO: 293 - RNA, 36 nt, Artificial Sequence; identical to SEQ ID NO: 145
  Description of Artificial Sequence Synthetic oligonucleotide

SEQ ID NO: 294 - RNA, 36 nt, Artificial Sequence; identical to SEQ ID NO: 145
  Description of Artificial Sequence Synthetic oligonucleotide

SEQ ID NO: 295 - RNA, 36 nt, Artificial Sequence; identical to SEQ ID NO: 145
  Description of Artificial Sequence Synthetic oligonucleotide

SEQ ID NO: 296 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 297 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 298 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 299 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 300 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 301 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 302 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 303 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 304 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 305 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 306 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 307 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 308 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 309 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 310 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 311 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 312 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 313 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 314 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 315 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 316 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 317 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 318 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 319 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 320 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 321 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 322 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 323 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 324 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 325 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 326 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 327 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 328 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 329 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 330 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 331 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 332 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 333 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 334 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 335 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 336 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 337 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 338 - RNA, 36 nt, Artificial Sequence; identical to SEQ ID NO: 248
  Description of Artificial Sequence Synthetic oligonucleotide

SEQ ID NO: 339 - RNA, 36 nt, Artificial Sequence; identical to SEQ ID NO: 248
  Description of Artificial Sequence Synthetic oligonucleotide

SEQ ID NO: 340 - RNA, 36 nt, Artificial Sequence; identical to SEQ ID NO: 248
  Description of Artificial Sequence Synthetic oligonucleotide

SEQ ID NO: 341 - RNA, 36 nt, Artificial Sequence; identical to SEQ ID NO: 248
  Description of Artificial Sequence Synthetic oligonucleotide

SEQ ID NO: 342 - RNA, 36 nt, Artificial Sequence; identical to SEQ ID NO: 248
  Description of Artificial Sequence Synthetic oligonucleotide

SEQ ID NO: 343 - RNA, 36 nt, Artificial Sequence; identical to SEQ ID NO: 248
  Description of Artificial Sequence Synthetic oligonucleotide

SEQ ID NO: 344 - RNA, 36 nt, Artificial Sequence; identical to SEQ ID NO: 248
  Description of Artificial Sequence Synthetic oligonucleotide

SEQ ID NO: 345 - RNA, 36 nt, Artificial Sequence; identical to SEQ ID NO: 248
  Description of Artificial Sequence Synthetic oligonucleotide

SEQ ID NO: 346 - RNA, 36 nt, Artificial Sequence; identical to SEQ ID NO: 248
  Description of Artificial Sequence Synthetic oligonucleotide

SEQ ID NO: 347 - RNA, 36 nt, Artificial Sequence; identical to SEQ ID NO: 248
  Description of Artificial Sequence Synthetic oligonucleotide

SEQ ID NO: 348 - RNA, 36 nt, Artificial Sequence; identical to SEQ ID NO: 248
  Description of Artificial Sequence Synthetic oligonucleotide

SEQ ID NO: 349 - RNA, 36 nt, Artificial Sequence; identical to SEQ ID NO: 248
  Description of Artificial Sequence Synthetic oligonucleotide

SEQ ID NO: 350 - RNA, 36 nt, Artificial Sequence; identical to SEQ ID NO: 248
  Description of Artificial Sequence Synthetic oligonucleotide

SEQ ID NO: 351 - RNA, 36 nt, Artificial Sequence; identical to SEQ ID NO: 248
  Description of Artificial Sequence Synthetic oligonucleotide

SEQ ID NO: 352 - RNA, 36 nt, Artificial Sequence; identical to SEQ ID NO: 248
  Description of Artificial Sequence Synthetic oligonucleotide

SEQ ID NO: 353 - RNA, 36 nt, Artificial Sequence; identical to SEQ ID NO: 248
  Description of Artificial Sequence Synthetic oligonucleotide

SEQ ID NO: 354 - RNA, 36 nt, Artificial Sequence; identical to SEQ ID NO: 248
  Description of Artificial Sequence Synthetic oligonucleotide

SEQ ID NO: 355 - RNA, 36 nt, Artificial Sequence; identical to SEQ ID NO: 248
  Description of Artificial Sequence Synthetic oligonucleotide

SEQ ID NO: 356 - RNA, 36 nt, Artificial Sequence; identical to SEQ ID NO: 248
  Description of Artificial Sequence Synthetic oligonucleotide

SEQ ID NO: 357 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 358 - RNA, 36 nt, Artificial Sequence
  Description of Artificial Sequence Synthetic oligonucleotide
cgaaguuauauuaaggguucguuuuagagcuaugcu

SEQ ID NO: 359 - RNA, 36 nt, Artificial Sequence; identical to SEQ ID NO: 358
  Description of Artificial Sequence Synthetic oligonucleotide

SEQ ID NO: 360 - RNA, 36 nt, Artificial Sequence; identical to SEQ ID NO: 358
  Description of Artificial Sequence Synthetic oligonucleotide

SEQ ID NO: 361 - RNA, 36 nt, Artificial Sequence; identical to SEQ ID NO: 358
  Description of Artificial Sequence Synthetic oligonucleotide

SEQ ID NO: 362 - RNA, 36 nt, Artificial Sequence; identical to SEQ ID NO: 358
  Description of Artificial Sequence Synthetic oligonucleotide

SEQ ID NO: 363 - RNA, 36 nt, Artificial Sequence; identical to SEQ ID NO: 358
  Description of Artificial Sequence Synthetic oligonucleotide

SEQ ID NO: 364 - RNA, 36 nt, Artificial Sequence; identical to SEQ ID NO: 358
  Description of Artificial Sequence Synthetic oligonucleotide

SEQ ID NO: 365 - RNA, 36 nt, Artificial Sequence; identical to SEQ ID NO: 358
  Description of Artificial Sequence Synthetic oligonucleotide

SEQ ID NO: 366 - RNA, 36 nt, Artificial Sequence; identical to SEQ ID NO: 358
  Description of Artificial Sequence Synthetic oligonucleotide

SEQ ID NO: 367 - RNA, 36 nt, Artificial Sequence; identical to SEQ ID NO: 358
  Description of Artificial Sequence Synthetic oligonucleotide

SEQ ID NO: 368 - RNA, 36 nt, Artificial Sequence; identical to SEQ ID NO: 358
  Description of Artificial Sequence Synthetic oligonucleotide

SEQ ID NO: 369 - RNA, 36 nt, Artificial Sequence; identical to SEQ ID NO: 358
  Description of Artificial Sequence Synthetic oligonucleotide

SEQ ID NO: 370 - RNA, 36 nt, Artificial Sequence; identical to SEQ ID NO: 358
  Description of Artificial Sequence Synthetic oligonucleotide

SEQ ID NO: 371 - RNA, 36 nt, Artificial Sequence; identical to SEQ ID NO: 358
  Description of Artificial Sequence Synthetic oligonucleotide

SEQ ID NO: 372 - RNA, 36 nt, Artificial Sequence; identical to SEQ ID NO: 248
  Description of Artificial Sequence Synthetic oligonucleotide

SEQ ID NO: 373 - RNA, 36 nt, Artificial Sequence; identical to SEQ ID NO: 248
  Description of Artificial Sequence Synthetic oligonucleotide

SEQ ID NO: 374 - RNA, 36 nt, Artificial Sequence; identical to SEQ ID NO: 248
  Description of Artificial Sequence Synthetic oligonucleotide

SEQ ID NO: 375 - RNA, 36 nt, Artificial Sequence; identical to SEQ ID NO: 248
  Description of Artificial Sequence Synthetic oligonucleotide

SEQ ID NO: 376 - RNA, 36 nt, Artificial Sequence; identical to SEQ ID NO: 248
  Description of Artificial Sequence Synthetic oligonucleotide

SEQ ID NO: 377 - RNA, 36 nt, Artificial Sequence; identical to SEQ ID NO: 248
  Description of Artificial Sequence Synthetic oligonucleotide

SEQ ID NO: 378 - RNA, 36 nt, Artificial Sequence
  Description of Artificial Sequence Synthetic oligonucleotide
uuuaccguauuccacgaggcguuuuagagcuaugcu

SEQ ID NO: 379 - RNA, 36 nt, Artificial Sequence
  Description of Artificial Sequence Synthetic oligonucleotide
cgcccaucuucuagaaagacguuuuagagcuaugcu

SEQ ID NO: 380 - RNA, 36 nt, Artificial Sequence; identical to SEQ ID NO: 379
  Description of Artificial Sequence Synthetic oligonucleotide

SEQ ID NO: 381 - RNA, 36 nt, Artificial Sequence; identical to SEQ ID NO: 248
  Description of Artificial Sequence Synthetic oligonucleotide

SEQ ID NO: 382 - RNA, 36 nt, Artificial Sequence; identical to SEQ ID NO: 248
  Description of Artificial Sequence Synthetic oligonucleotide

SEQ ID NO: 383 - RNA, 36 nt, Artificial Sequence; identical to SEQ ID NO: 248
  Description of Artificial Sequence Synthetic oligonucleotide

SEQ ID NO: 384 - RNA, 36 nt, Artificial Sequence; identical to SEQ ID NO: 248
  Description of Artificial Sequence Synthetic oligonucleotide

SEQ ID NO: 385 - RNA, 36 nt, Artificial Sequence; identical to SEQ ID NO: 248
  Description of Artificial Sequence Synthetic oligonucleotide

SEQ ID NO: 386 - RNA, 36 nt, Artificial Sequence; identical to SEQ ID NO: 248
  Description of Artificial Sequence Synthetic oligonucleotide

SEQ ID NO: 387 - RNA, 36 nt, Artificial Sequence; identical to SEQ ID NO: 248
  Description of Artificial Sequence Synthetic oligonucleotide

SEQ ID NO: 388 - RNA, 36 nt, Artificial Sequence; identical to SEQ ID NO: 248
  Description of Artificial Sequence Synthetic oligonucleotide

SEQ ID NO: 389 - RNA, 36 nt, Artificial Sequence; identical to SEQ ID NO: 248
  Description of Artificial Sequence Synthetic oligonucleotide

SEQ ID NO: 390 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 391 - RNA, 36 nt, Artificial Sequence; identical to SEQ ID NO: 248
  Description of Artificial Sequence Synthetic oligonucleotide

SEQ ID NO: 392 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 393 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 394 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 395 - RNA, 36 nt, Artificial Sequence; identical to SEQ ID NO: 248
  Description of Artificial Sequence Synthetic oligonucleotide

SEQ ID NO: 396 - DNA, 36 bp, Artificial Sequence; identical to SEQ ID NO: 250
  Description of Artificial Sequence Synthetic oligonucleotide
  Description of Combined DNA/RNA Molecule Synthetic oligonucleotide

SEQ ID NO: 397 - RNA, 36 nt, Artificial Sequence; identical to SEQ ID NO: 248
  Description of Artificial Sequence Synthetic oligonucleotide

SEQ ID NO: 398 - RNA, 36 nt, Artificial Sequence; identical to SEQ ID NO: 248
  Description of Artificial Sequence Synthetic oligonucleotide

SEQ ID NO: 399 - RNA, 36 nt, Artificial Sequence; identical to SEQ ID NO: 378
  Description of Artificial Sequence Synthetic oligonucleotide

SEQ ID NO: 400 - RNA, 36 nt, Artificial Sequence; identical to SEQ ID NO: 378
  Description of Artificial Sequence Synthetic oligonucleotide

SEQ ID NO: 401 - RNA, 36 nt, Artificial Sequence; identical to SEQ ID NO: 378
  Description of Artificial Sequence Synthetic oligonucleotide

SEQ ID NO: 402 - RNA, 36 nt, Artificial Sequence; identical to SEQ ID NO: 378
  Description of Artificial Sequence Synthetic oligonucleotide

SEQ ID NO: 403 - RNA, 36 nt, Artificial Sequence; identical to SEQ ID NO: 378
  Description of Artificial Sequence Synthetic oligonucleotide

SEQ ID NO: 404 - RNA, 36 nt, Artificial Sequence; identical to SEQ ID NO: 378
  Description of Artificial Sequence Synthetic oligonucleotide

SEQ ID NO: 405 - RNA, 36 nt, Artificial Sequence; identical to SEQ ID NO: 378
  Description of Artificial Sequence Synthetic oligonucleotide

SEQ ID NO: 406 - RNA, 36 nt, Artificial Sequence; identical to SEQ ID NO: 378
  Description of Artificial Sequence Synthetic oligonucleotide

SEQ ID NO: 407 - RNA, 36 nt, Artificial Sequence; identical to SEQ ID NO: 378
  Description of Artificial Sequence Synthetic oligonucleotide

SEQ ID NO: 408 - RNA, 36 nt, Artificial Sequence; identical to SEQ ID NO: 378
  Description of Artificial Sequence Synthetic oligonucleotide

SEQ ID NO: 409 - RNA, 36 nt, Artificial Sequence; identical to SEQ ID NO: 378
  Description of Artificial Sequence Synthetic oligonucleotide

SEQ ID NO: 410 - RNA, 36 nt, Artificial Sequence; identical to SEQ ID NO: 378
  Description of Artificial Sequence Synthetic oligonucleotide

SEQ ID NO: 411 - RNA, 36 nt, Artificial Sequence; identical to SEQ ID NO: 378
  Description of Artificial Sequence Synthetic oligonucleotide

SEQ ID NO: 412 - RNA, 36 nt, Artificial Sequence; identical to SEQ ID NO: 378
  Description of Artificial Sequence Synthetic oligonucleotide

SEQ ID NO: 413 - RNA, 36 nt, Artificial Sequence; identical to SEQ ID NO: 378
  Description of Artificial Sequence Synthetic oligonucleotide

SEQ ID NO: 414 - DNA, 36 bp, Artificial Sequence
  Description of Artificial Sequence Synthetic oligonucleotide
  Description of Combined DNA/RNA Molecule Synthetic oligonucleotide
uuuaccguauuccacgaggcguuuuagagcuaugcu

SEQ ID NO: 415 - RNA, 36 nt, Artificial Sequence; identical to SEQ ID NO: 378
  Description of Artificial Sequence Synthetic oligonucleotide

SEQ ID NO: 416 - DNA, 36 bp, Artificial Sequence; identical to SEQ ID NO: 414
  Description of Artificial Sequence Synthetic oligonucleotide
  Description of Combined DNA/RNA Molecule Synthetic oligonucleotide

SEQ ID NO: 417 - DNA, 36 bp, Artificial Sequence; identical to SEQ ID NO: 414
  Description of Artificial Sequence Synthetic oligonucleotide
  Description of Combined DNA/RNA Molecule Synthetic oligonucleotide

SEQ ID NO: 418 - DNA, 36 bp, Artificial Sequence; identical to SEQ ID NO: 414
  Description of Artificial Sequence Synthetic oligonucleotide
  Description of Combined DNA/RNA Molecule Synthetic oligonucleotide

SEQ ID NO: 419 - RNA, 36 nt, Artificial Sequence; identical to SEQ ID NO: 378
  Description of Artificial Sequence Synthetic oligonucleotide

SEQ ID NO: 420 - DNA, 36 bp, Artificial Sequence; identical to SEQ ID NO: 414
  Description of Artificial Sequence Synthetic oligonucleotide
  Description of Combined DNA/RNA Molecule Synthetic oligonucleotide

SEQ ID NO: 421 - RNA, 36 nt, Artificial Sequence; identical to SEQ ID NO: 252
  Description of Artificial Sequence Synthetic oligonucleotide

SEQ ID NO: 422 - RNA, 36 nt, Artificial Sequence; identical to SEQ ID NO: 252
  Description of Artificial Sequence Synthetic oligonucleotide

SEQ ID NO: 423 - RNA, 36 nt, Artificial Sequence; identical to SEQ ID NO: 252
  Description of Artificial Sequence Synthetic oligonucleotide

SEQ ID NO: 424 - RNA, 36 nt, Artificial Sequence; identical to SEQ ID NO: 252
  Description of Artificial Sequence Synthetic oligonucleotide

SEQ ID NO: 425 - RNA, 36 nt, Artificial Sequence; identical to SEQ ID NO: 252
  Description of Artificial Sequence Synthetic oligonucleotide

SEQ ID NO: 426 - DNA, 36 bp, Artificial Sequence; identical to SEQ ID NO: 255
  Description of Artificial Sequence Synthetic oligonucleotide
  Description of Combined DNA/RNA Molecule Synthetic oligonucleotide

SEQ ID NO: 427 - DNA, 36 bp, Artificial Sequence; identical to SEQ ID NO: 255
  Description of Artificial Sequence Synthetic oligonucleotide
  Description of Combined DNA/RNA Molecule Synthetic oligonucleotide

SEQ ID NO: 428 - RNA, 36 nt, Artificial Sequence
  Description of Artificial Sequence Synthetic oligonucleotide
cccauaccuuggagcaaccgguuuuagagcuaugcu

SEQ ID NO: 429 - RNA, 20 nt, Artificial Sequence
  Description of Artificial Sequence Synthetic oligonucleotide
gagacaaaucaccugccucg

SEQ ID NO: 430 - RNA, 20 nt, Unknown
  Description of Unknown MCV1a target sequence
uuuaccguauuccacgaggc

SEQ ID NO: 431 - RNA, 20 nt, Unknown
  Description of Unknown MCV1b target sequence
cgaaguuauauuaaggguuc

SEQ ID NO: 432 - RNA, 36 nt, Artificial Sequence; identical to SEQ ID NO: 145
  Description of Artificial Sequence Synthetic oligonucleotide

SEQ ID NO: 433 - RNA, 67 nt, Artificial Sequence; identical to SEQ ID NO: 2
  Description of Artificial Sequence Synthetic oligonucleotide

SEQ ID NO: 434 - RNA, 36 nt, Artificial Sequence; identical to SEQ ID NO: 145
  Description of Artificial Sequence Synthetic oligonucleotide

SEQ ID NO: 435 - RNA, 67 nt, Artificial Sequence; identical to SEQ ID NO: 2
  Description of Artificial Sequence Synthetic oligonucleotide

(19) United States
(12) Patent Application Publication
(10) Pub. No.: US 2021/0388348 A1
(43) Pub. Date: Dec. 16, 2021

(54) MODIFIED GUIDE RNAS FOR CRISPR GENOME EDITING

(71) Applicant: UNIVERSITY OF MASSACHUSETTS, Boston, MA (US)

(72) Inventors: Erik Joseph Sontheimer, Auburndale, MA (US); Anastasia Khvorova, Westborough, MA (US); Jonathan Kenneth Watts, Worcester, MA (US); Nadia Amrani, Shrewsbury, MA (US); Zexiang Chen, Worcester, MA (US); Matthew Hassler, Boston, MA (US); Dimas Echeverria Moreno, Worcester, MA (US); Julia Frances Alterman, Boston, MA (US); Scot Wolfe, Boston, MA (US); Ken Yamada, Boston, MA (US); Gitali Devi, Boston, MA (US); Han Zhang, Boston, MA (US)

(21) Appl. No.: 17/318,846

(22) Filed: May 12, 2021

Related U.S. Application Data
(60) Provisional application No. 63/023,313, filed on May 12, 2020.

(51) Int. Cl. C12N 15/11; C12N 15/90; C12N 9/22

(52) U.S. Cl. CPC C12N 15/11; C12N 15/907; C12N 9/22; C12N 2800/80; C12N 2310/315; C12N 2310/322; C12N 2310/20

(57) ABSTRACT

Chemically modified crRNAs and tracrRNAs are provided. crRNAs and tracrRNAs with 5′ and/or 3′ conjugated moieties are provided. crRNAs and tracrRNAs with modifications in the repeat region of the crRNA or the anti-repeat region of the tracrRNA are provided. Methods of using the crRNAs and tracrRNAs for genome editing with a CRISPR nuclease and kits for performing the same are also provided.

### CROSS-REFERENCE TO RELATED APPLICATION

This application claims the benefit of U.S. Provisional Application Ser. No. 63/023,313, filed May 12, 2020, the entire disclosure of which is incorporated herein by reference.

### STATEMENT REGARDING FEDERALLY SPONSORED RESEARCH OR DEVELOPMENT

This invention was made with government support under grant no. TR002668 awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Aug. 18, 2021, is named 718188_UM9-257_ST25.txt and is 135,382 bytes in size.

### FIELD OF THE INVENTION

This disclosure relates to compositions and methods of modified guide RNAs for CRISPR genome editing.

### BACKGROUND

CRISPR RNA-guided genome engineering has revolutionized research into human genetic disease and many other aspects of biology. Numerous CRISPR-based in vivo or ex vivo genome editing therapies are nearing clinical trials. At the heart of this revolution are the microbial effector proteins found in class II CRISPR-Cas systems such as Cas9 (type II) and Cas12a/Cpf1 (type V) (Jinek et al. Science 337, 816-821 (2012); Gasiunas et al. PNAS 109, E2579-E2586 (2012); Zetsche et al. Cell 163, 759-771 (2015)).

The most widely used genome editing tool is the type II-A Cas9 from Streptococcus pyogenes strain SF370 (SpCas9) (Jinek et al, supra). Cas9 forms a ribonucleoprotein (RNP) complex with a CRISPR RNA (crRNA) and a trans-activating crRNA (tracrRNA) for efficient DNA cleavage both in bacteria and eukaryotes (FIG. 1). The crRNA contains a guide sequence that directs the Cas9 RNP to a specific locus via base-pairing with the target DNA to form an R-loop. This process requires the prior recognition of a protospacer adjacent motif (PAM), which for SpCas9 is NGG. R-loop formation activates the His-Asn-His (HNH) and RuvC-like endonuclease domains that cleave the target strand and the non-target strand of the DNA, respectively, resulting in a double-strand break (DSB).

For mammalian applications, Cas9 and its guide RNAs can be expressed from DNA (e.g. a viral vector), RNA (e.g. Cas9 mRNA plus guide RNAs in a lipid nanoparticle), or introduced as a ribonucleoprotein (RNP). Viral delivery of Cas9 results in efficient editing, but can be problematic because long-term expression of Cas9 and its guides can result in off-target editing, and viral vectors can elicit strong host immune responses (Mingozzi et al. Blood 122, 23-36 (2013)). RNA and RNP delivery platforms of Cas9 are suitable alternatives to viral vectors for many applications and have recently been shown to be effective genome editing tools in vivo (Yin et al. Nature Biotechnology 35, 1179 (2017); Lee et al. eLife 6, e25312 (2017)). RNP delivery of Cas9 also bypasses the requirement for Cas9 expression, leading to faster editing. Furthermore, Cas9 delivered as mRNA or RNP exists only transiently in cells and therefore exhibits reduced off-target editing. For instance, Cas9 RNPs were successfully used to correct hypertrophic cardiomyopathy (HCM) in human embryos without measurable off-target effects (Ma et al. Nature 548, 413 (2017).

The versatility of Cas9 for genome editing derives from its RNA-guided nature. The crRNA of SpCas9 usually includes a 20-nucleotide guide region followed by a 16-nucleotide repeat region (FIG. 1). The tracrRNA consists of an anti-repeat region that pairs with the crRNA, and also includes three stem-loops. All of these secondary structure elements are required for efficient editing in mammalian systems (Hsu et al. Nature Biotechnology 31, 827 (2013). Nevertheless, existing guide RNAs suffer from several limitations, which limit their utility in therapeutic applications. For example, existing guide RNAs may be subject to rapid degradation in circulation and within cells. Moreover, chemical modifications of guide RNAs may reduce stability and editing efficiency. Accordingly, there exists a need in the art for optimized guide RNAs that retain efficient genome editing activity in vivo and ex vivo when paired with a CRISPR nuclease, such as Cas9.

### SUMMARY

The present disclosure provides chemically modified guide RNAs for CRISPR genome editing. In certain embodiments, the guide RNAs of the disclosure are heavily or fully chemically modified. The guide RNA of the disclosure may confer several advantages in vivo or ex vivo, including stability, improved potency, and/or reduced off-target effects. Furthermore, in certain embodiments, the modified RNAs of the disclosure have reduced immunogenicity, e.g., a reduced ability to induce innate immune responses.

In certain aspects, the disclosure provides a chemically modified guide RNA comprising: (a) a crRNA portion comprising (i) a guide sequence capable of hybridizing to a target polynucleotide sequence, and (ii) a repeat sequence; and (b) a tracrRNA portion comprising an anti-repeat nucleotide sequence that is complementary to the repeat sequence, wherein the crRNA portion comprises at least 50% modified nucleotides; and wherein the crRNA portion comprises between one and ten 2′-deoxy modified ribose groups.

In an embodiment, the modified nucleotides each independently comprise a modification of a ribose group, a phosphate group, a nucleobase, or a combination thereof.

In an embodiment, each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC}).

In an embodiment, at least 80% of the ribose groups are chemically modified. In an embodiment, at least 90% of the ribose groups are chemically modified. In an embodiment, 100% of the ribose groups are chemically modified.

In an embodiment, each modification of the phosphate group is independently selected from the group consisting of a phosphorothioate, phosphonoacetate (PACE), thiophosphonoacetate (thioPACE), amide, triazole, phosphonate, and phosphotriester modification.

In an embodiment, each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

In an embodiment, the guide RNA comprises at least 90% modified nucleotide. In an embodiment, the guide RNA comprises 100% modified nucleotides.

In an embodiment, at least one nucleotide of the crRNA portion comprises each of a 2′-deoxy chemical modification and a phosphorothioate chemical modification.

In an embodiment, one or more of the nucleotides at positions 4, 5, 6, 12, 15, 16, 19, 22, 23, and 24 from the 5′ end of the crRNA portion comprise a 2′-deoxy chemical modification (e.g., one or more of the nucleotides at positions 4, 5, 6, 12, 15, 16, 19, 22, 23, and 24 from the 5′ end of the crRNA portion as set forth in SEQ ID NO: 1). In an embodiment, the nucleotides at positions 4, 5, and 6 from the 5′ end of the crRNA portion comprise each of a 2′-deoxy chemical modification and a phosphorothioate chemical modification. In an embodiment, the nucleotide at position 12 from the 5′ end of the crRNA portion comprises each of a 2′-deoxy chemical modification and a phosphorothioate chemical modification. In an embodiment, the nucleotides at positions 15, 16, and 19 from the 5′ end of the crRNA portion comprise each of a 2′-deoxy chemical modification and a phosphorothioate chemical modification. In an embodiment, the nucleotides at positions 22, 23, and 24 from the 5′ end of the crRNA portion comprise each of a 2′-deoxy chemical modification and a phosphorothioate chemical modification.

In one embodiment, the chemically modified guide RNA comprises at least one 2′-deoxy modification.

In an embodiment, the chemically modified guide RNA comprises a crRNA portion modification pattern of crRNA 38, crRNA 40, crRNA 41, crRNA 42, crRNA 44, crRNA 52, crRNA 53, crRNA 54, crRNA 55, crRNA 56, crRNA 57, crRNA 58, crRNA 59, crRNA 60, crRNA 61, crRNA 62, crRNA 63, crRNA 64, crRNA 65, crRNA 66, crRNA 67, crRNA 68, crRNA 69, crRNA 70, crRNA 71, crRNA 72, crRNA 73, crRNA 74, crRNA 75, crRNA 76, crRNA 77, crRNA 78, crRNA 79, crRNA 80, crRNA 81, crRNA 82, crRNA 83, crRNA 84, crRNA 85, crRNA 86, crRNA 87, crRNA 88, crRNA 89, crRNA 90, crRNA 91, crRNA 92, or crRNA 93, as recited in Table 1.

In an embodiment, the chemically modified guide RNA comprises a crRNA portion modification pattern selected from the group consisting of:

(SEQ ID NO: 5)
mN#mN#mN#dN#dN#dN#mNmNmNmNfNfNfNfNrN#rN#fNfNrN#mNm
GrU#rU#rU#mUmAmGmAmGmCmUmAmU#mG#mC#mU (crRNA 38);
(SEQ ID NO: 6)
mN#mN#mN#dN#dN#dN#mNmNmNmNfNfNfNINfNfNfNfNfNmNmGfU
fUfUfUfAmGmAmGmCmUmAmU#mG#mC#mU (crRNA 40);
(SEQ ID NO: 7)
mN#mN#mN#mNmNmNmNmNmNmNfNfNfNfNfdN#dN#fNfNdN#mNmGr
U#rU#rU#fUfAmGmAmGmCmUmAmU#mG#mC#mU (crRNA 41);
(SEQ ID NO: 8)
mN#mN#mN#mNmNmNmNmNmNmNfNfNfNfNfN#rN#fNfNfN#mNmGdU#
dU#dU#fUfAmGmAmGmCmUmAmU#mG#mC#mU (crRNA 42);
and
(SEQ ID NO: 9)
mN#mN#mN#mNmNmNmNmNmNmNfNdN#fNfNrN#fN#fNfNrN#mNmGr
U#rU#rU#fUfAmGmAmGmCmUmAmU#mG#mC#mU (crRNA 44),
wherein rN = RNA,mN = 2′-O-methyl RNA,
fN = 2′-fluoro RNA, dN = 2′-deoxy RNA, N#N =
phosphorothioate linkage, and N = any nucleotide.

In an embodiment, the chemically modified guide RNA comprises a tracrRNA portion modification pattern selected from any of tracrRNAs 1-116 of Table 2.

In an embodiment, the chemically modified guide RNA comprises a tracrRNA portion modification pattern selected from the group consisting of:

| Name | Sequence |
|---|---|
| tracrRNA 1 | mA#mG#mC#mAmUmAmGrCrArArGrUrUmArArArArUr |
| | ArArGmGrCrUmArGrUrCmCrGrUrUrArUrCrAmAmCmU |
| | mUmGmAmAmAmAmAmGmUrGrGrCrAmCmCmGrArGr |
| | UrCrGmGmUmGmC#mU#mU#mU (SEQ ID NO: 10) |
| tracrRNA 2 | mA#mG#mC#mAmUmAmGmCmAmAmGrUrUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 11) |
| tracrRNA 3 | mA#mG#mC#mAmUmAmGmCmAmAmGrU4U#mArA# |
| | mAmArU#mAmAmGmGrC#rU#mArG4U#rC#mCrG#rU#r |
| | U#mAmUmCmAmAmCmUmUmGmAmAmAmAmAmGm |
| | UmGmGmCmAmCmCmGmAmGmUmCmGmGmUmGmC |
| | #mU#mU#mU (SEQ ID NO: 12) |
| tracrRNA 4 | mA#mG#mC#mAmUmAmGmCmAmAmGrUrUmAmAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 13) |
| tracrRNA 5 | mA#mG#mC#mAmUmAmGmCmAmAmGrUrUmAmAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 13) |
| tracrRNA 6 | mA#mG#mC#mAmUmAmGmCmAmAmGrUrUmArAmA |
| | mAfUmAmAmGmGfCfUmArGfUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 14) |
| tracrRNA 7 | mA#mG#mC#mAmUmAmGmCmAmAmGrUfUmArAmA |
| | mAfUmAmAmGmGfCfUmAfGfUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 15) |
| tracrRNA 8 | mA#mG#mC#mAmUmAmGmCmAmAmGfUfUmAfAmA |
| | mAfUmAmAmGmGfCfUmAfGfUfCmCfGfUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 16) |
| tracrRNA 9 | mA#mG#mC#mAmUmAmGmCmAmAmGfUrUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 17) |
| tracrRNA 10 | mA#mG#mC#mAmUmAmGmCmAmAmGrUfUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 18) |
| tracrRNA 11 | mA#mG#mC#mAmUmAmGmCmAmAmGrUrUmAfAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 19) |
| tracrRNA 12 | mA#mG#mC#mAmUmAmGmCmAmAmGrUrUmArAmA |
| | mAfUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 20) |
| tracrRNA 13 | mA#mG#mC#mAmUmAmGmCmAmAmGrUrUmArAmA |
| | mArUmAmAmGmGfCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 21) |
| tracrRNA 14 | mA#mG#mC#mAmUmAmGmCmAmAmGrUrUmArAmA |
| | mArUmAmAmGmGrCfUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 22) |
| tracrRNA 15 | mA#mG#mC#mAmUmAmGmCmAmAmGrUrUmArAmA |
| | mArUmAmAmGmGrCrUmAfGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 23) |
| tracrRNA 16 | mA#mG#mC#mAmUmAmGmCmAmAmGrUrUmArAmA |
| | mArUmAmAmGmGrCrUmArGfUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 24) |
| tracrRNA 17 | mA#mG#mC#mAmUmAmGmCmAmAmGrUrUmArAmA |
| | mArUmAmAmGmGrCrUmArGrUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 25) |
| tracrRNA 18 | mA#mG#mC#mAmUmAmGmCmAmAmGrUrUmArAmA |
| | mArUmAmAmGmGrCrUmArGrUrCmCfGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 26) |
| tracrRNA 19 | mA#mG#mC#mAmUmAmGmCmAmAmGrUrUmArAmA |
| | mArUmAmAmGmGrCrUmArGrUrCmCrGfUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 27) |
| tracrRNA 20 | mA#mG#mC#mAmUmAmGmCmAmAmGrUrUmArAmA |
| | mArUmAmAmGmGrCrUmArGrUrCmCrGrUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 28) |
| tracrRNA 21 | mA#mG#mC#mAmUmAmGmCmAmAmGrU4U#mArA# |
| | mAmAfUmAmAmGmGfCfUmArG#fUfCmCrG#rU#rU#m |
| | AmUmCmAmAmCmUmUmGmAmAmAmAmAmGmUmG |
| | mGmCmAmCmCmGmAmGmUmCmGmGmUmGmC#mU# |
| | mU#mU (SEQ ID NO: 29) |
| tracrRNA 22 | mA#mG#mC#mAmUmAmGmCmAmAmGmUrUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 30) |
| tracrRNA 23 | mA#mG#mC#mAmUmAmGmCmAmAmGrUmUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 31) |
| tracrRNA 24 | mA#mG#mC#mAmUmAmGmCmAmAmGrUrUmAmAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 13) |
| tracrRNA 25 | mA#mG#mC#mAmUmAmGmCmAmAmGrUrUmArAmA |
| | mAmUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 32) |
| tracrRNA 26 | mA#mG#mC#mAmUmAmGmCmAmAmGrUrUmArAmA |
| | mArUmAmAmGmGmCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 33) |
| tracrRNA 27 | mA#mG#mC#mAmUmAmGmCmAmAmGrUrUmArAmA |
| | mArUmAmAmGmGrCmUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 34) |
| tracrRNA 28 | mA#mG#mC#mAmUmAmGmCmAmAmGrUrUmArAmA |
| | mArUmAmAmGmGrCrUmAmGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 35) |
| tracrRNA 29 | mA#mG#mC#mAmUmAmGmCmAmAmGrUrUmArAmA |
| | mArUmAmAmGmGrCrUmArGmUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 36) |
| tracrRNA 30 | mA#mG#mC#mAmUmAmGmCmAmAmGrUrUmArAmA |
| | mArUmAmAmGmGrCrUmArGrUmCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 37) |
| tracrRNA 31 | mA#mG#mC#mAmUmAmGmCmAmAmGrUrUmArAmA |
| | mArUmAmAmGmGrCrUmArGrUrCmCmGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 38) |
| tracrRNA 32 | mA#mG#mC#mAmUmAmGmCmAmAmGrUrUmArAmA |
| | mArUmAmAmGmGrCrUmArGrUrCmCrGmUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 39) |
| tracrRNA 33 | mA#mG#mC#mAmUmAmGmCmAmAmGrUrUmArAmA |
| | mArUmAmAmGmGrCrUmArGrUrCmCrGrUmUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 40) |
| tracrRNA 34 | mA#mG#mC#mAmUmAmGmCmAmAmGrU#rUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 41) |
| tracrRNA 35 | mA#mG#mC#mAmUmAmGmCmAmAmGrUrU#mArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 42) |
| tracrRNA 36 | mA#mG#mC#mAmUmAmGmCmAmAmGrUrUmArA#mA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 43) |
| tracrRNA 37 | mA#mG#mC#mAmUmAmGmCmAmAmGrUrUmArAmA |
| | mArU#mAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 44) |
| tracrRNA 38 | mA#mG#mC#mAmUmAmGmCmAmAmGrUrUmArAmA |
| | mArUmAmAmGmGrC#rUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 45) |
| tracrRNA 39 | mA#mG#mC#mAmUmAmGmCmAmAmGrUrUmArAmA |
| | mArUmAmAmGmGrCrU#mArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 46) |
| tracrRNA 40 | mA#mG#mC#mAmUmAmGmCmAmAmGrUrUmArAmA |
| | mArUmAmAmGmGrCrUmArG#rUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 47) |
| tracrRNA 41 | mA#mG#mC#mAmUmAmGmCmAmAmGrUrUmArAmA |
| | mArUmAmAmGmGrCrUmArGrU4CmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 48) |
| tracrRNA 42 | mA#mG#mC#mAmUmAmGmCmAmAmGrUrUmArAmA |
| | mArUmAmAmGmGrCrUmArGrUrC#mCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 49) |
| tracrRNA 43 | mA#mG#mC#mAmUmAmGmCmAmAmGrUrUmArAmA |
| | mArUmAmAmGmGrCrUmArGrUrCmCrG#rUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 50) |
| tracrRNA 44 | mA#mG#mC#mAmUmAmGmCmAmAmGrUrUmArAmA |
| | mArUmAmAmGmGrCrUmArGrUrCmCrGrU#rUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 51) |
| tracrRNA 45 | mA#mG#mC#mAmUmAmGmCmAmAmGrUrUmArAmA |
| | mArUmAmAmGmGrCrUmArGrUrCmCrGrUrU#mAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 52) |
| tracrRNA 46 | mA#mG#mC#mAmUmAmGmCmAmAmGfUfUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 53) |
| tracrRNA 47 | mA#mG#mC#mAmUmAmGmCmAmAmGrUrUmAfAmA |
| | mAmUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 54) |
| tracrRNA 48 | mA#mG#mC#mAmUmAmGmCmAmAmGfUfUmAfAmA |
| | mAfUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 55) |
| tracrRNA 49 | mA#mG#mC#mAmUmAmGmCmAmAmGrUrUmArAmA |
| | mArUmAmAmGmGfCfUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 56) |
| tracrRNA 50 | mA#mG#mC#mAmUmAmGmCmAmAmGrUrUmArAmA |
| | mArUmAmAmGmGrCrUmAfGfUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 57) |
| tracrRNA 51 | mA#mG#mC#mAmUmAmGmCmAmAmGrUrUmArAmA |
| | mArUmAmAmGmGrCrUmArGrUrCmCfGfUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 58) |
| tracrRNA 52 | mA#mG#mC#mAmUmAmGmCmAmAmGrUrUmArAmA |
| | mArUmAmAmGmGfCfUmArGrUrCmCfGfUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 59) |
| tracrRNA 53 | mA#mG#mC#mAmUmAmGmCmAmAmGrUrUmArAmA |
| | mArUmAmAmGmGfCfUmAfGfUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 60) |
| tracrRNA 54 | mA#mG#mC#mAmUmAmGmCmAmAmGrUrUmArAmA |
| | mArUmAmAmGmGrCrUmAfGfUfCmCfGfUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 61) |
| tracrRNA 55 | mA#mG#mC#mAmUmAmGmCmAmAmGrUrUmArAmA |
| | mArUmAmAmGmGrCrUmAfGfUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 57) |
| tracrRNA 56 | mA#mG#mC#mAmUmAmGmCmAmAmGrUrUmArAmA |
| | mArUmAmAmGmGrCrUmArGrUrCmCfGrUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 62) |
| tracrRNA 57 | mA#mG#mC#mAmUmAmGmCmAmAmGrUrUmArAmA |
| | mArUmAmAmGmGfCfUmAfGrUfCmCfGrUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 63) |
| tracrRNA 58 | mA#mG#mC#mAmUmAmGmCmAmAmGrUrUmArAmA |
| | mArUmAmAmGmGrCrUmAfGrUfCmCfGrUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 64) |
| tracrRNA 59 | mA#mG#mC#mAmUmAmGmCmAmAmGrUrUmArAmA |
| | mArUmAmAmGmGfCrUmAfGrUfCmCfGrUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 65) |
| tracrRNA 60 | mA#mG#mC#mAmUmAmGmCmAmAmGmUmUmArAm |
| | AmArUmAmAmGmGrCrUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 66) |
| | mA#mG#mC#mAmUmAmGmCmAmAmGrUrUmAmAmA |
| tracrRNA 61 | mAmUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 67) |
| tracrRNA 62 | mA#mG#mC#mAmUmAmGmCmAmAmGmUmUmAmAm |
| | AmAmUmAmAmGmGrCrUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 68) |
| tracrRNA 63 | mA#mG#mC#mAmUmAmGmCmAmAmGrUrUmArAmA |
| | mArUmAmAmGmGmCmUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 69) |
| tracrRNA 64 | mA#mG#mC#mAmUmAmGmCmAmAmGrUrUmArAmA |
| | mArUmAmAmGmGrCrUmAmGmUmCmCrGrUrUmAmU |
| | mCmAmAmCmUmUmGmAmAmAmAmAmGmUmGmGm |
| | CmAmCmCmGmAmGmUmCmGmGmUmGmC#mU#mU# |
| | mU (SEQ ID NO: 70) |
| tracrRNA 65 | mA#mG#mC#mAmUmAmGmCmAmAmGrUrUmArAmA |
| | mArUmAmAmGmGrCrUmArGrUrCmCmGmUmUmAmU |
| | mCmAmAmCmUmUmGmAmAmAmAmAmGmUmGmGm |
| | CmAmCmCmGmAmGmUmCmGmGmUmGmC#mU#mU# |
| | mU (SEQ ID NO: 71) |
| tracrRNA 66 | mA#mG#mC#mAmUmAmGmCmAmAmGrUrUmArAmA |
| | mArUmAmAmGmGmCmUmArGrUrCmCmGmUmUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 72) |
| tracrRNA 67 | mA#mG#mC#mAmUmAmGmCmAmAmGrUrUmArAmA |
| | mArUmAmAmGmGmCmUmAmGmUmCmCrGrUrUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 73) |
| tracrRNA 68 | mA#mG#mC#mAmUmAmGmCmAmAmGrUrUmArAmA |
| | mArUmAmAmGmGrCrUmAmGmUmCmCmGmUmUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 74) |
| tracrRNA 69 | mA#mG#mC#mAmUmAmGmCmAmAmGrUrUmArAmA |
| | mArUmAmAmGmGrCrUmAmGrUmCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 75) |
| tracrRNA 70 | mA#mG#mC#mAmUmAmGmCmAmAmGrUrUmArAmA |
| | mArUmAmAmGmGrCrUmArGrUrCmCmGrUmUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 76) |
| tracrRNA 71 | mA#mG#mC#mAmUmAmGmCmAmAmGrUrUmArAmA |
| | mArUmAmAmGmGmCmUmAmGrUmCmCmGrUmUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 77) |
| tracrRNA 72 | mA#mG#mC#mAmUmAmGmCmAmAmGrUrUmArAmA |
| | mArUmAmAmGmGrCrUmAmGrUmCmCmGrUmUmAmU |
| | mCmAmAmCmUmUmGmAmAmAmAmAmGmUmGmGm |
| | CmAmCmCmGmAmGmUmCmGmGmUmGmC#mU#mU# |
| | mU (SEQ ID NO: 78) |
| tracrRNA 73 | mA#mG#mC#mAmUmAmGmCmAmAmGrUrUmArAmA |
| | mArUmAmAmGmGmCrUmAmGrUmCmCmGrUmUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 79) |
| tracrRNA 74 | mA#mG#mC#mAmUmAmGmCmAmAmGdUdUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 80) |
| tracrRNA 75 | mA#mG#mC#mAmUmAmGmCmAmAmGrUrUmAdAmA |
| | mAdUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 81) |
| tracrRNA 76 | mA#mG#mC#mAmUmAmGmCmAmAmGdUdUmAdAmA |
| | mAdUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 82) |
| tracrRNA 77 | mA#mG#mC#mAmUmAmGmCmAmAmGrUrUmArAmA |
| | mArUmAmAmGmGdCdUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 83) |
| tracrRNA 78 | mA#mG#mC#mAmUmAmGmCmAmAmGrUrUmArAmA |
| | mArUmAmAmGmGrCrUmAdGdUdCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 84) |
| tracrRNA 79 | mA#mG#mC#mAmUmAmGmCmAmAmGrUrUmArAmA |
| | mArUmAmAmGmGrCrUmArGrUrCmCdGdUdUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 85) |
| tracrRNA 80 | mA#mG#mC#mAmUmAmGmCmAmAmGrUrUmArAmA |
| | mArUmAmAmGmGdCdUmArGrUrCmCdGdUdUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 86) |
| tracrRNA 81 | mA#mG#mC#mAmUmAmGmCmAmAmGrUrUmArAmA |
| | mArUmAmAmGmGdCdUmAdGdUdCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 87) |
| tracrRNA 82 | mA#mG#mC#mAmUmAmGmCmAmAmGrUrUmArAmA |
| | mArUmAmAmGmGrCrUmAdGdUdCmCdGdUdUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 88) |
| tracrRNA 83 | mA#mG#mC#mAmUmAmGmCmAmAmGrUrUmArAmA |
| | mArUmAmAmGmGrCrUmAdGrUdCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 89) |
| tracrRNA 84 | mA#mG#mC#mAmUmAmGmCmAmAmGrUrUmArAmA |
| | mArUmAmAmGmGrCrUmArGrUrCmCdGrUdUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 90) |
| tracrRNA 85 | mA#mG#mC#mAmUmAmGmCmAmAmGrUrUmArAmA |
| | mArUmAmAmGmGdCdUmAdGrUdCmCdGrUdUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 91) |
| tracrRNA 86 | mA#mG#mC#mAmUmAmGmCmAmAmGrUrUmArAmA |
| | mArUmAmAmGmGrCrUmAdGrUdCmCdGrUdUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 92) |
| tracrRNA 87 | mA#mG#mC#mAmUmAmGmCmAmAmGrUrUmArAmA |
| | mArUmAmAmGmGdCrUmAdGrUdCmCdGrUdUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 93) |
| tracrRNA 88 | mA#mG#mC#mAmUmAmGmCmAmAmGrU#rU#mArAm |
| | AmArUmAmAmGmGrCrUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 94) |
| tracrRNA 89 | mA#mG#mC#mAmUmAmGmCmAmAmGrUrUmArA#mA |
| | mArU#mAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 95) |
| tracrRNA 90 | mA#mG#mC#mAmUmAmGmCmAmAmGrU#rU#mArA# |
| | mAmArU#mAmAmGmGrCrUmArGrUrCmCrGrUrUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 96) |
| tracrRNA 91 | mA#mG#mC#mAmUmAmGmCmAmAmGrUrUmArAmA |
| | mArUmAmAmGmGrC#rU#mArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 97) |
| tracrRNA 92 | mA#mG#mC#mAmUmAmGmCmAmAmGrUrUmArAmA |
| | mArUmAmAmGmGrCrUmArG#rU#rC#mCrGrUrUmAmU |
| | mCmAmAmCmUmUmGmAmAmAmAmAmGmUmGmGm |
| | CmAmCmCmGmAmGmUmCmGmGmUmGmC#mU#mU# |
| | mU (SEQ ID NO: 98) |
| tracrRNA 93 | mA#mG#mC#mAmUmAmGmCmAmAmGrUrUmArAmA |
| | mArUmAmAmGmGrCrUmArGrUrCmCrG#rU4U#mAmU |
| | mCmAmAmCmUmUmGmAmAmAmAmAmGmUmGmGm |
| | CmAmCmCmGmAmGmUmCmGmGmUmGmC#mU#mU# |
| | mU (SEQ ID NO: 99) |
| tracrRNA 94 | mA#mG#mC#mAmUmAmGmCmAmAmGrUrUmArAmA |
| | mArUmAmAmGmGrC#rU#mArGrUrCmCrG#rUrU#mAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 100) |
| tracrRNA 95 | mA#mG#mC#mAmUmAmGmCmAmAmGrUrUmArAmA |
| | mArUmAmAmGmGrC#rU#mArG#rU#rC#mCrGrUrUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 101) |
| tracrRNA 96 | mA#mG#mC#mAmUmAmGmCmAmAmGrUrUmArAmA |
| | mArUmAmAmGmGrCrUmArG#rU#rC#mCrG#rU#rU#mA |
| | mUmCmAmAmCmUmUmGmAmAmAmAmAmGmUmGm |
| | GmCmAmCmCmGmAmGmUmCmGmGmUmGmC#mU#m |
| | U#mU (SEQ ID NO: 102) |
| tracrRNA 97 | mA#mG#mC#mAmUmAmGmCmAmAmGrUrUmArAmA |
| | mArUmAmAmGmGrCrUmArG#rUrC#mCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 103) |
| tracrRNA 98 | mA#mG#mC#mAmUmAmGmCmAmAmGrUrUmArAmA |
| | mArUmAmAmGmGrCrUmArGrUrCmCrG#rUrU#mAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 104) |
| tracrRNA 99 | mA#mG#mC#mAmUmAmGmCmAmAmGrUrUmArAmA |
| | mArUmAmAmGmGrC#rU#mArG#rU#CmCrG#rUrU#mA |
| | mUmCmAmAmCmUmUmGmAmAmAmAmAmGmUmGm |
| | GmCmAmCmCmGmAmGmUmCmGmGmUmGmC#mU#m |
| | U#mU (SEQ ID NO: 105) |
| tracrRNA | mA#mG#mC#mAmUmAmGmCmAmAmGrUrUmArAmA |
| 100 | mArUmAmAmGmGrCrUmArG#rU#rCmCrG#rUrU#mAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 106) |
| tracrRNA | mA#mG#mC#mAmUmAmGmCmAmAmGrUrUmArAmA |
| 101 | mArUmAmAmGmGrC#rUmArG#rU#rCmCrG#rUrU#mAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 107) |
| tracrRNA | mA#mG#mC#mAmUmAmGmCmAmAmGrUrUmArAmA |
| 104 | mAdUmAmAmGmGdCdUmArGdUdCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 108) |
| tracrRNA | mA#mG#mC#mAmUmAmGmCmAmAmGrUdUmArAmA |
| 105 | mAdUmAmAmGmGdCdUmAdGdUdCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 109) |
| tracrRNA | mA#mG#mC#mAmUmAmGmCmAmAmGdUdUmAdAmA |
| 106 | mAdUmAmAmGmGdCdUmAdGdUdCmCdGdUdUmAmU |
| | mCmAmAmCmUmUmGmAmAmAmAmAmGmUmGmGm |
| | CmAmCmCmGmAmGmUmCmGmGmUmGmC#mU#mU# |
| | mU (SEQ ID NO: 110) |
| tracrRNA | mA#mG#mC#mAmUmAmGmCmAmAmGsUsUmArAmA |
| 107 | mAsUmAmAmGmGrCsUmArGsUrCmCrGsUsUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 111) |
| tracrRNA | mA#mG#mC#mAmUmAmGmCmAmAmGsUsUmArAmA |
| 108 | mAsUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 112) |
| tracrRNA | mA#mG#mC#mAmUmAmGmCmAmAmGrUrUmArAmA |
| 109 | mArUmAmAmGmGrCsUmArGsUrCmCrGsUsUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 113) |
| tracrRNA | mA#mG#mC#mAmUmAmGmCmAmAmGsUrUmArAmA |
| 110 | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 114) |
| tracrRNA | mA#mG#mC#mAmUmAmGmCmAmAmGrUsUmArAmA |
| 111 | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 115) |
| tracrRNA | mA#mG#mC#mAmUmAmGmCmAmAmGrUrUmArAmA |
| 112 | mAsUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 116) |
| tracrRNA | mA#mG#mC#mAmUmAmGmCmAmAmGrUrUmArAmA |
| 113 | mArUmAmAmGmGrCsUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 117) |
| tracrRNA | mA#mG#mC#mAmUmAmGmCmAmAmGrUrUmArAmA |
| 114 | mArUmAmAmGmGrCrUmArGsUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 118) |
| tracrRNA | mA#mG#mC#mAmUmAmGmCmAmAmGrUrUmArAmA |
| 115 | mArUmAmAmGmGrCrUmArGrUrCmCrGsUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 119) |
| tracrRNA | mA#mG#mC#mAmUmAmGmCmAmAmGrUrUmArAmA |
| 116 | mArUmAmAmGmGrCrUmArGrUrCmCrGrUsUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 120) |

wherein
rN = RNA, mN = 2′-O-methyl RNA, fN = 2′-fluoro RNA, dN = 2′-deoxy RNA, aN = 2′-NH2 (2′-amino RNA), sN = 4′-thio RNA, N#N =phosphorothioate linkage, and N = any nucleotide.

In one aspect, the disclosure provides a chemically modified guide RNA comprising: (a) a crRNA portion comprising (i) a guide sequence capable of hybridizing to a target polynucleotide sequence, and (ii) a repeat sequence; and (b) a tracrRNA portion comprising an anti-repeat nucleotide sequence that is complementary to the repeat sequence, wherein the nucleotides at positions 4, 5, and 6 from the 5′ end of the crRNA portion comprise a 2′-fluoro chemical modification or a phosphorothioate chemical modification.

In an embodiment, the chemically modified guide RNA comprises one or more additional chemical modifications, selected from a modification of a ribose group, a phosphate group, a nucleobase, or a combination thereof.

In an embodiment, each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, or a 2′-O,4′-C-aminomethylene bridged nucleic acid (2′,4′-BNA^{NC}).

In an embodiment, at least 80% of the ribose groups are chemically modified. In an embodiment, at least 90% of the ribose groups are chemically modified. In an embodiment, 100% of the ribose groups are chemically modified.

In an embodiment, each modification of the phosphate group is independently selected from the group consisting of a phosphorothioate, phosphonoacetate (PACE), thiophosphonoacetate (thioPACE), amide, triazole, phosphonate, and phosphotriester modification.

In an embodiment, each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

In an embodiment, the guide RNA comprises at least 90% modified nucleotide. In an embodiment, the guide RNA comprises 100% modified nucleotides.

In an embodiment, the nucleotides at positions 4, 5, and 6 from the 5′ end of the crRNA portion comprise a 2′-fluoro chemical modification.

In an embodiment, the chemically modified guide RNA further comprises a 2′-fluoro chemical modification at one or more of positions 15, 16, 19, 22, 23, and 24 from the 5′ end of the crRNA portion (e.g., one or more of positions 15, 16, 19, 22, 23, and 24 from the 5′ end of the crRNA portion as set forth in SEQ ID NO: 1). In an embodiment, the chemically modified guide RNA further comprises a 2′-fluoro chemical modification at positions 15, 16, 19, 22, 23, and 24 from the 5′ end of the crRNA portion.

In an embodiment, the nucleotides at positions 4, 5, and 6 from the 5′ end of the crRNA portion comprise a phosphorothioate chemical modification.

In an embodiment, the chemically modified guide RNA further comprises a 2′-fluoro chemical modification at one or more of positions 15, 16, 19, 22, 23, and 24 from the 5′ end of the crRNA portion. In an embodiment, the chemically modified guide RNA further comprises a 2′-fluoro chemical modification at positions 15, 16, 19, 22, 23, and 24 from the 5′ end of the crRNA portion.

In an embodiment, the chemically modified guide RNA comprises a crRNA portion modification pattern selected from the group consisting of:

(SEQ ID NO: 121)
mN#mN#mN#rN#rN#rN#mNmNmNmNrN#rN#rN#rN#rN#rN#rN#rN
#rN#mNmGrU#rU#rU#rU#rA#mGmAmGmCmUmAmU#mG#mC#mU
(crRNA 33);
(SEQ ID NO: 122)
mN#mN#mN#rNrN#rN#mNmNmNmNrN#rN#rN#rN#rN#rN#rN#rN#
rN#mNmGrUrUrUrUrAmGmAmGmCmUmAmU#mG#mC#mU (crRNA
34);
(SEQ ID NO: 123)
mN#mN#mN#rN#rN#rN#mNmNmNmNrN#rN#rN#rN#rN#rN#rN#
rN#rN#mNmGrUrUrUmUmAmGmAmGmCmUmAmU#mG#mC#mU (crRNA
36);
(SEQ ID NO: 124)
mN#mN#mN#rN#rN#rN#mNmNmNmNfNfNfNfNrN#rN#fNfNrN#
mNmGrU#U#rU#mUmAmGmAmGmCmUmAmU#mG#mC#mU (crRNA 37);
(SEQ ID NO: 125)
mN#mN#mN#rN#rN#rN#mNmNmNmNfNfNfNfNfNfNfNfNfNmNm
GfUfUfUfUfAmGmAmGmCmUmAmU#mG#mC#mU (crRNA 39);
and
(SEQ ID NO: 126)
mN#mN#mN#fNfNfNmNmNmNmNfNfNfNfNfNfNfNfNfNmNmGfU
fUfUfUfAmGmAmGmCmUmAmU#mG#mC#mU (crRNA 45),
wherein
rN = RNA, mN = 2′-O-methyl RNA, fN = 2′-fluoro RNA,
dN = 2′-deoxy RNA, N#N = phosphorothioate linkage,
and
N = any nucleotide.

In an embodiment, the chemically modified guide RNA comprises a tracrRNA portion modification pattern selected from any one of tracrRNAs 1-116 of Table 2.

In an embodiment, the chemically modified guide RNA comprises a tracrRNA portion modification pattern selected from the group consisting of:

| Name | Sequence |
|---|---|
| tracrRNA 1 | mA#mG#mC#mAmUmAmGrCrArArGrUrUmArArArArUr |
| | ArArGmGrCrUmArGrUrCmCrGrUrUrArUrCrAmAmCm |
| | UmUmGmAmAmAmAmAmGmUrGrGrCrAmCmCmGrArGr |
| | UrCrGmGmUmGmC#mU#mU#mU (SEQ ID NO: 10) |
| tracrRNA 2 | mA#mG#mC#mAmUmAmGmCmAmAmGrUrUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 11) |
| tracrRNA 3 | mA#mG#mC#mAmUmAmGmCmAmAmGrU#rU#mArA# |
| | mAmArU#mAmAmGmGrC#rU#mArG#rU#rC#mCrG#rU |
| | U#r#mAmUmCmAmAmCmUmUmGmAmAmAmAmAmGm |
| | UmGmGmCmAmCmCmGmAmGmUmCmGmGmUmGmC |
| | #mU#mU#mU (SEQ ID NO: 12) |
| tracrRNA 4 | mA#mG#mC#mAmUmAmGmCmAmAmGrUrUmAmAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 13) |
| tracrRNA 5 | mA#mG#mC#mAmUmAmGmCmAmAmGrUrUmAmAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU- |
| | TegChol (SEQ ID NO: 127) |
| tracrRNA 6 | mA#mG#mC#mAmUmAmGmCmAmAmGrUrUmArAmA |
| | mAfUmAmAmGmGfCfUmArGfUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 14) |
| tracrRNA 7 | mA#mG#mC#mAmUmAmGmCmAmAmGrUfUmArAmA |
| | mAfUmAmAmGmGfCfUmAfGfUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 15) |
| tracrRNA 8 | mA#mG#mC#mAmUmAmGmCmAmAmGfUfUmAfAmA |
| | mAfUmAmAmGmGfCfUmAfGfUfCmCfGfUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 16) |
| tracrRNA 9 | mA#mG#mC#mAmUmAmGmCmAmAmGfUrUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 17) |
| tracrRNA 10 | mA#mG#mC#mAmUmAmGmCmAmAmGrUfUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 18) |
| tracrRNA 11 | mA#mG#mC#mAmUmAmGmCmAmAmGrUrUmAfAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 19) |
| tracrRNA 12 | mA#mG#mC#mAmUmAmGmCmAmAmGrUrUmArAmA |
| | mAfUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 20) |
| tracrRNA 13 | mA#mG#mC#mAmUmAmGmCmAmAmGrUrUmArAmA |
| | mArUmAmAmGmGfCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 21) |
| tracrRNA 14 | mA#mG#mC#mAmUmAmGmCmAmAmGrUrUmArAmA |
| | mArUmAmAmGmGrCfUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 22) |
| tracrRNA 15 | mA#mG#mC#mAmUmAmGmCmAmAmGrUrUmArAmA |
| | mArUmAmAmGmGrCrUmAfGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 23) |
| tracrRNA 16 | mA#mG#mC#mAmUmAmGmCmAmAmGrUrUmArAmA |
| | mArUmAmAmGmGrCrUmArGfUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 24) |
| tracrRNA 17 | mA#mG#mC#mAmUmAmGmCmAmAmGrUrUmArAmA |
| | mArUmAmAmGmGrCrUmArGrUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 25) |
| tracrRNA 18 | mA#mG#mC#mAmUmAmGmCmAmAmGrUrUmArAmA |
| | mArUmAmAmGmGrCrUmArGrUrCmCfGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 26) |
| tracrRNA 19 | mA#mG#mC#mAmUmAmGmCmAmAmGrUrUmArAmA |
| | mArUmAmAmGmGrCrUmArGrUrCmCrGfUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 27) |
| tracrRNA 20 | mA#mG#mC#mAmUmAmGmCmAmAmGrUrUmArAmA |
| | mArUmAmAmGmGrCrUmArGrUrCmCrGrUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 28) |
| tracrRNA 21 | mA#mG#mC#mAmUmAmGmCmAmAmGrU#rU#mArA# |
| | mAmAfUmAmAmGmGfCfUmArG#fUfCmCrG#rU#rU#m |
| | AmUmCmAmAmCmUmUmGmAmAmAmAmAmGmUmG |
| | mGmCmAmCmCmGmAmGmUmCmGmGmUmGmC#mU#mU |
| | #mU (SEQ ID NO: 29) |
| tracrRNA 22 | mA#mG#mC#mAmUmAmGmCmAmAmGmUrUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 30) |
| tracrRNA 23 | mA#mG#mC#mAmUmAmGmCmAmAmGrUmUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 31) |
| tracrRNA 24 | mA#mG#mC#mAmUmAmGmCmAmAmGrUrUmAmAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 13) |
| tracrRNA 25 | mA#mG#mC#mAmUmAmGmCmAmAmGrUrUmArAmA |
| | mAmUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 32) |
| tracrRNA 26 | mA#mG#mC#mAmUmAmGmCmAmAmGrUrUmArAmA |
| | mArUmAmAmGmGmCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 33) |
| tracrRNA 27 | mA#mG#mC#mAmUmAmGmCmAmAmGrUrUmArAmA |
| | mArUmAmAmGmGrCmUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 34) |
| tracrRNA 28 | mA#mG#mC#mAmUmAmGmCmAmAmGrUrUmArAmA |
| | mArUmAmAmGmGrCrUmAmGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 35) |
| tracrRNA 29 | mA#mG#mC#mAmUmAmGmCmAmAmGrUrUmArAmA |
| | mArUmAmAmGmGrCrUmArGmUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 36) |
| tracrRNA 30 | mA#mG#mC#mAmUmAmGmCmAmAmGrUrUmArAmA |
| | mArUmAmAmGmGrCrUmArGrUmCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 37) |
| tracrRNA 31 | mA#mG#mC#mAmUmAmGmCmAmAmGrUrUmArAmA |
| | mArUmAmAmGmGrCrUmArGrUrCmCmGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 38) |
| tracrRNA 32 | mA#mG#mC#mAmUmAmGmCmAmAmGrUrUmArAmA |
| | mArUmAmAmGmGrCrUmArGrUrCmCrGmUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 39) |
| tracrRNA 33 | mA#mG#mC#mAmUmAmGmCmAmAmGrUrUmArAmA |
| | mArUmAmAmGmGrCrUmArGrUrCmCrGrUmUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 40) |
| tracrRNA 34 | mA#mG#mC#mAmUmAmGmCmAmAmGrU#rUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 41) |
| tracrRNA 35 | mA#mG#mC#mAmUmAmGmCmAmAmGrUrU#mArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 42) |
| tracrRNA 36 | mA#mG#mC#mAmUmAmGmCmAmAmGrUrUmArA#mA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 43) |
| tracrRNA 37 | mA#mG#mC#mAmUmAmGmCmAmAmGrUrUmArAmA |
| | mArU#mAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 44) |
| tracrRNA 38 | mA#mG#mC#mAmUmAmGmCmAmAmGrUrUmArAmA |
| | mArUmAmAmGmGrC#rUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 45) |
| tracrRNA 39 | mA#mG#mC#mAmUmAmGmCmAmAmGrUrUmArAmA |
| | mArUmAmAmGmGrCrU#mArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 46) |
| tracrRNA 40 | mA#mG#mC#mAmUmAmGmCmAmAmGrUrUmArAmA |
| | mArUmAmAmGmGrCrUmArG#rUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 47) |
| tracrRNA 41 | mA#mG#mC#mAmUmAmGmCmAmAmGrUrUmArAmA |
| | mArUmAmAmGmGrCrUmArGrU#rCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 48) |
| tracrRNA 42 | mA#mG#mC#mAmUmAmGmCmAmAmGrUrUmArAmA |
| | mArUmAmAmGmGrCrUmArGrUrC#mCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 49) |
| tracrRNA 43 | mA#mG#mC#mAmUmAmGmCmAmAmGrUrUmArAmA |
| | mArUmAmAmGmGrCrUmArGrUrCmCrG#rUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 50) |
| tracrRNA 44 | mA#mG#mC#mAmUmAmGmCmAmAmGrUrUmArAmA |
| | mArUmAmAmGmGrCrUmArGrUrCmCrGrU#rUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 51) |
| tracrRNA 45 | mA#mG#mC#mAmUmAmGmCmAmAmGrUrUmArAmA |
| | mArUmAmAmGmGrCrUmArGrUrCmCrGrUrU#mAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 52) |
| tracrRNA 46 | mA#mG#mC#mAmUmAmGmCmAmAmGfUfUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 53) |
| tracrRNA 47 | mA#mG#mC#mAmUmAmGmCmAmAmGrUrUmAfAmA |
| | mAmUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 54) |
| tracrRNA 48 | mA#mG#mC#mAmUmAmGmCmAmAmGfUfUmAfAmA |
| | mAfUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 55) |
| tracrRNA 49 | mA#mG#mC#mAmUmAmGmCmAmAmGrUrUmArAmA |
| | mArUmAmAmGmGfCfUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 56) |
| tracrRNA 50 | mA#mG#mC#mAmUmAmGmCmAmAmGrUrUmArAmA |
| | mArUmAmAmGmGrCrUmAfGfUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 57) |
| tracrRNA 51 | mA#mG#mC#mAmUmAmGmCmAmAmGrUrUmArAmA |
| | mArUmAmAmGmGrCrUmArGrUrCmCfGfUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 58) |
| tracrRNA 52 | mA#mG#mC#mAmUmAmGmCmAmAmGrUrUmArAmA |
| | mArUmAmAmGmGfCfUmArGrUrCmCfGfUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 59) |
| tracrRNA 53 | mA#mG#mC#mAmUmAmGmCmAmAmGrUrUmArAmA |
| | mArUmAmAmGmGfCfUmAfGfUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 60) |
| tracrRNA 54 | mA#mG#mC#mAmUmAmGmCmAmAmGrUrUmArAmA |
| | mArUmAmAmGmGrCrUmAfGfUfCmCfGfUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 61) |
| tracrRNA 55 | mA#mG#mC#mAmUmAmGmCmAmAmGrUrUmArAmA |
| | mArUmAmAmGmGrCrUmAfGfUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 57) |
| tracrRNA 56 | mA#mG#mC#mAmUmAmGmCmAmAmGrUrUmArAmA |
| | mArUmAmAmGmGrCrUmArGrUrCmCfGrUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 62) |
| tracrRNA 57 | mA#mG#mC#mAmUmAmGmCmAmAmGrUrUmArAmA |
| | mArUmAmAmGmGfCfUmAfGrUfCmCfGrUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 63) |
| tracrRNA 58 | mA#mG#mC#mAmUmAmGmCmAmAmGrUrUmArAmA |
| | mArUmAmAmGmGrCrUmAfGrUfCmCfGrUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 64) |
| tracrRNA 59 | mA#mG#mC#mAmUmAmGmCmAmAmGrUrUmArAmA |
| | mArUmAmAmGmGfCrUmAfGrUfCmCfGrUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 65) |
| tracrRNA 60 | mA#mG#mC#mAmUmAmGmCmAmAmGmUmUmArAm |
| | AmArUmAmAmGmGrCrUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 66) |
| tracrRNA 61 | mA#mG#mC#mAmUmAmGmCmAmAmGrUrUmAmAmA |
| | mAmUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 67) |
| tracrRNA 62 | mA#mG#mC#mAmUmAmGmCmAmAmGmUmUmAmAm |
| | AmAmUmAmAmGmGrCrUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#mU |
| | (SEQ ID NO: 68) |
| tracrRNA 63 | mA#mG#mC#mAmUmAmGmCmAmAmGrUrUmArAmA |
| | mArUmAmAmGmGmCmUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#mU |
| | (SEQ ID NO: 69) |
| tracrRNA 64 | mA#mG#mC#mAmUmAmGmCmAmAmGrUrUmArAmA |
| | mArUmAmAmGmGrCrUmAmGmUmCmCrGrUrUmAmU |
| | mCmAmAmCmUmUmGmAmAmAmAmAmGmUmGmGm |
| | CmAmCmCmGmAmGmUmCmGmGmUmGmC#mU#mU#mU |
| | (SEQ ID NO: 70) |
| tracrRNA 65 | mA#mG#mC#mAmUmAmGmCmAmAmGrUrUmArAmA |
| | mArUmAmAmGmGrCrUmArGrUrCmCmGmUmUmAmU |
| | mCmAmAmCmUmUmGmAmAmAmAmAmGmUmGmGm |
| | CmAmCmCmGmAmGmUmCmGmGmUmGmC#mU#mU#mU |
| | (SEQ ID NO: 71) |
| tracrRNA 66 | mA#mG#mC#mAmUmAmGmCmAmAmGrUrUmArAmA |
| | mArUmAmAmGmGmCmUmArGrUrCmCmGmUmUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 72) |
| tracrRNA 67 | mA#mG#mC#mAmUmAmGmCmAmAmGrUrUmArAmA |
| | mArUmAmAmGmGmCmUmAmGmUmCmCrGrUrUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 73) |
| tracrRNA 68 | mA#mG#mC#mAmUmAmGmCmAmAmGrUrUmArAmA |
| | mArUmAmAmGmGrCrUmAmGmUmCmCmGmUmUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 74) |
| tracrRNA 69 | mA#mG#mC#mAmUmAmGmCmAmAmGrUrUmArAmA |
| | mArUmAmAmGmGrCrUmAmGrUmCmCrGrUrUmAmUm |
| | CmAmAmCmUmUmGmAmAmAmAmAmGmUmGmGmC |
| | mAmCmCmGmAmGmUmCmGmGmUmGmC#mU#mU#mU |
| | (SEQ ID NO: 75) |
| tracrRNA 70 | mA#mG#mC#mAmUmAmGmCmAmAmGrUrUmArAmA |
| | mArUmAmAmGmGrCrUmArGrUrCmCmGrUmUmAmUm |
| | CmAmAmCmUmUmGmAmAmAmAmAmGmUmGmGmC |
| | mAmCmCmGmAmGmUmCmGmGmUmGmC#mU#mU#mU |
| | (SEQ ID NO: 76) |
| tracrRNA 71 | mA#mG#mC#mAmUmAmGmCmAmAmGrUrUmArAmA |
| | mArUmAmAmGmGmCmUmAmGrUmCmCmGrUmUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 77) |
| tracrRNA 72 | mA#mG#mC#mAmUmAmGmCmAmAmGrUrUmArAmA |
| | mArUmAmAmGmGrCrUmAmGrUmCmCmGrUmUmAmU |
| | mCmAmAmCmUmUmGmAmAmAmAmAmGmUmGmGm |
| | CmAmCmCmGmAmGmUmCmGmGmUmGmC#mU#mU#mU |
| | (SEQ ID NO: 78) |
| tracrRNA 73 | mA#mG#mC#mAmUmAmGmCmAmAmGrUrUmArAmA |
| | mArUmAmAmGmGmCrUmAmGrUmCmCmGrUmUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 79) |
| tracrRNA 74 | mA#mG#mC#mAmUmAmGmCmAmAmGdUdUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 80) |
| tracrRNA 75 | mA#mG#mC#mAmUmAmGmCmAmAmGrUrUmAdAmA |
| | mAdUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 81) |
| tracrRNA 76 | mA#mG#mC#mAmUmAmGmCmAmAmGdUdUmAdAmA |
| | mAdUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 82) |
| tracrRNA 77 | mA#mG#mC#mAmUmAmGmCmAmAmGrUrUmArAmA |
| | mArUmAmAmGmGdCdUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 83) |
| tracrRNA 78 | mA#mG#mC#mAmUmAmGmCmAmAmGrUrUmArAmA |
| | mArUmAmAmGmGrCrUmAdGdUdCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 84) |
| tracrRNA 79 | mA#mG#mC#mAmUmAmGmCmAmAmGrUrUmArAmA |
| | mArUmAmAmGmGrCrUmArGrUrCmCdGdUdUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 85) |
| tracrRNA 80 | mA#mG#mC#mAmUmAmGmCmAmAmGrUrUmArAmA |
| | mArUmAmAmGmGdCdUmArGrUrCmCdGdUdUmAmUm |
| | CmAmAmCmUmUmGmAmAmAmAmAmGmUmGmGmC |
| | mAmCmCmGmAmGmUmCmGmGmUmGmC#mU#mU#mU |
| | (SEQ ID NO: 86) |
| tracrRNA 81 | mA#mG#mC#mAmUmAmGmCmAmAmGrUrUmArAmA |
| | mArUmAmAmGmGdCdUmAdGdUdCmCrGrUrUmAmUm |
| | CmAmAmCmUmUmGmAmAmAmAmAmGmUmGmGmC |
| | mAmCmCmGmAmGmUmCmGmGmUmGmC#mU#mU#mU |
| | (SEQ ID NO: 87) |
| tracrRNA 82 | mA#mG#mC#mAmUmAmGmCmAmAmGrUrUmArAmA |
| | mArUmAmAmGmGrCrUmAdGdUdCmCdGdUdUmAmUm |
| | CmAmAmCmUmUmGmAmAmAmAmAmGmUmGmGmC |
| | mAmCmCmGmAmGmUmCmGmGmUmGmC#mU#mU#mU |
| | (SEQ ID NO: 88) |
| tracrRNA 83 | mA#mG#mC#mAmUmAmGmCmAmAmGrUrUmArAmA |
| | mArUmAmAmGmGrCrUmAdGrUdCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 89) |
| tracrRNA 84 | mA#mG#mC#mAmUmAmGmCmAmAmGrUrUmArAmA |
| | mArUmAmAmGmGrCrUmArGrUrCmCdGrUdUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 90) |
| tracrRNA 85 | mA#mG#mC#mAmUmAmGmCmAmAmGrUrUmArAmA |
| | mArUmAmAmGmGdCdUmAdGrUdCmCdGrUdUmAmUm |
| | CmAmAmCmUmUmGmAmAmAmAmAmGmUmGmGmC |
| | mAmCmCmGmAmGmUmCmGmGmUmGmC#mU#mU#mU |
| | (SEQ ID NO: 91) |
| tracrRNA 86 | mA#mG#mC#mAmUmAmGmCmAmAmGrUrUmArAmA |
| | mArUmAmAmGmGrCrUmAdGrUdCmCdGrUdUmAmUm |
| | CmAmAmCmUmUmGmAmAmAmAmAmGmUmGmGmC |
| | mAmCmCmGmAmGmUmCmGmGmUmGmC#mU#mU#mU |
| | (SEQ ID NO: 92) |
| tracrRNA 87 | mA#mG#mC#mAmUmAmGmCmAmAmGrUrUmArAmA |
| | mArUmAmAmGmGdCrUmAdGrUdCmCdGrUdUmAmUm |
| | CmAmAmCmUmUmGmAmAmAmAmAmGmUmGmGmC |
| | mAmCmCmGmAmGmUmCmGmGmUmGmC#mU#mU#mU |
| | (SEQ ID NO: 93) |
| tracrRNA 88 | mA#mG#mC#mAmUmAmGmCmAmAmGrU#rU#mArAm |
| | AmArUmAmAmGmGrCrUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#mU |
| | (SEQ ID NO: 94) |
| tracrRNA 89 | mA#mG#mC#mAmUmAmGmCmAmAmGrUrUmArA#mA |
| | mArU#mAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 95) |
| tracrRNA 90 | mA#mG#mC#mAmUmAmGmCmAmAmGrU#rU#mArA# |
| | mAmArU#mAmAmGmGrCrUmArGrUrCmCrGrUrUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 96) |
| tracrRNA 91 | mA#mG#mC#mAmUmAmGmCmAmAmGrUrUmArAmA |
| | mArUmAmAmGmGrC#rU#mArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#mU |
| | (SEQ ID NO: 97) |
| tracrRNA 92 | mA#mG#mC#mAmUmAmGmCmAmAmGrUrUmArAmA |
| | mArUmAmAmGmGrCrUmArG#rU#rC#mCrGrUrUmAmU |
| | mCmAmAmCmUmUmGmAmAmAmAmAmGmUmGmGm |
| | CmAmCmCmGmAmGmUmCmGmGmUmGmC#mU#mU |
| | #mU (SEQ ID NO: 98) |
| tracrRNA 93 | mA#mG#mC#mAmUmAmGmCmAmAmGrUrUmArAmA |
| | mArUmAmAmGmGrCrUmArGrUrCmCrG#rU#rU#mAmU |
| | mCmAmAmCmUmUmGmAmAmAmAmAmGmUmGmGm |
| | CmAmCmCmGmAmGmUmCmGmGmUmGmC#mU#mU |
| | #mU (SEQ ID NO: 99) |
| tracrRNA 94 | mA#mG#mC#mAmUmAmGmCmAmAmGrUrUmArAmA |
| | mArUmAmAmGmGrC#rU#mArGrUrCmCrG#rU#rU#mAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 100) |
| tracrRNA 95 | mA#mG#mC#mAmUmAmGmCmAmAmGrUrUmArAmA |
| | mArUmAmAmGmGrC#rU#mArG#rU#rC#mCrGrUrUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 101) |
| tracrRNA 96 | mA#mG#mC#mAmUmAmGmCmAmAmGrUrUmArAmA |
| | mArUmAmAmGmGrCrUmArG#rU#rC#mCrG#rU#rU#mA |
| | mUmCmAmAmCmUmUmGmAmAmAmAmAmGmUmGm |
| | GmCmAmCmCmGmAmGmUmCmGmGmUmGmC#mU#mU |
| | #mU (SEQ ID NO: 102) |
| tracrRNA 97 | mA#mG#mC#mAmUmAmGmCmAmAmGrUrUmArAmA |
| | mArUmAmAmGmGrCrUmArG#rUrC#mCrGrUrUmAmUm |
| | CmAmAmCmUmUmGmAmAmAmAmAmGmUmGmGmC |
| | mAmCmCmGmAmGmUmCmGmGmUmGmC#mU#mU#mU |
| | (SEQ ID NO: 103) |
| tracrRNA 98 | mA#mG#mC#mAmUmAmGmCmAmAmGrUrUmArAmA |
| | mArUmAmAmGmGrCrUmArGrUrCmCrG#rUrU#mAmUm |
| | CmAmAmCmUmUmGmAmAmAmAmAmGmUmGmGmC |
| | mAmCmCmGmAmGmUmCmGmGmUmGmC#mU#mU#mU |
| | (SEQ ID NO: 104) |
| tracrRNA 99 | mA#mG#mC#mAmUmAmGmCmAmAmGrUrUmArAmA |
| | mArUmAmAmGmGrC#rU#mArG#rU#rCmCrG#rUrU#mA |
| | mUmCmAmAmCmUmUmGmAmAmAmAmAmGmUmGm |
| | GmCmAmCmCmGmAmGmUmCmGmGmUmGmC#mU#mU |
| | #mU (SEQ ID NO: 105) |
| tracrRNA | mA#mG#mC#mAmUmAmGmCmAmAmGrUrUmArAmA |
| 100 | mArUmAmAmGmGrCrUmArG#rU#rCmCrG#rUrU#mAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 106) |
| tracrRNA | mA#mG#mC#mAmUmAmGmCmAmAmGrUrUmArAmA |
| 101 | mArUmAmAmGmGrC#rUmArG#rU#rCmCrG#rUrU#mAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 107) |
| tracrRNA | mA#mG#mC#mAmUmAmGmCmAmAmGrUrUmArAmA |
| 104 | mAdUmAmAmGmGdCdUmArGdUdCmCrGrUrUmAmUm |
| | CmAmAmCmUmUmGmAmAmAmAmAmGmUmGmGmC |
| | mAmCmCmGmAmGmUmCmGmGmUmGmC#mU#mU#mU |
| | (SEQ ID NO: 108) |
| tracrRNA | mA#mG#mC#mAmUmAmGmCmAmAmGrUdUmArAmA |
| 105 | mAdUmAmAmGmGdCdUmAdGdUdCmCrGrUrUmAmUm |
| | CmAmAmCmUmUmGmAmAmAmAmAmGmUmGmGmC |
| | mAmCmCmGmAmGmUmCmGmGmUmGmC#mU#mU#mU |
| | (SEQ ID NO: 109) |
| tracrRNA | mA#mG#mC#mAmUmAmGmCmAmAmGdUdUmAdAmA |
| 106 | mAdUmAmAmGmGdCdUmAdGdUdCmCdGdUdUmAmU |
| | mCmAmAmCmUmUmGmAmAmAmAmAmGmUmGmGm |
| | CmAmCmCmGmAmGmUmCmGmGmUmGmC#mU#mU |
| | #mU (SEQ ID NO: 110) |
| tracrRNA | mA#mG#mC#mAmUmAmGmCmAmAmGsUsUmArAmA |
| 107 | mAsUmAmAmGmGrCsUmArGsUrCmCrGsUsUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 111) |
| tracrRNA | mA#mG#mC#mAmUmAmGmCmAmAmGsUsUmArAmA |
| 108 | mAsUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 112) |
| tracrRNA | mA#mG#mC#mAmUmAmGmCmAmAmGrUrUmArAmA |
| 109 | mArUmAmAmGmGrCsUmArGsUrCmCrGsUsUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 113) |
| tracrRNA | mA#mG#mC#mAmUmAmGmCmAmAmGsUrUmArAmA |
| 110 | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 114) |
| tracrRNA | mA#mG#mC#mAmUmAmGmCmAmAmGrUsUmArAmA |
| 111 | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 115) |
| tracrRNA | mA#mG#mC#mAmUmAmGmCmAmAmGrUrUmArAmA |
| 112 | mAsUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 116) |
| tracrRNA | mA#mG#mC#mAmUmAmGmCmAmAmGrUrUmArAmA |
| 113 | mArUmAmAmGmGrCsUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 117) |
| tracrRNA | mA#mG#mC#mAmUmAmGmCmAmAmGrUrUmArAmA |
| 114 | mArUmAmAmGmGrCrUmArGsUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 118) |
| tracrRNA | mA#mG#mC#mAmUmAmGmCmAmAmGrUrUmArAmA |
| 115 | mArUmAmAmGmGrCrUmArGrUrCmCrGsUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 119) |
| tracrRNA | mA#mG#mC#mAmUmAmGmCmAmAmGrUrUmArAmA |
| 116 | mArUmAmAmGmGrCrUmArGrUrCmCrGrUsUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 120) |

wherein rN = RNA, mN = 2′-O-methyl RNA, fN = 2′-fluoro RNA,
dN = 2′-deoxy RNA, sN = 4′-thio RNA, N#N = phosphorothioate linkage, and N = any nucleotide.

In another aspect, the disclosure provides a chemically modified guide RNA comprising: (a) a crRNA portion comprising (i) a guide sequence capable of hybridizing to a target polynucleotide sequence, and (ii) a repeat sequence; and (b) a tracrRNA portion comprising an anti-repeat nucleotide sequence that is complementary to the repeat sequence, wherein the crRNA portion comprises a modification pattern selected from the group consisting of:

(SEQ ID NO: 128)
mN#mN#mN#mNmNmNmNmNmNmNfNfNfNfNmNrN#fNfNrN#mNmGrU#rU#rU
#fUfAmGmAmGmCmUmAmU#mG#mC#mU (crRNA 23);
(SEQ ID NO: 129)
mN#mN#mN#mNmNmNmNmNmNmNfNfNfNfNrN#fNfNfNrN#mNmGrU#rU#rU
#fUfAmGmAmGmCmUmAmU#mG#mC#mU (crRNA 24);
(SEQ ID NO: 130)
mN#mN#mN#mNmNmNmNmNmNmNfNfNfNfNrN#rN#fNfNfNmNmGrU#rU#rU
#fUfAmGmAmGmCmUmAmU#mG#mC#mU (crRNA 25);
(SEQ ID NO: 131)
mN#mN#mN#mNmNmNmNmNmNmNfNfNfNfNrN#rN#fNfNrN#mNmGfUrU#rU
#fUfAmGmAmGmCmUmAmU#mG#mC#mU (crRNA 26);
(SEQ ID NO: 132)
mN#mN#mN#mNmNmNmNmNmNmNfNfNfNfNrN#rN#fNfNrN#mNmGrU#fUrU
#fUfAmGmAmGmCmUmAmU#mG#mC#mU (crRNA 27);
(SEQ ID NO: 133)
mN#mN#mN#mNmNmNmNmNmNmNfNfNfNfNrN#rN#fNfNrN#mNmGrU#rU#r
UfUfAmGmAmGmCmUmAmU#mG#mC#mU (crRNA 28);
(SEQ ID NO: 134)
mN#mN#mN#mNmNmNmNmNmNmNfNfNfNfNrN#fNfNfNfNmNmGfUfUfUfUf
AmGmAmGmCmUmAmU#mG#mC#mU (crRNA 29);
(SEQ ID NO: 135)
mN#mN#mN#rNrNrNmNmNmNmNrNrNrNrNrNrNrNrNrNmNmGrUrUrUrUrAmG
mAmGmCmUmAmU#mG#mC#mU (crRNA 30);
(SEQ ID NO: 136)
mN#mN#mN#rNrNrNmNmNmNmNmNrNrNrNrNrNrNrNrNmNmGrUrUrUrUrAm
GmAmGmCmUmAmU#mG#mC#mU (crRNA 31);
(SEQ ID NO: 137)
mN#mN#mN#rNrNrNmNmNmNmNmNrNmNmNrNrNrNrNrNmNmGrUrUrUrUrA
mGmAmGmCmUmAmU#mG#mC#mU (crRNA 32);
(SEQ ID NO: 138)
mN#mN#mN#rNrNrNmNmNmNmNrNrNrNrNrNrNrNrNrNmNmGrUrUrUmUmAm
GmAmGmCmUmAmU#mG#mC#mU (crRNA 35);
(SEQ ID NO: 139)
mN#mN#mN#mNmNmNmNmNmNmNfNrN#fNfNrN#rN#fNfNrN#mNmGrU#rU#r
UffUfAmGmAmGmCmUmAmU#mG#mC#mU (crRNA 43);
(SEQ ID NO: 140)
mN#mN#mN#mNmNmNmNmNmNmNfNfNfNfNmNrN#fNfNrN#mNmGrU#rUrU#
fUfAmGmAmGmCmUmAmU#mG#mC#mU (crRNA 46);
(SEQ ID NO: 141)
mN#mN#mN#mNmNmNmNmNmNmNfNfNfNfNrN#mNfNfNrN#mNmGrU#rUrU#
fUfAmGmAmGmCmUmAmU#mG#mC#mU (crRNA 47);
(SEQ ID NO: 142 )
mN#mN#mN#mNmNmNmNmNmNmNfNfNfNfNrN#mNfNfNmNmNmGrU#rUrU#
fUfAmGmAmGmCmUmAmU#mG#mC#mU (crRNA 48);
(SEQ ID NO: 143)
mN#mN#mN#mNmNmNmNmNmNmNfNfNfNfNrN#rN#fNfNrN#mNmGmUrU#rU
#fUfAmGmAmGmCmUmAmU#mG#mC#mU (crRNA 49);
(SEQ ID NO: 144)
mN#mN#mN#mNmNmNmNmNmNmNfNfNfNfNrN#rN#fNfNrN#mNmGrU#mUrU
#fUfAmGmAmGmCmUmAmU#mG#mC#mU (crRNA 50);
and
(SEQ ID NO: 145)
mG#mG#mU#mGmAmGmCmUmCmUfUfAfUfUrU#rG#fCfGrU#mAmGrU#rU#m
UfUfAmGmAmGmCmUmAmU#mG#mC#mU (crRNA 51),
wherein rN = RNA, mN = 2′-O-methyl RNA, fN = 2′-fluoro RNA,
dN = 2′-deoxy RNA, N#N = phosphorothioate linkage,
and N = any nucleotide.

In an embodiment, the tracr portion comprises one or more modified nucleotides each independently selected from a modification of a ribose group, a phosphate group, a nucleobase, or a combination thereof.

In an embodiment, each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC}).

In an embodiment, at least 50% of the ribose groups are chemically modified. In an embodiment, at least 80% of the ribose groups are chemically modified. In an embodiment, 100% of the ribose groups are chemically modified.

In an embodiment, each modification of the phosphate group is independently selected from the group consisting of a phosphorothioate, phosphonoacetate (PACE), thiophosphonoacetate (thioPACE), amide, triazole, phosphonate, and phosphotriester modification.

In an embodiment, each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

In an embodiment, tracrRNA portion comprises at least 50% modified nucleotides. In an embodiment, tracrRNA portion comprises at least 80% modified nucleotides. In an embodiment, tracrRNA portion comprises at least 90% modified nucleotides. In an embodiment, tracrRNA portion comprises 100% chemically modified nucleotides.

In an embodiment, the chemically modified guide RNA comprises a tracrRNA portion modification pattern selected from any of tracrRNAs 1-116 of Table 2.

In one aspect, the disclosure provides a chemically modified guide RNA comprising: (a) a crRNA portion comprising (i) a guide sequence capable of hybridizing to a target polynucleotide sequence, and (ii) a repeat sequence; and (b) a tracrRNA portion comprising an anti-repeat nucleotide sequence that is complementary to the repeat sequence, wherein: the crRNA portion comprises a modification pattern selected from the group consisting of:

(SEQ ID NO: 134)
mN#mN#mN#mNmNmNmNmNmNmNfNfNfNfNrN#fNfNfNfNmNmGfUfU
fUfUfAmGmAmGmCmUmAmU#mG#mC#mU (crRNA 29);
(SEQ ID NO: 125)
mN#mN#mN#rN#rN#rN#mNmNmNmNfNfNfNfNfNfNfNfNfNmNmGfU
fUfUfUfAmGmAmGmCmUmAmU#mG#mC#mU (crRNA 39);
(SEQ ID NO: 6)
mN#mN#mN#dN#dN#dN#mNmNmNmNfNfNfNfNfNfNfNfNfNmNmGfU
fUfUfUfAmGmAmGmCmUmAmU#mG#mC#mU (crRNA 40);
and
(SEQ ID NO: 126)
mN#mN#mN#fNfNfNmNmNmNmNfNfNfNfNfNfNfNfNfNmNmGfUfUf
UfUfAmGmAmGmCmUmAmU#mG#mC#mU (crRNA 45);

and the tracrRNA portion comprises a modification pattern selected from the group consisting of:

(SEQ ID NO: 16)
mA#mG#mC#mAmUmAmGmCmAmAmGfUfUmAfAmAmAfUmAmAmGmGfCf
UmAfGfUfCmCfGfUfUmAmUmCmAmAmCmUmUmGmAmAmAmAmAmGmUm
GmGmCmAmCmCmGmAmGmUmCmGmGmUmGmC#mU#mU#mU (tracrRNA
8);
(SEQ ID NO: 17)
mA#mG#mC#mAmUmAmGmCmAmAmGfUrUmArAmAmArUmAmAmGmGrCr
UmArGrUrCmCrGrUrUmAmUmCmAmAmCmUmUmGmAmAmAmAmAmGmUm
GmGmCmAmCmCmGmAmGmUmCmGmGmUmGmC#mU#mU#mU (tracrRNA
9);
(SEQ ID NO: 20)
mA#mG#mC#mAmUmAmGmCmAmAmGrUrUmArAmAmAfUmAmAmGmGrCr
UmArGrUrCmCrGrUrUmAmUmCmAmAmCmUmUmGmAmAmAmAmAmGmUm
GmGmCmAmCmCmGmAmGmUmCmGmGmUmGmC#mU#mU#mU (tracrRNA
12);
(SEQ ID NO: 25)
mA#mG#mC#mAmUmAmGmCmAmAmGrUrUmArAmAmArUmAmAmGmGrCr
UmArGrUfCmCrGrUrUmAmUmCmAmAmCmUmUmGmAmAmAmAmAmGmUm
GmGmCmAmCmCmGmAmGmUmCmGmGmUmGmC#mU#mU#mU (tracrRNA
17);
(SEQ ID NO: 26)
mA#mG#mC#mAmUmAmGmCmAmAmGrUrUmArAmAmArUmAmAmGmGrCr
UmArGrUrCmCfGrUrUmAmUmCmAmAmCmUmUmGmAmAmAmAmAmGmUm
GmGmCmAmCmCmGmAmGmUmCmGmGmUmGmC#mU#mU#mU (tracrRNA
18);
(SEQ ID NO: 44)
mA#mG#mC#mAmUmAmGmCmAmAmGrUrUmArAmAmArU#mAmAmGmGrCr
UmArGrUrCmCrGrUrUmAmUmCmAmAmCmUmUmGmAmAmAmAmAmGmUm
GmGmCmAmCmCmGmAmGmUmCmGmGmUmGmC#mU#mU#mU (tracrRNA
37);
(SEQ ID NO: 45)
mA#mG#mC#mAmUmAmGmCmAmAmGrUrUmArAmAmArUmAmAmGmGrC#r
UmArGrUrCmCrGrUrUmAmUmCmAmAmCmUmUmGmAmAmAmAmAmGmUm
GmGmCmAmCmCmGmAmGmUmCmGmGmUmGmC#mU#mU#mU (tracrRNA
38);
(SEQ ID NO: 48)
mA#mG#mC#mAmUmAmGmCmAmAmGrUrUmArAmAmArUmAmAmGmGrCr
UmArGrU#rCmCrGrUrUmAmUmCmAmAmCmUmUmGmAmAmAmAmAmGmU
mGmGmCmAmCmCmGmAmGmUmCmGmGmUmGmC#mU#mU#mU
(tracrRNA 41);
(SEQ ID NO: 56)
mA#mG#mC#mAmUmAmGmCmAmAmGrUrUmArAmAmArUmAmAmGmGfCf
UmArGrUrCmCrGrUrUmAmUmCmAmAmCmUmUmGmAmAmAmAmAmGmUm
GmGmCmAmCmCmGmAmGmUmCmGmGmUmGmC#mU#mU#mU (tracrRNA
49);
(SEQ ID NO: 98)
mA#mG#mC#mAmUmAmGmCmAmAmGrUrUmArAmAmArUmAmAmGmGrCr
UmArG#rU#rC#mCrGrUrUmAmUmCmAmAmCmUmUmGmAmAmAmAmAmG
mUmGmGmCmAmCmCmGmAmGmUmCmGmGmUmGmC#mU#mU#mU
(tracrRNA 92);
and
(SEQ ID NO: 101)
mA#mG#mC#mAmUmAmGmCmAmAmGrUrUmArAmAmArUmAmAmGmGrC#r
U#mArG#rU#rC#mCrGrUrUmAmUmCmAmAmCmUmUmGmAmAmAmAmAmG
mUmGmGmCmAmCmCmGmAmGmUmCmGmGmUmGmC#mU#mU#mU
(tracrRNA 95),
wherein
rN = RNA, mN = 2′-O-methyl RNA, fN = 2′-fluoro RNA,
dN = 2′-deoxy RNA, N#N = phosphorothioate linkage,
and
N = any nucleotide.

In one aspect, the disclosure provides a chemically modified guide RNA comprising: (a) a crRNA portion comprising (i) a guide sequence capable of hybridizing to a target polynucleotide sequence, and (ii) a repeat sequence; and (b) a tracrRNA portion comprising an anti-repeat nucleotide sequence that is complementary to the repeat sequence, wherein: the crRNA portion and the tracrRNA portion each independently comprise at least one chemically modified nucleotide; and the tracrRNA portion comprises at least one 2′-deoxy modified ribose group.

In an embodiment, the modified nucleotides each independently comprise a modification of a ribose group, a phosphate group, a nucleobase, or a combination thereof.

In an embodiment, each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC}).

In an embodiment, at least 80% of the ribose groups are chemically modified. In an embodiment, at least 90% of the ribose groups are chemically modified. In an embodiment, 100% of the ribose groups are chemically modified.

In an embodiment, each modification of the phosphate group is independently selected from the group consisting of a phosphorothioate, phosphonoacetate (PACE), thiophosphonoacetate (thioPACE), amide, triazole, phosphonate, and phosphotriester modification.

In an embodiment, each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

In an embodiment, the guide RNA comprises at least 90% modified nucleotide. In an embodiment, the guide RNA comprises 100% modified nucleotides.

In an embodiment, the chemically modified guide RNA comprises a tracrRNA portion modification pattern selected from the group consisting of:

(SEQ ID NO: 80)
mA#mG#mC#mAmUmAmGmCmAmAmGdUdUmArAmAmArUmAmAmGmGrCr
UmArGrUrCmCrGrUrUmAmUmCmAmAmCmUmUmGmAmAmAmAmAmGmUm
GmGmCmAmCmCmGmAmGmUmCmGmGmUmGmC#mU#mU#mU
(tracrRNA 74);
(SEQ ID NO: 81)
mA#mG#mC#mAmUmAmGmCmAmAmGrUrUmAdAmAmAdUmAmAmGmGrCr
UmArGrUrCmCrGrUrUmAmUmCmAmAmCmUmUmGmAmAmAmAmAmGmUm
GmGmCmAmCmCmGmAmGmUmCmGmGmUmGmC#mU#mU#mU
(tracrRNA 75);
(SEQ ID NO: 82)
mA#mG#mC#mAmUmAmGmCmAmAmGdUdUmAdAmAmAdUmAmAmGmGrCr
UmArGrUrCmCrGrUrUmAmUmCmAmAmCmUmUmGmAmAmAmAmAmGmUm
GmGmCmAmCmCmGmAmGmUmCmGmGmUmGmC#mU#mU#mU
(tracrRNA 76);
(SEQ ID NO: 83)
mA#mG#mC#mAmUmAmGmCmAmAmGrUrUmArAmAmArUmAmAmGmGdCd
UmArGrUrCmCrGrUrUmAmUmCmAmAmCmUmUmGmAmAmAmAmAmGmUm
GmGmCmAmCmCmGmAmGmUmCmGmGmUmGmC#mU#mU#mU
(tracrRNA 77);
(SEQ ID NO: 84)
mA#mG#mC#mAmUmAmGmCmAmAmGrUrUmArAmAmArUmAmAmGmGrCr
UmAdGdUdCmCrGrUrUmAmUmCmAmAmCmUmUmGmAmAmAmAmAmGmU
mGmGmCmAmCmCmGmAmGmUmCmGmGmUmGmC#mU#mU#mU
(tracrRNA 78);
(SEQ ID NO: 85)
mA#mG#mC#mAmUmAmGmCmAmAmGrUrUmArAmAmArUmAmAmGmGrCr
UmArGrUrCmCdGdUdUmAmUmCmAmAmCmUmUmGmAmAmAmAmAmGmU
mGmGmCmAmCmCmGmAmGmUmCmGmGmUmGmC#mU#mU#mU
(tracrRNA 79);
(SEQ ID NO: 86)
mA#mG#mC#mAmUmAmGmCmAmAmGrUrUmArAmAmArUmAmAmGmGdCd
UmArGrUrCmCdGdUdUmAmUmCmAmAmCmUmUmGmAmAmAmAmAmGmU
mGmGmCmAmCmCmGmAmGmUmCmGmGmUmGmC#mU#mU#mU
(tracrRNA 80);
(SEQ ID NO: 87)
mA#mG#mC#mAmUmAmGmCmAmAmGrUrUmArAmAmArUmAmAmGmGdCd
UmAdGdUdCmCrGrUrUmAmUmCmAmAmCmUmUmGmAmAmAmAmAmGmU
mGmGmCmAmCmCmGmAmGmUmCmGmGmUmGmC#mU#mU#mU
(tracrRNA 81);
(SEQ ID NO: 88)
mA#mG#mC#mAmUmAmGmCmAmAmGrUrUmArAmAmArUmAmAmGmGrCr
UmAdGdUdCmCdGdUdUmAmUmCmAmAmCmUmUmGmAmAmAmAmAmGm
UmGmGmCmAmCmCmGmAmGmUmCmGmGmUmGmC#mU#mU#mU
(tracrRNA 82);
(SEQ ID NO: 89)
mA#mG#mC#mAmUmAmGmCmAmAmGrUrUmArAmAmArUmAmAmGmGrCr
UmAdGrUdCmCrGrUrUmAmUmCmAmAmCmUmUmGmAmAmAmAmAmGmU
mGmGmCmAmCmCmGmAmGmUmCmGmGmUmGmC#mU#mU#mU
(tracrRNA 83);
(SEQ ID NO: 90)
mA#mG#mC#mAmUmAmGmCmAmAmGrUrUmArAmAmArUmAmAmGmGrCr
UmArGrUrCmCdGrUdUmAmUmCmAmAmCmUmUmGmAmAmAmAmAmGmU
mGmGmCmAmCmCmGmAmGmUmCmGmGmUmGmC#mU#mU#mU
(tracrRNA 84);
(SEQ ID NO: 91)
mA#mG#mC#mAmUmAmGmCmAmAmGrUrUmArAmAmArUmAmAmGmGdCd
UmAdGrUdCmCdGrUdUmAmUmCmAmAmCmUmUmGmAmAmAmAmAmGmU
mGmGmCmAmCmCmGmAmGmUmCmGmGmUmGmC#mU#mU#mU
(tracrRNA 85);
(SEQ ID NO: 92)
mA#mG#mC#mAmUmAmGmCmAmAmGrUrUmArAmAmArUmAmAmGmGrCr
UmAdGrUdCmCdGrUdUmAmUmCmAmAmCmUmUmGmAmAmAmAmAmGmU
mGmGmCmAmCmCmGmAmGmUmCmGmGmUmGmC#mU#mU#mU
(tracrRNA 86);
(SEQ ID NO: 93)
mA#mG#mC#mAmUmAmGmCmAmAmGrUrUmArAmAmArUmAmAmGmGdCr
UmAdGrUdCmCdGrUdUmAmUmCmAmAmCmUmUmGmAmAmAmAmAmGmU
mGmGmCmAmCmCmGmAmGmUmCmGmGmUmGmC#mU#mU#mU
(tracrRNA 87);
(SEQ ID NO: 108)
mA#mG#mC#mAmUmAmGmCmAmAmGrUrUmArAmAmAdUmAmAmGmGdCd
UmArGdUdCmCrGrUrUmAmUmCmAmAmCmUmUmGmAmAmAmAmAmGmU
mGmGmCmAmCmCmGmAmGmUmCmGmGmUmGmC#mU#mU#mU
(tracrRNA 104);
(SEQ ID NO: 109)
mA#mG#mC#mAmUmAmGmCmAmAmGrUdUmArAmAmAdUmAmAmGmGdCd
UmAdGdUdCmCrGrUrUmAmUmCmAmAmCmUmUmGmAmAmAmAmAmGmU
mGmGmCmAmCmCmGmAmGmUmCmGmGmUmGmC#mU#mU#mU
(tracrRNA 105);
and
(SEQ ID NO: 110)
mA#mG#mC#mAmUmAmGmCmAmAmGdUdUmAdAmAmAdUmAmAmGmGdC
dUmAdGdUdCmCdGdUdUmAmUmCmAmAmCmUmUmGmAmAmAmAmAmGm
UmGmGmCmAmCmCmGmAmGmUmCmGmGmUmGmC#mU#mU#mU
(tracrRNA 106),
wherein
rN = RNA, mN = 2′-O-methyl RNA, fN = 2′-fluoro RNA,
dN = 2′-deoxy RNA, N#N = phosphorothioate linkage,
and
N = any nucleotide.

In an embodiment, the chemically modified guide RNA comprises a crRNA portion modification pattern selected from any one of crRNAs 1-134 of Table 1.

In an embodiment, the chemically modified guide RNA comprises a crRNA portion modification pattern selected from the group consisting of:

| Name | Sequence |
|---|---|
| crRNA 1 | mN#mN#mN#mNmNmNmNmNmNmNrNrNrNrNrNmNmN |
| | mNrNmNmGrUrUrUmUmAmGmAmGmCmUmAmU#mG# |
| | mCmU (SEQ ID NO: 146) |
| crRNA 2 | rNrNrNrNrNrNmNmNmNmNrNrNrNrNrNrNrNrNrNm |
| | GNmrUrUrUrUrAmGmAmGmCmUmAmU#mG#mCmU |
| | (SEQ ID NO: 147) |
| crRNA 3 | rN#rN#rN#rNrNrNmNmNmNmNrNrNrNrNrNrNrNrN |
| | mrNNmGrUrUrUrUrAmGmAmGmCmUmAmU#mG#mCmU |
| | (SEQ ID NO: 148) |
| crRNA 4 | mN#mN#mN#mNmNmNmNmNmNmNrNrNrNrNrNrNrNr |
| | NrNmNmGrUrUrUrUrAmGmAmGmCmUmAmU#mG#mC |
| | #mU (SEQ ID NO: 149) |
| crRNA 5 | rN#rN#rN#rNrNrNmNmNmNmNrNrNrNrNrNrNmNmN |
| | rNmNmGrUrUrUrUrAmGmAmGmCmUmAmU#mG#mC#mU |
| | (SEQ ID NO: 150) |
| crRNA 6 | rN#rN#rN#rNrNrNmNmNmNmNrNrNrNrNrNrNrNrN |
| | mrNNmGrUrUrUmUmAmGmAmGmCmUmAmU#mG#mC#mU |
| | (SEQ ID NO: 151) |
| crRNA 7 | mN#mN#mN#mNmNmNmNmNmNmNrNrNrNrNrNrNmN |
| | mNrNmNmGrUrUrUrUrAmGmAmGmCmUmAmU#mG#m |
| | C#mU (SEQ ID NO: 152) |
| crRNA 8 | mN#mN#mN#mNmNmNmNmNmNmNrNrNrNrNrNrNrNr |
| | NrNmNmGrUrUrUmUmAmGmAmGmCmUmAmU#mG#m |
| | C#mU (SEQ ID NO: 153) |
| crRNA 9 | mN#mN#mN#mNmNmNmNmNmNmNrNrNrNrNrN#rN#r |
| | NrNrN#mNmGrU#rU#rU#mUmAmGmAmGmCmUmAmU |
| | #mG#mC#mU (SEQ ID NO: 154) |
| crRNA 10 | mN#mN#mN#mNmNmNmNmNmNmNfNfNfNfNrN#rN#f |
| | NfNrN#mNmGrU#rU#rU#mUmAmGmAmGmCmUmAmU |
| | #mG#mC#mU (SEQ ID NO: 155) |
| crRNA 11 | mN#mN#mN#mNmNmNmNmNmNmNfNfNfNfNrN#rN#r |
| | N#rN#rN#mNmGrU#rU#rU#mUmAmGmAmGmCmUmAm |
| | U#mG#mC#mU (SEQ ID NO: 156) |
| crRNA 17 | mN#mN#mN#mNmNmNmNmNmNmNfNfNfNfNrN#rN#f |
| | NfNrN#mNmGrU#rU#rU#mUrA#mGmAmGmCmUmAmU |
| | #mG#mC#mU (SEQ ID NO: 157) |
| crRNA 18 | mN#mN#mN#mNmNmNmNmNmNmNfNfNfNfNfN#rN#f |
| | NfNrN#mNmGrU#rU#rU#rU#mAmGmAmGmCmUmAmU |
| | #mG#mC#mU (SEQ ID NO: 158) |
| crRNA 19 | mN#mN#mN#mNmNmNmNmNmNmNfNfNfNfNrN#rN#f |
| | NfNrN#mNmGrU#rU#rU#rU#rA#mGmAmGmCmUmAmU |
| | #mG#mC#mU (SEQ ID NO: 159) |
| crRNA 20 | mN#mN#mN#mNmNmNmNmNmNmNfNfNfNfNrN#rN#f |
| | NfNrN#mNmGrU#rU#rU#fUfAmGmAmGmCmUmAmU# |
| | mG#mC#mU (SEQ ID NO: 160) |
| crRNA 21 | mN#mN#mN#mNmNmNmNmNmNmNfNfNfNfNfNfNfNf |
| | NfNmNmGfUfUfUfUfAmGmAmGmCmUmAmU#mG#mC |
| | #mU (SEQ ID NO: 161) |
| crRNA 22 | mN#mN#mN#mNmNmNmNmNmNmNfNfNfNfNrN#rN#f |
| | NfNrN#mNmGfUrUgUfUfAmGmAmGmCmUmAmU#mG |
| | #mC#mU (SEQ ID NO: 162) |
| crRNA 23 | mN#mN#mN#mNmNmNmNmNmNmNfNfNfNfNmNrN#f |
| | NfNrN#mNmGrU#rU#rU#fUfAmGmAmGmCmUmAmU# |
| | mG#mC#mU (SEQ ID NO: 128) |
| crRNA 24 | mN#mN#mN#mNmNmNmNmNmNmNfNfNfNfNrN#fNfNf |
| | NrN#mNmGrU#rU#rU#fUfAmGmAmGmCmUmAmU#mG |
| | #mC#mU (SEQ ID NO: 129) |
| crRNA 25 | mN#mN#mN#mNmNmNmNmNmNmNfNfNfNfNrN#rN#f |
| | NfNfNmNmGrU#rU#rU#fUfAmGmAmGmCmUmAmU#m |
| | G#mC#mU (SEQ ID NO: 130) |
| crRNA 26 | mN#mN#mN#mNmNmNmNmNmNmNfNfNfNfNrN#rN#f |
| | NfNrN#mNmGfUrU#rU#fUfAmGmAmGmCmUmAmU#m |
| | G#mC#mU (SEQ ID NO: 131) |
| crRNA 27 | mN#mN#mN#mNmNmNmNmNmNmNfNfNfNfNrN#rN#f |
| | NfNrN#NAmGrU#fUrU#fUfAmGmAmGmCmUmAmU#m |
| | G#mC#mU (SEQ ID NO: 163) |
| crRNA 28 | mN#mN#mN#mNmNmNmNmNmNmNfNfNfNfNrN#rN#f |
| | NfNrN#mNmGrU#rU#rUfUfAmGmAmGmCmUmAmU#m |
| | G#mC#Mu (SEQ ID NO: 133) |
| crRNA 29 | mN#mN#mN#mNmNmNmNmNmNmNfNfNfNfNrN#fNfNf |
| | NfNmNmGfUfUfUfUfAmGmAmGmCmUmAmU#mG#mC |
| | #mU (SEQ ID NO: 134) |
| crRNA 30 | mN#mN#mN#rNrNrNmNmNmNmNrNrNrNrNrNrNrNrN |
| | mrNNmGrUrUrUrUrAmGmAmGmCmUmAmU#mG#mC#mU |
| | (SEQ ID NO: 135) |
| crRNA 31 | mN#mN#mN#rNrNrNmNmNmNmNmNrNrNrNrNrNrNrNr |
| | NmNmGrUrUrUrUrAmGmAmGmCmUmAmU#mG#mC#m |
| | U (SEQ ID NO: 136) |
| crRNA 32 | mN#mN#mN#rNrNrNmNmNmNmNmNrNmNmNrNrNrNr |
| | NrNmNmGrUrUrUrUrAmGmAmGmCmUmAmU#mG#mC |
| | #mU (SEQ ID NO: 137) |
| crRNA 33 | mN#mN#mN#rN#rN#rN#mNmNmNmNrN#rN#rN#rN#rN |
| | #rN#rN#rN#rN#mNmGrU#rU#rU#rU#rA#mGmAmGmC |
| | mUmAmU#mG#mC#mU (SEQ ID NO: 121) |
| crRNA 34 | mN#mN#mN#rN#rN#rN#mNmNmNmNrN#rN#rN#rN#rN |
| | #rN#rN#rN#rN#mNmGrUrUrUrUrAmGmAmGmCmUmAm |
| | U#mG#mC#mU (SEQ ID NO: 122) |
| crRNA 35 | mN#mN#mN#rNrNrNmNmNmNmNrNrNrNrNrNrNrNrNrN |
| | mNmGrUrUrUmUmAmGmAmGmCmUmAmU#mG#mC# |
| | mU (SEQ ID NO: 138) |
| crRNA 36 | mN#mN#mN#rN#rN#rN#mNmNmNmNrN#rN#rN#rN#rN |
| | #rN#N#rN#rN#mNmGrUrUrUmUmAmGmAmGmCmUmAm |
| | U#mG#mC#mU (SEQ ID NO: 123) |
| crRNA 37 | mN#mN#mN#N#rN#rN#mNmNmNmNfNfNfNfNfrN#rN |
| | N#ffNrN#mNmGrU#rU#rU#mUmAmGmAmGmCmUmAmU |
| | #mG#mC#mU (SEQ ID NO: 124) |
| crRNA 38 | mN#mN#mN#dN#dN#dN#mNmNmNmNfNfNfNfNrN#rN# |
| | fNfNrN#mNmGrU#rU#rU#mUmAmGmAmGmCmUmAmU |
| | #mG#mC#mU (SEQ ID NO: 5) |
| crRNA 39 | mN#mN#mN#rN#rN#rN#mNmNmNmNfNfNfNfNfNfNfNf |
| | NfNmNmGfUfUfUfUfAmGmAmGmCmUmAmU#mG#mC |
| | #mU (SEQ ID NO: 125) |
| crRNA 40 | mN#mN#mN#dN#dN#dN#mNmNmNmNfNfNfNfNfNfNfN |
| | fNfNmNmGfUfUfUfUfAmGmAmGmCmUmAmU#mG#mC |
| | #mU (SEQ ID NO: 6) |
| crRNA 41 | mN#mN#mN#mNmNmNmNmNmNmNfNfNfNfNdN#dN#f |
| | NfNdN#mNmGrU#rU#rU#fUfAmGmAmGmCmUmAmU# |
| | mG#mC#mU (SEQ ID NO: 7) |
| crRNA 42 | mN#mN#mN#mNmNmNmNmNmNmNfNfNfNfNrN#rN#f |
| | NfNrN#mNmGdU#dU#dU#fUfAmGmAmGmCmUmAmU# |
| | mG#mC#mU (SEQ ID NO: 8) |
| crRNA 43 | mN#mN#mN#mNmNmNmNmNmNmNfNrN#fNfNrN#rN#f |
| | NfNrN#mNmGrU#rU#rU#fUfAmGmAmGmCmUmAmU# |
| | mG#mC#mU (SEQ ID NO: 139) |
| crRNA 44 | mN#mN#mN#mNmNmNmNmNmNmNfNdN#fNfNrN#rN# |
| | fNfNrN#mNmGrU#1-U#1-U#fUfAmGmAmGmCmUmAmU# |
| | mG#mC#mU (SEQ ID NO: 9) |
| crRNA 45 | mN#mN#mN#fNfNfNmNmNmNmNfNfNfNfNfNfNfNfNfN |
| | mNmGfUfUfUfUfAmGmAmGmCmUmAmU#mG#mC#mU |
| | (SEQ ID NO: 126) |
| crRNA 46 | mN#mN#mN#mNmNmNmNmNmNmNfNfNfNfNmNrN#f |
| | NfNrN#mNmGrU#rUrU#fUfAmGmAmGmCmUmAmU#m |
| | G#mC#mU (SEQ ID NO: 140) |
| crRNA 47 | mN#mN#mN#mNmNmNmNmNmNmNfNfNfNfNrN#mNf |
| | NfNrN#mNmGrU#rUrU#fUfAmGmAmGmCmUmAmU#m |
| | G#mC#mU (SEQ ID NO: 141) |
| crRNA 48 | mN#mN#mN#mNmNmNmNmNmNmNfNfNfNfNrN#mNf |
| | NfNmNmNmGrU#rUrU#fUfAmGmAmGmCmUmAmU#m |
| | G#mC#mU (SEQ ID NO: 142) |
| crRNA 49 | mN#mN#mN#mNmNmNmNmNmNmNfNfNfNfNrN#rN#f |
| | NfNrN#mNmGmUrU#rUgUfAmGmAmGmCmUmAmU#m |
| | G#mC#mU (SEQ ID NO: 143) |
| crRNA 50 | mN#mN#mN#mNmNmNmNmNmNmNfNfNfNfNrN#rN#f |
| | NfNrN#mNmGrU#mUrUgUfAmGmAmGmCmUmAmU#m |
| | G#mC#mU (SEQ ID NO: 144) |
| crRNA 51 | mN#mN#mN#mNmNmNmNmNmNmNfNfNfNfNrN#rN#f |
| | NfNrN#mNmGrU#rU#mUfUfAmGmAmGmCmUmAmU#m |
| | G#mC#mU (SEQ ID NO: 164) |
| crRNA 52 | mN#mN#mN#dN#dN#dN#mNmNmNmNfNfNfNfNfN#fN# |
| | fNfNfN#mNmGfU#fU#fU#fU#fA#mGmAmGmCmUmAm |
| | U#mG#mC#mU (SEQ ID NO: 165) |
| crRNA 53 | mN#mN#mN#dN#dN#dN#mN#mNmNmNfNfNfNfNfNfNf |
| | NfNfNmNmGfUfUfUfUfAmGmAmGmCmUmAmU#mG# |
| | mC#mU (SEQ ID NO: 166) |
| crRNA 54 | mN#mN#mN#dN#dN#dN#mNmN#mNmNfNfNfNfNfNfNf |
| | NfNfNmNmGfUfUfUfUfAmGmAmGmCmUmAmU#mG# |
| | mC#mU (SEQ ID NO: 167) |
| crRNA 55 | mN#mN#mN#dN#dN#dN#mNmNmN#mNfNfNfNfNfNfNf |
| | NfNfNmNmGfUfUfUfUfAmGmAmGmCmUmAmU#mG# |
| | mC#mU (SEQ ID NO: 168) |
| crRNA 56 | mN#mN#mN#dN#dN#dN#mNmNmNmN#fNfNfNfNfNfNf |
| | NfNfNmNmGfUfUfUfUfAmGmAmGmCmUmAmU#mG# |
| | mC#mU (SEQ ID NO: 169) |
| crRNA 57 | mN#mN#mN#dN#dN#dN#mNmNmNmNfN#fNfNINfNfNf |
| | NfNfNmNmGfUfUfUfUfAmGmAmGmCmUmAmU#mG# |
| | mC#mU (SEQ ID NO: 170) |
| crRNA 58 | mN#mN#mN#dN#dN#dN#mNmNmNmNfNfN#fNfNfNfNf |
| | NfNfNmNmGfUfUfUfUfAmGmAmGmCmUmAmU#mG# |
| | mC#mU (SEQ ID NO: 171) |
| crRNA 59 | mN#mN#mN#dN#dN#dN#mNmNmNmNfNfNfN#fNfNfNf |
| | NfNfNmNmGfUfUfUfUfAmGmAmGmCmUmAmU#mG# |
| | mC#mU (SEQ ID NO: 172) |
| crRNA 60 | mN#mN#mN#dN#dN#dN#mNmNmNmNfNfNfNfN#fNfNf |
| | NfNfNmNmGfUfUfUfUfAmGmAmGmCmUmAmU#mG# |
| | mC#mU (SEQ ID NO: 173) |
| crRNA 61 | mN#mN#mN#dN#dN#dN#mNmNmNmNfNfNfNfNfNfNfN |
| | #fNfNmNmGfUfUfUfUfAmGmAmGmCmUmAmU#mG#m |
| | C#mU (SEQ ID NO: 174) |
| crRNA 62 | mN#mN#mN#dN#dN#dN#mNmNmNmNfNfNfNfNfNfNfN |
| | fN#fNmNmGfUfUfUfUfAmGmAmGmCmUmAmU#mG#m |
| | C#mU (SEQ ID NO: 175) |
| crRNA 63 | mN#mN#mN#dN#dN#dN#mNmNmNmNfNfNfNfNfNfNfN |
| | fNfNmN#mGfUfUfUfUfAmGmAmGmCmUmAmU#mG#m |
| | C#mU (SEQ ID NO: 176) |
| crRNA 64 | mN#mN#mN#dN#dN#dN#mNmNmNmNINfNfNfNfNfNfN |
| | fNfNmNmG#fUfUfUfUfAmGmAmGmCmUmAmU#mG#m |
| | C#mU (SEQ ID NO: 177) |
| crRNA 65 | mN#mN#mN#dN#dN#dN#mNmNmNmNfNfNfNfNfNfNfN |
| | fNfNmNmGfUfUfUfUfAmG#mAmGmCmUmAmU#mG#m |
| | C#mU (SEQ ID NO: 178) |
| crRNA 66 | mN#mN#mN#dN#dN#dN#mNmNmNmNfNfNfNfNfNfNfN |
| | fNfNmNmGfUfUfUfUfAmGmA#mGmCmUmAmU#mG#m |
| | C#mU (SEQ ID NO: 179) |
| crRNA 67 | mN#mN#mN#dN#dN#dN#mNmNmNmNfNfNfNfNfNfNfN |
| | fNfNmNmGfUfUfUfUfAmGmAmG#mCmUmAmU#mG#m |
| | C#mU (SEQ ID NO: 180) |
| crRNA 68 | mN#mN#mN#dN#dN#dN#mNmNmNmNfNfNfNfNfNfNfN |
| | fNfNmNmGfUfUfUfUfAmGmAmGmC#mUmAmU#mG#m |
| | C#mU (SEQ ID NO: 181) |
| crRNA 69 | mN#mN#mN#dN#dN#dN#mNmNmNmNfNfNfNfNfNfNfN |
| | fNfNmNmGfUfUfUfUfAmGmAmGmCmU#mAmU#mG#m |
| | C#mU (SEQ ID NO: 182) |
| crRNA 70 | mN#mN#mN#dN#dN#dN#mNmNmNmNfNfNfNfNfNfNfN |
| | fNfNmNmGfUfUfUfUfAmGmAmGmCmUmA#mU#mG#m |
| | C#mU (SEQ ID NO: 183) |
| crRNA 71 | mN#mN#mN#dN#dN#dN#mN#mN#mN#mNfNfNfNfNfN#f |
| | N#fNfNfN#mNmGfU#fU#fU#fU#fA#mGmAmGmCmUmA |
| | mU#mG#mC#mU (SEQ ID NO: 184) |
| crRNA 72 | mN#mN#mN#dN#dN#dN#mNmNmNmN#fN#fN#fNfNfN#f |
| | N#fNfNfN#mNmGfU#fU#fU#fU#fA#mGmAmGmCmUmA |
| | mU#mG#mC#mU (SEQ ID NO: 185) |
| crRNA 73 | mN#mN#mN#dN#dN#dN#mNmNmNmNfNfNfN#fN#fN#f |
| | N#fNfNfN#mNmGfU#fU#fU#fU#fA#mGmAmGmCmUmA |
| | mU#mG#mC#mU (SEQ ID NO: 186) |
| crRNA 74 | mN#mN#mN#dN#dN#dN#mNmNmNmNfNfNfNfNfN#fN# |
| | fN#fN#fN#mNmGfU#fU#fU#fU#fA#mGmAmGmCmUmA |
| | mU#mG#mC#mU (SEQ ID NO: 187) |
| crRNA 75 | mN#mN#mN#dN#dN#dN#mNmNmNmNfNfNfNfNfN#fN# |
| | fNfNfN#mN#mGfU#fU#fU#fU#fA#mGmAmGmCmUmAm |
| | U#mG#mC#mU (SEQ ID NO: 188) |
| crRNA 76 | mN#mN#mN#dN#dN#dN#mN#mN#mN#mNfNfNfNfNfNf |
| | NfNfNfNmNmGfUfUfUfUfAmGmAmGmCmUmAmU#mG |
| | #mC#mU (SEQ ID NO: 189) |
| crRNA 77 | mN#mN#mN#dN#dN#dN#mN#mN#mN#mN#fN#fN#fNfNf |
| | NfNfNfNfNmNmGfUfUfUfUfAmGmAmGmCmUmAmU# |
| | mG#mC#mU (SEQ ID NO: 190) |
| crRNA 78 | mN#mN#mN#dN#dN#dN#mN#mN#mN#mN#fN#fN#fN#f |
| | N#fN#fN#fNfNfNmNmGfUfUfUfUfAmGmAmGmCmUmA |
| | mU#mG#mC#mU (SEQ ID NO: 191) |
| crRNA 79 | mN#mN#mN#dN#dN#dN#mN#mN#mN#mN#fN#fN#fN#f |
| | N#fN#fN#fN#fN#fN#mNmGfUfUfUfUfAmGmAmGmCmU |
| | mAmU#mG#mC#mU (SEQ ID NO: 192) |
| crRNA 80 | mN#mN#mN#dN#dN#dN#mN#mN#mN#mN#fN#fN#fN#f |
| | N#fN#fN#fN#fN#fN#mN#mG#fU#fU#fU#fU#fA#mGm |
| | AmGmCmUmAmU#mG#mC#mU (SEQ ID NO: 193) |
| crRNA 81 | mN#mN#mN#dN#dN#dN#mNmNmNmNfNfNfNfNfN#fN# |
| | fNfNfN#mNmGfU#fU#fU#fU#fA#mGmAmGmC#mU#mA |
| | #mU#mG#mC#mU (SEQ ID NO: 194) |
| crRNA 82 | mN#mN#mN#dN#dN#dN#mNmNmNmNfNfNfNfNfN#fN# |
| | fNfNfN#mNmGfU#fU#fU#fU#fA#mG#mA#mG#mCmUm |
| | AmU#mG#mC#mU (SEQ ID NO: 195) |
| crRNA 83 | mN#mN#mN#dN#dN#dN#mNmNmNmNfNffNfNfNfN#fN# |
| | fNfNfN#mNmG#fU#fU#fU#fU#fA#mGmAmGmCmUmAm |
| | U#mG#mC#mU (SEQ ID NO: 196) |
| crRNA 84 | mN#mN#mN#dN#dN#dN#mNmNmNmNfNfNfNfNfNfNfN |
| | fNfNmNmGfUfUfUfUfAmGmAmGmC#mU#mA#mU#mG |
| | #mC#mU (SEQ ID NO: 197) |
| crRNA 85 | mN#mN#mN#dN#dN#dN#mNmNmNmNfNfNfNfNfNfMN |
| | fNfNmNmGfUfUfUfUfA#mG#mA#mG#mC#mU#mA#mU |
| | #mG#mC#mU (SEQ ID NO: 198) |
| crRNA 86 | mN#mN#mN#dN#dN#dN#mNmNmNmNfNfNfNfNfNfMN |
| | fNfNmNmGfU#fU#fU#fU#fA#mG#mA#mG#mC#mU#mA |
| | #mU#mG#mC#mU (SEQ ID NO: 199) |
| crRNA 87 | mN#mN#mN#dN#dN#dN#mN#mN#mNmNfNfNfNfNfNfN |
| | fNfNfNmNmGfUfUfUfUfAmGmAmGmCmU#mA#mU#m |
| | G#mC#mU (SEQ ID NO: 200) |
| crRNA 88 | mN#mN#mN#dN#dN#dN#mN#mN#mN#mN#fNfNfNfNfNf |
| | NfNfNfNmNmGfUfUfUfUfAmGmAmG#mC#mU#mA#mU |
| | #mG#mC#mU (SEQ ID NO: 201) |
| crRNA 89 | mN#mN#mN#dN#dN#dN#mN#mN#mN#mN#fN#fN#fNfNf |
| | NfNfNfNfNmNmGfUfUfUfUfAmG#mA#mG#mC#mU#mA |
| | #mU#mG#mC#mU (SEQ ID NO: 202) |
| crRNA 90 | mN#mN#mN#dN#dN#dN#mN#mN#mN#mN#fN#N#N#f |
| | N#fNfNfNfNfNmNmGfUfUfUfU#fA#mG#mA#mG#mC#m |
| | U#mA#mU#mG#mC#mU (SEQ ID NO: 203) |
| crRNA 91 | mN#mN#mN#dN#dN#dN#mN#mN#mN#mN#fN#N#N#f |
| | N#fN#fN#fNfNfNmNmGfUfU#fU#fU#fA#mG#mA#mG |
| | #mC#mU#mA#mU#mG#mC#mU (SEQ ID NO: 204) |
| crRNA 92 | mN#mN#mN#dN#dN#dN#mN#mN#mN#mN#fN#N#N#f |
| | N#fN#fN#fN#fN#fN#mNmGfU#fU#fU#fU#fA#mG#mA |
| | #mG#mC#mU#mA#mU#mG#mC#mU (SEQ ID NO: 205) |
| crRNA 93 | mN#mN#mN#dN#dN#dN#mN#mN#mN#mN#fN#N#N#f |
| | N#fN#fN#fN#fN#fN#mN#mG#fU#fU#fU#fU#fA#mG |
| | #mA#mG#mC#mU#mA#mU#mG#mC#mU (SEQ ID NO: |
| | 206) |
| crRNA 94 | mN#mN#mN#mN#mN#mN#mNmNmNmNfNfNfNfNrN#r |
| | N#fNfNrN#mNmGrU#rU#rU#fUfAmGmAmGmCmUmAm |
| | U#mG#mC#mU (SEQ ID NO: 207) |
| crRNA 95 | mN#mN#mN#mN#mN#mN#mN#mNmNmNfNfNfNfNrN#r |
| | N#fNfNrN#mNmGrU#rU#rU#fUfAmGmAmGmCmUmAm |
| | U#mG#mC#mU (SEQ ID NO: 208) |
| crRNA 96 | mN#mN#mN#mN#mN#mN#mNmNmNmNfNfNfNfNrN#r |
| | N#fNfNrN#mNmGrU#rU#rU#fUfAmGmAmGmC#mUmA |
| | mU#mG#mC#mU (SEQ ID NO: 209) |
| crRNA 97 | mN#mN#mN#mN#mN#mN#mNmNmNmNfNfNfNfNrN#r |
| | N#fNfNrN#mNmGrU#rU#rU#fUfAmGmAmGmCmU#mA |
| | mU#mG#mC#mU (SEQ ID NO: 210) |
| crRNA 98 | mN#mN#mN#mN#mN#mN#mNmNmNmNfNfNfNfNrN#r |
| | N#fNfNrN#mGNmGrU#rU#rU#fUfAmGmAmGmCmUmA |
| | #mU#mG#mC#mU (SEQ ID NO: 211) |
| crRNA 99 | mN#mN#mN#mN#mN#mN#mNmNmNmNfNfNfNfNrN#r |
| | N#fNfNrN#mNmGrU#rU#rU#fU#fA#mGmAmGmCmUmA |
| | mU#mG#mC#mU (SEQ ID NO: 212) |
| crRNA 100 | mN#mN#mN#mN#mN#mN#mNmNmNmNfNfNfNfNrN#r |
| | N#fNfNrN#mNmGrU#rU#rU#fU#fA#mGmAmGmC#mU# |
| | mA#mU#mG#mC#mU (SEQ ID NO: 213) |
| crRNA 101 | mN#mN#mN#mN#mN#mN#mNmNmNmNfNfNfNfNrN#r |
| | N#fNfNrN#mNmGrU#rU#rU#fU#fA#mG#mA#mG#mCmU |
| | mAmU#mG#mC#mU (SEQ ID NO: 214) |
| crRNA 102 | mN#mN#mN#mN#mN#mN#mNmNmNmNfNfNfNfNrN#r |
| | N#fNfNrN#mNmG#rU#rU#rU#fU#fA#mGmAmGmCmUm |
| | AmU#mG#mC#mU (SEQ ID NO: 215) |
| crRNA 103 | mN#mN#mN#mN#mN#mN#mNmNmNmNfNfNfNfNrN#r |
| | N#fNfNrN#mNmGrU#rU#rU#fUfAmGmAmGmC#mU#mA |
| | #mU#mG#mC#mU (SEQ ID NO: 216) |
| crRNA 104 | mN#mN#mN#mN#mN#mN#mNmNmNmNfNfNfNfNrN#r |
| | N#fNfNrN#mNmGrU#rU#rU#fUfA#mG#mA#mG#mC#mU |
| | #mA#mU#mG#mC#mU (SEQ ID NO: 217) |
| crRNA 105 | mN#mN#mN#rN#rN#rN#mN#mNmNmNfNfNfNfNfNfNfN |
| | fNfNmNmGfUfUfUfUfAmGmAmGmCmUmAmU#mG#mC |
| | #mU (SEQ ID NO: 218) |
| crRNA 106 | mN#mN#mN#rN#rN#rN#mNmNmNmNfNfNfNfNfNfNfNf |
| | NfNmNmGfUfUfUfUfAmGmAmGmCmUmA#mU#mG#m |
| | C#mU (SEQ ID NO: 219) |
| crRNA 107 | mN#mN#mN#rN#rN#rN#mNmNmNmNfNfNfNfNfN#fN#f |
| | N#fN#fN#mNmGfU#fU#fU#fU#fA#mGmAmGmCmUmA |
| | mU#mG#mC#mU (SEQ ID NO: 220) |
| crRNA 108 | mN#mN#mN#rN#rN#rN#mNmNmNmNfNfNfNfNfN#fN |
| | #fNfNfN#mNmGfU#fU#fU#fU#fA#mGmAmGmC#mU#mA |
| | #mU#mG#mC#mU (SEQ ID NO: 221) |
| crRNA 109 | mN#mN#mN#rN#rN#rN#mNmNmNmNfNfNfNfNfN#fN |
| | #fNfNfN#mNmGfU#fU#fU#fU#fA#mG#mA#mG#mCm |
| | UmAmU#mG#mC#mU (SEQ ID NO: 222) |
| crRNA 110 | mN#mN#mN#rN#rN#rN#mNmNmNmNfNfNfNfNfN#fN# |
| | fNfNfN#mNmG#fU#fU#fU#fU#fA#mGmAmGmCmUmAm |
| | U#mG#mC#mU (SEQ ID NO: 223) |
| crRNA 111 | mN#mN#mN#rN#rN#rN#mNmNmNmNfNfNfNfNfNfNfN |
| | fNfNmNmGfUfUfUfUfAmGmAmGmC#mU#mAmU#mG#m |
| | C#mU (SEQ ID NO: 224) |
| crRNA 112 | mN#mN#mN#rN#rN#rN#mNmNmNmNfNfNfNfNfNfNfN |
| | fNfNmNmGfUfUfUfUfA#mG#mA#mG#mC#mU#mAmU#m |
| | G#mC#mU (SEQ ID NO: 225) |
| crRNA 113 | mN#mN#mN#dN#dN#dN#mNmNmNmNfNfNfNfNdN#dN |
| | #fNfNdN#mNmGdU#dU#dU#fUfAmGmAmGmCmUmAm |
| | U#mG#mC#mU (SEQ ID NO: 226) |
| crRNA 114 | mN#mN#mN#mNmNmNmNmNmNmNfNfNfNfNrN#rN#f |
| | NfNaNmNmGaUaUaUfUfAmGmAmGmCmUmAmU#mG# |
| | mC#mU (SEQ ID NO: 227) |
| crRNA 115 | mN#mN#mN#mNmNmNmNmNmNmNfNfNfNfNrN#rN#f |
| | NfNaNmNmGrU#rU#rU#fUfAmGmAmGmCmUmAmU#m |
| | G#mC#mU (SEQ ID NO: 228) |
| crRNA 116 | mN#mN#mN#mNmNmNmNmNmNmNfNfNfNfNrN#rN#f |
| | NfNrN#mNmGaUrU#rU#fUfAmGmAmGmCmUmAmU#m |
| | G#mC#mU (SEQ ID NO: 229) |
| crRNA 117 | mN#mN#mN#mNmNmNmNmNmNmNfNfNfNfNrN#rN#f |
| | NfNrN#mNmGrU#aUrU#fUfAmGmAmGmCmUmAmU#m |
| | G#mC#mU (SEQ ID NO: 230) |
| crRNA 118 | mN#mN#mN#mNmNmNmNmNmNmNfNfNfNfNrN#rN#f |
| | NfNrN#mNmGrU#rU#aUfUfAmGmAmGmCmUmAmU#m |
| | G#mC#mU (SEQ ID NO: 231) |
| crRNA 119 | mN#mN#mN#mNmNmNmNmNmNmNfNfNfNfNrN#rN#f |
| | NfNsN#mNmGsU#sU#sU#fUfAmGmAmGmCmUmAmU# |
| | mG#mC#mU (SEQ ID NO: 232) |
| crRNA 120 | mN#mN#mN#mNmNmNmNmNmNmNfNfNfNfNrN#rN#f |
| | NfNsNmNmGsUsUsUfUfAmGmAmGmCmUmAmU#mG# |
| | mC#mU (SEQ ID NO: 233) |
| crRNA 121 | mN#mN#mN#mNmNmNmNmNmNmNfNfNfNfNrN#rN#f |
| | NfNsNmNmGrU#rU#rU#fUfAmGmAmGmCmUmAmU#m |
| | G#mC#mU (SEQ ID NO: 234) |
| crRNA 122 | mN#mN#mN#mNmNmNmNmNmNmNfNfNfNfNrN#rN#f |
| | NfNrN#mNmGsUrU#rU#fUfAmGmAmGmCmUmAmU#m |
| | G#mC#mU (SEQ ID NO: 235) |
| crRNA 123 | mN#mN#mN#mNmNmNmNmNmNmNfNfNfNfNrN#rN#f |
| | NfNrN#mNmGrU#sUrU#fUfAmGmAmGmCmUmAmU#m |
| | G#mC#mU (SEQ ID NO: 236) |
| crRNA 124 | mN#mN#mN#mNmNmNmNmNmNmNfNfNfNfNrN#rN#f |
| | NfNrN#mNmGrU#rU#sUfUfAmGmAmGmCmUmAmU#m |
| | G#mC#mU (SEQ ID NO: 237) |
| crRNA 125 | mN#mN#mN#mNmNmNmNmNmNmNfNfNfNfNrN#rN#f |
| | NfNrN#mNmGsUrU#sUfUfAmGmAmGmCmUmAmU#mG |
| | #mC#mU (SEQ ID NO: 238) |
| crRNA 126 | mN#mN#mN#mNmNmNmNmNmNmNfNfNfNfNrN#rN#f |
| | NfNrN#mNmGsUsUrU#fUfAmGmAmGmCmUmAmU#mG |
| | #mC#mU (SEQ ID NO: 239) |
| crRNA 127 | mN#mN#mN#mNmNmNmNmNmNmNfNfNfNfNrN#rN#f |
| | NfNrN#mNmGrU#sUsUfUfAmGmAmGmCmUmAmU#mG |
| | #mC#mU (SEQ ID NO: 240) |
| crRNA 128 | mN#mN#mN#mNmNmNmNmNmNmNfNfNfNfNrN#rN#f |
| | NfNrN#mNmGaUaUaUfUfAmGmAmGmCmUmAmU#mG |
| | #mC#mU (SEQ ID NO: 241) |
| crRNA 129 | mN#mN#mN#mNmNmNmNmNmNmNfNfNfNfNrN#rN#f |
| | NfNrN#mNmGsU#sU#sU#fUfAmGmAmGmCmUmAmU# |
| | mG#mC#mU (SEQ ID NO: 242) |
| crRNA 130 | mN#mN#mN#mNmNmNmNmNmNmNfNfNfNfNrN#rN#f |
| | NfNrN#mNmGsUsUsUfUfAmGmAmGmCmUmAmU#mG# |
| | mC#mU (SEQ ID NO: 243) |
| crRNA 131 | mN#mN#mN#mNmNmNmNmNmNmNfNfNfNfNrN#aNfN |
| | fNrN#mNmGrU#rU#rU#fUfAmGmAmGmCmUmAmU#m |
| | G#mC#mU (SEQ ID NO: 244) |
| crRNA 132 | mN#mN#mN#mNmNmNmNmNmNmNfNfNfNfNrN#rN#f |
| | NfNaNmNmGrU#rU#rU#fUfAmGmAmGmCmUmAmU#m |
| | G#mC#mU (SEQ ID NO: 228) |
| crRNA 133 | mN#mN#mN#mNmNmNmNmNmNmNfNfNfNfNrN#aN#f |
| | NfNaN#mNmGrU#rU#rU#fUfAmGmAmGmCmUmAmU# |
| | mG#mC#mU (SEQ ID NO: 245) |
| crRNA 134 | mN#mN#mN#mNmNmNmNmNmNmNfNfNfNfNrN#rN#f |
| | NfNrN#mNmGaUaUaUfUfAmGmAmGmCmUmAmU#mG |
| | #mC#mU (SEQ ID NO: 241) |

wherein rN = RNA, mN = 2′-O-methyl RNA, fN = 2′-fluoro RNA, dN = 2′-deoxy RNA, aN = 2′-NH₂ (2′-amino RNA), sN = 4′-thio RNA, N#N = phosphorothioate linkage, and N = any nucleotide.

In one aspect, the disclosure provides a chemically modified guide RNA comprising: (a) a crRNA portion comprising (i) a guide sequence capable of hybridizing to a target polynucleotide sequence, and (ii) a repeat sequence; and (b) a tracrRNA portion comprising an anti-repeat nucleotide sequence that is complementary to the repeat sequence, wherein the tracrRNA portion comprises a modification pattern selected from any one of tracrRNAs 21-116 of Table 2.

In one aspect, the disclosure provides a chemically modified guide RNA comprising: (a) a crRNA portion comprising (i) a guide sequence capable of hybridizing to a target polynucleotide sequence, and (ii) a repeat sequence; and (b) a tracrRNA portion comprising an anti-repeat nucleotide sequence that is complementary to the repeat sequence, wherein the tracrRNA portion comprises a modification pattern selected from the group consisting of:

| Name | Sequence |
|---|---|
| tracrRNA | mA#mG#mC#mAmUmAmGmCmAmAmGrU#rU#mArA# |
| 21 | mAmAfUmAmAmGmGfCfUmArG#fUfCmCrG#rU# |
| | rU#mAmUmCraAmAmCmUmUmGmAmAmAmAmAmGmU |
| | mGmGmCmAmCmCmGmAmGmUmCmGmGmUmGmC#mU# |
| | mU#mU (SEQ ID NO: 29) |
| tracrRNA | mA#mG#mC#mAmUmAmGmCmAmAmGmUrUmArAmA |
| 22 | mArUmAmAmGmGrCrUmArGrUrCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 30) |
| tracrRNA | mA#mG#mC#mAmUmAmGmCmAmAmGrUmUmArAmA |
| 23 | mArUmAmAmGmGrCrUmArGrUrCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 31) |
| tracrRNA | mA#mG#mC#mAmUmAmGmCmAmAmGrUrUmAmAmA |
| 24 | mArUmAmAmGmGrCrUmArGrUrCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 13) |
| tracrRNA | mA#mG#mC#mAmUmAmGmCmAmAmGrUrUmArAmA |
| 25 | mAmUmAmAmGmGrCrUmArGrUrCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 32) |
| tracrRNA | mA#mG#mC#mAmUmAmGmCmAmAmGrUrUmArAmA |
| 26 | mArUmAmAmGmGmCrUmArGrUrCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 33) |
| tracrRNA | mA#mG#mC#mAmUmAmGmCmAmAmGrUrUmArAmA |
| 27 | mArUmAmAmGmGrCmUmArGrUrCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 34) |
| tracrRNA | mA#mG#mC#mAmUmAmGmCmAmAmGrUrUmArAmA |
| 28 | mArUmAmAmGmGrCrUmAmGrUrCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 35) |
| tracrRNA | mA#mG#mC#mAmUmAmGmCmAmAmGrUrUmArAmA |
| 29 | mArUmAmAmGmGrCrUmArGmUrCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 36) |
| tracrRNA | mA#mG#mC#mAmUmAmGmCmAmAmGrUrUmArAmA |
| 30 | mArUmAmAmGmGrCrUmArGrUmCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 37) |
| tracrRNA | mA#mG#mC#mAmUmAmGmCmAmAmGrUrUmArAmA |
| 31 | mArUmAmAmGmGrCrUmArGrUrCmCmGrUrUmAmU |
| | mCmAmAmCmUmUmGmAmAmAmAmAmGmUmGmGmCmAm |
| | CmCmGmAmGmUmCmGmGmUmGmC#mU#mU#mU |
| | (SFQ ID NO: 38) |
| tracrRNA | mA#mG#mC#mAmUmAmGmCmAmAmGrUrUmArAmA |
| 32 | mArUmAmAmGmGrCrUmArGrUrCmCrGmUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 39) |
| tracrRNA | mA#mG#mC#mAmUmAmGmCmAmAmGrUrUmArAmA |
| 33 | mArUmAmAmGmGrCrUmArGrUrCmCrGrUmUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 40) |
| tracrRNA | mA#mG#mC#mAmUmAmGmCmAmAmGrU#rUmArAmA |
| 34 | mArUmAmAmGmGrCrUmArGrUrCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 41) |
| tracrRNA | mA#mG#mC#mAmUmAmGmCmAmAmGrUrU#mArAmA |
| 35 | mArUmAmAmGmGrCrUmArGrUrCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 42) |
| tracrRNA | mA#mG#mC#mAmUmAmGmCmAmAmGrUrUmArA#mA |
| 36 | mArUmAmAmGmGrCrUmArGrUrCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SFQIDNO43) |
| tracrRNA | mA#mG#mC#mAmUmAmGmCmAmAmGrUrUmArAmA |
| 37 | mArU#mAmAmGmGrCrUmArGrUrCmCrGrUrUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 44) |
| tracrRNA | mA#mG#mC#mAmUmAmGmCmAmAmGrUrUmArAmA |
| 38 | mArUmAmAmGmGrC#rUmArGrUrCmCrGrUrUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 45) |
| tracrRNA | mA#mG#mC#mAmUmAmGmCmAmAmGrUrUmArAmA |
| 39 | mArUmAmAmGmGrCrU#mArGrUrCmCrGrUrUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 46) |
| tracrRNA | mA#mG#mC#mAmUmAmGmCmAmAmGrUrUmArAmA |
| 40 | mArUmAmAmGmGrCrUmArG#rUrCmCrGrUrUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 47) |
| tracrRNA | mA#mG#mC#mAmUmAmGmCmAmAmGrUrUmArAmA |
| 41 | mArUmAmAmGmGrCrUmArGrU#rCmCrGrUrUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 48) |
| tracrRNA | mA#mG#mC#mAmUmAmGmCmAmAmGrUrUmArAmA |
| 42 | mArUmAmAmGmGrCrUmArGrUrC#mCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 49) |
| tracrRNA | mA#mG#mC#mAmUmAmGmCmAmAmGrUrUmArAmA |
| 43 | mArUmAmAmGmGrCrUmArGrUrCmCrG#rUrUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 50) |
| tracrRNA | mA#mG#mC#mAmUmAmGmCmAmAmGrUrUmArAmA |
| 44 | mArUmAmAmGmGrCrUmArGrUrCmCrGrU#rUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 51) |
| tracrRNA | mA#mG#mC#mAmUmAmGmCmAmAmGrUrUmArAmA |
| 45 | mArUmAmAmGmGrCrUmArGrUrCmCrGrUrU#mAm |
| | UmCmAmAmCmUmUmGmAmAmAmAmAmGmUmGmGmCm |
| | AmCmCmGmAmGmUmCmGmGmUmGmC#mU#mU#mU |
| | (SFQIDNO:52) |
| tracrRNA | mA#mG#mC#mAmUmAmGmCmAmAmGfUfUmArAmA |
| 46 | mArUmAmAmGmGrCrUmArGrUrCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 53) |
| tracrRNA | mA#mG#mC#mAmUmAmGmCmAmAmGrUrUmAfAmA |
| 47 | mAmUmAmAmGmGrCrUmArGrUrCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 54) |
| tracrRNA | mA#mG#mC#mAmUmAmGmCmAmAmGfUfUmAfAmA |
| 48 | mCmAfUmAmAmGmGrCrUmArGrUrCmCrGrUrUmA |
| | mUmAmAmCmUmUmGmAmAmAmAmAmGmUmGmGmCm |
| | AmCmCmGmAmGmUmCmGmGmUmGmC#mU#mU#mU |
| | (SEQ ID NO: 55) |
| tracrRNA | mA#mG#mC#mAmUmAmGmCmAmAmGrUrUmArAmA |
| 49 | mArUmAmAmGmGfCfUmArGrUrCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 56) |
| tracrRNA | mA#mG#mC#mAmUmAmGmCmAmAmGrUrUmArAmA |
| 50 | mArUmAmAmGmGrCrUmAfGfUfCmCrGrUrUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 57) |
| tracrRNA | mA#mG#mC#mAmUmAmGmCmAmAmGrUrUmArAmA |
| 51 | mArUmAmAmGmGrCrUmArGrUrCmCfGfUfUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 58) |
| tracrRNA | mA#mG#mC#mAmUmAmGmCmAmAmGrUrUmArAmA |
| 52 | mArUmAmAmGmGfCfUmArGrUrCmCfGfUfUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 59) |
| tracrRNA | mA#mG#mC#mAmUmAmGmCmAmAmGrUrUmArAmA |
| 53 | mArUmAmAmGmGfCfUmAfGfUfCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 60) |
| tracrRNA | mA#mG#mC#mAmUmAmGmCmAmAmGrUrUmArAmA |
| 54 | mArUmAmAmGmGrCrUmArGfUfCmCfGfUfUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 61) |
| tracrRNA | mA#mG#mC#mAmUmAmGmCmAmAmGrUrUmArAmA |
| 55 | mArUmAmAmGmGrCrUmAfGfUfCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 57) |
| tracrRNA | mA#mG#mC#mAmUmAmGmCmAmAmGrUrUmArAmA |
| 56 | mArUmAmAmGmGrCrUmArGrUrCmCfGrUfUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 62) |
| tracrRNA | mA#mG#mC#mAmUmAmGmCmAmAmGrUrUmArAmA |
| 57 | mArUmAmAmGmGfCfUmAfGrUfCmCfGrUfUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 63) |
| tracrRNA | mA#mG#mC#mAmUmAmGmCmAmAmGrUrUmArAmA |
| 58 | mArUmAmAmGmGrCrUmAfGrUfCmCfGrUfUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 64) |
| tracrRNA | mA#mG#mC#mAmUmAmGmCmAmAmGrUrUmArAmA |
| 59 | mArUmAmAmGmGfCrUmAfGrUfCmCfGrUfUmAm |
| | UmCmAmAmCmUmUmGmAmAmAmAmAmGmUmGmGmCm |
| | AmCmCmGmAmGmUmCmGmGmUmGmC#mU#mU#mU |
| | (SFQIDNO:65) |
| tracrRNA | mA#mG#mC#mAmUmAmGmCmAmAmGmUmUmArAm |
| 60 | AmArUmAmAmGmGrCrUmArGrUrCmCrGrUrUm |
| | AmUmCmAmAmCmUmUmGmAmAmAmAmAmGmUmGmGm |
| | CmAmCmCmGmAmGmUmCmGmGmUmGmC#mU#mU#m |
| | U (SEQ ID NO: 66) |
| tracrRNA | mA#mG#mC#mAmUmAmGmCmAmAmGrUrUmAmAmA |
| 61 | mAmUmAmAmGmGrCrUmArGrUrCmCrGrUrUmAmU |
| | mCmAmAmCmUmUmGmAmAmAmAmAmGmUmGmGmCm |
| | AmCmCmGmAmGmUmCmGmGmUmGmC#mU#mU#mU |
| | (SEQ ID NO: 67) |
| tracrRNA | mA#mG#mC#mAmUmAmGmCmAmAmGmUmUmAmAm |
| 62 | AmAmUmAmAmGmGrCrUmArGrUrCmCrGrUrUmAm |
| | UmCmAmAmCmUmUmGmAmAmAmAmAmGmUmGmGmC |
| | mAmCmCmGmAmGmUmCmGmGmUmGmC#mU#mU#m |
| | U (SEQ ID NO: 68) |
| tracrRNA | mA#mG#mC#mAmUmAmGmCmAmAmGrUrUmArAmA |
| 63 | mArUmAmAmGmGmCmUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 69) |
| tracrRNA | mA#mG#mC#mAmUmAmGmCmAmAmGrUrUmArAmA |
| 64 | mArUmAmAmGmGrCrUmAmGmUmCmCrGrUrUmAmU |
| | mCmAmAmCmUmUmGmAmAmAmAmAmGmUmGmGm |
| | CmAmCmCmGmAmGmUmCmGmGmUmGmC#mU#mU# |
| | mU (SEQ ID NO: 70) |
| tracrRNA | mA#mG#mC#mAmUmAmGmCmAmAmGrUrUmArAmA |
| 65 | mArUmAmAmGmGrCrUmArGrUrCmCmGmUmUmAmU |
| | mCmAmAmCmUmUmGmAmAmAmAmAmGmUmGmGm |
| | CmAmCmCmGmAmGmUmCmGmGmUmGmC#mU#mU# |
| | mU (SEQ ID NO: 71) |
| tracrRNA | mA#mG#mC#mAmUmAmGmCmAmAmGrUrUmArAmA |
| 66 | mArUmAmAmGmGmCmUmArGrUrCmCmGmUmUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 72) |
| tracrRNA | mA#mG#mC#mAmUmAmGmCmAmAmGrUrUmArAmA |
| 67 | lnArUmAmAmGmGmCmUmAmGmUmCmCrGrUrUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 73) |
| tracrRNA | mA#mG#mC#mAmUmAmGmCmAmAmGrUrUmArAmA |
| 68 | mArUmAmAmGmGrCrUmAmGmUmCmCmGmUmUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 74) |
| tracrRNA | mA#mG#mC#mAmUmAmGmCmAmAmGrUrUmArAmA |
| 69 | mArUmAmAmGmGrCrUmAmGrUmCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 75) |
| tracrRNA | mA#mG#mC#mAmUmAmGmCmAmAmGrUrUmArAmA |
| 70 | mArUmAmAmGmGrCrUmArGrUrCmCmGrUmUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 76) |
| tracrRNA | mA#mG#mC#niAmUmAmGmCmAmAmGrUrUmArAmA |
| 71 | mArUmAmAmGmGmCmUmAmGrUmCmCmGrUmUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 77) |
| tracrRNA | mA#mG#mC#mAmUmAmGmCmAmAmGrUrUmArAmA |
| 72 | mArUmAmAmGmGrCrUmAmGrUmCmCmGrUmUmAmU |
| | mCmAmAmCmUmUmGmAmAmAmAmAmGmUmGmGm |
| | CmAmCmCmGmAmGmUmCmGmGmUmGmC#mU#mU# |
| | mU (SEQ ID NO: 78) |
| tracrRNA 73 | mA#mG#mC#mAmUmAmGmCmAmAmGrUrUmArAmA |
| | mArUmAmAmGmGmCrUmAmGrUmCmCmGrUmUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 79) |
| tracrRNA | mA#mG#mC#mAmUmAmGmCmAmAmGdUdUmArAmA |
| 74 | mArUmAmAmGmGrCrUmArGrUrCmCrGrUrUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 80) |
| tracrRNA | mA#mG#mC#mAmUmAmGmCmAmAmGrUrUmAdAmA |
| 75 | mAdUmAmAmGmGrCrUmArGrUrCmCrGrUrUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 81) |
| tracrRNA | mA#mG#mC#mAmUmAmGmCmAmAmGdUdUmAdAmA |
| 76 | mAdUmAmAmGmGrCrUmArGrUrCmCrGrUrUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 82) |
| tracrRNA | mA#mG#mC#mAmUmAmGmCmAmAmGrUrUmArAmA |
| 77 | mArUmAmAmGmGdCdUmArGrUrCmCrGrUrUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 83) |
| tracrRNA | mA#mG#mC#mAmUmAmGmCmAmAmGrUrUmArAmA |
| | mArUmAmAmGmGrCrUmAdGdUdCmCrGrUrUmAm |
| 78 | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 84) |
| tracrRNA | mA#mG#mC#mAmUmAmGmCmAmAmGrUrUmArAmA |
| 79 | mArUmAmAmGmGrCrUmArGrUrCmCdGdUdUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 85) |
| tracrRNA | mA#mG#mC#mAmUmAmGmCmAmAmGrUrUmArAmA |
| 80 | mArUmAmAmGmGdCdUmArGrUrCmCdGdUdUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 86) |
| tracrRNA | mA#mG#mC#mAmUmAmGmCmAmAmGrUrUmArAmA |
| 81 | mArUmAmAmGmGdCdUmAdGdUdCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 87) |
| tracrRNA | mA#mG#mC#mAmUmAmGmCmAmAmGrUrUmArAmA |
| 82 | mArUmAmAmGmGrCrUmAdGdUdCmCdGdUdUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 88) |
| tracrRNA | mA#mG#mC#mAmUmAmGmCmAmAmGrUrUmArAmA |
| 83 | mArUmAmAmGmGrCrUmAdGrUdCmCrGrUrUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 89) |
| tracrRNA | mA#mG#mC#mAmUmAmGmCmAmAmGrUrUmArAmA |
| 84 | mArUmAmAmGmGrCrUmArGrUrCmCdGrUdUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 90) |
| tracrRNA | mA#mG#mC#mAmUmAmGmCmAmAmGrUrUmArAmA |
| 85 | mArUmAmAmGmGdCdUmAdGrUdCmCdGrUdUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 91) |
| tracrRNA | mA#mG#mC#mAmUmAmGmCmAmAmGrUrUmArAmA |
| 86 | mArUmAmAmGmGrCrUmAdGrUdCmCdGrUdUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 92) |
| tracrRNA | mA#mG#mC#mAmUmAmGmCmAmAmGrUrUmArAm |
| 87 | AmArUmAmAmGmGdCrUmAdGrUdCmCdGrUdUm |
| | AmUmCmAmAmCmUmUmGmAmAmAmAmAmGmUmGm |
| | GmCmAmCmCmGmAmGmUmCmGmGmUmGmC#mU#m |
| | U#Mu |
| | (SEQ ID NO: 93) |
| tracrRNA | mA#mG#mC#mAmUmAmGmCmAmAmGrU#rU#mArAm |
| 88 | AmArUmAmAmGmGrCrUmArGrUrCmCrGrUrUmAm |
| | UmCmAmAmCmUmUmGmAmAmAmAmAmGmUmGmGmC |
| | mAmCmCmGmAmGmUmCmGmGmUmGmC#mU#mU#m |
| | U (SEQ ID NO: 94) |
| tracrRNA | mA#mG#mC#mAmUmAmGmCmAmAmGrUrUmArA#mA |
| 89 | mArU#mAmAmGmGrCrUmArGrUrCmCrGrUrUmAm |
| | UmCmAmAmCmUmUmGmAmAmAmAmAmGmUmGmGmCm |
| | AmCmCmGmAmGmUmCmGmGmUmGmC#mU#mU#mU |
| | (SEQ ID NO: 95) |
| tracrRNA | mA#mG#mC#mAmUmAmGmCmAmAmGrU#rU#mArA# |
| 90 | mAmArU#mAmAmGmGrCrUmArGrUrCmCrGrUrUm |
| | AmUmCmAmAmCmUmUmGmAmAmAmAmAmGmUmGmG |
| | mCmAmCmCmGmAmGmUmCmGmGmUmGmCHmU#mU |
| | #mU (SEQ ID NO: 96) |
| tracrRNA | mA#mG#mC#mAmUmAmGmCmAmAmGrUrUmArAmA |
| 91 | mArUmAmAmGmGrC#rU#mArGrUrCmCrGrUrUm |
| | AmUmCmAmAmCmUmUmGmAmAmAmAmAmGmUmGmGm |
| | CmAmCmCmGmAmGmUmCmGmGmUmGmC#mU#mU#m |
| | U (SEQ ID NO: 97) |
| tracrRNA | mA#mG#mC#mAmUmAmGmCmAmAmGrUrUmArAmA |
| 92 | mArUmAmAmGmGrCrUmArG#rU#rC#mCrGrUrU |
| | mAmUmCmAmAmCmUmUmGmAmAmAmAmAmGmUmGmGm |
| | CmAmCmCmGmAmGmUmCmGmGmUmGmC#mU#mU# |
| | mU (SEQ ID NO: 98) |
| tracrRNA | mA#mG#mC#mAmUmAmGmCmAmAmGrUrUmArAmA |
| 93 | mArUmAmAmGmGrCrUmArGrUrCmCrG#rU#rU# |
| | mAmUmCmAmAmCmUmUmGmAmAmAmAmAmGmUmGmGm |
| | CmAmCmCmGmAmGmUmCmGmGmUmGmC#mU#mU# |
| | mU (SEQ ID NO: 99) |
| tracrRNA | mA#mG#mC#mAmUmAmGmCmAmAmGrUrUmArAmA |
| 94 | mArUmAmAmGmGrC#rU#mArGrUrCmCrG#rU#rU# |
| | mAmUmCmAmAmCmUmUmGmAmAmAmAmAmGmUmGmG |
| | mCmAmCmCmGmAmGmUmCmGmGmUmGmC#mU#mU |
| | #mU (SEQ ID NO: 100) |
| tracrRNA | mA#mG#mC#mAmUmAmGmCmAmAmGrUrUmArAmA |
| 95 | mArUmAmAmGmGrC#rU#mArG#rU#rC#mCrGrUr |
| | UmAmUmCmAmAmCmUmUmGmAmAmAmAmAmGmUmGmG |
| | mCmAmCmCmGmAmGmUmCmGmGmUmGmC#mU#mU |
| | #mU (SEQ ID NO: 101) |
| tracrRNA | mA#mG#mC#mAmUmAmGmCmAmAmGrUrUmArAmA |
| 96 | mArUmAmAmGmGrCrUmArG#rU#rC#mCrG#rU#rU |
| | #mAmUmCmAmAmCmUmUmGmAmAmAmAmAmGmUmGm |
| | GmCmAmCmCmGmAmGmUmCmGmGmUmGmC#mU#m |
| | U#mU (SEQ ID NO: 102) |
| tracrRNA | mA#mG#mC#mAmUmAmGmCmAmAmGrUrUmArAmAmA |
| 97 | rUmAmAmGmGrCrUmArG#rUrC#mCrGrUrUmAm |
| | UmCmAmAmCmUmUmGmAmAmAmAmAmGmUmGmGmC |
| | mAmCmCmGmAmGmUmCmGmGmUmGmC#mU#mU#m |
| | U (SEQ ID NO: 103) |
| tracrRNA | mA#mG#mC#mAmUmAmGmCmAmAmGrUrUmArAmA |
| 98 | mArUmAmAmGmGrCrUmArGrUrCmCrG#rUrU#m |
| | AmUmCmAmAmCmUmUmGmAmAmAmAmAmGmUmGmGmC |
| | mAmCmCmGmAmGmUmCmGmGmUmGmC#mU#mU#m |
| | U (SEQ ID NO: 104) |
| tracrRNA | mA#mG#mC#niAmUmAmGmCmAmAmGrUrUmArAmA |
| 99 | mArUmAmAmGmGrC#rU#mArG#rU#rCmCrG#rUr |
| | U#mAmUmCmAmAmCmUmUmGmAmAmAmAmAmGmUmGm |
| | GmCmAmCmCmGmAmGmUmCmGmGmUmGmC#mU#m |
| | U#mU (SEQ ID NO: 105) |
| tracrRNA | mA#mG#mC#mAmUmAmGmCmAmAmGrUrUmArAmA |
| 100 | mArUmAmAmGmGrCrUmArG#rU#rCmCrG#rUrU# |
| | mAmUmCmAmAmCmUmUmGmAmAmAmAmAmGmUmGmG |
| | mCmAmCmCmGmAmGmUmCmGmGmUmGmC#mU#mU |
| | #mU (SEQ ID NO: 106) |
| tracrRNA | mA#mG#mC#mAmUmAmGmCmAmAmGrUrUmArAmA |
| 101 | mArUmAmAmGmGrC#rUmArG#rU#rCmCrG#rUrU# |
| | mAmUmCmAmAmCmUmUmGmAmAmAmAmAmGmUmGmG |
| | mCmAmCmCmGmAmGmUmCmGmGmUmGmC#mU#mU |
| | #mU (SEQ ID NO: 107) |
| tracrRNA | mA#mG#mC#mAmUmAmGmCmAmAmGrUrUmArAmA |
| 104 | mAdUmAmAmGmGdCdUmArGdUdCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 108) |
| tracrRNA | mA#mG#mC#mAmUmAmGmCmAmAmGrUdUmArAmA |
| 105 | mAdUmAmAmGmGdCdUmAdGdUdCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 109) |
| tracrRNA | mA#mG#mC#mAmUmAmGmCmAmAmGdUdUmAdAmA |
| 106 | mAdUmAmAmGmGdCdUmAdGdUdCmCdGdUdUmAmU |
| | mCmAmAmCmUmUmGmAmAmAmAmAmGmUmGmGm |
| | CmAmCmCmGmAmGmUmCmGmGmUmGmC#mU#mU# |
| | mU (SEQ ID NO: 110) |
| tracrRNA | mA#mG#mC#mAmUmAmGmCmAmAmGsUsUmArAmA |
| 107 | mAsUmAmAmGmGrCsUmArGsUrCmCrGsUsUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 111) |
| tracrRNA | mA#mG#mC#mAmUmAmGmCmAmAmGsUsUmArAmA |
| 108 | mAsUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 112) |
| tracrRNA | mA#mG#mC#mAmUmAmGmCmAmAmGrUrUmArAmA |
| 109 | mArUmAmAmGmGrCsUmArGsUrCmCrGsUsUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 113) |
| tracrRNA | mA#mG#mC#mAmUmAmGmCmAmAmGsUrUmArAmA |
| 110 | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 114) |
| tracrRNA | mA#mG#mC#mAmUmAmGmCmAmAmGrUsUmArAmA |
| 111 | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 115) |
| tracrRNA | mA#mG#mC#mAmUmAmGmCmAmAmGrUrUmArAmA |
| 112 | mAsUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mil |
| | (SEQ ID NO: 116) |
| tracrRNA | mA#mG#mC#mAmUmAmGmCmAmAmGrUrUmArAmA |
| 113 | mArUmAmAmGmGrCsUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 117) |
| tracrRNA | mA#mG#mC#mAmUmAmGmCmAmAmGrUrUmArAmA |
| 114 | mArUmAmAmGmGrCrUmArGsUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 118) |
| tracrRNA | mA#mG#mC#mAmUmAmGmCmAmAmGrUrUmArAmA |
| 115 | mArUmAmAmGmGrCrUmArGrUrCmCrGsUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 119) |
| tracrRNA | mA#mG#mC#mAmUmAmGmCmAmAmGrUrUmArAmA |
| 116 | mArUmAmAmGmGrCrUmArGrUrCmCrGrUsUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 120) |

wherein rN = RNA, mN = 2′-O-methyl RNA, fN = 2′-fluoro RNA, dN = 2′-deoxy RNA, sN = 4′-thio RNA, N#N = phosphorothioate linkage, and N = any nucleotide.

In an embodiment, each modification of the ribose group is independently selected from the group consisting of 2′-O-methyl, 2′-fluoro, 2′-deoxy, 2′-O-(2-methoxyethyl) (MOE), 2′-NH₂ (2′-amino),4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC}).

In an embodiment, at least 50% of the ribose groups are chemically modified. In an embodiment, at least 80% of the ribose groups are chemically modified. In an embodiment, 100% of the ribose groups are chemically modified.

In an embodiment, each modification of the phosphate group is independently selected from the group consisting of a phosphorothioate, phosphonoacetate (PACE), thiophosphonoacetate (thioPACE), amide, triazole, phosphonate, and phosphotriester modification.

In an embodiment, each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

In an embodiment, the crRNA portion comprises at least 50% modified nucleotides. In an embodiment, the crRNA portion comprises at least 80% modified nucleotides. In an embodiment, the crRNA portion comprises at least 90% modified nucleotides. In an embodiment, the crRNA portion comprises 100% chemically modified nucleotides.

In an embodiment, the chemically modified guide RNA comprises a crRNA portion modification pattern selected any one of crRNAs 1-134 of Table 1.

In one aspect, the disclosure provides a chemically modified guide RNA comprising: (a) a crRNA portion comprising (i) a guide sequence capable of hybridizing to a target polynucleotide sequence, and (ii) a repeat sequence; and (b) a tracrRNA portion comprising an anti-repeat nucleotide sequence that is complementary to the repeat sequence, wherein the crRNA portion comprises at least one 2′-NH₂ (2′-amino RNA) modification.

In another aspect, the disclosure provides a chemically modified crRNA comprising at least one 2′-NH₂ (2′-amino RNA) modification.

In certain embodiments, a pyrimidine nucleotide comprises the 2′-NH₂ modification. In certain embodiments, a purine nucleotide comprises the 2′-NH₂ modification.

In certain embodiments, the crRNA portion comprises a 2′-NH₂ (2′-amino RNA) modification at one of more positions 16, 19, 22, 23, and 24 from the 5′ end of the crRNA portion (e.g., one of more positions 16, 19, 22, 23, and 24 from the 5′ end of the crRNA portion as set forth in SEQ ID NO: 1). In certain embodiments, the crRNA portion comprises a 2′-NH₂ (2′-amino) modification at position 16 from the 5′ end of the crRNA portion. In certain embodiments, the crRNA portion comprises a 2′-NH₂ (2′-amino) modification at position 19 from the 5′ end of the crRNA portion. In certain embodiments, the crRNA portion comprises a 2′-NH₂ (2′-amino) modification at position 22 from the 5′ end of the crRNA portion. In certain embodiments, the crRNA portion comprises a 2′-NH₂ (2′-amino) modification at position 23 from the 5′ end of the crRNA portion. In certain embodiments, the crRNA portion comprises a 2′-NH₂ (2′-amino) modification at position 24 from the 5′ end of the crRNA portion. In certain embodiments, the crRNA portion comprises a 2′-NH₂ (2′-amino) modification at positions 22, 23, and 24 from the 5′ end of the crRNA portion. In certain embodiments, the crRNA portion comprises a 2′-NH₂ (2′-amino) modification at positions 19, 22, 23, and 24 from the 5′ end of the crRNA portion. In certain embodiments, the crRNA portion comprises a 2′-NH₂ (2′-amino) modification at positions 16 and 19 from the 5′ end of the crRNA portion.

In certain embodiments, the crRNA portion further comprises one or more additional modified nucleotides, each independently selected from a modification of a ribose group, a phosphate group, a nucleobase, or a combination thereof.

In certain embodiments, each modification of the ribose group is independently selected from the group consisting of 2′-O-methyl, 2′-fluoro, 2′-deoxy, 2′-O-(2-methoxyethyl) (MOE), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC}).

In certain embodiments, each modification of the phosphate group is independently selected from the group consisting of a phosphorothioate, phosphonoacetate (PACE), thiophosphonoacetate (thioPACE), amide, triazole, phosphonate, or phosphotriester modification.

In certain embodiments, each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

In certain embodiments, the crRNA portion comprises at least 50% modified nucleotides (e.g., 50% modified nucleotides, 55% modified nucleotides, 60% modified nucleotides, 65% modified nucleotides, 70% modified nucleotides, 75% modified nucleotides, 80% modified nucleotides, 85% modified nucleotides, 90% modified nucleotides, 95% modified nucleotides, or 100% modified nucleotides).

In certain embodiments, the chemically modified guide RNA comprises a crRNA portion modification pattern selected from the group consisting of:

(crRNA 114)
(SEQ ID NO: 227)
mN#mN#mN#mNrnNmNmNmNmNmNfNfNfNfNr
N#rN#fNfNaNmNmGaUaUaUfUfAmGmAmGmC
mUmAmU#mG#mC#mU;
(crRNA 115)
(SEQ ID NO: 228)
mN#mN#mN#mNrnNmNmNmNmNmNfNfNfNfNr
N#rN#fNfNaNmNmGrU#rU#rU#fUfAmGmAm
GmCmUmAmU#mG#mC#mU;
(crRNA 116)
(SEQ ID NO: 229)
mN#mN#mN#mNmNmNmNrnNmNmNfNfNfNfN
rN#rN#fNfNrN#mNmGaUrU#rU#fUfAmGm
AmGmCmUmAmU#mG#mC#mU;
(crRNA 117)
(SEQ ID NO: 230)
mN#mN#mN#mNmNmNmNmNmNmNfNfNfNfNrN
rN#fNfNrN#mNmGrU#aUrU#fUfAmGmAm
GmCmUmAmU#mG#mC#mU;
(crRNA 118)
(SEQ ID NO: 231)
mN#mN#mN#rnNmNmNmNmNmNmNfNfNfNfNr
N#rN#fNfNrN#mNmGrU#rU#aUfUfAmGmAm
GmCmUmAmU#mG#mC#mU;
and
(crRNA 128)
(SEQ ID NO: 241)
mN#mN#mN#mNmNmNmNmNmNmNfNfNfNfNr
N#rN#fNfNrN#mNmGaUaUaUfUfAmGmAm
GmCmUmAmU#mG#mC#mU,

wherein rN = RNA, mN = 2′-O-methyl RNA, fN = 2′-fluoro RNA, dN = 2′-deoxy RNA, aN = 2′-NH₂ (2′-amino RNA), N#N = phosphorothioate linkage, and N = any nucleotide.

In certain embodiments, the tracrRNA portion comprises one or more modified nucleotides, each independently selected from a modification of a ribose group, a phosphate group, a nucleobase, or a combination thereof.

In certain embodiments, each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC}).

In certain embodiments, each modification of the phosphate group is independently selected from the group consisting of a phosphorothioate, phosphonoacetate (PACE), thiophosphonoacetate (thioPACE), amide, triazole, phosphonate, or phosphotriester modification.

In certain embodiments, each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

In certain embodiments, the tracrRNA portion comprises at least 50% modified nucleotides (e.g., 50% modified nucleotides, 55% modified nucleotides, 60% modified nucleotides, 65% modified nucleotides, 70% modified nucleotides, 75% modified nucleotides, 80% modified nucleotides, 85% modified nucleotides, 90% modified nucleotides, 95% modified nucleotides, or 100% modified nucleotides).

In certain embodiments, the tracrRNA portion comprises a modification pattern selected from the group consisting of: tracrRNA 1 through tracrRNA 116 of Table 2 (e.g., tracrRNA 1, tracrRNA 2, tracrRNA 3, tracrRNA 4, tracrRNA 5, tracrRNA 6, tracrRNA 7, tracrRNA 8, tracrRNA 9, tracrRNA 10, tracrRNA 11, tracrRNA 12, tracrRNA 13, tracrRNA 14, tracrRNA 15, tracrRNA 16, tracrRNA 17, tracrRNA 18, tracrRNA 19, tracrRNA 20, tracrRNA 21, tracrRNA 22, tracrRNA 23, tracrRNA 24, tracrRNA 25, tracrRNA 26, tracrRNA 27, tracrRNA 28, tracrRNA 29, tracrRNA 30, tracrRNA 31, tracrRNA 32, tracrRNA 33, tracrRNA 34, tracrRNA 35, tracrRNA 36, tracrRNA 37, tracrRNA 38, tracrRNA 39, tracrRNA 40, tracrRNA 41, tracrRNA 42, tracrRNA 43, tracrRNA 44, tracrRNA 45, tracrRNA 46, tracrRNA 47, tracrRNA 48, tracrRNA 49, tracrRNA 50, tracrRNA 51, tracrRNA 52, tracrRNA 53, tracrRNA 54, tracrRNA 55, tracrRNA 56, tracrRNA 57, tracrRNA 58, tracrRNA 59, tracrRNA 60, tracrRNA 61, tracrRNA 62, tracrRNA 63, tracrRNA 64, tracrRNA 65, tracrRNA 66, tracrRNA 67, tracrRNA 68, tracrRNA 69, tracrRNA 70, tracrRNA 71, tracrRNA 72, tracrRNA 73, tracrRNA 74, tracrRNA 75, tracrRNA 76, tracrRNA 77, tracrRNA 78, tracrRNA 79, tracrRNA 80, tracrRNA 81, tracrRNA 82, tracrRNA 83, tracrRNA 84, tracrRNA 85, tracrRNA 86, tracrRNA 87, tracrRNA 88, tracrRNA 89, tracrRNA 90, tracrRNA 91, tracrRNA 92, tracrRNA 93, tracrRNA 94, tracrRNA 95, tracrRNA 96, tracrRNA 97, tracrRNA 98, tracrRNA 99, tracrRNA 100, tracrRNA 101, tracrRNA 102, tracrRNA 103, tracrRNA 104, tracrRNA 105, tracrRNA 106, tracrRNA 107, tracrRNA 108, tracrRNA 109, tracrRNA 110, tracrRNA 111, tracrRNA 112, tracrRNA 113, tracrRNA 114, tracrRNA 115, or tracrRNA 116).

In one aspect, the disclosure provides a chemically modified guide RNA comprising: (a) a crRNA portion comprising (i) a guide sequence capable of hybridizing to a target polynucleotide sequence, and (ii) a repeat sequence; and (b) a tracrRNA portion comprising an anti-repeat nucleotide sequence that is complementary to the repeat sequence, wherein one or both of the crRNA portion and tracrRNA portion comprises at least one 4′-thio RNA modification.

In another aspect, the disclosure provides a chemically modified crRNA comprising at least one 4′-thio RNA modification.

In yet another aspect, the disclosure provides a chemically modified tracrRNA comprising at least one 4′-thio RNA modification.

In certain embodiments, the crRNA portion comprises a 4′-thio RNA modification at one of more positions 19, 22, 23, and 24 from the 5′ end of the crRNA portion (e.g., one of more positions 19, 22, 23, and 24from the 5′ end of the crRNA portion as set forth in SEQ ID NO: 1). In certain embodiments, the crRNA portion comprises a 4′-thio RNA modification at position 19 from the 5′ end of the crRNA portion. In certain embodiments, the crRNA portion comprises a 4′-thio RNA modification at position 22 from the 5′ end of the crRNA portion. In certain embodiments, the crRNA portion comprises a 4′-thio RNA modification at position 23 from the 5′ end of the crRNA portion. In certain embodiments, the crRNA portion comprises a 4′-thio RNA modification at position 24 from the 5′ end of the crRNA portion. In certain embodiments, the crRNA portion comprises a 4′-thio RNA modification at positions 22 and 23 from the 5′ end of the crRNA portion. In certain embodiments, the crRNA portion comprises a 4′-thio RNA modification at positions 22 and 24 from the 5′ end of the crRNA portion. In certain embodiments, the crRNA portion comprises a 4′-thio RNA modification at positions 23 and 24 from the 5′ end of the crRNA portion. In certain embodiments, the crRNA portion comprises a 4′-thio RNA modification at positions 19, 22, 23, and 24 from the 5′ end of the crRNA portion.

In certain embodiments, the tracrRNA portion comprises a 4′-thio RNA modification at one of more positions 12, 13, 18, 24, 27, 31, and 32 from the 5′ end of the tracrRNA portion (e.g., one of more positions 12, 13, 18, 24, 27, 31, and 32 from the 5′ end of the tracrRNA portion as set forth in SEQ ID NO: 2). In certain embodiments, the tracrRNA portion comprises a 4′-thio RNA modification at position 12 from the 5′ end of the tracrRNA portion. In certain embodiments, the tracrRNA portion comprises a 4′-thio RNA modification at position 13 from the 5′ end of the tracrRNA portion. In certain embodiments, the tracrRNA portion comprises a 4′-thio RNA modification at position 18 from the 5′ end of the tracrRNA portion. In certain embodiments, the tracrRNA portion comprises a 4′-thio RNA modification at position 24 from the 5′ end of the tracrRNA portion. In certain embodiments, the tracrRNA portion comprises a 4′-thio RNA modification at position 27 from the 5′ end of the tracrRNA portion. In certain embodiments, the tracrRNA portion comprises a 4′-thio RNA modification at position 31 from the 5′ end of the tracrRNA portion. In certain embodiments, the tracrRNA portion comprises a 4′-thio RNA modification at position 32 from the 5′ end of the tracrRNA portion. In certain embodiments, the tracrRNA portion comprises a 4′-thio RNA modification at positions 12, 13, and 18 from the 5′ end of the tracrRNA portion. In certain embodiments, the tracrRNA portion comprises a 4′-thio RNA modification at positions 24, 27, 31, and 32 from the 5′ end of the tracrRNA portion. In certain embodiments, the tracrRNA portion comprises a 4′-thio RNA modification at positions 12, 13, 18, 24, 27, 31, and 32 from the 5′ end of the tracrRNA portion.

In certain embodiments, the crRNA portion and/or the tracrRNA portion further comprise one or more additional modified nucleotides, each independently selected from a modification of a ribose group, a phosphate group, a nucleobase, or a combination thereof.

In certain embodiments, each modification of the ribose group is independently selected from the group consisting of 2′-O-methyl, 2′-fluoro, 2′-deoxy, 2′-O-(2-methoxyethyl) (MOE), 2′-NH₂ (2′-amino), a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC}).

In certain embodiments, each modification of the phosphate group is independently selected from the group consisting of a phosphorothioate, phosphonoacetate (PACE), thiophosphonoacetate (thioPACE), amide, triazole, phosphonate, or phosphotriester modification.

In certain embodiments, each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

In certain embodiments, the crRNA portion and/or the tracrRNA portion comprises at least 50% modified nucleotides (e.g., 50% modified nucleotides, 55% modified nucleotides, 60% modified nucleotides, 65% modified nucleotides, 70% modified nucleotides, 75% modified nucleotides, 80% modified nucleotides, 85% modified nucleotides, 90% modified nucleotides, 95% modified nucleotides, or 100% modified nucleotides).

In certain embodiments, the chemically modified guide RNA comprises a crRNA portion modification pattern selected from the group consisting of:

(crRNA 119)
(SEQ ID NO: 232)
mN#mN#mN#mNmNmNmNmNmNmNfNfNfNfNrN#rN#
fNfNsN#mNmGsU#sU#sU#fUfAmGmAmGmCmUmAm
U#mG#mC#mU;
(crRNA 120)
(SEQ ID NO: 233)
mN#mN#mN#mNmNmNmNmNmNmNfNfNfNfNrN#rN#
fNfNsNmNmGsUsUsUfUfAmGmAmGmCmUmAmU#
mGmC#mU;
(crRNA 121)
(SEQ ID NO: 234)
mN#mN#mN#mNmNmNmNmNmNmNfNfNfNfNrN#rN
fNfNsNmNmGrU#rU#rU#fUfAmGmAmGmCmUmA
mU#mG#mC#mU;
(crRNA 122)
(SEQ ID NO: 235)
mN#mN#mN#mNmNmNmNmNmNmNfNfNfNfNrN#r
NfNfNrN#mNmGsUrU#rU#fUfAmGmAmGmCmUmA
mU#mG#mC#mU;
(crRNA 123)
(SEQ ID NO: 236)
mN#mN#mN#mNmNmNmNmNmNmNfNfNfNfNrN#r
NfNfNrN#mNmGrU#sUrU#fUfAmGmAmGmCmUm
AmU#mG#mC#mU;
(crRNA 124)
(SEQ ID NO: 237)
mN#mN#mN#mNmNmNmNmNmNmNfNfNfNfNrN#rN
fNfNrN#mNmGrU#rU#sUfUfAmGmAmGmCmUmA
mU#mG#mC#mU;
(crRNA 125)
(SEQ ID NO: 238)
mN#mN#mN#mNmNmNmNmNmNmNfNfNfNfNrN#r
NfNfNrN#mNmGsUrU#sUfUfAmGmAmGmCmUmA
mU#mG#mC#mU;
(crRNA 126)
(SEQ ID NO: 239)
mN#mN#mN#mNmNmNmNmNmNmNfNfNfNfNrN#r
NfNfNrN#mNmGsUsUrU#fUfAmGmAmGmCmUmA
mU#mG#mC#mU;
(crRNA 127)
(SEQ ID NO: 240)
mN#mN#mN#mNmNmNmNmNmNmNfNfNfNfNrN#r
NfNfNrN#mNmGrU#sUsUfUfAmGmAmGmCmUmA
mU#mG#mC#mU;
(crRNA 129)
(SEQ ID NO: 242)
mN#mN#mN#mNmNmNmNmNmNmNfNfNfNfNrN#r
NfNfNrN#mNmGsU#sU#sU#fUfAmGmAmGmCmU
mAmU#mG#mC#mU;
and
(crRNA 130)
(SEQ ID NO: 243)
mN#mN#mN#mNmNmNmNmNmNmNfNfNfNfNrN#r
NfNfNrN#mNmGsUsUsUfUfAmGmAmGmCmUmAm
UmG#mC#mU,

wherein rN = RNA, mN = 2′-O-methyl RNA, fN = 2′-fluoro RNA, sN = 4′-thio RNA, N#N = phosphorothioate linkage, and N = any nucleotide.

In certain embodiments, the tracrRNA portion comprises a modification pattern selected from the group consisting of: tracrRNA 1 through tracrRNA 116 of Table 2 (e.g., tracrRNA 1, tracrRNA 2, tracrRNA 3, tracrRNA 4, tracrRNA 5, tracrRNA 6, tracrRNA 7, tracrRNA 8, tracrRNA 9, tracrRNA 10, tracrRNA 11, tracrRNA 12, tracrRNA 13, tracrRNA 14, tracrRNA 15, tracrRNA 16, tracrRNA 17, tracrRNA 18, tracrRNA 19, tracrRNA 20, tracrRNA 21, tracrRNA 22, tracrRNA 23, tracrRNA 24, tracrRNA 25, tracrRNA 26, tracrRNA 27, tracrRNA 28, tracrRNA 29, tracrRNA 30, tracrRNA 31, tracrRNA 32, tracrRNA 33, tracrRNA 34, tracrRNA 35, tracrRNA 36, tracrRNA 37, tracrRNA 38, tracrRNA 39, tracrRNA 40, tracrRNA 41, tracrRNA 42, tracrRNA 43, tracrRNA 44, tracrRNA 45, tracrRNA 46, tracrRNA 47, tracrRNA 48, tracrRNA 49, tracrRNA 50, tracrRNA 51, tracrRNA 52, tracrRNA 53, tracrRNA 54, tracrRNA 55, tracrRNA 56, tracrRNA 57, tracrRNA 58, tracrRNA 59, tracrRNA 60, tracrRNA 61, tracrRNA 62, tracrRNA 63, tracrRNA 64, tracrRNA 65, tracrRNA 66, tracrRNA 67, tracrRNA 68, tracrRNA 69, tracrRNA 70, tracrRNA 71, tracrRNA 72, tracrRNA 73, tracrRNA 74, tracrRNA 75, tracrRNA 76, tracrRNA 77, tracrRNA 78, tracrRNA 79, tracrRNA 80, tracrRNA 81, tracrRNA 82, tracrRNA 83, tracrRNA 84, tracrRNA 85, tracrRNA 86, tracrRNA 87, tracrRNA 88, tracrRNA 89, tracrRNA 90, tracrRNA 91, tracrRNA 92, tracrRNA 93, tracrRNA 94, tracrRNA 95, tracrRNA 96, tracrRNA 97, tracrRNA 98, tracrRNA 99, tracrRNA 100, tracrRNA 101, tracrRNA 102, tracrRNA 103, tracrRNA 104, tracrRNA 105, tracrRNA 106, tracrRNA 107, tracrRNA 108, tracrRNA 109, tracrRNA 110, tracrRNA 111, tracrRNA 112, tracrRNA 113, tracrRNA 114, tracrRNA 115, or tracrRNA 116).

In certain embodiments, the chemically modified guide RNA comprises a tracrRNA portion modification pattern selected from the group consisting of:

(tracrRNA 107)
(SEQ ID NO: 111)
mA#mG#mC#mAmUmAmGmCmAmAmG
sUsUmArAmAmAsUmAmAmGmGrCs
UmArGsUrCmCrGsUsUmAmUmCmA
mAmCmUmUmGmAmAmAmAmAmGmUm
GmGmCmAmCmCmGmAmGmUmCmGmG
mUmGmC#mU#mU#mU;
(tracrRNA 108)
(SEQ ID NO: 112)
mA#mG#mC#mAmUmAmGmCmAmAmG
sUsUmArAmAmAsUmAmAmGmGrCr
UmArGrUrCmCrGrUrUmAmUmCmA
mAmCmUmUmGmAmAmAmAmAmGmUm
GmGmCmAmCmCmGmAmGmUmCmGmG
mUmGmC#mU#mU#mU;
(tracrRNA 109)
(SEQ ID NO: 113)
mA#mG#mC#mAmUmAmGmCmAmAmG
rUrUmArAmAmArUmAmAmGmGrCs
UmArGsUrCmCrGsUsUmAmUmCmA
mAmCmUmUmGmAmAmAmAmAmGmUm
GmGmCmAmCmCmGmAmGmUmCmGmG
mUmGmC#mU#mU#mU;
(tracrRNA 110)
(SEQ ID NO: 114)
mA#mG#mC#mAmUmAmGmCmAmAmG
sUrUmArAmAmArUmAmAmGmGrCr
UmArGrUrCmCrGrUrUmAmUmCmA
mAmCmUmUmGmAmAmAmAmAmGmUm
GmGmCmAmCmCmGmAmGmUmCmGmG
mUmGmC#mU#mU#mU;
(tracrRNA 111)
(SEQ ID NO: 115)
mA#mG#mC#mAmUmAmGmCmAmAmG
rUsUmArAmAmArUmAmAmGmGrCr
UmArGrUrCmCrGrUrUmAmUmCmA
mAmCmUmUmGmAmAmAmAmAmGmUm
GmGmCmAmCmCmGmAmGmUmCmGmG
mUmGmC#mU#mU#mU;
(SEQ ID NO: 116)
(tracrRNA 112)
mA#mG#mC#mAmUmAmGmCmAmAmG
rUrUmArAmAmAsUmAmAmGmGrCr
UmArGrUrCmCrGrUrUmAmUmCmA
mAmCmUmUmGmAmAmAmAmAmGmUm
GmGmCmAmCmCmGmAmGmUmCmGmG
mUmGmC#mU#mU#mU;
(tracrRNA 113)
(SEQ ID NO: 117)
mA#mG#mC#mAmUmAmGmCmAmAmG
rUrUmArAmAmArUmAmAmGmGrCs
UmArGrUrCmCrGrUrUmAmUmCmA
mAmCmUmUmGmAmAmAmAmAmGmUm
GmGmCmAmCmCmGmAmGmUmCmGmG
mUmGmC#mU#mU#mU;
(tracrRNA 114)
(SEQ ID NO: 118)
mA#mG#mC#mAmUmAmGmCmAmAmG
rUrUmArAmAmArUmAmAmGmGrCr
UmArGsUrCmCrGrUrUmAmUmCmA
mAmCmUmUmGmAmAmAmAmAmGmUm
GmGmCmAmCmCmGmAmGmUmCmGmG
mUmGmC#mU#mU#mU;
(tracrRNA 115)
(SEQ ID NO: 119)
mA#mG#mC#mAmUmAmGmCmAmAmG
rUrUmArAmAmArUmAmAmGmGrCr
UmArGrUrCmCrGsUrUmAmUmCmA
mAmCmUmUmGmAmAmAmAmAmGmU
mGmGmCmAmCmCmGmAmGmUmCmGm
GmUmGmC#mU#mU#mU;
and
(tracrRNA 116)
(SEQ ID NO: 120)
mA#mG#mC#mAmUmAmGmCmAmAm
GrUrUmArAmAmArUmAmAmGmGr
CrUmArGrUrCmCrGrUsUmAmUm
CmAmAmCmUmUmGmAmAmAmAmAm
GmUmGmGmCmAmCmCmGmAmGmUm
CmGmGmUmGmC#mU#mU#mU,

wherein rN = RNA, mN = 2′-O-methyl RNA, fN = 2′-fluoro RNA, sN = 4′-thio RNA, N#N = phosphorothioate linkage, and N = any nucleotide.

In certain embodiments, the crRNA portion comprises a modification pattern selected from the group consisting of: crRNA 1 through crRNA 134 of Table 1 (e.g., crRNA 1, crRNA 2, crRNA 3, crRNA 4, crRNA 5, crRNA 6, crRNA 7, crRNA 8, crRNA 9, crRNA 10, crRNA 11, crRNA 12, crRNA 13, crRNA 14, crRNA 15, crRNA 16, crRNA 17, crRNA 18, crRNA 19, crRNA 20, crRNA 21, crRNA 22, crRNA 23, crRNA 24, crRNA 25, crRNA 26, crRNA 27, crRNA 28, crRNA 29, crRNA 30, crRNA 31, crRNA 32, crRNA 33, crRNA 34, crRNA 35, crRNA 36, crRNA 37, crRNA 38, crRNA 39, crRNA 40, crRNA 41, crRNA 42, crRNA 43, crRNA 44, crRNA 45, crRNA 46, crRNA 47, crRNA 48, crRNA 49, crRNA 50, crRNA 51, crRNA 52, crRNA 53, crRNA 54, crRNA 55, crRNA 56, crRNA 57, crRNA 58, crRNA 59, crRNA 60, crRNA 61, crRNA 62, crRNA 63, crRNA 64, crRNA 65, crRNA 66, crRNA 67, crRNA 68, crRNA 69, crRNA 70, crRNA 71, crRNA 72, crRNA 73, crRNA 74, crRNA 75, crRNA 76, crRNA 77, crRNA 78, crRNA 79, crRNA 80, crRNA 81, crRNA 82, crRNA 83, crRNA 84, crRNA 85, crRNA 86, crRNA 87, crRNA 88, crRNA 89, crRNA 90, crRNA 91, crRNA 92, crRNA 93, crRNA 94, crRNA 95, crRNA 96, crRNA 97, crRNA 98, crRNA 99, crRNA 100, crRNA 101, crRNA 102, crRNA 103, crRNA 104, crRNA 105, crRNA 106, crRNA 107, crRNA 108, crRNA 109, crRNA 110, crRNA 111, crRNA 112, crRNA 113, crRNA 114, crRNA 115, crRNA 116, crRNA 117, crRNA 118, crRNA 119, crRNA 120, crRNA 121, crRNA 122, crRNA 123, crRNA 124, crRNA 125, crRNA 126, crRNA 127, crRNA 128, crRNA 129, crRNA 130, crRNA 131, crRNA 132, crRNA 133, or crRNA 134).

In an embodiment, the chemically modified guide RNA further comprises at least one moiety conjugated to the guide RNA. In an embodiment, the at least one moiety is conjugated to at least one of the 5′ end of the crRNA portion, the 3′ end of the crRNA portion, the 5′ end of the tracrRNA portion, or the 3′ end of the tracrRNA portion.

In an embodiment, the at least one moiety increases cellular uptake of the guide RNA. In an embodiment, the at least one moiety promotes specific tissue distribution of the guide RNA.

In an embodiment, the at least one moiety is selected from the group consisting of fatty acids, steroids, secosteroids, lipids, gangliosides analogs, nucleoside analogs, endocannabinoids, vitamins, receptor ligands, peptides, aptamers, and alkyl chains.

In an embodiment, the at least one moiety is selected from the group consisting of cholesterol, docosahexaenoic acid (DHA), docosanoic acid (DCA), lithocholic acid (LA), GalNAc, amphiphilic block copolymer (ABC), hydrophilic block copolymer (HBC), poloxamer, Cy5, and Cy3.

In an embodiment, the at least one moiety is conjugated to the guide RNA via a linker. In an embodiment, the linker is selected from the group consisting of an ethylene glycol chain, an alkyl chain, a polypeptide, a polysaccharide, and a block copolymer.

In an embodiment, the at least one moiety is a modified lipid. In an embodiment, the modified lipid is a branched lipid.

In an embodiment, the modified lipid is a branched lipid of Formula I, Formula I: X-MC(═Y)M-Z-[L-MC(═Y)M-R]n, where X is a moiety that links the lipid to the guide RNA, each Y is independently oxygen or sulfur, each M is independently CH₂, NH, O or S, Z is a branching group which allows two or three (“n”) chains to be joined to a chemically modified guide RNA, L is an optional linker moiety, and each R is independently a saturated, monounsaturated or polyunsaturated linear or branched moiety from 2 to 30 atoms in length, a sterol, or other hydrophobic group. In an embodiment, the modified lipid is a headgroup-modified lipid.

In an embodiment, the modified lipid is a headgroup-modified lipid of Formula II, Formula II: X-MC(═Y)M-Z-[L-MC(═Y)M-R]n-L-K-J, where X is a moiety that links the lipid to the guide RNA, each Y is independently oxygen or sulfur, each M is independently CH₂, NH, N-alkyl, O or S, Z is a branching group which allows two or three (“n”) chains to be joined to chemically modified guide RNA, each L is independently an optional linker moiety, and R is a saturated, monounsaturated or polyunsaturated linear or branched moiety from 2 to 30 atoms in length, a sterol, or other hydrophobic group, K is a phosphate, sulfate, or amide and J is an aminoalkane or quaternary aminoalkane group.

In an embodiment, the guide RNA binds to a Cas9 nuclease selected from the group consisting of S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

In an embodiment, the Cas9 is a variant Cas9 with altered activity.

In an embodiment, the variant Cas9 is selected from the group consisting of a Cas9 nickase (nCas9), a catalytically dead Cas9 (dCas9), a hyper accurate Cas9 (HypaCas9), a high fidelity Cas9 (Cas9-HF), an enhanced specificity Cas9 (eCas9), and an expanded PAM Cas9 (xCas9).

In an embodiment, the Cas9 off-target activity is reduced relative to an unmodified guide RNA.

In an embodiment, the Cas9 on-target activity is increased relative to an unmodified guide RNA.

In an embodiment, the chemically modified guide RNA further comprises a nucleotide or non-nucleotide loop or linker linking the 3′ end of the crRNA portion to the 5′ end of the tracrRNA portion.

In an embodiment, the non-nucleotide linker comprises an ethylene glycol oligomer linker. In an embodiment, the nucleotide loop is chemically modified. In an embodiment, the nucleotide loop comprises the nucleotide sequence of GAAA.

In an embodiment, the modified guide RNA comprises an increased GC nucleotide content in the repeat and anti-repeat region relative to an unmodified guide RNA.

In an embodiment, the modified guide RNA comprises ribose modifications in the repeat and anti-repeat region.

In an embodiment, the repeat and anti-repeat modifications enhance the stability of pairing between the crRNA portion and the tracrRNA portion.

In an embodiment, the crRNA portion comprises the guide RNA modification pattern of NNNNNNNNNNNNNNNNNNNNGUUUUAGAGCGAGCGC (SEQ ID NO: 3) and the tracrRNA portion comprises the guide RNA modification pattern of GCGCUCGCAAGUUAAAAUAAGGCUAGUCCGUUAUCAACUUGAAAAAGUGGCACCGAGUCGGUGCUUU (SEQ ID NO: 4), wherein N=any nucleotide.

In an embodiment, the crRNA portion comprises between 1 and 20 phosphorothioate modifications (i.e., 1, 2, 3, 4, 5, 6, 7, 8, 9, 10, 11, 12, 13, 14, 15, 16, 17, 18, 19, or 20 phosphorothioate modifications).

In an embodiment, the chemically modified guide RNA comprises at least about 50% activity relative to an unmodified guide RNA (e.g., 50% activity, 60% activity, 70% activity, 80% activity, 90% activity, 95% activity, or 100% activity, relative to an unmodified guide RNA).

In certain aspects, the disclosure provides a chemically modified guide RNA comprising:

(a) a crRNA portion comprising
(SEQ ID NO: 125)
mN#mN#mN#rN#rN#rN#mNmNmNmNfNfNfNfNfNfNfNfNfNmNmGfUfUfUfUfA
mGmAmGmCmUmAmU#mG#mC#mU (crRNA 39);
and
a tracrRNA portion comprising
(SEQ ID NO: 48)
mA#mG#mC#mAmUmAmGmCmAmAmGrUrUmArAmAmArUmAmAmGmGrCr
UmArGrU#rCmCrGrUrUmAmUmCmAmAmCmUmUmGmAmAmAmAmAmGmU
mGmGmCmAmCmCmGmAmGmUmCmGmGmUmGmC#mU#mU#mU (tracrRNA 41);
(b) a crRNA portion comprising
(SEQ ID NO: 6)
mN#mN#mN#dN#dN#dN#mNmNmNmNfNfNfNfNfNfNfNfNfNmNmGfUfUfUfUf
AmGmAmGmCmUmAmU#mG#mC#mU (crRNA 40);
and
a tracrRNA portion comprising
(SEQ ID NO: 48)
mA#mG#mC#mAmUmAmGmCmAmAmGrUrUmArAmAmArUmAmAmGmGrCr
UmArGrU#rCmCrGrUrUmAmUmCmAmAmCmUmUmGmAmAmAmAmAmGmU
mGmGmCmAmCmCmGmAmGmUmCmGmGmUmGmC#mU#mU#mU (tracrRNA 41);
(c) a crRNA portion comprising
(SEQ ID NO: 160)
mN#mN#mN#mNmNmNmNmNmNmNfNfNfNfNrN#rN#fNfNrN#mNmGrU#rU#r
U#fUfAmGmAmGmCmUmAmU#mG#mC#mU (crRNA 20);
and
a tracrRNA portion comprising
(SEQ ID NO: 48)
mA#mG#mC#mAmUmAmGmCmAmAmGrUrUmArAmAmArUmAmAmGmGrCr
UmArGrU#rCmCrGrUrUmAmUmCmAmAmCmUmUmGmAmAmAmAmAmGmU
mGmGmCmAmCmCmGmAmGmUmCmGmGmUmGmC#mU#mU#mU (tracrRNA 41);
(d) a crRNA portion comprising
(SEQ ID NO: 125)
mN#mN#mN#rN#rN#rN#mNmNmNmNfNfNfNfNfNfNfNfNfNmNmGfUfUfUfUfA
mGmAmGmCmUmAmU#mG#mC#mU (crRNA 39);
and
a tracrRNA portion comprising
(SEQ ID NO: 111)
mA#mG#mC#mAmUmAmGmCmAmAmGsUsUmArAmAmAsUmAmAmGmGrCs
UmArGsUrCmCrGsUsUmAmUmCmAmAmCmUmUmGmAmAmAmAmAmGmU
mGmGmCmAmCmCmGmAmGmUmCmGmGmUmGmC#mU#mU#mU (tracrRNA 107);
(e) a crRNA portion comprising
(SEQ ID NO: 6)
mN#mN#mN#dN#dN#dN#mNmNmNmNfNfNfNfNfNfNfNfNfNmNmGfUfUfUfUf
AmGmAmGmCmUmAmU#mG#mC#mU (crRNA 40);
and
a tracrRNA portion comprising
(SEQ ID NO: 111)
mA#mG#mC#mAmUmAmGmCmAmAmGsUsUmArAmAmAsUmAmAmGmGrCs
UmArGsUrCmCrGsUsUmAmUmCmAmAmCmUmUmGmAmAmAmAmAmGmU
mGmGmCmAmCmCmGmAmGmUmCmGmGmUmGmC#mU#mU#mU (tracrRNA 107);
or
(f) a crRNA portion comprising
(SEQ ID NO: 160)
mN#mN#mN#mNmNmNmNmNmNmNfNfNfNfNrN#rN#fNfNrN#mNmGrU#rU#r
U#fUfAmGmAmGmCmUmAmU#mG#mC#mU (crRNA 20);
and
a tracrRNA portion comprising
(SEQ ID NO: 111)
mA#mG#mC#mAmUmAmGmCmAmAmGsUsUmArAmAmAsUmAmAmGmGrCs
UmArGsUrCmCrGsUsUmAmUmCmAmAmCmUmUmGmAmAmAmAmAmGmU
mGmGmCmAmCmCmGmAmGmUmCmGmGmUmGmC#mU#mU#mU (tracrRNA 107).

In one aspect, the disclosure provides a method of altering expression of a target gene in a cell, comprising administering to said cell a genome editing system comprising: the chemically modified guide RNA of any of the embodiments recited above; and an RNA-guided nuclease or a polynucleotide encoding an RNA-guided nuclease.

In an embodiment, the target gene is in a cell in an organism.

In an embodiment, expression of the target gene is knocked out or knocked down.

In an embodiment, the sequence of the target gene is modified, edited, corrected or enhanced.

In an embodiment, the guide RNA and the RNA-guided nuclease comprise a ribonucleoprotein (RNP) complex.

In an embodiment, the RNA-guided nuclease is selected from the group consisting of S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

In an embodiment, the Cas9 is a variant Cas9 with altered activity. In an embodiment, the variant Cas9 is selected from the group consisting of a Cas9 nickase (nCas9), a catalytically dead Cas9 (dCas9), a hyper accurate Cas9 (HypaCas9), a high fidelity Cas9 (Cas9-HF), an enhanced specificity Cas9 (eCas9), and an expanded PAM Cas9 (xCas9).

In an embodiment, the polynucleotide encoding an RNA-guided nuclease comprises a vector. In an embodiment, the vector is a viral vector. In an embodiment, the viral vector is an adeno-associated virus (AAV) vector or a lentivirus (LV) vector. In an embodiment, the polynucleotide encoding an RNA-guided nuclease comprises a synthetic mRNA.

In an embodiment, expression of the target gene is reduced by at least about 20% (e.g., about 20%, about 30%, about 40%, about 50%, about 60%, about 70%, about 80%, about 90%, about 95%, or 100%).

In one aspect, the disclosure provides a CRISPR genome editing system comprising, a chemically modified guide RNA of any of the embodiments recited above; and an RNA-guided nuclease or a polynucleotide encoding an RNA-guided nuclease. In an embodiment, the RNA-guided nuclease is selected from the group consisting of S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9). In an embodiment, the Cas9 is a variant Cas9 with altered activity. In an embodiment, the variant Cas9 is selected from the group consisting of a Cas9 nickase (nCas9), a catalytically dead Cas9 (dCas9), a hyper accurate Cas9 (HypaCas9), a high fidelity Cas9 (Cas9-HF), an enhanced specificity Cas9 (eCas9), and an expanded PAM Cas9 (xCas9). In an embodiment, the Cas9 off-target activity is reduced relative to an unmodified guide RNA. In an embodiment, the Cas9 on-target activity is increased relative to an unmodified guide RNA.

### BRIEF DESCRIPTION OF THE DRAWINGS

The foregoing and other features and advantages of the present disclosure will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1A-FIG. 1C depict schematics of crRNA and tracrRNA. FIG. 1A is a crRNA (SEQ ID NO: 1) and tracrRNA (SEQ ID NO: 2) when paired with the target genomic DNA. FIG. 1B depicts the heavily modified crRNA C20 (SEQ ID NO: 432) and heavily modified tracrRNA T2 (SEQ ID NO: 433). FIG. 1C depicts the fully modified crRNA C21 (SEQ ID NO: 434)) and fully modified tracrRNA T8 (SEQ ID NO: 435).

FIG. 2A-FIG. 2C depict several additional chemically modified crRNAs (C10, C17-C22) tested in combination with several chemically modified tracrRNAs (T2, T6-T8) to form chemically modified crRNA:tracrRNA pairs. The various crRNA:tracrRNA pairs were used in a HEK293T TLR assay to determine genome editing efficiency. C0 and T0 represent an unmodified crRNA and an unmodified tracrRNA, respectively. Cells were transfected with 20 pmol (FIG. 2A), 100 pmol (FIG. 2B), and 8 pmol (FIG. 2C) of Cas9, crRNA, tracrRNA RNPs.

FIG. 3A-FIG. 3C depict several additional chemically modified tracrRNAs (T9-T20) tested in combination with the minimally modified crRNA C0 (FIG. 3A), the heavily modified crRNA C20 (FIG. 3B), and the fully modified crRNA C21 (FIG. 3C), to form chemically modified crRNA:tracrRNA pairs. The various crRNA:tracrRNA pairs were used in a HEK293T TLR assay to determine genome editing efficiency. Cells were transfected with 20 pmol of Cas9, crRNA, tracrRNA RNPs.

FIG. 4 depicts editing efficiencies several crRNAs tested (C23-C29). TracrRNAs T0, T2, and T3 were paired with the crRNAs. The Traffic Light Reporter Multi-Cas Variant 1 (TLR-MCV1) reporter was used. The graphs show the percentages of red fluorescent (RF) cells obtained by fluorescence activated cell sorting (FACS) analysis. Data are mean values of three biological replicates and error bars represent s.e.m.

FIG. 5 depicts editing efficiencies several crRNAs tested (C30-C44). TracrRNA T2 was paired with the crRNAs. The Traffic Light Reporter Multi-Cas Variant 1 (TLR-MCV1) reporter was used. The graphs show the percentages of red fluorescent (RF) cells obtained by fluorescence activated cell sorting (FACS) analysis. Data are mean values of three biological replicates and error bars represent s.e.m.

FIG. 6 depicts editing efficiencies crRNA C39, C40, and C45 paired with tracrRNAs T2, T9, T12, T17, T18, T38, T39, and T41. The Traffic Light Reporter Multi-Cas Variant 1 (TLR-MCV1) reporter was used. The graphs show the percentages of red fluorescent (RF) cells obtained by fluorescence activated cell sorting (FACS) analysis. Data are mean values of three biological replicates and error bars represent s.e.m.

FIG. 7 depicts editing efficiencies several tracrRNAs paired with crRNA C40. The Traffic Light Reporter Multi-Cas Variant 1a (TLR-MCV1a) reporter was used. The graphs show the percentages of red fluorescent (RF) cells obtained by fluorescence activated cell sorting (FACS) analysis. Data are mean values of three biological replicates and error bars represent s.e.m.

FIG. 8 depicts editing efficiencies tracrRNAs T46 to T106 paired with crRNA C40. The Traffic Light Reporter Multi-Cas Variant 1a (TLR-MCV1a) reporter was used. The graphs show the percentages of red fluorescent (RF) cells obtained by fluorescence activated cell sorting (FACS) analysis. Data are mean values of three biological replicates and error bars represent s.e.m.

FIG. 9 depicts editing efficiencies of modified crRNAs targeting endogenous Pcsk9. The RNA designs were tested by electroporation of Cas9 RNP in the mouse Hepa 1-6 cell line. The graphs show indel percentages based on Inference of CRISPR Edits (ICE) analysis of PCR and Sanger sequencing data of the locus. The data represent the means from three independent biological replicates and error bars represent s.e.m.

FIG. 10 depicts editing efficiencies several crRNAs tested (C52-C93). TracrRNA T2 was paired with the crRNAs. The Traffic Light Reporter Multi-Cas Variant 1 (TLR-MCV1) reporter was used. Each crRNA targeted the MCV1a sequence. The graphs show the percentages of red fluorescent (RF) cells obtained by fluorescence activated cell sorting (FACS) analysis. Data are mean values of three biological replicates and error bars represent s.e.m.

FIG. 11A-FIG. 11C depict editing efficiencies several crRNAs containing at least one 2′-amino modification or at least one thiol modification. TracrRNA T2 was paired with the crRNAs. The TLR-MCV1 reporter was used in FIG. 11A. A cell line stably expressing the TLR-MCV1 reporter, a SpCas9, and an unmodified tracrRNA was used in FIG. 11B. The mTmG reporter in mouse embryonic fibroblasts (MEFs) was used in FIG. 11C. The graphs show the percentages of fluorescent cells obtained by FACS analysis. Data are mean values of three biological replicates and error bars represent s.e.m.

FIG. 12 depicts editing efficiencies several tracrRNAs tested (T107-T116). CrRNA C20 was paired with the tracrRNAs. The TLR-MCV1 reporter or mTmG reporter was used. The graphs show the percentages of fluorescent cells obtained by FACS analysis. Data are mean values of three biological replicates and error bars represent s.e.m.

FIG. 13 depicts GFP immunohistochemical staining in the mTmG transgenic mouse six days after receiving an RNP containing the C20/T2 pair. A PBS injected mTmG transgenic mouse was used as a negative control.

FIG. 14 depicts GFP immunohistochemical staining in the mTmG transgenic mouse six days after receiving an RNP containing the C20/T41 pair. A PBS injected mTmG transgenic mouse was used as a negative control.

### DETAILED DESCRIPTION

Provided herewith are novel chemically modified crRNAs and tracrRNAs, including heavily or fully chemically modified crRNAs and tracrRNAs. In certain embodiments, crRNAs and tracrRNAs with 5′ and/or 3′ conjugated moieties are provided. In yet other embodiments, crRNAs and tracrRNAs with modifications in the repeat region of the crRNA or the anti-repeat region of the tracrRNA are provided. Methods of using the crRNAs and tracrRNAs of the disclosure for genome editing with a CRISPR nuclease and kits for performing the same are also provided.

Unless otherwise defined herein, nomenclature used in connection with cell and tissue culture, molecular biology, immunology, microbiology, genetics and protein and nucleic acid chemistry and hybridization described herein are those well-known and commonly used in the art. The methods and techniques provided herein are usually performed according to conventional methods well known in the art and as described in various general and more specific references that are cited and discussed throughout the present specification unless otherwise indicated. Enzymatic reactions and purification techniques are performed according to manufacturer's specifications unless otherwise specified, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art, unless otherwise specified. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the disclosure may be more readily understood, certain terms are first defined.

As used herein, the term “guide RNA” or “gRNA” refer to any nucleic acid that promotes the specific association (or “targeting”) of an RNA-guided nuclease such as a Cas9 to a target sequence (e.g., a genomic or episomal sequence) in a cell.

As used herein, a “modular” or “dual RNA” guide comprises more than one, and typically two, separate RNA molecules, such as a CRISPR RNA (crRNA) and a trans-activating crRNA (tracrRNA), which are usually associated with one another, for example by duplexing. gRNAs and their component parts are described throughout the literature (see, e.g., Briner et al. Mol. Cell, 56(2), 333-339 (2014), which is incorporated by reference).

As used herein, a “unimolecular gRNA,” “chimeric gRNA,” or “single guide RNA (sgRNA)” comprises a single RNA molecule. The sgRNA may be a crRNA and tracrRNA linked together. For example, the 3′ end of the crRNA may be linked to the 5′ end of the tracrRNA. A crRNA and a tracrRNA may be joined into a single unimolecular or chimeric gRNA, for example, by means of a four nucleotide (e.g., GAAA) “tetraloop” or “linker” sequence bridging complementary regions of the crRNA (at its 3′ end) and the tracrRNA (at its 5′ end).

As used herein, a “repeat” sequence or region is a nucleotide sequence at or near the 3′ end of the crRNA which is complementary to an anti-repeat sequence of a tracrRNA.

As used herein, an “anti-repeat” sequence or region is a nucleotide sequence at or near the 5′ end of the tracrRNA which is complementary to the repeat sequence of a crRNA.

Additional details regarding guide RNA structure and function, including the gRNA/Cas9 complex for genome editing may be found in, at least, Mali et al. Science, 339(6121), 823-826 (2013); Jiang et al. Nat. Biotechnol. 31(3). 233-239 (2013); and Jinek et al. Science, 337(6096), 816-821 (2012); which are incorporated by reference herein.

As used herein, a “guide sequence” or “targeting sequence” refers to the nucleotide sequence of a gRNA, whether unimolecular or modular, that is fully or partially complementary to a target domain or target polynucleotide within a DNA sequence in the genome of a cell where editing is desired. Guide sequences are typically 10-30 nucleotides in length, preferably 16-24 nucleotides in length (for example, 16, 17, 18, 19, 20, 21, 22, 23 or 24 nucleotides in length), and are at or near the 5′ terminus of a Cas9 gRNA.

As used herein, a “target domain” or “target polynucleotide sequence” is the DNA sequence in a genome of a cell that is complementary to the guide sequence of the gRNA.

In addition to the targeting domains, gRNAs typically include a plurality of domains that influence the formation or activity of gRNA/Cas9 complexes. For example, as mentioned above, the duplexed structure formed by first and secondary complementarity domains of a gRNA (also referred to as a repeat: anti-repeat duplex) interacts with the recognition (REC) lobe of Cas9 and may mediate the formation of Cas9/gRNA complexes (Nishimasu et al. Cell 156: 935-949 (2014); Nishimasu et al. Cell 162(2), 1113-1126 (2015), both incorporated by reference herein). It should be noted that the first and/or second complementarity domains can contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for example through the use of A-G swaps as described in Briner 2014, or A-U swaps. These and other similar modifications to the first and second complementarity domains are within the scope of the present disclosure.

Along with the first and second complementarity domains, Cas9 gRNAs typically include two or more additional duplexed regions that are necessary for nuclease activity in vivo but not necessarily in vitro (Nishimasu 2015, supra). A first stem-loop near the 3′ portion of the second complementarity domain is referred to variously as the “proximal domain,” “stem loop 1” (Nishimasu 2014, supra; Nishimasu 2015, supra) and the “nexus” (Briner 2014, supra). One or more additional stem loop structures are generally present near the 3′ end of the gRNA, with the number varying by species: S. pyogenes gRNAs typically include two 3′ stem loops (for a total of four stem loop structures including the repeat: anti-repeat duplex), while S. aureus and other species have only one (for a total of three). A description of conserved stem loop structures (and gRNA structures more generally) organized by species is provided in Briner 2014, which is incorporated herein by reference. Additional details regarding guide RNAs generally may be found in WO2018026976A1, which is incorporated herein by reference.

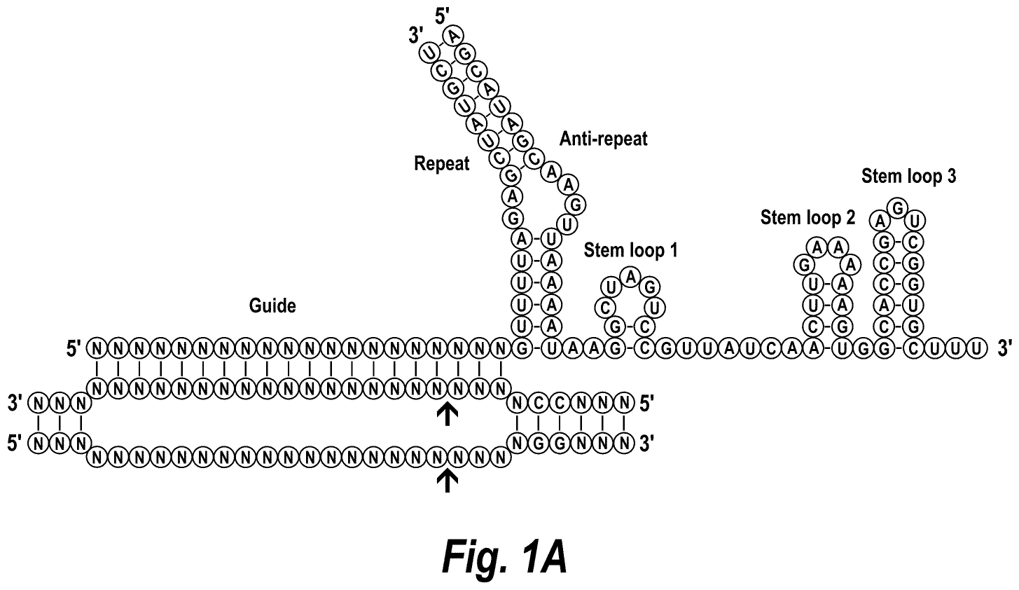

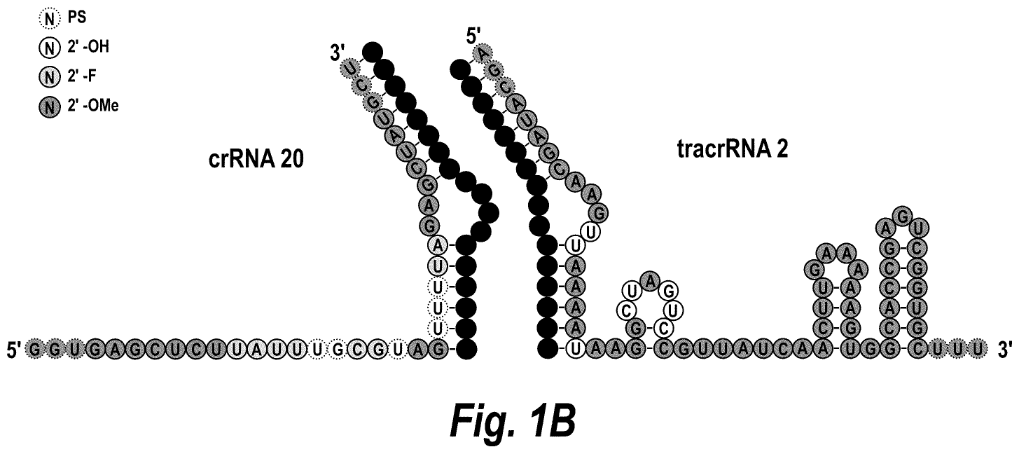

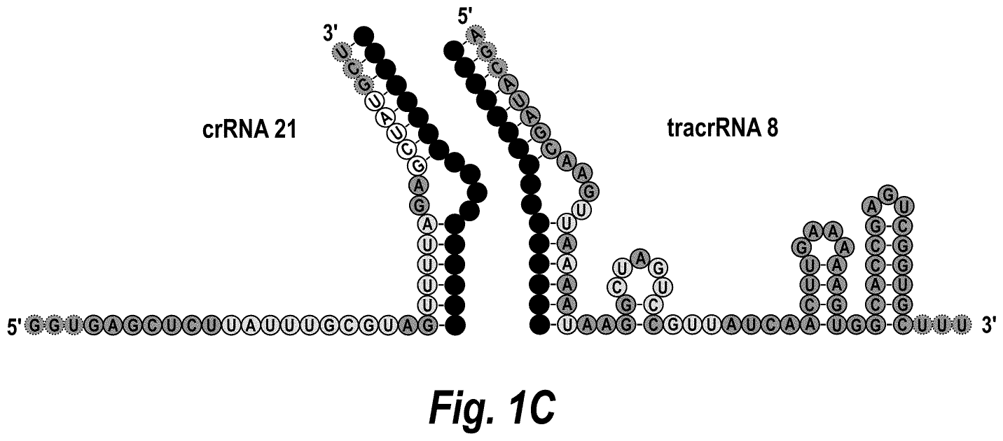

A representative guide RNA is shown in FIG. 1.

### Chemically Modified Guide RNA

The chemically modified guide RNAs of the disclosure possess improved in vivo stability, improved genome editing efficacy, and/or reduced immunotoxicity relative to unmodified or minimally modified guide RNAs.

Chemically modified guide RNAs of the disclosure contain one or more modified nucleotides comprising a modification in a ribose group, a phosphate group, a nucleobase, or a combination thereof.

Chemical modifications to the ribose group may include, but are not limited to, 2′-O-methyl, 2′-fluoro, 2′-deoxy, 2′-O-(2-methoxyethyl) (MOE), 2′-NH₂ (2′-amino), 4′-thio, 2′-O-Allyl, 2′-O-Ethylamine, 2′-O-Cyanoethyl, 2′-O-Acetalester, or a bicyclic nucleotide, such as locked nucleic acid (LNA), 2′-(S)-constrained ethyl (S-cEt), constrained MOE, or 2′-O,4′-C-aminomethylene bridged nucleic acid (2′,4′-BNA^{NC}).

The term “4′-thio” as used herein corresponds to a ribose group modification where the sugar ring oxygen of the ribose is replaced with a sulfur.

Chemical modifications to the phosphate group may include, but are not limited to, a phosphorothioate, phosphonoacetate (PACE), thiophosphonoacetate (thioPACE), amide, triazole, phosphonate, or phosphotriester modification.

In an embodiment, the crRNA portion of the chemically modified guide RNA comprises between 1 and 20 phosphorothioate modifications (i.e., 1, 2, 3, 4, 5, 6, 7, 8, 9, 10, 11, 12, 13, 14, 15, 16, 17, 18, 19, or 20 phosphorothioate modifications). In an embodiment, the crRNA portion of the chemically modified guide RNA comprises between 1 and 20 phosphorothioate modifications (i.e., 1, 2, 3, 4, 5, 6, 7, 8, 9, 10, 11, 12, 13, 14, 15, 16, 17, 18, 19, or 20 phosphorothioate modifications) and comprises at least about 50% activity relative to a guide RNA that does not comprise phosphorothioate modifications (e.g., 50% activity, 60% activity, 70% activity, 80% activity, 90% activity, 95% activity, or 100% activity, relative to a guide RNA that does not comprise phosphorothioate modifications).

Chemical modifications to the nucleobase may include, but are not limited to, 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, or halogenated aromatic groups.

The chemically modified guide RNAs may have one or more chemical modifications in the crRNA portion and/or the tracrRNA portion for a modular or dual RNA guide. The chemically modified guide RNAs may also have one or more chemical modifications in the single guide RNA for the unimolecular guide RNA.

The chemically modified guide RNAs may comprise at least about 50% to at least about 100% chemically modified nucleotides, at least about 60% to at least about 100% chemically modified nucleotides, at least about 70% to at least about 100% chemically modified nucleotides, at least about 80% to at least about 100% chemically modified nucleotides, at least about 90% to at least about 100% chemically modified nucleotides, and at least about 95% to at least about 100% chemically modified nucleotides.

The chemically modified guide RNAs may comprise at least about 50% chemically modified nucleotides, at least about 60% chemically modified nucleotides, at least about 70% chemically modified nucleotides, at least about 80% chemically modified nucleotides, at least about 90% chemically modified nucleotides, at least about 95% chemically modified nucleotides, at least about 99% chemically modified, or 100% (fully) chemically modified nucleotides.

The chemically modified guide RNAs may comprise at least about 80%, 81%, 82%, 83%, 84%, 85%, 86%, 87%, 88%, 89%, 90%, 91%, 92%, 93%, 94%, 95%, 96%, 97%, 98%, 99%, or 100% chemically modified nucleotides.

Guide RNAs that comprise at least about 80% chemically modified nucleotides to at least about 99% chemically modified nucleotides are considered “heavily” modified, as used herein.

Guide RNAs that comprise 100% chemically modified nucleotides are considered “fully” modified, as used herein.

In certain exemplary embodiments, the chemically modified guide RNAs may comprise a chemically modified ribose group at about 50% of the guide RNA nucleotides to about 100% of the guide RNA nucleotides, at about 60% of the guide RNA nucleotides to about 100% of the guide RNA nucleotides, at about 70% of the guide RNA nucleotides to about 100% of the guide RNA nucleotides, at about 80% of the guide RNA nucleotides to about 100% of the guide RNA nucleotides, at about 90% of the guide RNA nucleotides to about 100% of the guide RNA nucleotides, and at about 95% of the guide RNA nucleotides to about 100% of the guide RNA nucleotides

In certain exemplary embodiments, the chemically modified guide RNAs may comprise a chemically modified ribose group at about 50% of the guide RNA nucleotides, at about 60% of the guide RNA nucleotides, at about 70% of the guide RNA nucleotides, at about 80% of the guide RNA nucleotides, at about 90% of the guide RNA nucleotides, at about 95% of the guide RNA nucleotides, at about 99% of the guide RNA nucleotides, or at 100% of the guide RNA nucleotides.

In certain exemplary embodiments, the chemically modified guide RNAs may comprise a chemically modified ribose group at about 80%, 81%, 82%, 83%, 84%, 85%, 86%, 87%, 88%, 89%, 90%, 91%, 92%, 93%, 94%, 95%, 96%, 97%, 98%, 99%, or 100% of the guide RNA nucleotides.

Guide RNAs that have at least about 80% of the ribose groups chemically modified to at least about 99% of the ribose groups chemically modified are considered “heavily” modified, as used herein.

Guide RNAs that have 100% of the ribose groups chemically modified are considered “fully” modified, as used herein.

In certain exemplary embodiments, the chemically modified guide RNAs may comprise a chemically modified phosphate group at about 50% of the guide RNA nucleotides to about 100% of the guide RNA nucleotides, at about 60% of the guide RNA nucleotides to about 100% of the guide RNA nucleotides, at about 70% of the guide RNA nucleotides to about 100% of the guide RNA nucleotides, at about 80% of the guide RNA nucleotides to about 100% of the guide RNA nucleotides, at about 90% of the guide RNA nucleotides to about 100% of the guide RNA nucleotides, and at about 95% of the guide RNA nucleotides to about 100% of the guide RNA nucleotides

In certain exemplary embodiments, the chemically modified guide RNAs may comprise a chemically modified phosphate group at about 50% of the guide RNA nucleotides, at about 60% of the guide RNA nucleotides, at about 70% of the guide RNA nucleotides, at about 80% of the guide RNA nucleotides, at about 90% of the guide RNA nucleotides, at about 95% of the guide RNA nucleotides, at about 99% of the guide RNA nucleotides, or at 100% of the guide RNA nucleotides.

In certain exemplary embodiments, the chemically modified guide RNAs may comprise a chemically modified phosphate group at about 80%, 81%, 82%, 83%, 84%, 85%, 86%, 87%, 88%, 89%, 90%, 91%, 92%, 93%, 94%, 95%, 96%, 97%, 98%, 99%, or 100% of the guide RNA nucleotides.

Guide RNAs that have at least about 80% of the phosphate groups chemically modified to at least about 99% of the phosphate groups chemically modified are considered “heavily” modified, as used herein.

Guide RNAs that have 100% of the phosphate groups chemically modified are considered “fully” modified, as used herein.

In certain exemplary embodiments, the chemically modified guide RNAs may comprise a chemically modified nucleobase at about 50% of the guide RNA nucleotides to about 100% of the guide RNA nucleotides, at about 60% of the guide RNA nucleotides to about 100% of the guide RNA nucleotides, at about 70% of the guide RNA nucleotides to about 100% of the guide RNA nucleotides, at about 80% of the guide RNA nucleotides to about 100% of the guide RNA nucleotides, at about 90% of the guide RNA nucleotides to about 100% of the guide RNA nucleotides, and at about 95% of the guide RNA nucleotides to about 100% of the guide RNA nucleotides.

In certain exemplary embodiments, the chemically modified guide RNAs may comprise a chemically modified nucleobase at about 50% of the guide RNA nucleotides, at about 60% of the guide RNA nucleotides, at about 70% of the guide RNA nucleotides, at about 80% of the guide RNA nucleotides, at about 90% of the guide RNA nucleotides, at about 95% of the guide RNA nucleotides, at about 99% of the guide RNA nucleotides, or at 100% of the guide RNA nucleotides.

In certain exemplary embodiments, the chemically modified guide RNAs may comprise a chemically modified nucleobase at about 80%, 81%, 82%, 83%, 84%, 85%, 86%, 87%, 88%, 89%, 90%, 91%, 92%, 93%, 94%, 95%, 96%, 97%, 98%, 99%, or 100% of the guide RNA nucleotides.

Guide RNAs that have at least about 80% of the nucleobases chemically modified to at least about 99% of the nucleobases chemically modified are considered “heavily” modified, as used herein.

Guide RNAs that have 100% of the nucleobases chemically modified are considered “fully” modified, as used herein.

In certain exemplary embodiments, the chemically modified guide RNAs may comprise any combination of chemically modified ribose groups, chemically modified phosphate groups, and chemically modified nucleobases at about 50% of the guide RNA nucleotides to about 100% of the guide RNA nucleotides, at about 60% of the guide RNA nucleotides to about 100% of the guide RNA nucleotides, at about 70% of the guide RNA nucleotides to about 100% of the guide RNA nucleotides, at about 80% of the guide RNA nucleotides to about 100% of the guide RNA nucleotides, at about 90% of the guide RNA nucleotides to about 100% of the guide RNA nucleotides, and at about 95% of the guide RNA nucleotides to about 100% of the guide RNA nucleotides.

In certain exemplary embodiments, the chemically modified guide RNAs may comprise any combination of chemically modified ribose groups, chemically modified phosphate groups, and chemically modified nucleobases at about 50% of the guide RNA nucleotides, at about 60% of the guide RNA nucleotides, at about 70% of the guide RNA nucleotides, at about 80% of the guide RNA nucleotides, at about 90% of the guide RNA nucleotides, at about 95% of the guide RNA nucleotides, at about 99% of the guide RNA nucleotides, or at 100% of the guide RNA nucleotides.

In certain exemplary embodiments, the chemically modified guide RNAs may comprise any combination of chemically modified ribose groups, chemically modified phosphate groups, and chemically modified nucleobases at about 80%, 81%, 82%, 83%, 84%, 85%, 86%, 87%, 88%, 89%, 90%, 91%, 92%, 93%, 94%, 95%, 96%, 97%, 98%, 99%, or 100% of the guide RNA nucleotides.

Guide RNAs that have at least about 80% of any combination of the ribose groups, the phosphate groups, and the nucleobases chemically modified to at least about 99% of the nucleobases chemically modified are considered “heavily” modified, as used herein.

Guide RNAs that have 100% of any combination of the ribose groups, the phosphate groups, and the nucleobases chemically modified are considered “fully” modified, as used herein.

The heavily and fully chemically modified guide RNAs of the disclosure possess several advantages over the minimally modified guide RNAs in the art. Heavily and fully chemically modified guide RNAs are expected to ease chemical synthesis, further enhance in vivo stability, and provide a scaffold for terminally appended chemical functionalities that facilitate delivery and efficacy during clinical applications to genome editing.

The chemical modification pattern used in the guide RNA is such that activity of the guide RNA is maintained when paired with an RNA-guided DNA endonuclease, e.g., Cas9.

In an embodiment, the chemically modified guide RNAs of the disclosure comprise at least about 50% activity relative to an unmodified guide RNA (e.g., 50% activity, 60% activity, 70% activity, 80% activity, 90% activity, 95% activity, or 100% activity, relative to an unmodified guide RNA).

The activity of a guide RNA can be readily determined by any means known in the art. In an embodiment, % activity is measured with the traffic light reporter (TLR) Multi-Cas Variant 1 system (TLR-MCV1), described below. The TLR-MCV1 system will provide a % fluorescent cells which is a measure of % activity.

Exemplary chemical modification patterns are described in Table 1 and Table 2 below.

TABLE 1
Exemplary chemical modification
patterns for crRNAs

| Name | Sequence |
|---|---|
| crRNA | mN#mN#mN#mNmNmNmNmNmNmNrNrNrNrNrNmNmN |
| 1 | mNrNmNmGrUrUrUmUmAmGmAmGmCmUmAmU#mG# |
| | mCmU |
| | (SEQ ID NO: 146) |
| crRNA | rNrNrNrNrNrNmNmNmNmNrNrNrNrNrNrNrNrNr |
| 2 | NmNmGrUrUrUrUrAmGmAmGmCmUmAmU#mG#mCmU |
| | (SEQ ID NO: 147) |
| crRNA | rN#rN#rN#rNrNrNmNmNmNmNrNrNrNrNrNrNrNr |
| 3 | NrNmNmGrUrUrUrUrAmGmAmGmCmUmAmU#mG#m |
| | CmU |
| | (SEQ ID NO: 148) |
| crRNA | mN#mN#mN#mNmNmNmNmNmNmNrNrNrNrNrNrNrNr |
| 4 | NrNmNmGrUrUrUrUrAmGmAmGmCmUmAmU#mG#mC |
| | #mU |
| | (SEQ ID NO: 149) |
| crRNA | rN#rN#rN#rNrNrNmNmNmNmNrNrNrNrNrNrNmNm |
| 5 | NrNmNmGrUrUrUrUrAmGmAmGmCmUmAmU#mG# |
| | mC#mU |
| | (SEQ ID NO: 150) |
| crRNA | rN#rN#rN#rNrNrNmNmNmNmNrNrNrNrNrNrNrNr |
| 6 | NrNmNmGrUrUrUmUmAmGmAmGmCmUmAmU#mG#m |
| | C#mU |
| | (SEQ ID NO: 151) |
| crRNA | mN#mN#mN#mNmNmNmNmNmNmNrNrNrNrNrNrNmN |
| 7 | mNrNmNmGrUrUrUrUrAmGmAmGmCmUmAmU#mG#m |
| | C#mU (SEQ ID NO: 152) |
| crRNA | mN#mN#mN#mNmNmNmNmNmNmNrNrNrNrNrNrNrNr |
| 8 | NrNmNmGrUrUrUmUmAmGmAmGmCmUmAmU#mG#m |
| | C#mU (SEQ ID NO: 153) |
| crRNA | mN#mN#mN#mNmNmNmNmNmNmNrNrNrNrNrN#rN#r |
| 9 | NrNrN#mNmGrU#rU#rU#mUmAmGmAmGmCmUmAmU |
| | #mG#mC#mU (SEQ ID NO: 154) |
| crRNA | mN#mN#mN#mNmNmNmNmNmNmNfNfNfNfNrN#rN#f |
| 10 | NfNrN#mNmGrU#rU#rU#mUmAmGmAmGmCmUmAmU |
| | #mG#mC#mU (SEQ ID NO: 155) |
| crRNA | mN#mN#mN#mNmNmNmNmNmNmNfNfNfNfNrN#rN#r |
| 11 | N#rN#rN#mNmGrU#rU#rU#mUmAmGmAmGmCmUmAm |
| | U#mG#mC#mU (SEQ ID NO: 156) |
| crRNA | mN#mN#mN#mNmNmNmNmNmNmNfNfNfNfNrN#rN#f |
| 17 | NfNrN#mNmGrU#rU#rU#mUrA#mGmAmGmCmUmAmU |
| | #mG#mC#mU (SEQ ID NO: 157) |
| crRNA | mN#mN#mN#mNmNmNmNmNmNmNfNfNfNfNrN#rN#f |
| 18 | NfNrN#mNmGrU#rU#rU#rU#mAmGmAmGmCmUmAmU |
| | #mG#mC#mU (SEQ ID NO: 158) |
| crRNA | mN#mN#mN#mNmNmNmNmNmNmNfNfNfNfNrN#rN#f |
| 19 | NfNrN#mNmGrU#rU#rU#rU#rA#mGmAmGmCmUmAmU |
| | #mG#mC#mU (SEQ ID NO: 159) |
| crRNA | mN#mN#mN#mNmNmNmNmNmNmNfNfNfNfNrN#rN#f |
| 20 | NfNrN#mNmGrU#rU#rU#fUfAmGmAmGmCmUmAmU# |
| | mG#mC#mU (SEQ ID NO: 160) |
| crRNA | mN#mN#mN#mNmNmNmNmNmNmNfNfNINfNfNfNfNf |
| 21 | NfNmNmGfUfUfUfUfAmGmAmGmCmUmAmU#mG#mC |
| | #mU (SEQ ID NO: 161) |
| crRNA | mN#mN#mN#mNmNmNmNmNmNmNfNfNfNfNrN#rN#f |
| 22 | NfNrN#mNmGfUrU#fUfUfAmGmAmGmCmUmAmU#mG |
| | #mC#mU (SEQ ID NO: 162) |
| crRNA | mN#mN#mN#mNmNmNmNmNmNmNfNfNfNfNmNrN#f |
| 23 | NfNrN#mNmGrU#rU#rU#fUfAmGmAmGmCmUmAmU# |
| | mG#mC#mU (SEQ ID NO: 128) |
| crRNA | mN#mN#mN#mNmNmNmNmNmNmNfNfNfNfNrN#fNfNf |
| 24 | NrN#mNmGrU#rU#rU#fUfAmGmAmGmCmUmAmU#mG |
| | #mC#mU (SEQ ID NO: 129) |
| crRNA | mN#mN#mN#mNmNmNmNmNmNmNfNfNfNfNrN#rN#f |
| 25 | NfNfNmNmGrU#rU#rU#fUfAmGmAmGmCmUmAmU#m |
| | G#mC#mU (SEQ ID NO: 130) |
| crRNA | mN#mN#mN#mNmNmNmNmNmNmNfNfNfNfNrN#rN#f |
| 26 | NfNrN#mNmGfUrU#rU#fUfAmGmAmGmCmUmAmU#m |
| | G#mC#mU (SEQ ID NO: 131) |
| crRNA | mN#mN#mN#mNmNmNmNmNmNmNfNfNfNfNrN#rN#f |
| 27 | NfNrN#NAmGrU#fUrU#fUfAmGmAmGmCmUmAmU#m |
| | G#mC#mU (SEQ ID NO: 163) |
| crRNA | mN#mN#mN#mNmNmNmNmNmNmNfNfNfNfNrN#rN#f |
| 28 | NfNrN#mNmGrU#rU#rUfUfAmGmAmGmCmUmAmU#m |
| | G#mC#Mu (SEQ ID NO: 133) |
| crRNA | mN#mN#mN#mNmNmNmNmNmNmNfNfNfNfNrN#fNfNf |
| 29 | NfNmNmGfUfUfUfUfAmGmAmGmCmUmAmU#mG#mC |
| | #mU (SEQ ID NO: 134) |
| crRNA | mN#mN#mN#rNrNrNmNmNmNmNrNrNrNrNrNrNrNrN |
| 30 | rNmNmGrUrUrUrUrAmGmAmGmCmUmAmU#mG#mC#mU |
| | (SEQ ID NO: 135) |
| crRNA | mN#mN#mN#rNrNrNmNmNmNmNmNrNrNrNrNrNrNr |
| 31 | NrNmNmGrUrUrUrUrAmGmAmGmCmUmAmU#mG#mC |
| | #mU (SEQ ID NO: 136) |
| crRNA | mN#mN#mN#rNrNrNmNmNmNmNmNrNmNmNrNrNrNr |
| 32 | NrNmNmGrUrUrUrUrAmGmAmGmCmUmAmU#mG#mC |
| | #mU (SEQ ID NO: 137) |
| crRNA | mN#mN#mN#rN#rN#rN#mNmNmNmNrN#rN#rN#rN#r |
| 33 | N#rN#rN#rN#rN#mNmGrU#rU#rU#rU#rA#mGmAm |
| | GmCmUmAmU#mG#mC#mU (SEQ ID NO: 121) |
| crRNA | mN#mN#mN#rN#rN#rN#mNmNmNmNrN#rN#rN#rN# |
| 34 | rN#rN#rN#rN#rN#mNmGrUrUrUrUrAmGmAmGmCm |
| | UmAmU#mG#mC#mU (SEQ ID NO: 122) |
| crRNA | mN#mN#mN#rNrNrNmNmNmNmNrNrNrNrNrNrNrNr |
| 35 | NrNmNmGrUrUrUmUmAmGmAmGmCmUmAmU#mG#mC# |
| | mU (SEQ ID NO: 138) |
| crRNA | mN#mN#mN#rN#rN#rN#mNmNmNmNrN#rN#rN#rN# |
| 36 | rN#rN#rN#rN#rN#mNmGrUrUrUmUmAmGmAmGmCm |
| | UmAmU#mG#mC#mU (SEQ ID NO: 123) |
| crRNA | mN#mN#mN#rN#rN#rN#mNmNmNmNfNfNfNfNrN#r |
| 37 | N#fNfNrN#mNmGrU#rU#rU#mUmAmGmAmGmCmUmA |
| | mU#mG#mC#mU (SEQ ID NO: 124) |
| crRNA | mN#mN#mN#dN#dN#dN#mNmNmNmNfNfNfNfNrN#r |
| 38 | N#fNfNrN#mNmGrU#rU#rU#mUmAmGmAmGmCmUmA |
| | mU#mG#mC#mU (SEQ ID NO: 5) |
| crRNA | mN#mN#mN#rN#rN#rN#mNmNmNmNfNfNfNfNfNfNf |
| 39 | NfNfNmNmGfUfUfUfUfAmGmAmGmCmUmAmU#mG#mC |
| | #mU (SEQ ID NO: 125) |
| crRNA | mN#mN#mN#dN#dN#dN#mNmNmNmNfNfNfNfNfNf |
| 40 | NfNfNfNmNmGfUfUfUfUfAmGmAmGmCmUmAmU# |
| | mG#mC#mU (SEQ ID NO: 6) |
| crRNA | mN#mN#mN#mNmNmNmNmNmNmNfNfNfNfNdN#dN#f |
| 41 | NfNdN#mNmGrU#rU#rU#fUfAmGmAmGmCmUmAmU# |
| | mG#mC#mU (SEQ ID NO: 7) |
| crRNA | mN#mN#mN#mNmNmNmNmNrnNrnNfNfNfNfNrN#r |
| 42 | N#fNfNrN#mNmGdU#dU#dU#fUfAmGmAmGmCmUm |
| | AmU#mG#mC#mU (SEQ ID NO: 8) |
| crRNA | rnN#mN#mN#mNmNmNmNrnNmNmNfNrN#fNfNrN#r |
| 43 | N#fNfNrN#mNmGrU#rU#rU#fUfAmGmAmGmCmUmA |
| | mU#mG#mC#mU (SEQ ID NO: 139) |
| crRNA | mN#mN#nfN#mNmNmNniNmNmNrnNfNdN#fNfNrN#r |
| 44 | N#fNfNrN#mNmGrU#rU#rU#fUfAmGmAmGmCmUmA |
| | mU#mG#mC#mU (SEQ ID NO: 9) |
| crRNA | mN#mN#mN#fNfNfNmNmNmNmNfNfNfNfNfNfNfNfN |
| 45 | fNmNmGfUfUfUfUfAmGmAmGmCmUmAmU#mG#mC#mU |
| | (SEQ ID NO: 126) |
| crRNA | mN#mN#mN#mNmNmNmNmNmNmNfNfNfNfNmNrN#f |
| 46 | NfNrN#mNmGrU#rUrU#fUfAmGmAmGmCmUmAmU#m |
| | G#mC#mU (SEQ ID NO: 140) |
| crRNA | mN#mN#niN#mNmNmNmNmNmNmNfNfNfNfNrN#mNf |
| 47 | NfNrN#mNmGrU#rUrU#fUfAmGmAmGmCmUmAmU#m |
| | G#mC#mU (SEQ ID NO: 141) |
| crRNA | mN#rnN#mN#mNmNmNmNmNmNmNfNfNfNfNrN#mNf |
| 48 | NfNmNmNmGrU#rUrU#fUfAmGmAmGmCmUmAmU#m |
| | G#mC#mU (SEQ ID NO: 142) |
| crRNA | mN#mN#mN#mNmNmNmNmNmNmNfNfNfNfNrN#rN#f |
| 49 | NfNrN#mNmGmUrU#rU#fUfAmGmAmGmCmUmAmU#m |
| | G#mC#mU (SEQ ID NO: 143) |
| crRNA | mN#mN#mN#mNmNmNmNmNmNmNfNfNfNfNrN#rN#f |
| 50 | NfNrN#mNmGrU#mUrU#fUfAmGmAmGmCmUmAmU#m |
| | G#mC#mU (SEQ ID NO: 144) |
| crRNA | mN#mN#mN#mNmNmNmNmNmNmNfNfNfNfNrN#rN#f |
| 51 | NfNrN#mNmGrU#rU#mUfUfAmGmAmGmCmUmAmU#m |
| | G#mC#mU (SEQ ID NO: 164) |
| crRNA | mN#mN#mN#dN#dN#dN#mNmNmNmNfNfNfNfNfN# |
| 52 | fN#fNfNfN#mNmGfU#fU#fU#fU#fA#mGmAmGmC |
| | mUmAmU#mG#mC#mU (SEQ ID NO: 165) |
| crRNA | mN#mN#mN#dN#dN#dN#mN#mNmNmNfNfNfNfNfNfNf |
| 53 | NfNfNmNmGfUfUfUfUfAmGmAmGmCmUmAmU#mG# |
| | mC#mU (SEQ ID NO: 166) |
| crRNA | mN#mN#mN#dN#dN#dN#mNmN#mNmNfNfNfNfNfNfNf |
| 54 | NfNfNmNmGfUfUfUfUfAmGmAmGmCmUmAmU#mG# |
| | mC#mU (SEQ ID NO: 167) |
| crRNA | mN#mN#mN#dN#dN#dN#mNmNmN#mNfNfNfNfNfNfNf |
| 55 | NfNfNmNmGfUfUfUfUfAmGmAmGmCmUmAmU#mG# |
| | mC#mU (SEQ ID NO: 168) |
| crRNA | mN#mN#mN#dN#dN#dN#mNmNmNmN#fNfNfNfNfNfNf |
| 56 | NfNfNmNmGfUfUfUfUfAmGmAmGmCmUmAmU#mG# |
| | mC#mU (SEQ ID NO: 169) |
| crRNA | mN#mN#mN#dN#dN#dN#mNmNmNmNfN#fNfNfNfNfNf |
| 57 | NfNfNmNmGfUfUfUfUfAmGmAmGmCmUmAmU#mG# |
| | mC#mU (SEQ ID NO: 170) |
| crRNA | mN#mN#mN#dN#dN#dN#mNmNmNmNfNfN#fNfNfNfNf |
| 58 | NfNfNmNmGfUfUfUfUfAmGmAmGmCmUmAmU#mG# |
| | mC#mU (SEQ ID NO: 171) |
| crRNA | mN#mN#mN#dN#dN#dN#mNmNmNmNfNfNfN#fNfNfNf |
| 59 | NfNfNmNmGfUfUfUfUfAmGmAmGmCmUmAmU#mG# |
| | mC#mU (SEQ ID NO: 172) |
| crRNA | mN#mN#mN#dN#dN#dN#mNmNmNmNfNfNfNfN#fNfNf |
| 60 | NfNfNmNmGfUfUfUfUfAmGmAmGmCmUmAmU#mG# |
| | mC#mU (SEQ ID NO: 173) |
| crRNA | mN#mN#mN#dN#dN#dN#mNmNmNmNfNfNfNfNfNfNfN |
| 61 | #fNfNmNmGfUfUfUfUfAmGmAmGmCmUmAmU#mG#m |
| | C#mU (SEQ ID NO: 174) |
| crRNA | mN#mN#mN#dN#dN#dN#mNmNmNmNfNfNfNfNfNfNfN |
| 62 | fN#fNmNmGfUfUfUfUfAmGmAmGmCmUmAmU#mG#m |
| | C#mU (SEQ ID NO: 175) |
| crRNA | mN#mN#mN#dN#dN#dN#mNmNmNmNfNfNfNfNfNfNfN |
| 63 | fNfNmN#mGfUfUfUfUfAmGmAmGmCmUmAmU#mG#m |
| | C#mU (SEQ ID NO: 176) |
| crRNA | mN#mN#mN#dN#dN#dN#mNmNmNmNfNfNfNfNfNfNfN |
| 64 | fNfNmNmG#fUfUfUfUfAmGmAmGmCmUmAmU#mG#m |
| | C#mU (SEQ ID NO: 177) |
| crRNA | mN#mN#mN#dN#dN#dN#mNmNmNmNfNfNfNfNfNfNfN |
| 65 | fNfNmNmGfUfUfUfUfAmG#mAmGmCmUmAmU#mG#m |
| | C#mU (SEQ ID NO: 178) |
| crRNA | mN#mN#mN#dN#dN#dN#mNmNmNmNfNfNfNfNfNfNfN |
| 66 | fNfNmNmGfUfUfUfUfAmGmA#mGmCmUmAmU#mG#m |
| | C#mU (SEQ ID NO: 179) |
| crRNA | mN#mN#mN#dN#dN#dN#mNmNmNmNfNfNfNfNfNfNfN |
| 67 | fNfNmNmGfUfUfUfUfAmGmAmG#mCmUmAmU#mG#m |
| | C#mU (SEQ ID NO: 180) |
| crRNA | mN#mN#mN#dN#dN#dN#mNmNmNmNfNfNfNfNfNfNfN |
| 68 | fNfNmNmGfUfUfUfUfAmGmAmGmC#mUmAmU#mG#m |
| | C#mU (SEQ ID NO: 181) |
| crRNA | mN#mN#mN#dN#dN#dN#mNmNmNmNfNfNfNfNfNfNfN |
| 69 | fNfNmNmGfUfUfUfUfAmGmAmGmCmU#mAmU#mG#m |
| | C#mU (SEQ ID NO: 182) |
| crRNA | mN#mN#mN#dN#dN#dN#mNmNmNmNfNfNfNfNfNfNfN |
| 70 | fNfNmNmGfUfUfUfUfAmGmAmGmCmUmA#mU#mG#m |
| | C#mU (SEQ ID NO: 183) |
| crRNA | mN#mN#mN#dN#dN#dN#mN#mN#mN#mNfNfNfNfNfN# |
| 71 | fN#fNfNfN#mNmGfU#fU#fU#fU#fA#mGmAmGmCmU |
| | mAmU#mG#mC#mU (SEQ ID NO: 184) |
| crRNA | mN#mN#mN#dN#dN#dN#mNmNmNmN#fN#fN#fNfNfN |
| 72 | #fN#fNfNfN#mNmGfU#fU#fU#fU#fA#mGmAmGmCm |
| | UmAmU#mG#mC#mU (SEQ ID NO: 185) |
| crRNA | mN#mN#mN#dN#dN#dN#mNmNmNmNfNfNfN#fN#fN#f |
| 73 | N#fNfNfN#mNmGfU#fU#fU#fU#fA#mGmAmGmCmUmA |
| | mU#mG#mC#mU (SEQ ID NO: 186) |
| crRNA | mN#mN#mN#dN#dN#dN#mNmNmNmNfNfNfNfNfN#fN# |
| 74 | fN#fN#fN#mNmGfU#fU#fU#fU#fA#mGmAmGmCmUmA |
| | mU#mG#mC#mU (SEQ ID NO: 187) |
| crRNA | mN#mN#mN#dN#dN#dN#mNmNmNmNfNfNfNfNfN#f# |
| 75 | Nf#fNfNfN#mN#mGfU#fU#fU#fU#fA#mGmAmGmCm |
| | UmAmU#mG#mC#mU (SEQ ID NO: 188) |
| crRNA | mN#mN#mN#dN#dN#dN#mN#mN#mN#mNfNfNfNfNfNf |
| 76 | NfNfNfNmNmGfUfUfUfUfAmGmAmGmCmUmAmU#mG |
| | #mC#mU (SEQ ID NO: 189) |
| crRNA | mN#mN#mN#dN#dN#dN#mN#mN#mN#mN#fN#fN#fNf |
| 77 | NfNfNfNfNfNmNmGfUfUfUfUfAmGmAmGmCmUmAmU# |
| | mG#mC#mU (SEQ ID NO: 190) |
| crRNA | mN#mN#mN#dN#dN#dN#mN#mN#mN#mN#fN#fN#fN#f |
| 78 | N#fN#fN#fNfNfNrnNmGfUfUfUfUfAmGmAmGmCmU |
| | mAmU#mG#mC#mU (SEQ ID NO: 191) |
| crRNA | mN#mN#mN#dN#dN#dN#mN#mN#mN#mN#fN#fN#fN#f |
| 79 | N#fN#fN#fN#fN#fN#mNmGfUfUfUfUfAmGmAmGmC |
| | mUmAmU#mG#mC#mU (SEQ ID NO: 192) |
| crRNA | mN#mN#mN#dN#dN#dN#mN#mN#mN#mN#fN#fN#fN#f |
| 80 | N#fN#fN#fN#fN#fN#mN#mG#fU#fU#fU#fU#fA#m |
| | GmAmGmCmUmAmU#mG#mC#mU (SEQ ID NO: 193) |
| crRNA | mN#mN#mN#dN#dN#dN#mNmNmNmNfNfNfNfNfN#fN# |
| 81 | fNfNfN#mNmGfU#fU#fU#fU#fA#mGmAmGmC#mU#mA |
| | #mU#mG#mC#mU (SEQ ID NO: 194) |
| crRNA | mN#mN#mN#dN#dN#dN#mNmNmNmNfNfNfNfNfN#fN# |
| 82 | fNfNfN#mNmGfU#fU#fU#fU#fA#mG#mA#mG#mCmUm |
| | AmU#mG#mC#mU (SEQ ID NO: 195) |
| crRNA | mN#mN#mN#dN#dN#dN#mNmNmNmNfNfNfNfNfN#fN# |
| 83 | fNfNfN#mNmG#fU#fU#fU#fU#fA#mGmAmGmCmUmAm |
| | U#mG#mC#mU (SEQ ID NO: 196) |
| crRNA | mN#mN#mN#dN#dN#dN#mNmNmNmNfNfNfNfNfNfNfN |
| 84 | fNfNmNmGfUfUfUfUfAmGmAmGmC#mU#mA#mU#mG |
| | #mC#mU (SEQ ID NO: 197) |
| crRNA | mN#mN#mN#dN#dN#dN#mNmNmNmNfNfNfNfNfNfNfN |
| 85 | fNfNmNmGfUfUfUfUfA#mG#mA#mG#mC#mU#mA#mU |
| | #mG#mC#mU (SEQ ID NO: 198) |
| crRNA | mN#mN#mN#dN#dN#dN#mNmNmNmNfNfNfNfNfNfNfN |
| 86 | fNfNmNmGfU#fU#fU#fU#fA#mG#mA#mG#mC#mU#mA |
| | #mU#mG#mC#mU (SEQ ID NO: 199) |
| crRNA | mN#mN#mN#dN#dN#dN#mN#mN#mNmNfNfNfNfNfNfN |
| 87 | fNfNfNmNmGfUfUfUfUfAmGmAmGmCmU#mA#mU#m |
| | G#mC#mU (SEQ ID NO: 200) |
| crRNA | mN#mN#mN#dN#dN#dN#mN#mN#mN#mN#fNfNfNfNf |
| 88 | NfNfNfNfNmNmGfUfUfUfUfAmGmAmG#mC#mU#mA# |
| | mU#mG#mC#mU |
| | (SEQ ID NO: 201) |
| crRNA | mN#mN#mN#dN#dN#dN#mN#mN#mN#mN#fN#fN#fNf |
| 89 | NfNfNfNfNfNmNmGfUfUfUfUfAmG#mA#mG#mC#mU |
| | #mA#mU#mG#mC#mU |
| | (SEQ ID NO: 202) |
| crRNA | mN#mN#mN#dN#dN#dN#mN#mN#mN#mN#fN#fN#fN#f |
| 90 | N#fNfNfNfNfNmNmGfUfUfUfU#fA#mG#mA#mG#m |
| | C#mU#mA#mU#mG#mC#mU |
| | (SEQ ID NO: 203) |
| crRNA | mN#mN#mN#dN#dN#dN#mN#mN#mN#mN#fN#fN#fN#f |
| 91 | N#fN#fN#fNfNfNmNmGfUfU#fU#fU#fA#mG#mA#mG |
| | #mC#mU#mA#mU#mG#mC#mU |
| | (SEQ ID NO: 204) |
| crRNA | mN#mN#mN#dN#dN#dN#mN#mN#mN#mN#fN#fN#fN#f |
| 92 | N#fN#fN#fN#fN#fN#mNmGfU#fU#fU#fU#fA#mG# |
| | mA#mG#mC#mU#mA#mU#mG#mC#mU |
| | (SEQ ID NO: 205) |
| crRNA | mN#mN#mN#dN#dN#dN#mN#mN#mN#mN#fN#fN#fN#f |
| 93 | N#fN#fN#fN#fN#fN#mN#mG#fU#fU#fU#fU#fA#m |
| | G#mA#mG#mC#mU#mA#mU#mG#mC#mU |
| | (SEQ ID NO: 206) |
| crRNA | mN#mN#mN#mN#mN#mN#mNmNmNmNfNfNfNfNrN#r |
| 94 | N#fNfNrN#mNmGrU#rU#rU#fUfAmGmAmGmCmUmAm |
| | U#mG#mC#mU (SEQ ID NO: 207) |
| crRNA | mN#mN#mN#mN#mN#mN#mN#mNmNmNfNfNfNfNrN#r |
| 95 | N#fNfNrN#mNmGrU#rU#rU#fUfAmGmAmGmCmUmAm |
| | U#mG#mC#mU (SEQ ID NO: 208) |
| crRNA | mN#mN#mN#mN#mN#mN#mNmNmNmNfNfNfNfNrN#r |
| 96 | N#fNfNrN#mNmGrU#rU#rU#fUfAmGmAmGmC#mUmA |
| | mU#mG#mC#mU (SEQ ID NO: 209) |
| crRNA | mN#mN#mN#mN#mN#mN#mNmNmNmNfNfNfNfNrN#r |
| 97 | N#fNfNrN#mNmGrU#rU#rU#fUfAmGmAmGmCmU#mA |
| | mU#mG#mC#mU (SEQ ID NO: 210) |
| crRNA | mN#mN#mN#mN#mN#mN#mNmNmNmNfNfNfNfNrN#r |
| 98 | N#fNfNrN#mGNmGrU#rU#rU#fUfAmGmAmGmCmUmA |
| | #mU#mG#mC#mU (SEQ ID NO: 211) |
| crRNA | mN#mN#mN#mN#mN#mN#mNmNmNmNfNfNfNfNrN#r |
| 99 | N#fNfNrN#mNmGrU#rU#rU#fU#fA#mGmAmGmCmU |
| | mAmU#mG#mC#mU (SEQ ID NO: 212) |
| crRNA | mN#mN#mN#mN#mN#mN#mNmNmNmNfNfNfNfNrN#r |
| 100 | N#fNfNrN#mNmGrU#rU#rU#fU#fA#mGmAmGmC#mU# |
| | mA#mU#mG#mC#mU (SEQ ID NO: 213) |
| crRNA | mN#mN#mN#mN#mN#mN#mNmNmNmNfNfNfNfNrN#r |
| 101 | N#fNfNrN#mNmGrU#rU#rU#fU#fA#mG#mA#mG#mC |
| | mUmAmU#mG#mC#mU (SEQ ID NO: 214) |
| crRNA | mN#mN#mN#mN#mN#mN#mNmNmNmNfNfNfNfNrN#r |
| 102 | N#fNfNrN#mNmG#rU#rU#rU#fU#fA#mGmAmGmCm |
| | UmAmU#mG#mC#mU (SEQ ID NO: 215) |
| crRNA | mN#mN#mN#mN#mN#mN#mNmNmNmNfNfNfNfNrN#r |
| 103 | N#fNfNrN#mNmGrU#rU#rU#fUfAmGmAmGmC#mU# |
| | mA#mU#mG#mC#mU (SEQ ID NO: 216) |
| crRNA | mN#mN#mN#mN#mN#mN#mNmNmNmNfNfNfNfNrN#r |
| 104 | N#fNfNrN#mNmGrU#rU#rU#fUfA#mG#mA#mG#mC# |
| | mU#mA#mU#mG#mC#mU (SEQ ID NO: 217) |
| crRNA | mN#mN#mN#rN#rN#rN#mN#mNmNmNfNfNfNfNfNfN |
| 105 | fNfNfNrnNmGfUfUfUfUfAmGmAmGmCmUmAmU#mG# |
| | mC#mU (SEQ ID NO: 218) |
| crRNA | mN#mN#mN#rN#rN#rN#mNmNmNmNfNfNfNfNfNfNf |
| 106 | NfNfNmNmGfUfUfUfUfAmGmAmGmCmUmA#mU#mG#m |
| | C#mU (SEQ ID NO: 219) |
| crRNA | mN#mN#mN#rN#rN#rN#mNmNmNrnNfNfNfNfNfN#f |
| 107 | N#fN#fN#fN#mNmGfU#fU#fU#fU#fA#mGmAmGmCmU |
| | mAmU#mG#mC#mU (SEQ ID NO: 220) |
| crRNA | mN#mN#mN#rN#rN#rN#mNmNmNmNfNfNfNfNfN#fN# |
| 108 | fNfNfN#mNmGfU#fU#fU#fU#fA#mGmAmGmC#mU#m |
| | A#mU#mG#mC#mU (SEQ ID NO: 221) |
| crRNA | mN#mN#mN#rN#rN#rN#mNmNmNmNfNfNfNfNfN#fN |
| 109 | #fNfNfN#mNmGfU#fU#fU#fU#fA#mG#mA#mG#mCm |
| | UmAmU#mG#mC#mU (SEQ ID NO: 222) |
| crRNA | mN#mN#mN#rN#rN#rN#mNmNmNmNfNfNfNfNfN#fN# |
| 110 | fNfNfN#mNmG#fU#fU#fU#fU#fA#mGmAmGmCmUmAm |
| | U#mG#mC#mU (SEQ ID NO: 223) |
| crRNA | mN#mN#mN#rN#rN#rN#mNmNmNmNfNfNfNfNfNfNf |
| 111 | NfNfNmNmGfUfUfUfUfAmGmAmGmC#mU#mAmU#mG#m |
| | C#mU (SEQ ID NO: 224) |
| crRNA | mN#mN#mN#rN#rN#rN#mNmNmNmNfNfNfNfNfNfNfN |
| 112 | fNfNmNmGfUfUfUfUfA#mG#mA#mG#mC#mU#mAmU#m |
| | G#mC#mU (SEQ ID NO: 225) |
| crRNA | mN#mN#mN#dN#dN#dN#mNmNmNmNfNfNfNfNdN#dN |
| 113 | #fNfNdN#mNmGdU#dU#dU#fUfAmGmAmGmCmUmAm |
| | U#mG#mC#mU (SEQ ID NO: 226) |
| crRNA | mN#mN#mN#mNmNmNmNmNmNmNfNfNfNfNrN#rN#f |
| 114 | NfNaNmNmGaUaUaUfUfAmGmAmGmCmUmAmU#mG# |
| | mC#mU (SEQ ID NO: 227) |
| crRNA | mN#mN#mN#mNmNmNmNrnNmNmNfNfNfNfNrN#rN#f |
| 115 | NfNaNmNmGrU#rU#rU#fUfAmGmAmGmCmUmAmU#m |
| | G#mC#mU (SEQ ID NO: 228) |
| crRNA | mN#mN#mN#mNmNmNmNmNmNmNfNfNfNfNrN#rN#f |
| 116 | NfNrN#mNmGaUrU#rU#fUfAmGmAmGmCmUmAmU#m |
| | G#mC#mU (SEQ ID NO: 229) |
| crRNA | mN#mN#mN#mNmNmNmNmNmNmNfNfNfNfNrN#rN#f |
| 117 | NfNrN#mNmGrU#aUrU#fUfAmGmAmGmCmUmAmU#m |
| | G#mC#mU (SEQ ID NO: 230) |
| crRNA | mN#mN#mN#mNmNmNmNmNmNmNlMNfNfNrN#rN#f |
| 118 | NfNrN#mNmGrU#rU#aUfUfAmGmAmGmCmUmAmU#m |
| | G#mC#mU (SEQ ID NO: 231) |
| crRNA | mN#mN#mN#mNmNmNmNmNmNmNfNfNfNfNrN#rN#f |
| 119 | NfNsN#mNmGsU#sU#sU#fUfAmGmAmGmCmUmAmU# |
| | mG#mC#mU (SEQ ID NO: 232) |
| crRNA | mN#mN#mN#mNmNmNmNmNmNmNfNfNfNfNrN#rN#f |
| 120 | NfNsNmNmGsUsUsUfUfAmGmAmGmCmUmAmU#mG# |
| | mC#mU (SEQ ID NO: 233) |
| crRNA | mN#mN#mN#mNmNmNmNmNmNmNfNfNfNfNrN#rN#f |
| 121 | NfNsNmNmGrU#rU#rU#fXJfAmGmAmGmCmUmAmU# |
| | mG#mC#mU (SEQ ID NO: 234) |
| crRNA | mN#mN#mN#mNmNmNmNmNmNmNfNfNfNfNrN#rN#f |
| 122 | NfNrN#mNmGsUrU#rU#fUfAmGmAmGmCmUmAmU#m |
| | G#mC#mU (SEQ ID NO: 235) |
| crRNA | mN#mN#mN#mNmNmNmNmNmNmNfNfNfNfNrN#rN#f |
| 123 | NfNrN#mNmGrU#sUrU#fUfAmGmAmGmCmUmAmU#m |
| | G#mC#mU (SEQ ID NO: 236) |
| crRNA | mN#mN#mN#mNmNmNmNmNmNmNfNfNfNfNrN#rN#f |
| 124 | NfNrN#mNmGrU#rU#sUfUfAmGmAmGmCmUmAmU#m |
| | G#mC#mU (SEQ ID NO: 237) |
| crRNA | mN#mN#mN#mNmNmNmNmNmNmNfNfNfNfNrN#rN#f |
| 125 | NfNrN#mNmGsUrU#sUfUfAmGmAmGmCmUmAmU#mG |
| | #mC#mU (SEQ ID NO: 238) |
| crRNA | mN#mN#mN#mNmNmNmNmNmNmNfNfNfNfNrN#rN#f |
| 126 | NfNrN#mNmGsUsUrU#fUfAmGmAmGmCmUmAmU#mG |
| | #mC#mU (SEQ ID NO: 239) |
| crRNA | mN#mN#mN#mNmNmNmNmNmNmNfNfNfNfNrN#rN#f |
| 127 | NfNrN#mNmGrU#sUsUfUfAmGmAmGmCmUmAmU#mG |
| | #mC#mU (SEQ ID NO: 240) |
| crRNA | mN#mN#mN#mNmNmNmNmNmNmNfNfNfNfNrN#rN#f |
| 128 | NfNrN#mNmGaUaUaUfUfAmGmAmGmCmUmAmU#mG |
| | #mC#mU (SEQ ID NO: 241) |
| crRNA | mN#mN#mN#mNmNmNmNmNmNmNfNfNfNfNrN#rN#f |
| 129 | NfNrN#mNmGsU#sU#sU#fUfAmGmAmGmCmUmAmU# |
| | mG#mC#mU (SEQ ID NO: 242) |
| crRNA | mN#mN#mN#mNmNmNmNmNmNmNfNfNfNfNrN#rN#f |
| 130 | NfNrN#mNmGsUsUsUfUfAmGmAmGmCmUmAmU#mG# |
| | mC#mU (SEQ ID NO: 243) |
| crRNA | mN#mN#mN#mNmNmNmNmNmNmNfNfNfNfNrN#aNfN |
| 131 | fNrN#mNmGrU#rU#rU#fUfAmGmAmGmCmUmAmU#m |
| | G#mC#mU (SEQ ID NO: 244) |
| crRNA | mN#mN#mN#mNmNmNmNmNmNmNfNfNfNfNrN#rN#f |
| 132 | NfNaNmNmGrU#rU#rU#fUfAmGmAmGmCmUmAmU#m |
| | G#mC#mU (SEQ ID NO: 228) |
| crRNA | mN#mN#mN#mNmNmNmNmNmNmNfNfNfNfNrN#aN#f |
| 133 | NfNaN#mNmGrU#rU#rU#fUfAmGmAmGmCmUmAmU# |
| | mG#mC#mU (SEQ ID NO: 245) |
| crRNA | mN#mN#mN#mNmNmNmNmNmNmNfNfNfNfNrN#rN#f |
| 134 | NfNrN#mNmGaUaUaUfUfAmGmAmGmCmUmAmU#mG |
| | #mC#mU (SEQ ID NO: 241) |

KEY:
rN = RNA, mN = 2′-O-methyl RNA, fN = 2′-fluoro RNA, aN = 2′-NH₂ (2′-amino RNA), sN = 4′-thio RNA, dN = T-deoxy RNA, N = A, U, G, or C N#N = phosphorothioate linkage

TABLE 2
Exemplary chemical modification patterns for tracrRNAs

| | rN = RNA, mN = 2′-O-methyl RNA, fN =2′-fluoro RNA, |
|---|---|
| | sN = 4′-thio RNA, dN =2′-deoxy RNA, N = A, U, G, or C |
| KEY: | N#N = phosphorothioate linkage |
| Name | Sequence |
| tracrRNA 1 | mA#mG#mC#mAmUmAmGrCrArArGrUrUmArArArArUr |
| | ArArGmGrCrUmArGrUrCmCrGrUrUrArUrCrAmAmCmU |
| | mUmGmAmAmAmAmAmGmUrGrGrCrAmCmCmGrArGr |
| | UrCrGmGmUmGmC#mU#mU#mU (SEQ ID NO: 10) |
| tracrRNA 2 | mA#mG#mC#mAmUmAmGmCmAmAmGrUrUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 11) |
| tracrRNA 3 | mA#mG#mC#mAmUmAmGmCmAmAmGrU#rU#mArA# |
| | mAmArU#mAmAmGmGrC#rU#mArG#rU#rC#mCrG#rU#r |
| | U#mAmUmCmAmAmCmUmUmGmAmAmAmAmAmGm |
| | UmGmGmCmAmCmCmGmAmGmUmCmGmGmUmGmC |
| | #mU#mU#mU (SEQ ID NO: 12) |
| tracrRNA 4 | mA#mG#mC#mAmUmAmGmCmAmAmGrUrUmAmAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 13) |
| tracrRNA 5 | mA#mG#mC#mAmUmAmGmCmAmAmGrUrUmAmAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 13) |
| tracrRNA 6 | mA#mG#mC#mAmUmAmGmCmAmAmGrUrUmArAmA |
| | mAfUmAmAmGmGfCfUmArGfUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 14) |
| tracrRNA 7 | mA#mG#mC#mAmUmAmGmCmAmAmGrUfUmArAmA |
| | mAfUmAmAmGmGfCfUmAfGfUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 15) |
| tracrRNA 8 | mA#mG#mC#mAmUmAmGmCmAmAmGfUfUmAfAmA |
| | mAfUmAmAmGmGfCfUmAfGfUfCmCfGfUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 16) |
| tracrRNA 9 | mA#mG#mC#mAmUmAmGmCmAmAmGfUrUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 17) |
| tracrRNA 10 | mA#mG#mC#mAmUmAmGmCmAmAmGrUfUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 18) |
| tracrRNA 11 | mA#mG#mC#mAmUmAmGmCmAmAmGrUrUmAfAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 19) |
| tracrRNA 12 | mA#mG#mC#mAmUmAmGmCmAmAmGrUrUmArAmA |
| | mAfUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 20) |
| tracrRNA 13 | mA#mG#mC#mAmUmAmGmCmAmAmGrUrUmArAmA |
| | mArUmAmAmGmGfCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 21) |
| tracrRNA 14 | mA#mG#mC#mAmUmAmGmCmAmAmGrUrUmArAmA |
| | mArUmAmAmGmGrCfUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 22) |
| tracrRNA 15 | mA#mG#mC#mAmUmAmGmCmAmAmGrUrUmArAmA |
| | mArUmAmAmGmGrCrUmAfGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 23) |
| tracrRNA 16 | mA#mG#mC#mAmUmAmGmCmAmAmGrUrUmArAmA |
| | mArUmAmAmGmGrCrUmArGfUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 24) |
| tracrRNA 17 | mA#mG#mC#mAmUmAmGmCmAmAmGrUrUmArAmA |
| | mArUmAmAmGmGrCrUmArGrUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 25) |
| tracrRNA 18 | mA#mG#mC#mAmUmAmGmCmAmAmGrUrUmArAmA |
| | mArUmAmAmGmGrCrUmArGrUrCmCfGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 26) |
| tracrRNA 19 | mA#mG#mC#mAmUmAmGmCmAmAmGrUrUmArAmA |
| | mArUmAmAmGmGrCrUmArGrUrCmCrGfUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 27) |
| tracrRNA 20 | mA#mG#mC#mAmUmAmGmCmAmAmGrUrUmArAmA |
| | mArUmAmAmGmGrCrUmArGrUrCmCrGrUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 28) |
| tracrRNA 21 | mA#mG#mC#mAmUmAmGmCmAmAmGrU#rU#mArA# |
| | mAmAfUmAmAmGmGfCfUmArG#fUfCmCrG#rU#rU#m |
| | AmUmCmAmAmCmUmUmGmAmAmAmAmAmGmUmG |
| | mGmCmAmCmCmGmAmGmUmCmGmGmUmGmC#mU# |
| | mU#mU (SEQ ID NO: 29) |
| tracrRNA 22 | mA#mG#mC#mAmUmAmGmCmAmAmGmUrUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 30) |
| tracrRNA 23 | mA#mG#mC#mAmUmAmGmCmAmAmGrUmUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 31) |
| tracrRNA 24 | mA#mG#mC#mAmUmAmGmCmAmAmGrUrUmAmAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 13) |
| tracrRNA 25 | mA#mG#mC#mAmUmAmGmCmAmAmGrUrUmArAmA |
| | mAmUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 32) |
| tracrRNA 26 | mA#mG#mC#mAmUmAmGmCmAmAmGrUrUmArAmA |
| | mArUmAmAmGmGmCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 33) |
| tracrRNA 27 | mA#mG#mC#mAmUmAmGmCmAmAmGrUrUmArAmA |
| | mArUmAmAmGmGrCmUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 34) |
| tracrRNA 28 | mA#mG#mC#mAmUmAmGmCmAmAmGrUrUmArAmA |
| | mArUmAmAmGmGrCrUmAmGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 35) |
| tracrRNA 29 | mA#mG#mC#mAmUmAmGmCmAmAmGrUrUmArAmA |
| | mArUmAmAmGmGrCrUmArGmUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 36) |
| tracrRNA 30 | mA#mG#mC#mAmUmAmGmCmAmAmGrUrUmArAmA |
| | mArUmAmAmGmGrCrUmArGrUmCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 37) |
| tracrRNA 31 | mA#mG#mC#mAmUmAmGmCmAmAmGrUrUmArAmA |
| | mArUmAmAmGmGrCrUmArGrUrCmCmGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 38) |
| tracrRNA 32 | mA#mG#mC#mAmUmAmGmCmAmAmGrUrUmArAmA |
| | mArUmAmAmGmGrCrUmArGrUrCmCrGmUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 39) |
| tracrRNA 33 | mA#mG#mC#mAmUmAmGmCmAmAmGrUrUmArAmA |
| | mArUmAmAmGmGrCrUmArGrUrCmCrGrUmUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 40) |
| tracrRNA 34 | mA#mG#mC#mAmUmAmGmCmAmAmGrU#rUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 41) |
| tracrRNA 35 | mA#mG#mC#mAmUmAmGmCmAmAmGrUrU#mArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 42) |
| tracrRNA 36 | mA#mG#mC#mAmUmAmGmCmAmAmGrUrUmArA#mA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 43) |
| tracrRNA 37 | mA#mG#mC#mAmUmAmGmCmAmAmGrUrUmArAmA |
| | mArU#mAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 44) |
| tracrRNA 38 | mA#mG#mC#mAmUmAmGmCmAmAmGrUrUmArAmA |
| | mArUmAmAmGmGrC#rUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 45) |
| tracrRNA 39 | mA#mG#mC#mAmUmAmGmCmAmAmGrUrUmArAmA |
| | mArUmAmAmGmGrCrU#mArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 46) |
| tracrRNA 40 | mA#mG#mC#mAmUmAmGmCmAmAmGrUrUmArAmA |
| | mArUmAmAmGmGrCrUmArG#rUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 47) |
| tracrRNA 41 | mA#mG#mC#mAmUmAmGmCmAmAmGrUrUmArAmA |
| | mArUmAmAmGmGrCrUmArGrU#rCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 48) |
| tracrRNA 42 | mA#mG#mC#mAmUmAmGmCmAmAmGrUrUmArAmA |
| | mArUmAmAmGmGrCrUmArGrUrC#mCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 49) |
| tracrRNA 43 | mA#mG#mC#mAmUmAmGmCmAmAmGrUrUmArAmA |
| | mArUmAmAmGmGrCrUmArGrUrCmCrG#rUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 50) |
| tracrRNA 44 | mA#mG#mC#mAmUmAmGmCmAmAmGrUrUmArAmA |
| | mArUmAmAmGmGrCrUmArGrUrCmCrGrU#rUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 51) |
| tracrRNA 45 | mA#mG#mC#mAmUmAmGmCmAmAmGrUrUmArAmA |
| | mArUmAmAmGmGrCrUmArGrUrCmCrGrUrU#mAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 52) |
| tracrRNA 46 | mA#mG#mC#mAmUmAmGmCmAmAmGfUfUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 53) |
| tracrRNA 47 | mA#mG#mC#mAmUmAmGmCmAmAmGrUrUmAfAmA |
| | mAmUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 54) |
| tracrRNA 48 | mA#mG#mC#mAmUmAmGmCmAmAmGfUfUmAfAmA |
| | mAfUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 55) |
| tracrRNA 49 | mA#mG#mC#mAmUmAmGmCmAmAmGrUrUmArAmA |
| | mArUmAmAmGmGfCfUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 56) |
| tracrRNA 50 | mA#mG#mC#mAmUmAmGmCmAmAmGrUrUmArAmA |
| | mArUmAmAmGmGrCrUmAfGfUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 57) |
| tracrRNA 51 | mA#mG#mC#mAmUmAmGmCmAmAmGrUrUmArAmA |
| | mArUmAmAmGmGrCrUmArGrUrCmCfGfUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 58) |
| tracrRNA 52 | mA#mG#mC#mAmUmAmGmCmAmAmGrUrUmArAmA |
| | mArUmAmAmGmGfCfUmArGrUrCmCfGfUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 59) |
| tracrRNA 53 | mA#mG#mC#mAmUmAmGmCmAmAmGrUrUmArAmA |
| | mArUmAmAmGmGfCfUmAfGfUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 60) |
| tracrRNA 54 | mA#mG#mC#mAmUmAmGmCmAmAmGrUrUmArAmA |
| | mArUmAmAmGmGrCrUmAfGfUfCmCfGfUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 61) |
| tracrRNA 55 | mA#mG#mC#mAmUmAmGmCmAmAmGrUrUmArAmA |
| | mArUmAmAmGmGrCrUmAfGfUfCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 57) |
| tracrRNA 56 | mA#mG#mC#mAmUmAmGmCmAmAmGrUrUmArAmA |
| | mArUmAmAmGmGrCrUmArGrUrCmCfGrUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 62) |
| tracrRNA 57 | mA#mG#mC#mAmUmAmGmCmAmAmGrUrUmArAmA |
| | mArUmAmAmGmGfCfUmAfGrUfCmCfGrUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 63) |
| tracrRNA 58 | mA#mG#mC#mAmUmAmGmCmAmAmGrUrUmArAmA |
| | mArUmAmAmGmGrCrUmAfGrUfCmCfGrUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 64) |
| tracrRNA 59 | mA#mG#mC#mAmUmAmGmCmAmAmGrUrUmArAmA |
| | mArUmAmAmGmGfCrUmAfGrUfCmCfGrUfUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 65) |
| tracrRNA 60 | mA#mG#mC#mAmUmAmGmCmAmAmGmUmUmArAm |
| | AmArUmAmAmGmGrCrUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 66) |
| tracrRNA 61 | mA#mG#mC#mAmUmAmGmCmAmAmGrUrUmAmAmA |
| | mAmUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 67) |
| tracrRNA 62 | mA#mG#mC#mAmUmAmGmCmAmAmGmUmUmAmAm |
| | AmAmUmAmAmGmGrCrUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 68) |
| tracrRNA 63 | mA#mG#mC#mAmUmAmGmCmAmAmGrUrUmArAmA |
| | mArUmAmAmGmGmCmUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 69) |
| tracrRNA 64 | mA#mG#mC#mAmUmAmGmCmAmAmGrUrUmArAmA |
| | mArUmAmAmGmGrCrUmAmGmUmCmCrGrUrUmAmU |
| | mCmAmAmCmUmUmGmAmAmAmAmAmGmUmGmGm |
| | CmAmCmCmGmAmGmUmCmGmGmUmGmC#mU#mU# |
| | mU (SEQ ID NO: 70) |
| tracrRNA 65 | mA#mG#mC#mAmUmAmGmCmAmAmGrUrUmArAmA |
| | mArUmAmAmGmGrCrUmArGrUrCmCmGmUmUmAmU |
| | mCmAmAmCmUmUmGmAmAmAmAmAmGmUmGmGm |
| | CmAmCmCmGmAmGmUmCmGmGmUmGmC#mU#mU# |
| | mU (SEQ ID NO: 71) |
| tracrRNA 66 | mA#mG#mC#mAmUmAmGmCmAmAmGrUrUmArAmA |
| | mArUmAmAmGmGmCmUmArGrUrCmCmGmUmUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 72) |
| tracrRNA 67 | mA#mG#mC#mAmUmAmGmCmAmAmGrUrUmArAmA |
| | mArUmAmAmGmGmCmUmAmGmUmCmCrGrUrUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 73) |
| tracrRNA 68 | mA#mG#mC#mAmUmAmGmCmAmAmGrUrUmArAmA |
| | mArUmAmAmGmGrCrUmAmGmUmCmCmGmUmUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 74) |
| tracrRNA 69 | mA#mG#mC#mAmUmAmGmCmAmAmGrUrUmArAmA |
| | mArUmAmAmGmGrCrUmAmGrUmCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 75) |
| tracrRNA 70 | mA#mG#mC#mAmUmAmGmCmAmAmGrUrUmArAmA |
| | mArUmAmAmGmGrCrUmArGrUrCmCmGrUmUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 76) |
| tracrRNA 71 | mA#mG#mC#mAmUmAmGmCmAmAmGrUrUmArAmA |
| | mArUmAmAmGmGmCmUmAmGrUmCmCmGrUmUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 77) |
| tracrRNA 72 | mA#mG#mC#mAmUmAmGmCmAmAmGrUrUmArAmA |
| | mArUmAmAmGmGrCrUmAmGrUmCmCmGrUmUmAmU |
| | mCmAmAmCmUmUmGmAmAmAmAmAmGmUmGmGm |
| | CmAmCmCmGmAmGmUmCmGmGmUmGmC#mU#mU# |
| | mU (SEQ ID NO: 78) |
| tracrRNA 73 | mA#mG#mC#mAmUmAmGmCmAmAmGrUrUmArAmA |
| | mArUmAmAmGmGmCrUmAmGrUmCmCmGrUmUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 79) |
| tracrRNA 74 | mA#mG#mC#mAmUmAmGmCmAmAmGdUdUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 80) |
| tracrRNA 75 | mA#mG#mC#mAmUmAmGmCmAmAmGrUrUmAdAmA |
| | mAdUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 81) |
| tracrRNA 76 | mA#mG#mC#mAmUmAmGmCmAmAmGdUdUmAdAmA |
| | mAdUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 82) |
| tracrRNA 77 | mA#mG#mC#mAmUmAmGmCmAmAmGrUrUmArAmA |
| | mArUmAmAmGmGdCdUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 83) |
| tracrRNA 78 | mA#mG#mC#mAmUmAmGmCmAmAmGrUrUmArAmA |
| | mArUmAmAmGmGrCrUmAdGdUdCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 84) |
| tracrRNA 79 | mA#mG#mC#mAmUmAmGmCmAmAmGrUrUmArAmA |
| | mArUmAmAmGmGrCrUmArGrUrCmCdGdUdUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 85) |
| tracrRNA 80 | mA#mG#mC#mAmUmAmGmCmAmAmGrUrUmArAmA |
| | mArUmAmAmGmGdCdUmArGrUrCmCdGdUdUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 86) |
| tracrRNA 81 | mA#mG#mC#mAmUmAmGmCmAmAmGrUrUmArAmA |
| | mArUmAmAmGmGdCdUmAdGdUdCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 87) |
| tracrRNA 82 | mA#mG#mC#mAmUmAmGmCmAmAmGrUrUmArAmA |
| | mArUmAmAmGmGrCrUmAdGdUdCmCdGdUdUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 88) |
| tracrRNA 83 | mA#mG#mC#mAmUmAmGmCmAmAmGrUrUmArAmA |
| | mArUmAmAmGmGrCrUmAdGrUdCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 89) |
| tracrRNA 84 | mA#mG#mC#mAmUmAmGmCmAmAmGrUrUmArAmA |
| | mArUmAmAmGmGrCrUmArGrUrCmCdGrUdUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 90) |
| tracrRNA 85 | mA#mG#mC#mAmUmAmGmCmAmAmGrUrUmArAmA |
| | mArUmAmAmGmGdCdUmAdGrUdCmCdGrUdUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 91) |
| tracrRNA 86 | mA#mG#mC#mAmUmAmGmCmAmAmGrUrUmArAmA |
| | mArUmAmAmGmGrCrUmAdGrUdCmCdGrUdUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 92) |
| tracrRNA 87 | mA#mG#mC#mAmUmAmGmCmAmAmGrUrUmArAmA |
| | mArUmAmAmGmGdCrUmAdGrUdCmCdGrUdUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 93) |
| trac rRNA 88 | mA#mG#mC#mAmUmAmGmCmAmAmGrU#rU#mArAm |
| | AmArUmAmAmGmGrCrUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 94) |
| tracrRNA 89 | mA#mG#mC#mAmUmAmGmCmAmAmGrUrUmArA#mA |
| | mArU#mAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 95) |
| tracrRNA 90 | mA#mG#mC#mAmUmAmGmCmAmAmGrU#rU#mArA# |
| | mAmArU#mAmAmGmGrCrUmArGrUrCmCrGrUrUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 96) |
| tracrRNA 91 | mA#mG#mC#mAmUmAmGmCmAmAmGrUrUmArAmA |
| | mArUmAmAmGmGrC#rU#mArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 97) |
| tracrRNA 92 | mA#mG#mC#mAmUmAmGmCmAmAmGrUrUmArAmA |
| | mArUmAmAmGmGrCrUmArG#rU#rC#mCrGrUrUmAmU |
| | mCmAmAmCmUmUmGmAmAmAmAmAmGmUmGmGm |
| | CmAmCmCmGmAmGmUmCmGmGmUmGmC#mU#mU# |
| | mU (SEQ ID NO: 98) |
| tracrRNA 93 | mA#mG#mC#mAmUmAmGmCmAmAmGrUrUmArAmA |
| | mArUmAmAmGmGrCrUmArGrUrCmCrG#rU4U#mAmU |
| | mCmAmAmCmUmUmGmAmAmAmAmAmGmUmGmGm |
| | CmAmCmCmGmAmGmUmCmGmGmUmGmC#mU#mU# |
| | mU (SEQ ID NO: 99) |
| tracrRNA 94 | mA#mG#mC#mAmUmAmGmCmAmAmGrUrUmArAmA |
| | mArUmAmAmGmGrC#rU#mArGrUrCmCrG#rU#rU#mAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 100) |
| tracrRNA 95 | mA#mG#mC#mAmUmAmGmCmAmAmGrUrUmArAmA |
| | mArUmAmAmGmGrC#rU#mArG#rU#rC#mCrGrUrUmAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 101) |
| tracrRNA 96 | mA#mG#mC#mAmUmAmGmCmAmAmGrUrUmArAmA |
| | mArUmAmAmGmGrCrUmArG#rU#rC#mCrG#rU#rU#mA |
| | mUmCmAmAmCmUmUmGmAmAmAmAmAmGmUmGm |
| | GmCmAmCmCmGmAmGmUmCmGmGmUmGmC#mU#m |
| | U#mU (SEQ ID NO: 102) |
| tracrRNA 97 | mA#mG#mC#mAmUmAmGmCmAmAmGrUrUmArAmA |
| | mArUmAmAmGmGrCrUmArG#rUrC#mCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 103) |
| tracrRNA 98 | mA#mG#mC#mAmUmAmGmCmAmAmGrUrUmArAmA |
| | mArUmAmAmGmGrCrUmArGrUrCmCrG#rUrU#mAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 104) |
| tracrRNA 99 | mA#mG#mC#mAmUmAmGmCmAmAmGrUrUmArAmA |
| | mArUmAmAmGmGrC#rU#mArG#rU#rCmCrG#rUrU#mA |
| | mUmCmAmAmCmUmUmGmAmAmAmAmAmGmUmGm |
| | GmCmAmCmCmGmAmGmUmCmGmGmUmGmC#mU#m |
| | U#mU (SEQ ID NO: 105) |
| tracrRNA | mA#mG#mC#mAmUmAmGmCmAmAmGrUrUmArAmA |
| 100 | mArUmAmAmGmGrCrUmArG#rU#rCmCrG#rUrU#mAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 106) |
| tracrRNA | mA#mG#mC#mAmUmAmGmCmAmAmGrUrUmArAmA |
| 101 | mArUmAmAmGmGrC#rUmArG#rU#rCmCrG#rUrU#mAm |
| | UmCmAmAmCmUmUmGmAmAmAmAmAmGmUmGmG |
| | mCmAmCmCmGmAmGmUmCmGmGmUmGmC#mU#mU |
| | #mU (SEQ ID NO: 107) |
| tracrRNA | mA#mG#mC#mAmUmAmGmCmAmAmGrUrUmArAmA |
| 104 | mAdUmAmAmGmGdCdUmArGdUdCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 108) |
| tracrRNA | mA#mG#mC#mAmUmAmGmCmAmAmGrUdUmArAmA |
| 105 | mAdUmAmAmGmGdCdUmAdGdUdCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U (SEQ ID NO: 109) |
| tracrRNA | mA#mG#mC#mAmUmAmGmCmAmAmGdUdUmAdAmA |
| 106 | mAdUmAmAmGmGdCdUmAdGdUdCmCdGdUdUmAmU |
| | mCmAmAmCmUmUmGmAmAmAmAmAmGmUmGmGm |
| | CmAmCmCmGmAmGmUmCmGmGmUmGmC#mU#mU# |
| | mU (SEQ ID NO: 110) |
| tracrRNA | mA#mG#mC#mAmUmAmGmCmAmAmGsUsUmArAmA |
| 107 | mAsUmAmAmGmGrCsUmArGsUrCmCrGsUsUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 111) |
| tracrRNA | mA#mG#mC#mAmUmAmGmCmAmAmGsUsUmArAmA |
| 108 | mAsUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 112) |
| tracrRNA | mA#mG#mC#mAmUmAmGmCmAmAmGrUrUmArAmA |
| 109 | mArUmAmAmGmGrCsUmArGsUrCmCrGsUsUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 113) |
| tracrRNA | mA#mG#mC#mAmUmAmGmCmAmAmGsUrUmArAmA |
| 110 | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 114) |
| tracrRNA | mA#mG#mC#mAmUmAmGmCmAmAmGrUsUmArAmA |
| 111 | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 115) |
| tracrRNA | mA#mG#mC#mAmUmAmGmCmAmAmGrUrUmArAmA |
| 112 | mAsUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 116) |
| tracrRNA | mA#mG#mC#mAmUmAmGmCmAmAmGrUrUmArAmA |
| 113 | mArUmAmAmGmGrCsUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 117) |
| tracrRNA | mA#mG#mC#mAmUmAmGmCmAmAmGrUrUmArAmA |
| 114 | mArUmAmAmGmGrCrUmArGsUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 118) |
| tracrRNA | mA#mG#mC#mAmUmAmGmCmAmAmGrUrUmArAmA |
| 115 | mArUmAmAmGmGrCrUmArGrUrCmCrGsUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 119) |
| tracrRNA | mA#mG#mC#mAmUmAmGmCmAmAmGrUrUmArAmA |
| 116 | mArUmAmAmGmGrCrUmArGrUrCmCrGrUsUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 120) |

It will be understood to those of skill in the art that the base sequence of the first 20 nucleotides of the exemplary crRNAs recited in Table 1 above are directed to a specific target. This 20-nucleotide base sequence may be changed based on the target nucleic acid, however the chemical modifications remain the same. An exemplary unmodified crRNA sequence, from 5′ to 3′, is NNNNNNNNNNNNNNNNNNNNGUUUUAGAGCUAUGCU (SEQ ID NO: 1), where “N” corresponds to any nucleotide (e.g., A, U, G, or C). An exemplary unmodified tracrRNA sequence, from 5′ to 3′, is AGCAUAGCAAGUUAAAAUAAGGCUAGUCCGUUAUCAACUUGAAAAAGUGGCACCGAGUCGGUGCUUU (SEQ ID NO: 2).

It will be further understood to those of skill in the art that the guide sequence may be 10-30 nucleotides in length, preferably 16-24 nucleotides in length (for example, 16, 17, 18, 19, 20, 21, 22, 23 or 24 nucleotides in length), and is at or near the 5′ terminus of a Cas9 gRNA.

### High-Affinity Repeat/Anti-Repeat Guide RNA Modifications

A crRNA and a tracrRNA hybridize together by forming a duplex between the repeat region of the crRNA and the anti-repeat region of the tracrRNA (see FIG. 1). In certain embodiments, modular, or dual RNA, guide RNAs are provided with modifications in the repeat region and the anti-repeat region to enhance the affinity between the two regions and form a stronger duplex.

The high-affinity interaction may be enhanced by increasing the GC nucleotide content in the duplex formed by the repeat regions and the anti-repeat region. Nucleotide modifications, such as 2′-Fluoro and 2′-O-Methyl modifications, may also be introduced, which increase the melting temperature (Tm) of the duplex. Further modifications include the use of orthogonal and non-naturally occurring nucleotides. The various repeat region/anti-repeat region modifications described herein enhance the stability of the duplex, helping to prevent the crRNA and tracrRNA from folding into sub-optimal structures and therefore promoting higher genome editing efficacy.

The use of a modular, or dual RNA, guide RNA approach over a single guide RNA (sgRNA) approach has several advantages, including the ease of making the shorter crRNA and tracrRNA relative to a longer sgRNA, and the reduced cost of manufacturing the dual RNAs relative to the sgRNA. Exemplary crRNAs and tracrRNAs with modifications in the repeat and anti-repeat region, including a high GC content and 2′-Fluoro modifications, are shown in Table 3 and Table 4 below.

TABLE 3
Exemplary modified repeat crRNAs.

| | rN = RNA, mN = 2′-O-methyl RNA, fN = 2′-fluoro RNA, |
|---|---|
| | N = A, U, G, or C |
| KEY: | N#N = phosphorothioate linkage |
| Name | Sequence |
| hiGC repeat | NNNNNNNNNNNNNNNNNNNNGUUUUAGAGCGA |
| crRNA | GCGC (SEQ ID NO: 3) |
| hiGC & 2′- | NNNNNNNNNNNNNNNNNNNNmGrU#rU#rU#fUfAm |
| fluoro repeat | GmAfGfCfGfAfG#fC#mG#mC (SEQ ID NO: 246) |
| crRNA | |

It will be understood that the hiGC repeat crRNA above may further comprise any of the crRNA chemical modification patterns as recited in Table 1 above.

TABLE 4
Exemplary modified repeat tracrRNAs

| | rN = RNA, mN = 2′-O-methyl RNA, fN = 2′-fluoro |
|---|---|
| | RNA, N = A, U, G, or C |
| KEY: | N#N = phosphorothioate linkage |
| Name | Sequence |
| hiGC anti-repeat | GCGCUCGCAAGUUAAAAUAAGGCUAGUCCGU |
| tracrRNA | UAUCAACUUGAAAAAGUGGCACCGAGUCGGU |
| | GCUUU (SEQ ID NO: 4) |
| hiGC & 2′-fluoro | mG#mC#fG#fCfUfCfGfCmAmAmGrUrUmArAmAm |
| anti-repeat | ArUmAmAmGmGrCrUmArGrUrCmCrGrUrUmAmU |
| tracrRNA | mCmAmAmCmUmUmGmAmAmAmAmAmGmUmG |
| | mGmCmAmCmCmGmAmGmUmCmGmGmUmGmC |
| | #mU#mU#mU (SEQ ID NO: 247) |

It will be understood that the hiGC anti-repeat tracrRNA above may further comprise any of the tracrRNA chemical modification patterns, as recited in Table 2 above.

### Guide RNA Conjugates

The chemically modified guide RNAs of the disclosure may be modified with terminally conjugated moieties. As used herein, a “terminally conjugated moiety” or “moiety” refers to a compound which may be linked or attached to the 5′ and/or 3′ end of the crRNA and/or tracrRNA of a guide RNA. Terminally conjugated moieties can provide increased stability, increased ability to penetrate cell membranes, increase cellular uptake, increase circulation time in vivo, act as a cell-specific directing reagent, and/or provide a means to monitor cellular or tissue-specific uptake.

In certain embodiments, the terminally conjugated moiety is conjugated to the 5′ end of the crRNA portion of a guide RNA. In certain embodiments, the terminally conjugated moiety is conjugated to the 3′ end of the crRNA portion of a guide RNA. In certain embodiments, the terminally conjugated moiety is conjugated to the 5′ end of the tracrRNA portion of a guide RNA. In certain embodiments, the terminally conjugated moiety is conjugated to the 3′ end of the tracrRNA portion of a guide RNA.

In certain exemplary embodiments, a terminally conjugated moiety includes, but is not limited to, fatty acid, steroid, secosteroid, lipid, ganglioside analog, nucleoside analogs, endocannabinoid, vitamin, receptor ligand, peptide, aptamer, alkyl chain, fluorophore, antibody, nuclear localization signal, and the like.

In certain exemplary embodiments, a terminally conjugated moiety includes, but is not limited to, cholesterol, cholesterol-triethylene glycol (TEGChol), docosahexaenoic acid (DHA), docosanoic acid (DCA), lithocholic acid (LA), GalNAc, amphiphilic block copolymer (ABC), hydrophilic block copolymer (HBC), poloxamer, Cy5, Cy3, and the like.

In certain exemplary embodiments, the at least one terminally conjugated moiety is a modified lipid, including a branched lipid (such as the structure shown in Formula I) or a headgroup-modified lipid (such as the structure shown in Formula II).

X-MC(═Y)M-Z-[L-MC(═Y)M-R]n Formula I:

where X is a moiety that links the lipid to the guide RNA, each Y is independently oxygen or sulfur, each M is independently CH₂, NH, O or S, Z is a branching group which allows two or three (“n”) chains to be joined to the rest of the structure, L is an optional linker moiety, and each R is independently a saturated, monounsaturated or polyunsaturated linear or branched moiety from 2 to 30 atoms in length, a sterol, or other hydrophobic group.

X-MC(═Y)M-Z-[L-MC(═Y)M-R]n-L-K-J Formula II:

where X is a moiety that links the lipid to the guide RNA, each Y is independently oxygen or sulfur, each M is independently CH₂, NH, N-alkyl, O or S, Z is a branching group which allows two or three (“n”) chains to be joined to the rest of the structure, each L is independently an optional linker moiety, and R is a saturated, monounsaturated or polyunsaturated linear or branched moiety from 2 to 30 atoms in length, a sterol, or other hydrophobic group, K is a phosphate, sulfate, or amide and J is an aminoalkane or quaternary aminoalkane group.

The moieties may be attached to the terminal nucleotides of the guide RNA via a linker. Exemplary linkers include, but are not limited to, an ethylene glycol chain, an alkyl chain, a polypeptide, a polysaccharide, a block copolymer, and the like.

In certain embodiments, the moiety is conjugated to the 5′ end and/or 3′ end of any one of crRNA 23 to crRNA 134 (i.e., crRNA 23, crRNA 24, crRNA 25, crRNA 26, crRNA 27, crRNA 28, crRNA 29, crRNA 30, crRNA 31, crRNA 32, crRNA 33, crRNA 34, crRNA 35, crRNA 36, crRNA 37, crRNA 38, crRNA 39, crRNA 40, crRNA 41, crRNA 42, crRNA 43, crRNA 44, crRNA 45, crRNA 46, crRNA 47, crRNA 48, crRNA 49, crRNA 50, crRNA 51, crRNA 52, crRNA 53, crRNA 54, crRNA 55, crRNA 56, crRNA 57, crRNA 58, crRNA 59, crRNA 60, crRNA 61, crRNA 62, crRNA 63, crRNA 64, crRNA 65, crRNA 66, crRNA 67, crRNA 68, crRNA 69, crRNA 70, crRNA 71, crRNA 72, crRNA 73, crRNA 74, crRNA 75, crRNA 76, crRNA 77, crRNA 78, crRNA 79, crRNA 80, crRNA 81, crRNA 82, crRNA 83, crRNA 84, crRNA 85, crRNA 86, crRNA 87, crRNA 88, crRNA 89, crRNA 90, crRNA 91, crRNA 92, crRNA 93, crRNA 94, crRNA 95, crRNA 96, crRNA 97, crRNA 98, crRNA 99, crRNA 100, crRNA 101, crRNA 102, crRNA 103, crRNA 104, crRNA 105, crRNA 106, crRNA 107, crRNA 108, crRNA 109, crRNA 110, crRNA 111, crRNA 112, crRNA 113, crRNA 114, crRNA 115, crRNA 116, crRNA 117, crRNA 118, crRNA 119, crRNA 120, crRNA 121, crRNA 122, crRNA 123, crRNA 124, crRNA 125, crRNA 126, crRNA 127, crRNA 128, crRNA 129, crRNA 130, crRNA 131, crRNA 132, crRNA 133, or crRNA 134).

In certain embodiments, the moiety is conjugated to the 5′ end and/or 3′ end of any one of tracrRNA 21 to tracrRNA 116 (i.e., tracrRNA 21, tracrRNA 22, tracrRNA 23, tracrRNA 24, tracrRNA 25, tracrRNA 26, tracrRNA 27, tracrRNA 28, tracrRNA 29, tracrRNA 30, tracrRNA 31, tracrRNA 32, tracrRNA 33, tracrRNA 34, tracrRNA 35, tracrRNA 36, tracrRNA 37, tracrRNA 38, tracrRNA 39, tracrRNA 40, tracrRNA 41, tracrRNA 42, tracrRNA 43, tracrRNA 44, tracrRNA 45, tracrRNA 46, tracrRNA 47, tracrRNA 48, tracrRNA 49, tracrRNA 50, tracrRNA 51, tracrRNA 52, tracrRNA 53, tracrRNA 54, tracrRNA 55, tracrRNA 56, tracrRNA 57, tracrRNA 58, tracrRNA 59, tracrRNA 60, tracrRNA 61, tracrRNA 62, tracrRNA 63, tracrRNA 64, tracrRNA 65, tracrRNA 66, tracrRNA 67, tracrRNA 68, tracrRNA 69, tracrRNA 70, tracrRNA 71, tracrRNA 72, tracrRNA 73, tracrRNA 74, tracrRNA 75, tracrRNA 76, tracrRNA 77, tracrRNA 78, tracrRNA 79, tracrRNA 80, tracrRNA 81, tracrRNA 82, tracrRNA 83, tracrRNA 84, tracrRNA 85, tracrRNA 86, tracrRNA 87, tracrRNA 88, tracrRNA 89, tracrRNA 90, tracrRNA 91, tracrRNA 92, tracrRNA 93, tracrRNA 94, tracrRNA 95, tracrRNA 96, tracrRNA 97, tracrRNA 98, tracrRNA 99, tracrRNA 100, tracrRNA 101, tracrRNA 102, tracrRNA 103, tracrRNA 104, tracrRNA 105, tracrRNA 106, tracrRNA 107, tracrRNA 108, tracrRNA 109, tracrRNA 110, tracrRNA 111, tracrRNA 112, tracrRNA 113, tracrRNA 114, tracrRNA 115, or tracrRNA 116).

Exemplary crRNAs with conjugated moieties may be found in Table 5 below.

TABLE 5
Exemplary crRNAs with conjugated moieties.

| | rN = RNA, mN = 2′-O-methyl RNA, fN = 2′-fluoro |
|---|---|
| | RNA, N = A, U, G, or C |
| | N#N = phosphorothioate linkage, GalNAc = N- |
| KEY: | Acetylgalactosamine |
| Name | Sequence |
| crRNA 29- | mG#mA#mG#mAmCmAmAmAmUmCfAfCfCfUrG#f |
| MCV1a | CfCfUfCmGmGfUfUfUfUfAmGmAmGmCmUmAmU |
| GalNAc | #mG#mC#mU-GalNAc (SEQ ID NO: 248) |
| crRNA 39- | mG#mA#mG#rA#rC#rA#mAmAmUmCfAfCfCfUfGfC |
| MCV1a | fCfUfCmGmGfUfUfUfUfAmGmAmGmCmUmAmU# |
| GalNAc | mG#mC#mU-GalNAc (SEQ ID NO: 249) |
| crRNA 40- | mG#mA#mG#dA#dC#dA#mAmAmUmCfAfCfCfUfGf |
| MCV1a | CfCfUfCmGmGfUfUfUfUfAmGmAmGmCmUmAmU |
| GalNAc | #mG#mC#mU-GalNAc (SEQ ID NO: 250) |
| crRNA 20- | mG#mA#mG#mAmCmAmAmAmUmCfAfCfCfUrG#r |
| MCV1a | C#fCfUrC#mGmGrU#rU#rUgUfAmGmAmGmCmUm |
| Cy3 | AmU#mG#mC#mU-Cy3 (SEQ ID NO: 251) |
| crRNA 20- | mC#mC#mC#mAmUmAmCmCmUmUfGfGfAfGrC#r |
| PCSK9b | A#fAfCrG#mGmGrU#rU#rU#fUfAmGmAmGmCmUm |
| GalNAc | AmU#mG#mC#mU-GalNAc (SEQ ID NO: 252) |
| crRNA 29- | mC#mC#mC#mAmUmAmCmCmUmUfGfGfAfGrC#f |
| PCSK9b | AfAfCfGmGmGfUfUfUfUfAmGmAmGmCmUmAmU |
| GalNAc | #mG#mC#mU-GalNAc (SEQ ID NO: 253) |
| crRNA 39- | mC#mC#mC#rA4U4A#mCmCmUmUfGfGfAfGfCfA |
| PCSK9b | fAfCfGmGmGfUfUfUfUfAmGmAmGmCmUmAmU# |
| GalNAc | mG#mC#mU-GalNAc (SEQ ID NO: 254) |
| crRNA 40- | mC#mC#mC#dA#dU#dA#mCmCmUmUfGfGfAfGfCf |
| PCSK9b | AfAfCfGmGmGfUfUfUfUfAmGmAmGmCmUmAmU |
| GalNAc | #mG#mC#mU-GalNAc (SEQ ID NO: 255) |
| crRNA 42- | mC#mC#mC#mAmUmAmCmCmUmUfGfGfAfGrC#r |
| PCSK9b | A#fAfCrG#mGmGdU#dU#dU#fUfAmGmAmGmCmU |
| GalNAc | mAmU#mG#mC#mU-GalNAc (SEQ ID NO: 256) |
| crRNA 20- | mG#mA#mG#mAmCmAmAmAmUmCfAfCfCfUrG#r |
| DHA | C#fCfUrC#mGmGrU#rU#rUgUfAmGmAmGmCmUm |
| | AmU#mG#mC#mU-DHA (SEQ ID NO: 257) |
| crRNA 39- | mG#mA# mG#rA#rC#rA#mAmAmUmCfAfCfCfUfGfC |
| DHA | fCfUfCmGmGfUfUfUfUfAmGmAmGmCmUmAmU# |
| | mG#mC#mU-DHA (SEQ ID NO: 258) |
| crRNA 40- | mG#mA#mG#dA#dC#dA#mAmAmUmCfAfCfCfUfGf |
| DHA | CfCfUfCmGmGfUfUfUfUfAmGmAmGmCmUmAmU |
| | #mG#mC#mU-DHA (SEQ ID NO: 259) |
| crRNA 42- | mG#mA#mG#mAmCmAmAmAmUmCfAfCfCfUrG#r |
| DHA | C#fCfUrC#mGmGdU#dU#dU#fUfAmGmAmGmCmU |
| | mAmU#mG#mC#mU-DHA (SEQ ID NO: 260) |
| crRNA 113- | mG#mA#mG#dA#dC#dA#mAmAmUmCfAfCfCfUdG# |
| DHA | dC#fCfUdC#mGmGdU#dU#dUffUfAmGmAmGmCm |
| | UmAmU#mG#mC#mU-DHA (SEQ ID NO: 261) |
| crRNA 20- | mG#mA#mG#mAmCmAmAmAmUmCfAfCfCfUrG#r |
| DCA | C#fCfUrC#mGmGrU#rU#rUgUfAmGmAmGmCmUm |
| | AmU#mG#mC#mUdTdT-DCA (SEQ ID NO: 262) |
| crRNA 39- | mG#mA#mG#rA#rC#rA#mAmAmUmCfAfCfCfUfGfC |
| DCA | fCfUfCmGmGfUfUfUfUfAmGmAmGmCmUmAmU# |
| | mG#mC#mUdTdT-DCA (SEQ ID NO: 263) |
| crRNA 40- | mG#mA#mG#dA#dC#dA#mAmAmUmCfAfCfCfUfGf |
| DCA | CfCfUfCmGmGfUfUfUfUfAmGmAmGmCmUmAmU |
| | #mG#mC#mUdTdT-DCA (SEQ ID NO: 264) |
| crRNA 42- | mG#mA#mG#mAmCmAmAmAmUmCfAfCfCfUrG#r |
| DCA | C#fCfUrC#mGmGdU#dU#dU#fUfAmGmAmGmCmU |
| | mAmU#mG#mC#mUdTdT-DCA (SEQ ID NO: 265) |
| crRNA 113- | mG#mA#mG#dA#dC#dA#mAmAmUmCfAfCfCfUdG# |
| DCA | dC#fCfUdC#mGmGdU#dU#dUffUfAmGmAmGmCm |
| | UmAmU#mG#mC#mUdTdT-DCA (SEQ ID NO: 266) |

where: GalNAc-(N-Acetylgalactosamine) 3-40 moieties; and Cy3-Cyanine 3 fluorescent dye

### Chemically Modified Single Guide RNA

As described herein, the chemically modified guide RNAs of the disclosure may be constructed as single guide RNAs (sgRNAs) by linking the 3′ end of a crRNA to the 5′ end of a tracrRNA. The linker may be an oligonucleotide loop, including a chemically modified oligonucleotide loop. In certain embodiments, the oligonucleotide loop comprises a GAAA tetraloop. The linker may be a non-nucleotide chemical linker, including, but not limited to, ethylene glycol oligomers (see, e.g., Pils et al. Nucleic Acids Res. 28(9): 1859-1863 (2000)).

### RNA-Guided Nucleases

RNA-guided nucleases according to the present disclosure include, without limitation, naturally-occurring Type II CRISPR nucleases such as Cas9, as well as other nucleases derived or obtained therefrom. Exemplary Cas9 nucleases that may be used in the present disclosure include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9). In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpfl), species (e.g., S. pyogenes vs. S. aureus) or variation (e.g., full-length vs. truncated or split; naturally-occurring PAM specificity vs. engineered PAM specificity).

Various RNA-guided nucleases may require different sequential relationships between PAMs and protospacers. In general, Cas9s recognize PAM sequences that are 5′ of the protospacer as visualized relative to the top or complementary strand.

In addition to recognizing specific sequential orientations of PAMs and protospacers, RNA-guided nucleases generally recognize specific PAM sequences. S. aureus Cas9, for example, recognizes a PAM sequence of NNGRRT, wherein the N sequences are immediately 3′ of the region recognized by the gRNA targeting domain. S. pyogenes Cas9 recognizes NGG PAM sequences. It should also be noted that engineered RNA-guided nucleases can have PAM specificities that differ from the PAM specificities of similar nucleases (such as the naturally occurring variant from which an RNA-guided nuclease is derived, or the naturally occurring variant having the greatest amino acid sequence homology to an engineered RNA-guided nuclease). Modified Cas9s that recognize alternate PAM sequences are described below.

RNA-guided nucleases are also characterized by their DNA cleavage activity: naturally-occurring RNA-guided nucleases typically form DSBs in target nucleic acids, but engineered variants have been produced that generate only SSBs (discussed above; see also Ran 2013, incorporated by reference herein), or that do not cut at all.

The RNA-guided nuclease Cas9 may be a variant of Cas9 with altered activity. Exemplary variant Cas9 nucleases include, but are not limited to, a Cas9 nickase (nCas9), a catalytically dead Cas9 (dCas9), a hyper accurate Cas9 (HypaCas9) (Chen et al. Nature, 550(7676), 407-410 (2017)), a high fidelity Cas9 (Cas9-HF) (Kleinstiver et al. Nature 529(7587), 490-495 (2016)), an enhanced specificity Cas9 (eCas9) (Slaymaker et al. Science 351(6268), 84-88 (2016)), and an expanded PAM Cas9 (xCas9) (Hu et al. Nature doi: 10.1038/nature26155 (2018)).

The RNA-guided nucleases may be combined with the chemically modified guide RNAs of the present disclosure to form a genome-editing system. The RNA-guided nucleases may be combined with the chemically modified guide RNAs to form an RNP complex that may be delivered to a cell where genome-editing is desired. The RNA-guided nucleases may be expressed in a cell where genome-editing is desired with the chemically modified guide RNAs delivered separately. For example, the RNA-guided nucleases may be expressed from a polynucleotide such as a vector or a synthetic mRNA. The vector may be a viral vector, including, be not limited to, an adeno-associated virus (AAV) vector or a lentivirus (LV) vector.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1

### Synthesis of Chemically Modified crRNA and tracrRNA

crRNAs and tracrRNAs were synthesized at 1 μmole scale on an Applied Biosystems 394 DNA synthesizer. BTT (0.25 M in acetonitrile, ChemGenes) was used as activator. 0.05 M iodine in pyridine:water (9:1) (TEDIA) was used as oxidizer. DDTT (0.1 M, ChemGenes) was used as sulfurizing agent. 3% TCA in DCM (TEDIA) was used as deblock solution. RNAs were grown on 1000 Å CPG functionalized with Unylinker (˜42 μmol/g). RNA and 2′-OMe phosphoramidites (ChemGenes) were dissolved in acetonitrile to 0.15 M; the coupling time was 10 min for each base. The nucleobases were deprotected with a 3:1 NH4OH:EtOH solution for 48 hours at room temperature. Deprotection of the TBDMS group was achieved with DMSO:NEt3.3HF (4:1) solution (500 μL) at 65° C. for 3 hours. RNA oligonucleotides were then recovered by precipitation in 3M NaOAc (25 μL) and n-BuOH (1 mL), and the pellet was washed with cold 70% EtOH and resuspended in 1 mL RNase-free water.

Purification of the crRNAs and tracrRNAs were carried out by high performance liquid chromatography using a 1260 infinity system with an Agilent PL-SAX 1000 Å column (150×7.5 mm, 8 μm). Buffer A: 30% acetonitrile in water; Buffer B: 30% acetonitrile in 1M NaCl4 (aq). Excess salt was removed with a Sephadex Nap-10 column.

crRNAs and tracrRNAs were analyzed on an Agilent 6530 Q-TOF LC/MS system with electrospray ionization and time of flight ion separation in negative ionization mode. The data were analyzed using Agilent Mass Hunter software. Buffer A: 100 mM hexafluoroisopropanol with 9 mM triethylamine in water; Buffer B: 100 mM hexafluoroisopropanol with 9 mM trimethylamine in methanol.

The crRNAs used in the Examples are recited below in Table 6. Table 2 above recites tracrRNAs used in the Examples.

TABLE 6
Exemplary crRNAs.

| | rN = RNA, mN = 2′-O-methyl RNA, fN = 2′-fluoro RNA, |
|---|---|
| | aN = 2′-NH2 (2′-amino RNA), sN = 4′-thio RNA, dN = 2′- |
| | deoxy RNA, N = A, U, G, or C |
| KEY: | N#N = phosphorothioate linkage |
| Name | Sequence |
| crRNA 1 | mG#mG#mU#mGmAmGmCmUmCmUrUrArUrUrUmGmC |
| | mGrUmAmGrUrUrUmUmAmGmAmGmCmUmAmU#mG# |
| | mCmU (SEQ ID NO: 267) |
| crRNA 2 | rGrGrUrGrArGmCmUmCmUrUrArUrUrUrGrCrGrUmAmG |
| | rUrUrUrUrAmGmAmGmCmUmAmU#mG#mCmU (SEQ |
| | ID NO: 268) |
| crRNA 3 | rG#rG#rU#rGrArGmCmUmCmUrUrArUrUrUrGrCrGrUmA |
| | mGrUrUrUrUrAmGmAmGmCmUmAmU#mG#mCmU |
| | (SEQ ID NO: 269) |
| crRNA 4 | mG#mG#mU#mGmAmGmCmUmCmUrUrArUrUrUrGrCrG |
| | rUmAmGrUrUrUrUrAmGmAmGmCmUmAmU#mG#mC# |
| | mU (SEQ ID NO: 270) |
| crRNA 5 | rG4G#rU#rGrArGmCmUmCmUrUrArUrUrUrGmCmGrU |
| | mAmGrUrUrUrUrAmGmAmGmCmUmAmU#mG#mC#mU |
| | (SEQ ID NO: 271) |
| crRNA 6 | rG#rG#rU#rGrArGmCmUmCmUrUrArUrUrUrGrCrGrUmA |
| | mGrUrUrUmUmAmGmAmGmCmUmAmU#mG#mC#mU |
| | (SEQ ID NO: 272) |
| crRNA 7 | mG#mG#mU#mGmAmGmCmUmCmUrUrArUrUrUrGmC |
| | mGrUmAmGrUrUrUrUrAmGmAmGmCmUmAmU#mG#m |
| | C#mU (SEQ ID NO: 273) |
| crRNA 8 | mG#mG#mU#mGmAmGmCmUmCmUrUrArUrUrUrGrCrG |
| | rUmAmGrUrUrUmUmAmGmAmGmCmUmAmU#mG#mC |
| | #mU (SEQ ID NO: 274) |
| crRNA 9 | mG#mG#mU#mGmAmGmCmUmCmUrUrArUrUrU#rG#rC |
| | rGrU#mAmGrU#rU#rU#mUmAmGmAmGmCmUmAmU# |
| | mG#mC#mU (SEQ ID NO: 275) |
| crRNA 10 | mG#mG#mU#mGmAmGmCmUmCmUfUfAfUfUrU#rG#fC |
| | fGrU#mAmGrU#rU#rU#mUmAmGmAmGmCmUmAmU# |
| | mG#mC#mU (SEQ ID NO: 276) |
| crRNA 11 | mG#mG#mU#mGmAmGmCmUmCmUfUfAfUfUrU#rG#rC |
| | #rG#rU#mAmGrU#rU#rU#mUmAmGmAmGmCmUmAmU |
| | #mG#mC#mU (SEQ ID NO: 277) |
| crRNA 17 | mG#mG#mU#mGmAmGmCmUmCmUfUfAfUfUrU#rG#fC |
| | fGrU#mAmGrU#rU#rU#mUrA#mGmAmGmCmUmAmU# |
| | mG#mC#mU (SEQ ID NO: 278) |
| crRNA 18 | mG#mG#mU#mGmAmGmCmUmCmUfUfAfUfUrU#rG#fC |
| | fGrU#mAmGrU#rU#rU#rU#mAmGmAmGmCmUmAmU# |
| | mG#mC#mU (SEQ ID NO: 279) |
| crRNA 19 | mG#mG#mU#mGmAmGmCmUmCmUfUfAfUfUrU#rG#fC |
| | fGrU#mAmGrU#rU#rU#rU#rA#mGmAmGmCmUmAmU# |
| | mG#mC#mU (SEQ ID NO: 280) |
| crRNA 20 | mG#mG#mU#mGmAmGmCmUmCmUfUfAfUfUrU#rG#fC |
| | fGrU#mAmGrU#rU#rU#fUfAmGmAmGmCmUmAmU#m |
| | G#mC#mU (SEQ ID NO: 281) |
| crRNA 21 | mG#mG#mU#mGmAmGmCmUmCmUfUfAfUfUfUfGfCfG |
| | fUmAmGfUfUfUfUfAmGmAmGmCmUmAmU#mG#mC# |
| | mU (SEQ ID NO: 282) |
| crRNA 22 | mG#mG#mU#mGmAmGmCmUmCmUfUfAfUfUrU#rG#fC |
| | fGrU#mAmGfUrU#fUfUfAmGmAmGmCmUmAmU#mG# |
| | mC#mU (SEQ ID NO: 283) |
| crRNA 23 | mG#mG#mU#mGmAmGmCmUmCmUfUfAfUfUmUrG#fC |
| | fGrU#mAmGrU#rU#rU#fUfAmGmAmGmCmUmAmU#m |
| | G#mC#mU (SEQ ID NO: 284) |
| crRNA 24 | mG#mG#mU#mGmAmGmCmUmCmUfUfAfUfUrU#fGfCf |
| | GrU#mAmGrU#rU#rU#fUfAmGmAmGmCmUmAmU#mG |
| | #mC#mU (SEQ ID NO: 285) |
| crRNA 25 | mG#mG#mU#mGmAmGmCmUmCmUfUfAfUfUrU#rG#fC |
| | fGfUmAmGrU#rU#rUgUfAmGmAmGmCmUmAmU#mG |
| | #mC#mU (SEQ ID NO: 286) |
| crRNA 26 | mG#mG#mU#mGmAmGmCmUmCmUfUfAfUfUrU#rG#fC |
| | fGrU#mAmGfUrU#rUgUfAmGmAmGmCmUmAmU#mG |
| | #mC#mU (SEQ ID NO: 287) |
| crRNA 27 | mG#mG#mU#mGmAmGmCmUmCmUfUfAfUfUrU#rG#fC |
| | fGrU#mAmGrUgUrUgUfAmGmAmGmCmUmAmU#mG |
| | #mC#mU (SEQ ID NO: 288) |
| crRNA 28 | mG#mG#mU#mGmAmGmCmUmCmUfUfAfUfUrU#rG#fC |
| | fGrU#mAmGrU#rU#rUfUfAmGmAmGmCmUmAmU#mG |
| | #mC#mU (SEQ ID NO: 289) |
| crRNA 29 | mG#mG#mU#mGmAmGmCmUmCmUfUfAfUfUrU#fGfCf |
| | GfUmAmGfUfUfUfUfAmGmAmGmCmUmAmU#mG#mC |
| | #mU (SEQ ID NO: 290) |
| crRNA 46 | mG#mG#mU#mGmAmGmCmUmCmUfUfAfUfUmUrG#fC |
| | fGrU#mAmGrU#rUrUgUfAmGmAmGmCmUmAmU#mG |
| | #mC#mU (SEQ ID NO: 291) |
| crRNA 47 | mG#mG#mU#mGmAmGmCmUmCmUfUfAfUfUrU#mGfC |
| | fGrU#mAmGrU#rUrUgUfAmGmAmGmCmUmAmU#mG |
| | #mC#mU (SEQ ID NO: 292) |
| crRNA 48 | mG#mG#mU#mGmAmGmCmUmCmUfUfAfUfUrU#mGfC |
| | fGmUmAmGrU#rUrUgUfAmGmAmGmCmUmAmU#mG# |
| | mC#mU (SEQ ID NO: 293) |
| crRNA 49 | mG#mG#mU#mGmAmGmCmUmCmUfUfAfUfUrU#rG#fC |
| | fGrU#mAmGmUrU#rU#fUfAmGmAmGmCmUmAmU#mG |
| | #mC#mU (SEQ ID NO: 294) |
| crRNA 50 | mG#mG#mU#mGmAmGmCmUmCmUfUfAfUfUrU#rG#fC |
| | fGrU#mAmGrU#mUrU#fUfAmGmAmGmCmUmAmU#mG |
| | #mC#mU (SEQ ID NO: 295) |
| crRNA 51 | mG#mG#mU#mGmAmGmCmUmCmUfUfAfUfUrU#rG#fC |
| | fGrU#mAmGrU#rU#mUfUfAmGmAmGmCmUmAmU#mG |
| | #mC#mU (SEQ ID NO: 145) |
| crRNA 52 | mG#mA#mG#dA#dC#dA#mAmAmUmCfAfCfCfUfG#fC#f |
| | CfUfC#mGmGfU#fU#fUgUgA#mGmAmGmCmUmAmU |
| | #mG#mC#mU (SEQ ID NO: 296) |
| crRNA 53 | mG#mA#mG#dA#dC#dA#mA#mAmUmCfAfCfCfUfGfCfC |
| | fUfCmGmGfUfUfUfUfAmGmAmGmCmUmAmU#mG#mC |
| | #mU (SEQ ID NO: 297) |
| crRNA 54 | mG#mA#mG#dA#dC#dA#mAmA#mUmCfAfCfCfUfGfCfC |
| | fUfCmGmGfUfUfUfUfAmGmAmGmCmUmAmU#mG#mC |
| | #mU (SEQ ID NO: 298) |
| crRNA 55 | mG#mA#mG#dA#dC#dA#mAmAmU#mCfAfCfCfUfGfCfC |
| | fUfCmGmGfUfUfUfUfAmGmAmGmCmUmAmU#mG#mC |
| | #mU (SEQ ID NO: 299) |
| crRNA 56 | mG#mA#mG#dA#dC#dA#mAmAmUmC#fAfCfCfUfGfCfC |
| | fUfCmGmGfUfUfUfUfAmGmAmGmCmUmAmU#mG#mC |
| | #mU (SEQ ID NO: 300) |
| crRNA 57 | mG#mA#mG#dA#dC#dA#mAmAmUmCfA#fCfCfUfGfCfC |
| | fUfCmGmGfUfUfUfUfAmGmAmGmCmUmAmU#mG#mC |
| | #mU (SEQ ID NO: 301) |
| crRNA 58 | mG#mA#mG#dA#dC#dA#mAmAmUmCfAfC#fCfUfGfCfC |
| | fUfCmGmGfUfUfUfUfAmGmAmGmCmUmAmU#mG#mC |
| | #mU (SEQ ID NO: 302) |
| crRNA 59 | mG#mA#mG#dA#dC#dA#mAmAmUmCfAfCfC#fUfGfCfC |
| | fUfCmGmGfUfUfUfUfAmGmAmGmCmUmAmU#mG#mC |
| | #mU (SEQ ID NO: 303) |
| crRNA 60 | mG#mA#mG#dA#dC#dA#mAmAmUmCfAfCfCfU#fGfCfC |
| | fUfCmGmGfUfUfUfUfAmGmAmGmCmUmAmU#mG#mC |
| | #mU (SEQ ID NO: 304) |
| crRNA 61 | mG#mA#mG#dA#dC#dA#mAmAmUmCfAfCfCfUfGfCfC# |
| | fUfCmGmGfUfUfUfUfAmGmAmGmCmUmAmU#mG#mC |
| | #mU (SEQ ID NO: 305) |
| crRNA 62 | mG#mA#mG#dA#dC#dA#mAmAmUmCfAfCfCfUfGfCfCf |
| | UffCmGmGfUfUfUfUfAmGmAmGmCmUmAmU#mG#m |
| | C#mU (SEQ ID NO: 306) |
| crRNA 63 | mG#mA#mG#dA#dC#dA#mAmAmUmCfAfCfCfUfGfCfCf |
| | UfCmG#mGfUfUfUfUfAmGmAmGmCmUmAmU#mG#m |
| | C#mU (SEQ ID NO: 307) |
| crRNA 64 | mG#mA#mG#dA#dC#dA#mAmAmUmCfAfCfCfUfGfCfCf |
| | UfCmGmG#fUfUfUfUfAmGmAmGmCmUmAmU#mG#m |
| | C#mU (SEQ ID NO: 308) |
| crRNA 65 | mG#mA#mG#dA#dC#dA#mAmAmUmCfAfCfCfUfGfCfCf |
| | UfCmGmGfUfUfUfUfAmG#mAmGmCmUmAmU#mG#m |
| | C#mU (SEQ ID NO: 309) |
| crRNA 66 | mG#mA#mG#dA#dC#dA#mAmAmUmCfAfCfCfUfGfCfCf |
| | UfCmGmGfUfUfUfUfAmGmA#mGmCmUmAmU#mG#m |
| | C#mU (SEQ ID NO: 310) |
| crRNA 67 | mG#mA#mG#dA#dC#dA#mAmAmUmCfAfCfCfUfGfCfCf |
| | UfCmGmGfUfUfUfUfAmGmAmG#mCmUmAmU#mG#m |
| | C#mU (SEQ ID NO: 311) |
| crRNA 68 | mG#mA#mG#dA#dC#dA#mAmAmUmCfAfCfCfUfGfCfCf |
| | UfCmGmGfUfUfUfUfAmGmAmGmC#mUmAmU#mG#m |
| | C#mU (SEQ ID NO: 312) |
| crRNA 69 | mG#mA#mG#dA#dC#dA#mAmAmUmCfAfCfCfUfGfCfCf |
| | UfCmGmGfUfUfUfUfAmGmAmGmCmU#mAmU#mG#m |
| | C#mU (SEQ ID NO: 313) |
| crRNA 70 | mG#mA#mG#dA#dC#dA#mAmAmUmCfAfCfCfUfGfCfCf |
| | UfCmGmGfUfUfUfUfAmGmAmGmCmUmA#mU#mG#m |
| | C#mU (SEQ ID NO: 314) |
| crRNA 71 | mG#mA#mG#dA#dC#dA#mA#mA#mU#mCfAfCfCfUfG#f |
| | C#fCfUfC#mGmGfU#fU#fU#fU#fA#mGmAmGmCmUmA |
| | mU#mG#mC#mU (SEQ ID NO: 315) |
| crRNA 72 | mG#mA#mG#dA#dC#dA#mAmAmUmC#fA#fC#fCfUfG#f |
| | C#fCfUfC#mGmGfU#fU#fU#fU#fA#mGmAmGmCmUmA |
| | mU#mG#mC#mU (SEQ ID NO: 316) |
| crRNA 73 | mG#mA#mG#dA#dC#dA#mAmAmUmCfAfCfC#fU#fG#fC |
| | #fCfUfC#mGmGfU#fU#fU#fUgA#mGmAmGmCmUmAm |
| | U#mG#mC#mU (SEQ ID NO: 317) |
| crRNA 74 | mG#mA#mG#dA#dC#dA#mAmAmUmCfAfCfCfUfG#fC#f |
| | C#fU#fC#mGmGfU#fU#fUgU#fA#mGmAmGmCmUmAm |
| | U#mG#mC#mU (SEQ ID NO: 318) |
| crRNA 75 | mG#mA#mG#dA#dC#dA#mAmAmUmCfAfCfCfUfG#fC#f |
| | CfUfC#mG#mGfU#fU#fU#fU#fA#mGmAmGmCmUmAm |
| | U#mG#mC#mU (SEQ ID NO: 319) |
| crRNA 76 | mG#mA#mG#dA#dC#dA#mA#mA#mU#mCfAfCfCfUfGfC |
| | fCfUfCmGmGfUfUfUfUfAmGmAmGmCmUmAmU#mG# |
| | mC#mU (SEQ ID NO: 320) |
| crRNA 77 | mG#mA#mG#dA#dC#dA#mA#mA#mU#mC#fA#fC#fCfUf |
| | GfCfCfUfCmGmGfUfUfUfUfAmGmAmGmCmUmAmU#m |
| | G#mC#mU (SEQ ID NO: 321) |
| crRNA 78 | mG#mA#mG#dA#dC#dA#mA#mA#mU#mC#fA#fC#fC#fU |
| | #fG#fC#fCfUfCmGmGfUfUfUfUfAmGmAmGmCmUmAm |
| | U#mG#mC#mU (SEQ ID NO: 322) |
| crRNA 79 | mG#mA#mG#dA#dC#dA#mA#mA#mU#mC#fA#fC#fC#fU |
| | #fG#fC#fC#fU#fC#mGmGfUfUfUfUfAmGmAmGmCmUm |
| | AmU#mG#mC#mU (SEQ ID NO: 323) |
| crRNA 80 | mG#mA#mG#dA#dC#dA#mA#mA#mU#mC#fA#fC#fC#fU |
| | #fG#fC#fC#fU#fC#mG#mG#fU#fU#fU#fU#fA#mGmAmG |
| | mCmUmAmU#mG#mC#mU (SEQ ID NO: 324) |
| crRNA 81 | mG#mA#mG#dA#dC#dA#mAmAmUmCfAfCfCfUfG#fC#f |
| | CfUfC#mGmGfU#fU#fU#fU#fA#mGmAmGmC#mU#mA# |
| | mU#mG#mC#mU (SEQ ID NO: 325) |
| crRNA 82 | mG#mA#mG#dA#dC#dA#mAmAmUmCfAfCfCfUfG#fC#f |
| | CfUfC#mGmGfU#fUgUgU#fA#mG#mA#mG#mCmUmA |
| | mU#mG#mC#mU (SEQ ID NO: 326) |
| crRNA 83 | mG#mA#mG#dA#dC#dA#mAmAmUmCfAfCfCfUfG#fC#f |
| | CfUfC#mGmG#fU#fU#fU#fU#fA#mGmAmGmCmUmAm |
| | U#mG#mC#mU (SEQ ID NO: 327) |
| crRNA 84 | mG#mA#mG#dA#dC#dA#mAmAmUmCfAfCfCfUfGfCfCf |
| | UfCmGmGfUfUfUfUfAmGmAmGmC#mU#mA#mU#mG# |
| | mC#mU (SEQ ID NO: 328) |
| crRNA 85 | mG#mA#mG#dA#dC#dA#mAmAmUmCfAfCfCfUfGfCfCf |
| | UfCmGmGfUfUfUfUfA#mG#mA#mG#mC#mU#mA#mU# |
| | mG#mC#mU (SEQ ID NO: 329) |
| crRNA 86 | mG#mA#mG#dA#dC#dA#mAmAmUmCfAfCfCfUfGfCfCf |
| | UfCmGmGfUgUgU#fU#fA#mG#mA#mG#mC#mU#mA# |
| | mU#mG#mC#mU (SEQ ID NO: 330) |
| crRNA 87 | mG#mA#mG#dA#dC#dA#mA#mA#mUmCfAfCfCfUfGfCf |
| | CfUfCmGmGfUfUfUfUfAmGmAmGmCmU#mA#mU#mG |
| | #mC#mU (SEQ ID NO: 331) |
| crRNA 88 | mG#mA#mG#dA#dC#dA#mA#mA#mU#mC#fAfCfCfUfGf |
| | CfCfUfCmGmGfUfUfUfUfAmGmAmG#mC#mU#mA#mU |
| | #mG#mC#mU (SEQ ID NO: 332) |
| crRNA 89 | mG#mA#mG#dA#dC#dA#mA#mA#mU#mC#fA#fC#fCfUf |
| | GfCfCfUfCmGmGfUfUfUfUfAmG#mA#mG#mC#mU#mA |
| | #mU#mG#mC#mU (SEQ ID NO: 333) |
| crRNA 90 | mG#mA#mG#dA#dC#dA#mA#mA#mU#mC#fA#fC#fC#fU |
| | #fGfCfCfUfCmGmGfUfUfUfU#fA#mG#mA#mG#mC#mU |
| | #mA#mU#mG#mC#mU (SEQ ID NO: 334) |
| crRNA 91 | mG#mA#mG#dA#dC#dA#mA#mA#mU#mC#fA#fC#fC#fU |
| | #fG#fC#fCfUfCmGmGfUfUgU#fU#fA#mG#mA#mG#mC |
| | #mU#mA#mU#mG#mC#mU (SEQ ID NO: 335) |
| crRNA 92 | mG#mA#mG#dA#dC#dA#mA#mA#mU#mC#fA#fC#fC#fU |
| | #fG#fC#fC#fU#fC#mGmGfUgU#fU#fUgA#mG#mA#mG |
| | #mC#mU#mA#mU#mG#mC#mU (SEQ ID NO: 336) |
| crRNA 93 | mG#mA#mG#dA#dC#dA#mA#mA#mU#mC#fA#fC#fC#fU |
| | #fG#fC#fC#fU#fC#mG#mG#fU#fU#fU#fU#fA#mG#mA#m |
| | G#mC#mU#mA#mU#mG#mC#mU (SEQ ID NO: 337) |
| crRNA 94 | mG#mA#mG#mA#mC#mA#mAmAmUmCfAfCfCfUrG#rC |
| | #fCfUrC#mGmGrU#rU#rU#fUfAmGmAmGmCmUmAmU# |
| | mG#mC#mU (SEQ ID NO: 338) |
| crRNA 95 | mG#mA#mG#mA#mC#mA#mA#mAmUmCfAfCfCfUrG#r |
| | C#fCfUrC#mGmGrU#rU#rUgUfAmGmAmGmCmUmAm |
| | U#mG#mC#mU (SEQ ID NO: 339) |
| crRNA 96 | mG#mA#mG#mA#mC#mA#mAmAmUmCfAfCfCfUrG#rC |
| | #fCfUrC#mGmGrU#rU#rUgUfAmGmAmGmC#mUmAmU |
| | #mG#mC#mU (SEQ ID NO: 340) |
| crRNA 97 | mG#mA#mG#mA#mC#mA#mAmAmUmCfAfCfCfUrG#rC |
| | #fCfUrC#mGmGrU#rU#rUgUfAmGmAmGmCmU#mAmU |
| | #mG#mC#mU (SEQ ID NO: 341) |
| crRNA 98 | mG#mA#mG#mA#mC#mA#mAmAmUmCfAfCfCfUrG#rC |
| | #fCfUrC#mGmGrU#rU#rUgUfAmGmAmGmCmUmA#mU |
| | #mG#mC#mU (SEQ ID NO: 342) |
| crRNA 99 | mG#mA#mG#mA#mC#mA#mAmAmUmCfAfCfCfUrG#rC |
| | #fCfUrC#mGmGrU#rU#rU#fUgA#mGmAmGmCmUmAm |
| | U#mG#mC#mU (SEQ ID NO: 343) |
| crRNA 100 | mG#mA#mG#mA#mC#mA#mAmAmUmCfAfCfCfUrG#rC |
| | #fCfUrC#mGmGrU#rU#rU#fU#fA#mGmAmGmC#mU#mA |
| | #mU#mG#mC#mU (SEQ ID NO: 344) |
| crRNA 101 | mG#mA#mG#mA#mC#mA#mAmAmUmCfAfCfCfUrG#rC |
| | #fCfUrC#mGmGrU#rU#rU#fU#fA#mG#mA#mG#mCmUm |
| | AmU#mG#mC#mU (SEQ ID NO: 345) |
| crRNA 102 | mG#mA#mG#mA#mC#mA#mAmAmUmCfAfCfCfUrG#rC |
| | #fCfUrC#mGmG#rU#rU#rUffU#fA#mGmAmGmCmUmA |
| | mU#mG#mC#mU (SEQ ID NO: 346) |
| crRNA 103 | mG#mA#mG#mA#mC#mA#mAmAmUmCfAfCfCfUrG#rC |
| | #fCfUrC#mGmGrU#rU#rU#fUfAmGmAmGmC#mU#mA# |
| | mU#mG#mC#mU (SEQ ID NO: 347) |
| crRNA 104 | mG#mA#mG#mA#mC#mA#mAmAmUmCfAfCfCfUrG#rC |
| | #fCfUrC#mGmGrU#rU#rU#fUfA#mG#mA#mG#mC#mU# |
| | mA#mU#mG#mC#mU (SEQ ID NO: 348) |
| crRNA 105 | mG#mA#mG#rA#rC#rA#mA#mAmUmCfAfCfCfUfGfCfCf |
| | UfCmGmGfUfUfUfUfAmGmAmGmCmUmAmU#mG#mC |
| | #mU (SEQ ID NO: 349) |
| crRNA 106 | mG#mA#mG#rA#rC#rA#mAmAmUmCfAfCfCfUfGfCfCfU |
| | fCmGmGfUfUfUfUfAmGmAmGmCmUmA#mU#mG#mC# |
| | mU (SEQ ID NO: 350) |
| crRNA 107 | mG#mA#mG#rA#rC#rA#mAmAmUmCfAfCfCfUfG#fC#fC |
| | #fU#fC#mGmGfUffU#fU#fU#fA#mGmAmGmCmUmAmU |
| | #mG#mC#mU (SEQ ID NO: 351) |
| crRNA 108 | mG#mA#mG#rA#rC#rA#mAmAmUmCfAfCfCfUfG#fC#fC |
| | fUfC#mGmGfUffUffU#fU#fA#mGmAmGmC#mU#mA#m |
| | U#mG#mC#mU (SEQ ID NO: 352) |
| crRNA 109 | mG#mA#mG#rA#rC#rA#mAmAmUmCfAfCfCfUfG#fC#fC |
| | fUfC#mGmGfU#fU#fUffU#fA#mG#mA#mG#mCmUmAm |
| | U#mG#mC#mU (SEQ ID NO: 353) |
| crRNA 110 | mG#mA#mG#rA#rC#rA#mAmAmUmCfAfCfCfUfGffC#fC |
| | fUfC#mGmG#fU#fU#fUffUffA#mGmAmGmCmUmAmU# |
| | mG#mC#mU (SEQ ID NO: 354) |
| crRNA 111 | mG#mA#mG#rA#rC#rA#mAmAmUmCfAfCfCfUfGfCfCfU |
| | fCmGmGfUfUfUfUfAmGmAmGmC#mU#mAmU#mG#mC |
| | #mU (SEQ ID NO: 355) |
| crRNA 112 | mG#mA#mG#rAffC#rA#mAmAmUmCfAfCfCfUfGfCfCfU |
| | fCmGmGfUfUfUfUfA#mG#mA#mG#mC#mU#mAmU#mG |
| | #mC#mU (SEQ ID NO: 356) |
| crRNA 113 | mG#mA#mG#dA#dC#dA#mAmAmUmCfAfCfCfUdG#dC# |
| | fCfUdC#mGmGdU#dU#dUffUfAmGmAmGmCmUmAmU |
| | #mG#mC#mU (SEQ ID NO: 357) |
| crRNA 114 | mC#mG#mA#mAmGmUmUmAmUmAfUfUfAfArGffGff |
| | GfUaUmCmGaUaUaUfUfAmGmAmGmCmUmAmU#mG# |
| | mC#mU (SEQ ID NO: 358) |
| crRNA 115 | mC#mG#mA#mAmGmUmUmAmUmAfUfUfAfArGffGff |
| | GfUaUmCmGrU#rU#rUffUfAmGmAmGmCmUmAmU#m |
| | G#mC#mU (SEQ ID NO: 359) |
| crRNA 116 | mC#mG#mA#mAmGmUmUmAmUmAfUfUfAfArG#rG#f |
| | GfUrU#mCmGaUrU#rUgUfAmGmAmGmCmUmAmU#m |
| | G#mC#mU (SEQ ID NO: 360) |
| crRNA 117 | mC#mG#mA#mAmGmUmUmAmUmAfUfUfAfArG#rG#f |
| | GfUrU#mCmGrU# aUrUgUfAmGmAmGmCmUmAmU#m |
| | G#mC#mU (SEQ ID NO: 361) |
| crRNA 118 | mC#mG#mA#mAmGmUmUmAmUmAfUfUfAfArG#rG#f |
| | GfUrU#mCmGrU#rU#aUfUfAmGmAmGmCmUmAmU#m |
| | G#mC#mU (SEQ ID NO: 362) |
| crRNA 119 | mC#mG#mA#mAmGmUmUmAmUmAfUfUfAfArG#rG#f |
| | GfUsU#mCmGsU#sU#sUgUfAmGmAmGmCmUmAmU# |
| | mG#mC#mU (SEQ ID NO: 363) |
| crRNA 120 | mC#mG#mA#mAmGmUmUmAmUmAfUfUfAfArG#rG#f |
| | GfUsUmCmGsUsUsUfUfAmGmAmGmCmUmAmU#mG# |
| | mC#mU (SEQ ID NO: 364) |
| crRNA 121 | mC#mG#mA#mAmGmUmUmAmUmAfUfUfAfArG#rG#f |
| | GfUsUmCmGrU#rU#rUgUfAmGmAmGmCmUmAmU#m |
| | G#mC#mU (SEQ ID NO: 365) |
| crRNA 122 | mC#mG#mA#mAmGmUmUmAmUmAfUfUfAfArG#rG#f |
| | GfUrU#mCmGsUrU#rU#fUfAmGmAmGmCmUmAmU#m |
| | G#mC#mU (SEQ ID NO: 366) |
| crRNA 123 | mC#mG#mA#mAmGmUmUmAmUmAfUfUfAfArG#rG#f |
| | GfUrU#mCmGrU#sUrU#fUfAmGmAmGmCmUmAmU#m |
| | G#mC#mU (SEQ ID NO: 367) |
| crRNA 124 | mC#mG#mA#mAmGmUmUmAmUmAfUfUfAfArG#rG#f |
| | GfUrU#mCmGrU#rU#sUfUfAmGmAmGmCmUmAmU#m |
| | G#mC#mU (SEQ ID NO: 368) |
| crRNA 125 | mC#mG#mA#mAmGmUmUmAmUmAfUfUfAfArG#rG#f |
| | GfUrU#mCmGsUrU#sUfUfAmGmAmGmCmUmAmU#mG |
| | #mC#mU (SEQ ID NO: 369) |
| crRNA 126 | mC#mG#mA#mAmGmUmUmAmUmAfUfUfAfArG#rG#f |
| | GfUrU#mCmGsUsUrU#fUfAmGmAmGmCmUmAmU#mG |
| | #mC#mU (SEQ ID NO: 370) |
| crRNA 127 | mC#mG#mA#mAmGmUmUmAmUmAfUfUfAfArG#rG#f |
| | GfUrU#mCmGrU#sUsUfUfAmGmAmGmCmUmAmU#mG |
| | #mC#mU (SEQ ID NO: 371) |
| crRNA 128 | mG#mA#mG#mAmCmAmAmAmUmCfAfCfCfUrG#rC#fC |
| | fUrC#mGmGaUaUaUfUfAmGmAmGmCmUmAmU#mG# |
| | mC#mU (SEQ ID NO: 372) |
| crRNA 129 | mG#mA#mG#mAmCmAmAmAmUmCfAfCfCfUrG#rC#fC |
| | fUrC#mGmGsU#sU#sU#fUfAmGmAmGmCmUmAmU#m |
| | G#mC#mU (SEQ ID NO: 373) |
| crRNA 130 | mG#mA#mG#mAmCmAmAmAmUmCfAfCfCfUrG#rC#fC |
| | fUrC#mGmGsUsUsUfUfAmGmAmGmCmUmAmU#mG#m |
| | C#mU (SEQ ID NO: 374) |
| crRNA 131 | mG#mA#mG#mAmCmAmAmAmUmCfAfCfCfUrG#aCfCf |
| | UrC#mGmGrU#rU#rU#fUfAmGmAmGmCmUmAmU#mG |
| | #mC#mU (SEQ ID NO: 375) |
| crRNA 132 | mG#mA#mG#mAmCmAmAmAmUmCfAfCfCfUrG#rC#fC |
| | fUaCmGmGrU#rU#rUgUfAmGmAmGmCmUmAmU#mG |
| | #mC#mU (SEQ ID NO: 376) |
| crRNA 133 | mG#mA#mG#mAmCmAmAmAmUmCfAfCfCfUrG#aC#fC |
| | fUaC#mGmGrU#rU#rU#fUfAmGmAmGmCmUmAmU#m |
| | G#mC#mU (SEQ ID NO: 377) |
| crRNA 134 | mU#mU#mU#mAmCmCmGmUmAmUfUfCfCfArC#rG#fA |
| | fGrG#mCmGaUaUaUfUfAmGmAmGmCmUmAmU#mG# |
| | mC#mU (SEQ ID NO: 378) |
| crRNA 22- | mC#mG#mC#mCmCmAmUmCmUmUfCfUfAfGrA#rA#fA |
| alt. seq | fGrA#mCmGfUrU#fUfUfAmGmAmGmCmUmAmU#mG# |
| | mC#mU (SEQ ID NO: 379) |
| crRNA 29- | mC#mG#mC#mCmCmAmUmCmUmUfCfUfAfGrA#fAfAf |
| alt. seq | GfAmCmGfUfUfUfUfAmGmAmGmCmUmAmU#mG#mCmU |
| | (SEQ ID NO: 380) |
| crRNA 29- | mG#mA#mG#mAmCmAmAmAmUmCfAfCfCfUrG#fCfCf |
| MCV1a | UfCmGmGfUfUfUfUfAmGmAmGmCmUmAmU#mG#mC |
| | #mU (SEQ ID NO: 381) |
| crRNA 29- | mG#mA#mG#mAmCmAmAmAmUmCfAfCfCfUrG#fCfCf |
| MCV1a | UfCmGmGfUfUfUfUfAmGmAmGmCmUmAmU#mG#mC |
| GalNAc | #mU-GalNAc (SEQ ID NO: 248) |
| conjugate | |
| crRNA 30- | mG#mA#mG#rArCrAmAmAmUmCrArCrCrUrGrCrCrUrC |
| MCV1a | mGmGrUrUrUrUrAmGmAmGmCmUmAmU#mG#mC#mU |
| | (SEQ ID NO: 382) |
| crRNA 31- | mG#mA#mG#rArCrAmAmAmUmCmArCrCrUrGrCrCrUrC |
| MCV1a | mGmGrUrUrUrUrAmGmAmGmCmUmAmU#mG#mC#mU |
| | (SEQ ID NO: 383) |
| crRNA 32- | mG#mA#mG#rArCrAmAmAmUmCmArCmCmUrGrCrCrU |
| MCV1a | rCmGmGrUrUrUrUrAmGmAmGmCmUmAmU#mG#mC# |
| | mU (SEQ ID NO: 384) |
| crRNA 33- | mG#mA#mG#rA#rC#rA#mAmAmUmCrA#rC#rC#rU#rG#r |
| MCV1a | C#rC#rU#rC#mGmGrU#rU#rU#rU#rA#mGmAmGmCmUm |
| | AmU#mG#mC#mU (SEQ ID NO: 385) |
| crRNA 34- | mG#mA#mG#rA#rC#rA#mAmAmUmCrA#rC#rC#rU#rG#r |
| MCV1a | C#rC#rU#rC#mGmGrUrUrUrUrAmGmAmGmCmUmAmU |
| | #mG#mC#mU (SEQ ID NO: 386) |
| crRNA 35- | mG#mA#mG#rArCrAmAmAmUmCrArCrCrUrGrCrCrUrC |
| MCV1a | mGmGrUrUrUmUmAmGmAmGmCmUmAmU#mG#mC# |
| | mU (SEQ ID NO: 387) |
| crRNA 36- | mG#mA#mG#rA#rC#rA#mAmAmUmCrA#rC#rC#rU#rG#r |
| MCV1a | C#rC#rU#rC#mGmGrUrUrUmUmAmGmAmGmCmUmAm |
| | U#mG#mC#mU (SEQ ID NO: 388) |
| crRNA 37- | mG#mA#mG#rA#rC#rA#mAmAmUmCfAfCfCfUrG#rCffC |
| MCV1a | fUrC#mGmGrU#rU#rU#mUmAmGmAmGmCmUmAmU# |
| | mG#mC#mU (SEQ ID NO: 389) |
| crRNA 38- | mG#mA#mG#dA#dC#dA#mAmAmUmCfAfCfCfUrG#rCff |
| MCV1a | CfUrC#mGmGrU#rU#rU#mUmAmGmAmGmCmUmAmU |
| | #mG#mC#mU (SEQ ID NO: 390) |
| crRNA 39- | mG#mA#mG#rA#rC#rA#mAmAmUmCfAfCfCfUfGfCfCfU |
| MCV1a | fCmGmGfUfUfUfUfAmGmAmGmCmUmAmU#mG#mC# |
| | mU (SEQ ID NO: 391) |
| crRNA 39- | mG#mA#mG#rA#rC#1-A#mAmAmUmCfAfCfCfUfGfCfCfU |
| MCV1a | fCmGmGfUfUfUfUfAmGmAmGmCmUmAmU#mG#mC# |
| GalNAc | mU-GalNAc (SEQ ID NO: 249) |
| conjugate | |
| crRNA 40- | mG#mA#mG#dA#dC#dA#mAmAmUmCfAfCfCfUfGfCfCf |
| MCV1a | UfCmGmGfUfUfUfUfAmGmAmGmCmUmAmU#mG#mC |
| | #mU (SEQ ID NO: 392) |
| crRNA 40- | mG#mA#mG#dA#dC#dA#mAmAmUmCfAfCfCfUfGfCfCf |
| MCV1a | UfCmGmGfUfUfUfUfAmGmAmGmCmUmAmU#mG#mC |
| GalNAc | #mU-GalNAc (SEQ ID NO: 250) |
| conjugate | |
| crRNA 41- | mG#mA#mG#mAmCmAmAmAmUmCfAfCfCfUdG#dCff |
| MCV1a | CfUdC#mGmGrU#rU#rUffUfAmGmAmGmCmUmAmU# |
| | mG#mC#mU (SEQ ID NO: 393) |
| crRNA 42- | mG#mA#mG#mAmCmAmAmAmUmCfAfCfCfUrG#rCffC |
| MCV1a | fUrC#mGmGdU#dUdU#fUfAmGmAmGmCmUmAmU#m |
| | G#mC#mU (SEQ ID NO: 394) |
| crRNA 43- | mG#mA#mG#mAmCmAmAmAmUmCfArCffCfUrG#rCff |
| MCV1a | CfUrC#mGmGrU#rU#rUffUfAmGmAmGmCmUmAmU#m |
| | G#mC#mU (SEQ ID NO: 395) |
| crRNA 44- | mG#mA#mG#mAmCmAmAmAmUmCfAdCffCfUrG#rCff |
| MCV1a | CfUrC#mGmGrU#rU#rUffUfAmGmAmGmCmUmAmU#m |
| | G#mC#mU (SEQ ID NO: 396) |
| crRNA 45- | mG#mA#mGffAfCfAmAmAmUmCfAfCfCfUfGfCfCfUfC |
| MCV1a | mGmGfUfUfUfUfAmGmAmGmCmUmAmU#mG#mC#mU |
| | (SEQ ID NO: 397) |
| crRNA 20- | mG#mA#mG#mAmCmAmAmAmUmCfAfCfCfUrG#rCffC |
| MCV1a | fUrC#mGmGrU#rU#rU#fUfAmGmAmGmCmUmAmU#mG |
| Cy3 | #mC#mU (SEQ ID NO: 398) |
| conjugate | |
| crRNA 2- | rUrUrUrArCrCmGmUmAmUrUrCrCrArCrGrArGrGmCmG |
| MCV1b | rUrUrUrUrAmGmAmGmCmUmAmU#mG#mCmU (SEQ |
| | ID NO: 399) |
| crRNA 3- | rU#rU#rU#rArCrCmGmUmAmUrUrCrCrArCrGrArGrGmC |
| MCV1b | mGrUrUrUrUrAmGmAmGmCmUmAmU#mG#mCmU |
| | (SEQ ID NO: 400) |
| crRNA 5- | rU#rU#rU#rArCrCmGmUmAmUrUrCrCrArCrGmAmGrGm |
| MCV1b | CmGrUrUrUrUrAmGmAmGmCmUmAmU#mG#mC#mU |
| | (SEQ ID NO: 401) |
| crRNA 6- | rU#rU#rU#rArCrCmGmUmAmUrUrCrCrArCrGrArGrGmC |
| MCV1b | mGrUrUrUmUmAmGmAmGmCmUmAmU#mG#mC#mU |
| | (SEQ ID NO: 402) |
| crRNA 10- | mU#mU#mU#mAmCmCmGmUmAmUfUfCfCfArC#rG#fA |
| MCV1b | fGrG#mCmGrU#rU#rU#mUmAmGmAmGmCmUmAmU# |
| | mG#mC#mU (SEQ ID NO: 403) |
| crRNA 20- | mU#mU#mU#mAmCmCmGmUmAmUfUfCfCfArC#rG#fA |
| MCV1b | fGrG#mCmGrU#rU#rU#fUfAmGmAmGmCmUmAmU#mG |
| | #mC#mU (SEQ ID NO: 404) |
| crRNA 21- | mU#mU#mU#mAmCmCmGmUmAmUfUfCfCfAfCfGfAfG |
| MCV1b | fGmCmGfUfUfUfUfAmGmAmGmCmUmAmU#mG#mC# |
| | mU (SEQ ID NO: 405) |
| crRNA 30- | mU#mU#mU#rArCrCmGmUmAmUrUrCrCrArCrGrArGrG |
| MCV1b | mCmGrUrUrUrUrAmGmAmGmCmUmAmU#mG#mC#mU |
| | (SEQ ID NO: 406) |
| crRNA 31- | mU#mU#mU#rArCrCmGmUmAmUmUrCrCrArCrGrArGr |
| MCV1b | GmCmGrUrUrUrUrAmGmAmGmCmUmAmU#mG#mC#m |
| | U (SEQ ID NO: 407) |
| crRNA 32- | mU#mU#mU#rArCrCmGmUmAmUmUrCmCmArCrGrArG |
| MCV1b | rGmCmGrUrUrUrUrAmGmAmGmCmUmAmU#mG#mC# |
| | mU (SEQ ID NO: 408) |
| crRNA 33- | mU#mU#mU#rA#rC#rC#mGmUmAmUrU#rC#rC#rA#rC#r |
| MCV1b | G#rA#rG##rG#mCmGrU#rU#rU#rU#rA#mGmAmGmCmU |
| | mAmU#mG#mC#mU (SEQ ID NO: 409) |
| crRNA 34- | mU#mU#mU#rA#rC#rC#mGmUmAmUrU#rC#rC#rA#rC#r |
| MCV1b | G#rA#rG#rG#mCmGrUrUrUrUrAmGmAmGmCmUmAmU |
| | #mG#mC#mU (SEQ ID NO: 410) |
| crRNA 35- | mU#mU#mU#rArCrCmGmUmAmUrUrCrCrArCrGrArGrG |
| MCV1b | mCmGrUrUrUmUmAmGmAmGmCmUmAmU#mG#mC#m |
| | U (SEQ ID NO: 411) |
| crRNA 36- | mU#mU#mU#rA#rC#rC#mGmUmAmUrU#rC#rC#rA#rC#r |
| MCV1b | G#rA#rG#rG#mCmGrUrUrUmUmAmGmAmGmCmUmAm |
| | U#mG#mC#mU (SEQ ID NO: 412) |
| crRNA 37- | mU#mU#mU#rA#rC#rC#mGmUmAmUfUfCfCfArC#rG#fA |
| MCV1b | fGrG#mCmGrU#rU#rU#mUmAmGmAmGmCmUmAmU# |
| | mG#mC#mU (SEQ ID NO: 413) |
| crRNA 38- | mU#mU#mU#dA#dC#dC#mGmUmAmUfUfCfCfArC#rG#f |
| MCV1b | AfGrG#mCmGrU#rU#rU#mUmAmGmAmGmCmUmAmU |
| | #mG#mC#mU (SEQ ID NO: 414) |
| crRNA 39- | mU#mU#mU#rA#rC#rC#mGmUmAmUfUfCfCfAfCfGfAf |
| MCV1b | GfGmCmGfUfUfUfUfAmGmAmGmCmUmAmU#mG#mC |
| | #mU (SEQ ID NO: 415) |
| crRNA 40- | mU#mU#mU#dA#dC#dC#mGmUmAmUfUfCfCfAfCfGfAf |
| MCV1b | GfGmCmGfUfUfUfUfAmGmAmGmCmUmAmU#mG#mC |
| | #mU (SEQ ID NO: 416) |
| crRNA 41- | mU#mU#mU#mAmCmCmGmUmAmUfUfCfCfAdC#dG#f |
| MCV1b | AfGdG#mCmGrU#rU#rUgUfAmGmAmGmCmUmAmU# |
| | mG#mC#mU (SEQ ID NO: 417) |
| crRNA 42- | mU#mU#mU#mAmCmCmGmUmAmUfUfCfCfArC#rG#fA |
| MCV1b | fGrG#mCmGdU#dU#dUffUfAmGmAmGmCmUmAmU#m |
| | G#mC#mU (SEQ ID NO: 418) |
| crRNA 43- | mU#mU#mU#mAmCmCmGmUmAmUfUrC#fCfArC#rG#f |
| MCV1b | AfGrG#mCmGrU#rU#rU#fUfAmGmAmGmCmUmAmU#m |
| | G#mC#mU (SEQ ID NO: 419) |
| crRNA 44- | mU#mU#mU#mAmCmCmGmUmAmUfUdC#fCfArC#rG#f |
| MCV1b | AfGrG#mCmGrU#rU#rU#fUfAmGmAmGmCmUmAmU#m |
| | G#mC#mU (SEQ ID NO: 420) |
| crRNA 20- | mC#mC#mC#mAmUmAmCmCmUmUfGfGfAfGrC#rA#fA |
| PCSK9b | fCrG#mGmGrU#rU#rU#fUfAmGmAmGmCmUmAmU#mG |
| | #mC#mU (SEQ ID NO: 421) |
| crRNA 20- | mC#mC#mC#mAmUmAmCmCmUmUfGfGfAfGrC#rA#fA |
| PCSK9b | fCrG#mGmGrU#rU#rU#fUfAmGmAmGmCmUmAmU#mG |
| GalNAc | #mC#mU-GalNAc (SEQ ID NO: 252) |
| conjugate | |
| crRNA 21- | mC#mC#mC#mAmUmAmCmCmUmUfGfGfAfGfCfAfAfC |
| PCSK9b | fGmGmGfUfUfUfUfAmGmAmGmCmUmAmU#mG#mC# |
| | mU (SEQ ID NO: 422) |
| crRNA 29- | mC#mC#mC#mAmUmAmCmCmUmUfGfGfAfGrC#fAfAf |
| PCSK9b | CfGmGmGfUfUfUfUfAmGmAmGmCmUmAmU#mG#mC |
| | #mU (SEQ ID NO: 423) |
| crRNA 29- | mC#mC#mC#mAmUmAmCmCmUmUfGfGfAfGrC#fAfAf |
| PCSK9b | CfGmGmGfUfUfUfUfAmGmAmGmCmUmAmU#mG#mC |
| GalNAc | #mU-GalNAc (SEQ ID NO: 253) |
| conjugate | |
| crRNA 30- | mC#mC#mC#rArUrAmCmCmUmUrGrGrArGrCrArArCrG |
| PCSK9b | mGmGrUrUrUrUrAmGmAmGmCmUmAmU#mG#mC#mU |
| | (SEQ ID NO: 424) |
| crRNA 39- | mC#mC#mC#rA#rU#rA#mCmCmUmUfGfGfAfGfCfAfAfC |
| PCSK9b | fGmGmGfUfUfUfUfAmGmAmGmCmUmAmU#mG#mC# |
| | mU (SEQ ID NO: 425) |
| crRNA 39- | mC#mC#mC#rA#rU#rA#mCmCmUmUfGfGfAfGfCfAfAfC |
| PCSK9b | fGmGmGfUfUfUfUfAmGmAmGmCmUmAmU#mG#mC# |
| GalNAc | mU-GalNAc (SEQ ID NO: 254) |
| conjugate | |
| crRNA 40- | mC#mC#mC#dA#dU#dA#mCmCmUmUfGfGfAfGfCfAfAf |
| PCSK9b | CfGmGmGfUfUfUfUfAmGmAmGmCmUmAmU#mG#mC |
| | #mU (SEQ ID NO: 426) |
| crRNA 40- | mC#mC#mC#dA#dU#dA#mCmCmUmUfGfGfAfGfCfAfAf |
| PCSK9b | CfGmGmGfUfUfUfUfAmGmAmGmCmUmAmU#mG#mC |
| GalNAc | #mU-GalNAc (SEQ ID NO: 255) |
| conjugate | |
| crRNA 42- | mC#mC#mC#mAmUmAmCmCmUmUfGfGfAfGrC#rA#fA |
| PCSK9b | fCrG#mGmGdU#dU#dUgUfAmGmAmGmCmUmAmU#m |
| | G#mC#mU (SEQ ID NO: 427) |
| crRNA 42- | mC#mC#mC#mAmUmAmCmCmUmUfGfGfAfGrC#rA#fA |
| PCSK9b | fCrG#mGmGdU#dU#dUgUfAmGmAmGmCmUmAmU#m |
| GalNAc | G#mC#mU-GalNAc (SEQ ID NO: 256) |
| conjugate | |
| crRNA 45- | mC#mC#mC#fAfUfAmCmCmUmUfGfGfAfGfCfAfAfCfC |
| PCSK9b | mGmGfUfUfUfUfAmGmAmGmCmUmAmU#mG#mC#mU |
| | (SEQ ID NO: 428) |

### Example 2

### Genome Editing Efficiency of Chemically Modified crRNA and tracrRNA

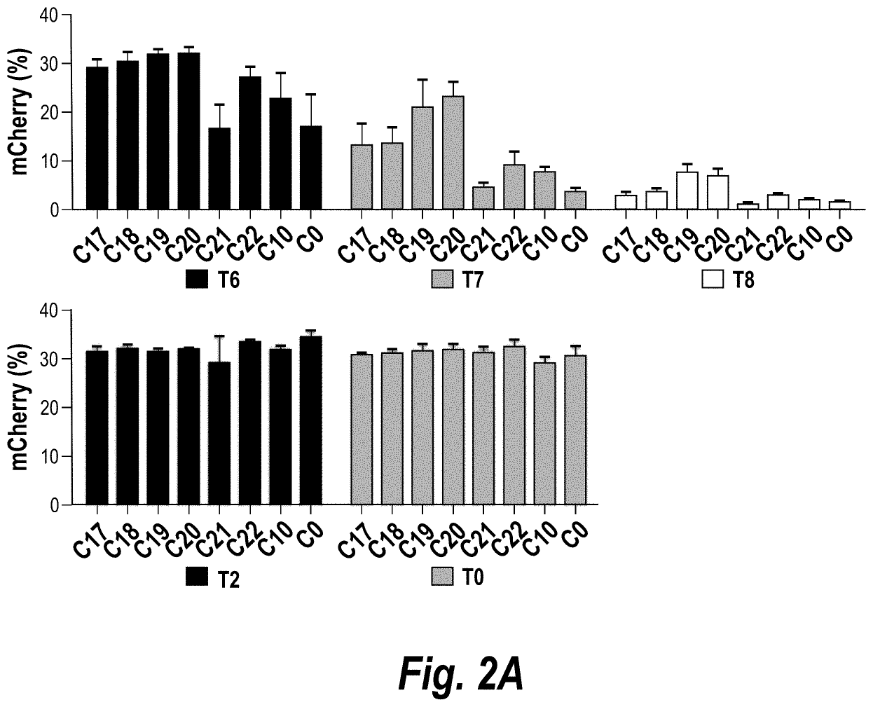

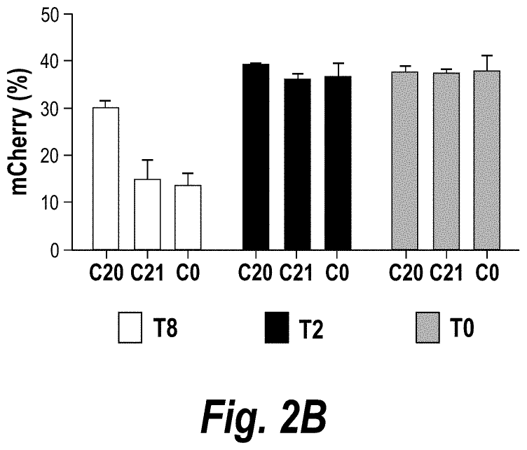

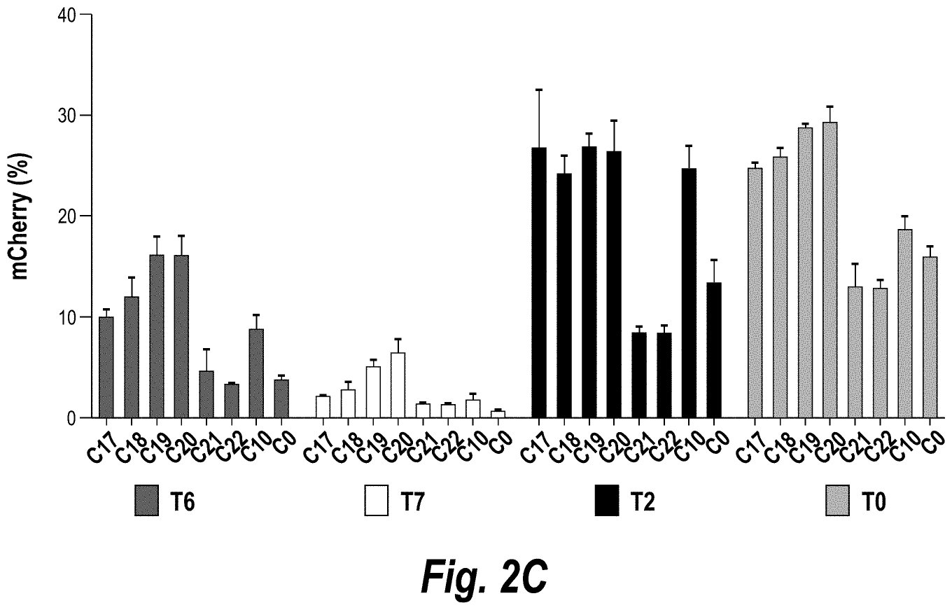

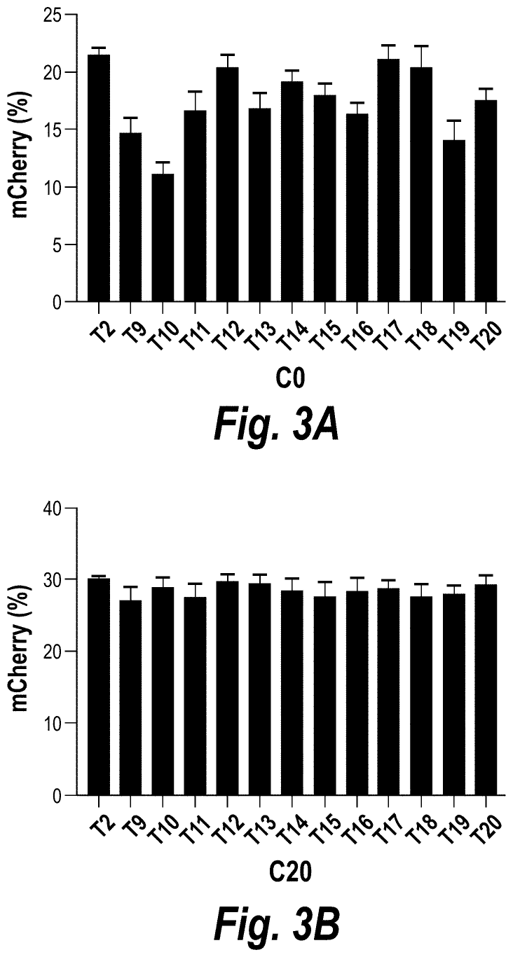

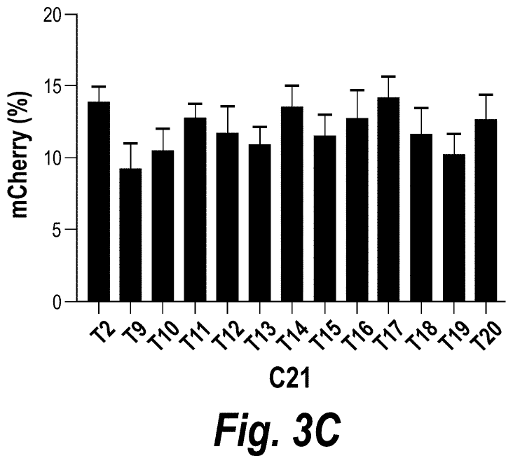

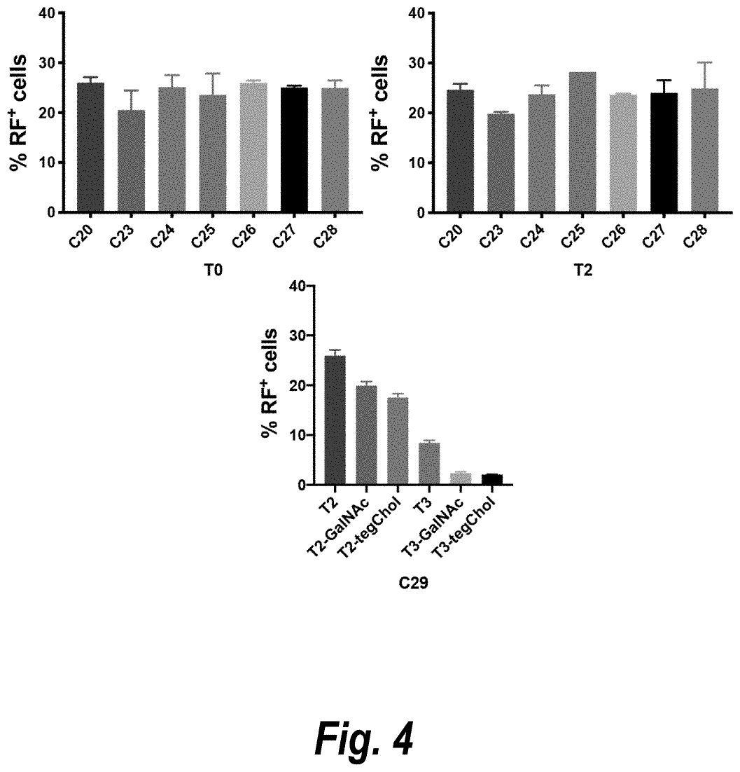

Prior work demonstrated that several chemical modification patterns of crRNA and tracrRNA were capable of being active while increasing serum stability (WO 2019/183000 A1, incorporated herein by reference). The modified crRNAs created previously were C1 to C22 and the modified tracrRNA created previously were T1-T20 (see Table 1 and Table 2 above). FIG. 2A-FIG. 2C demonstrate activity of several of the initial crRNAs paired with modified tracrRNAs. FIG. 3A-FIG. 3C demonstrate activity of several of the initial tracrRNAs paired with C0 (unmodified), C20, and C21. From this previous work, it was noted that certain heavily modified patterns and fully modified had a reduction in genome editing efficiency. The work described herein has led to the identification of new heavily and fully chemically modified guide RNA patterns that retain high genome editing efficiencies.

Chemically Modified crRNA and tracrRNA Screening Methods

Cell Culture

Screening was performed in a HEK293T stable cell line expressing the traffic light reporter (TLR) Multi-Cas Variant 1 system (TLR-MCV1). The HEK293T cells were cultured in Dulbecco-modified Eagle's Minimum Essential Medium (DMEM; Life Technologies). DMEM was also supplemented with 10% Fetal Bovine Serum (FBS; Sigma). Cells were grown in a humidified 37° C., 5% CO2 incubator.

Traffic Light Reporter (TLR) System

The traffic light reporter (TLR) system includes a GFP (containing an insertion), followed by an out-of-frame mCherry. Upon double stranded break induction, a subset of non-homologous end-joining (NHEJ) repair events generate indels that place mCherry in frame, leading to red fluorescence. Detection of the red fluorescence is therefore a readout of editing efficiency. This system was developed and further described in Certo et al. (Nat. Methods 8, 671 (2011)). This system was further developed for testing the modified crRNAs and tracrRNAs of the disclosure. The TLR Multi-Cas Variant 1 system (TLR-MCV1) was created to introduce protospacer adjacent motifs (PAMs) to multiple alternative CRISPR enzymes (Streptococcus pyogenes (SpyCas9), Neisseria meningiditis (Nme1Cas9 and Nme2Cas9), Campylobacter jejuni (CjeCas9), Staphylococcus aureus (SauCas9), Geobacillus stearothermophilus (GeoCas9), Lachnospiraceae bacterium ND2006 (LbaCas12a), Acidaminococcus sp. (AspCas12a), and Francisella novicida (FnoCas12)). An additional SpyCas9 editing site was introduced as well, producing editing sites MCV1a and MCV1b. The MCV1a target is GAGACAAAUCACCUGCCUCG (SEQ ID NO: 429) and the MCV1b target is UUUACCGUAUUCCACGAGGC (SEQ ID NO: 430). These overlapping SpyCas9 cleavage sites permit the evaluation of two different crRNA sequences targeting the same position.

mTmG Reporter System

The mTmG reporter system is a double-fluorescent Cre reporter that expresses membrane-targeted tandem dimer Tomato (mT) prior to Cre-mediated excision and membrane-targeted green fluorescent protein (mG) after excision. As an alternative, the tdTomato gene may be excised by introducing two CRISPR-mediated cuts at flanking positions. The two cut sites are identical, and can therefore be cleaved with a single guide RNA-Cas9 RNP. The reporter system can be used in vivo in a transgenic mouse, or in vitro in a cell line. Here the reporter was used in mouse embryonic fibroblasts (MEFs) for in vitro experiments, and in the transgenic mouse for in vivo experiments. When the reporter is unedited (i.e., no CRISPR editing), tdTomato is expressed, leading to red fluorescence. If the tdTomato gene is successfully edited out, a GFP gene is expressed. Accordingly, in the mTmG reporter system, higher levels of GFP fluorescence indicate successful editing by CRISPR. The crRNA portions of the chemically modified guide RNAs described herein have the guide sequence CGAAGUUAUAUUAAGGGUUC (SEQ ID NO: 431). The reporter is described in greater detail in Muzumdar et al. (Genesis. 45(9): 593-605. 2007), incorporated herein by reference.

Expression and Purification of Spy-Cas9

The pMCSG7 vector expressing the Cas9 from Streptococcus pyogenes was used. In this construct, the Cas9 also contains three nuclear localization signals (NLSs). Rosetta DE3 strain of Escherichia coli was transformed with the 3×NLS-SpyCas9 construct. For expression and purification of 3×NLS-SpyCas9, a previously described protocol was used (Jinek et al. Science, 337: 816 (2012)). The bacterial culture was grown at 37° C. until an OD600 of 0.6 was reached. Then, the bacterial culture was cooled to 18° C., and 1 mM Isopropyl β-D-1-thiogalactopyranoside (IPTG; Sigma) was added to induce protein expression. Cells were grown overnight for 16-20 hours.

The bacterial cells were harvested and resuspended in Lysis Buffer [50 mM Tris-HCl (pH 8.0), 5 mM imidazole]. 10 μg/mL of Lysozyme (Sigma) was then added to the mixture and incubated for 30 minutes at 4° C. This was followed by the addition of 1× HALT Protease Inhibitor Cocktail (ThermoFisher). The bacterial cells were then sonicated and centrifuged for 30 minutes at 18,000 rpm. The supernatant was then subjected to Nickel affinity chromatography. The elution fractions containing the SpyCas9 were then further purified using cation exchange chromatography using a 5 mL HiTrap S HP column (GE). This was followed by a final round of purification by size-exclusion chromatography using a Superdex-200 column (GE). The purified protein was concentrated and flash frozen for subsequent use.

Transfection of HEK293T Cells

The HEK293T cells were nucleofected using the Neon transfection system (ThermoFisher) according to the manufacturer's protocol. Briefly, 20 picomoles of 3×NLS-SpyCas9 was mixed with 25 picomoles of crRNA:tracrRNA in buffer R (ThermoFisher) and incubated at room temperature for 20-30 minutes. This Cas9 RNP complex was then mixed with approximately 100,000 cells which were already resuspended in buffer R. This mixture was nucleofected with a 10 μL Neon tip and then plated in 24-well plates containing 500 μL of DMEM and 10% FBS. The cells were stored in a humidified 37° C. and 5% CO2 incubator for 2-3 days.

Flow Cytometry Analysis

The nucleofected HEK293T cells were analyzed on MACSQuant® VYB from Miltenyi Biotec. For mCherry detection, the yellow laser (561 nm) was used for excitation and 615/20 nm filter used to detect emission. At least 20,000 events were recorded and the subsequent analysis was performed using FlowJo® v10.4.1. Cells were first sorted based on forward and side scattering (FSC-A vs SSC-A) to eliminate debris. Cells were then gated using FSC-A and FSC-H to select single cells. Finally, mCherry signal was used to select for mCherry-expressing cells. The percent of cells expressing mCherry was calculated and reported in this application as a measure of Cas9-based genome editing.

Indel Analysis by TIDE

The genomic DNA from HEK293T cells was harvested using DNeasy Blood and Tissue kit (Qiagen) as recommended by the manufacturer. Approximately 50 ng of genomic DNA was used to PCR-amplify a ˜700 base pair fragment that was subsequently purified using a QIAquick PCR Purification kit (Qiagen). The PCR fragment was then sequenced by Sanger sequencing and the trace files were subjected to indel analysis using the TIDE web tool (Brinkman et al. Nucleic Acids Research, 42: e168 (2014)). Results are reported as % Indel rate.

Screening of New Chemical Modification Patterns

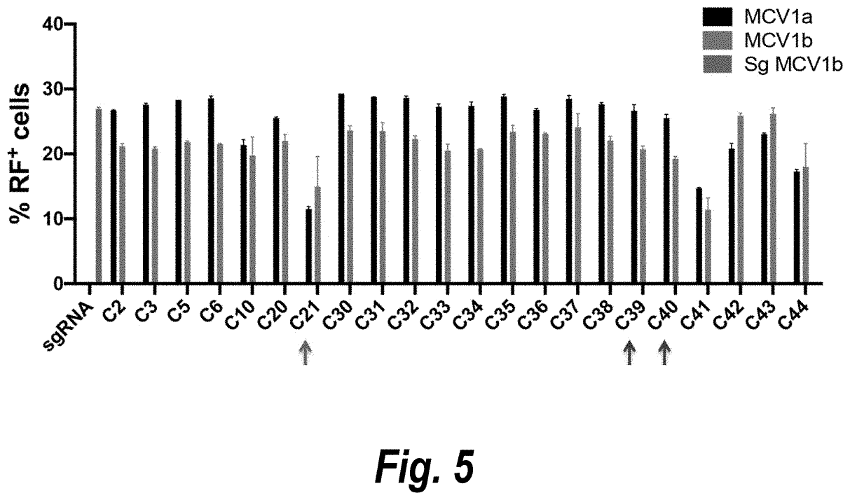

Structure-guided and systematic approaches were used to introduce 2′-OMe-RNA, 2′-F-RNA, 2′-deoxy, and PS modifications throughout guide RNAs. These modifications were chosen because they have been shown to improve stability, efficacy, and immunotoxicity associated with RNA. The strategy described herein yielded active RNP complexes with both extensively and fully modified versions of crRNAs and tracrRNAs. FIG. 4 and FIG. 5 depict a screen of crRNA patterns C23-C44, targeting both the MCV1a site and the MCV1b site. The crRNAs C29, C39 and C40 demonstrate efficacy similar to that of the previously developed crRNA, C20. The crRNAs C20, C29, and C39 are fully modified in the sense that every nucleotide that does not have a ribose modification has a phosphodiester linkage modification. However, C20 still contains six unmodified ribose residues, while the new crRNA C39 only has three unmodified riboses, and C29 has only one unmodified ribose. C40 is the newly developed, fully modified crRNA with no unmodified riboses in its composition. C45 is also a fully modified molecule with no unmodified ribose moieties. Like C40, this composition is expected to be very stable in vivo, though its activity is diminished somewhat in comparison to crRNA C20.

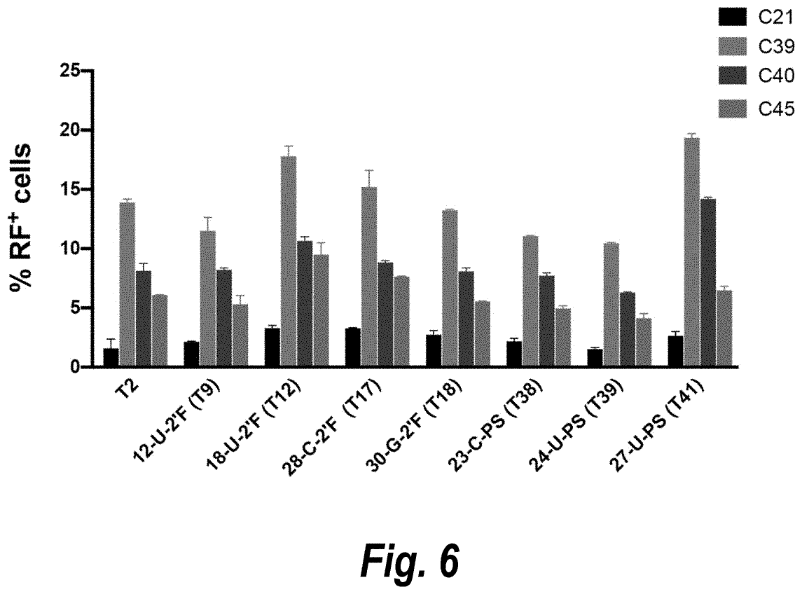

New tracrRNA chemical modification patterns were also developed. FIG. 6 depicts a screen of previously described tracrRNA patterns T2, T9, T12, T17, and T18, compared to new patterns T38, T39, and T41. The different tracrRNAs were paired with C21, C39, C40, or C45. The new crRNAs C39, C40, and C45 displayed higher editing efficiencies when paired with all tracrRNAs compared to the older C21 pattern.

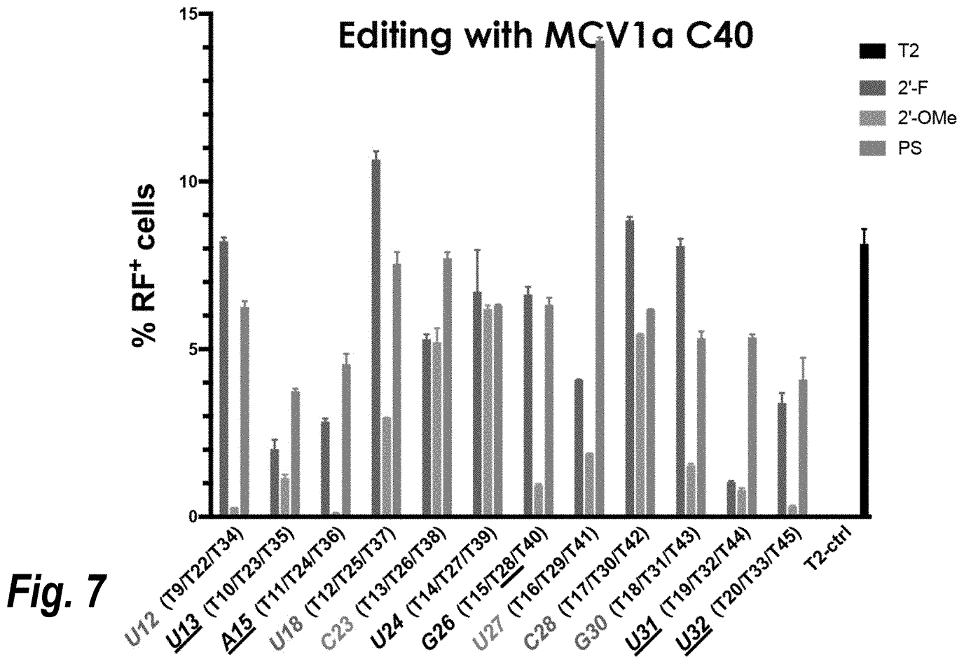

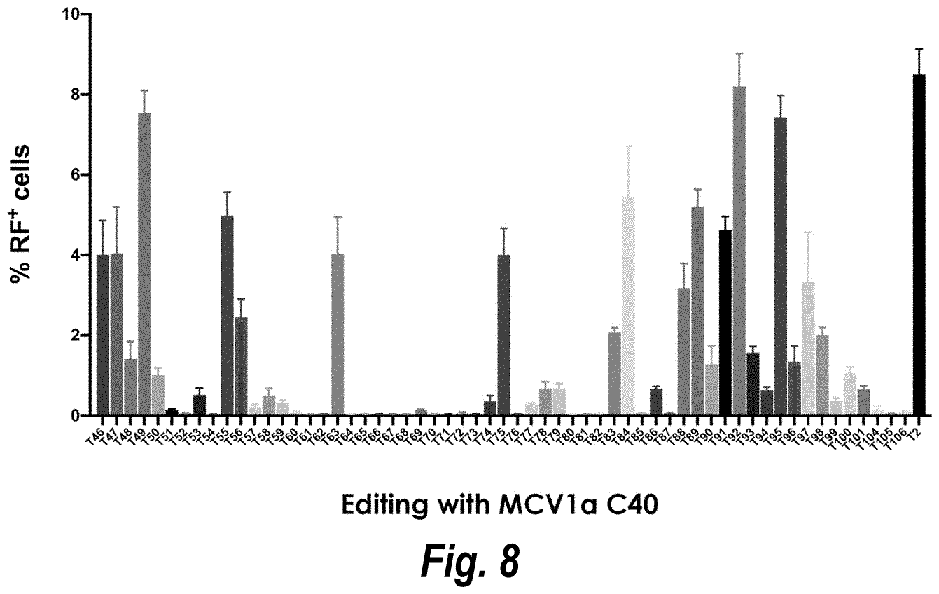

Several new tracrRNAs are more heavily modified than the previous tracrRNA T2. TracrRNA T41, T12 and T17 show higher activity than T2. TracrRNAs T9, T18, T37, T38 and T92 display similar efficiencies as T2, while T49 and T95 display slightly diminished activity than T2 (FIG. 7 and FIG. 8).

The loss in efficacy seen in human cells with the fully modified crRNA C45 and heavily modified tracrRNAs T49 and T95 compared to the previously developed crRNA C20 or tracrRNA T2 may be offset by higher in vivo stability. All of the newly developed RNAs are functional in multiple combinations when tested in human cells.

### Example 3

### Chemically Modified crRNA:tracrRNA Pairs with and without Conjugates Targeting Endogenous Human Genes

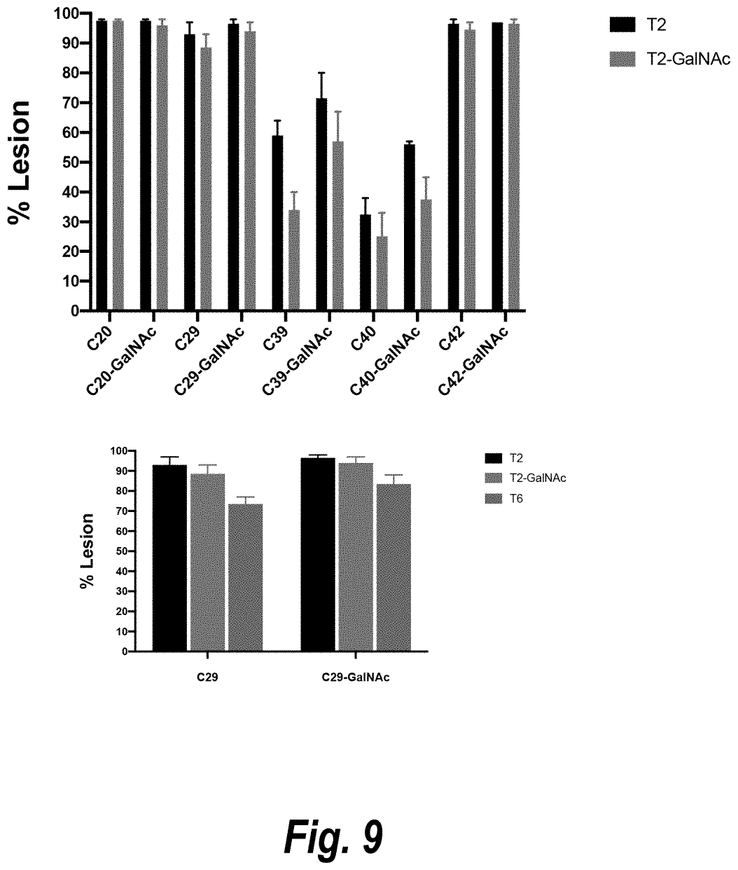

To verify that the crRNA and tracrRNA designs of the disclosure are compatible with different guide sequences, including those targeting endogenous human genes, the designs C29, C30, C40, C42, and C45 were tested by targeting the PCSK9 gene (FIG. 9). The crRNAs were paired with tracrRNA T2 or T6, and T2 was further used in a non-conjugate or GalNAc-conjugate form. C29, C39, C40, and C42 were also tested in a non-conjugate or GalNAc-conjugate form. The RNA designs were tested by electroporation of Cas9 RNP in the mouse Hepa 1-6 cell line. The graphs show indel percentages based on Inference of CRISPR Edits (ICE) analysis of PCR and Sanger sequencing data of the locus. The data represent the means from three independent biological replicates and error bars represent s.e.m.

These results demonstrate that the modified crRNA and tracrRNA designs are also applicable to endogenous target sites and function with conjugates on both the crRNA and tracrRNA.

### Example 4

### Chemically Modified crRNAs with Varied Phosphorothioate Content

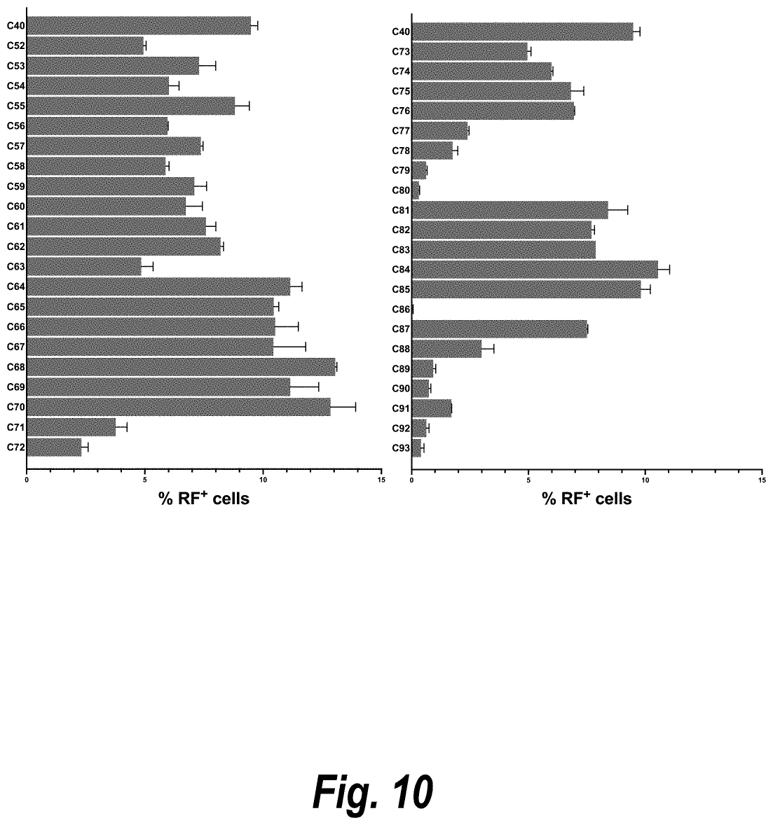

Additional chemically modified crRNAs were designed, synthesized, and tested for genome editing efficiency. crRNAs C52-C93 were tested in the TLR assay with the MCV1a target site. Each crRNA was paired with the T41 tracrRNA. 2 pmol of an RNP containing Cas9 with the various crRNAs and the tracrRNA were transfected into the TLR-MCV1 line described above and the % mCherry expression was detected as a proxy for genome editing efficiency. The crRNAs C52-C93 contained the same chemical modification pattern as C40, except with respect to phosphorothioate placement. The crRNA sequences are shown in Table 6. The screen revealed that crRNAs containing at least up to 20 phosphorothioate modifications are tolerated (FIG. 10).

### Example 5

### Chemically Modified crRNAs Containing 2′-amino RNA and/or 4′-thio RNA Modifications

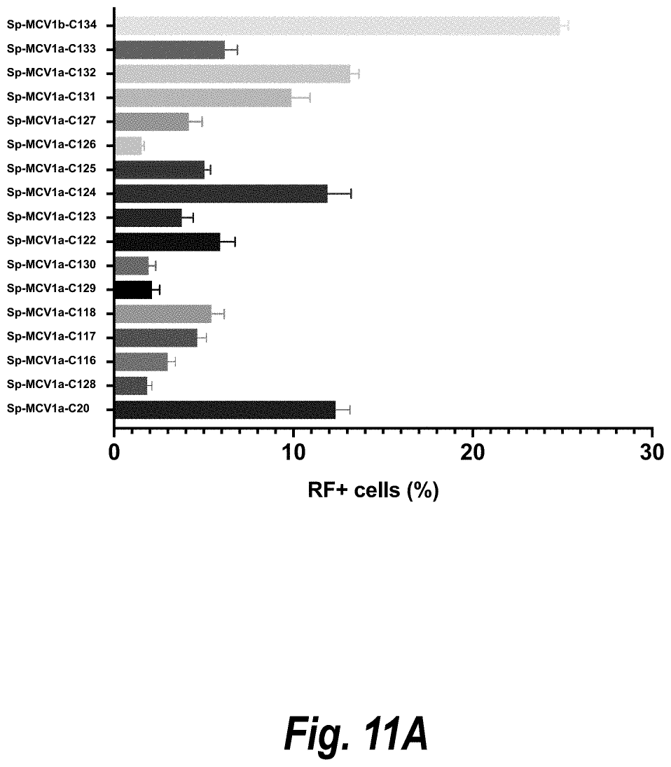

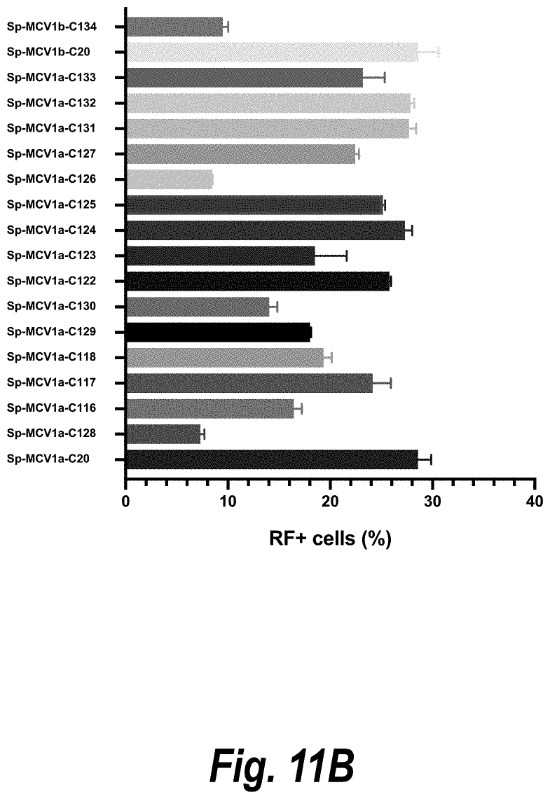

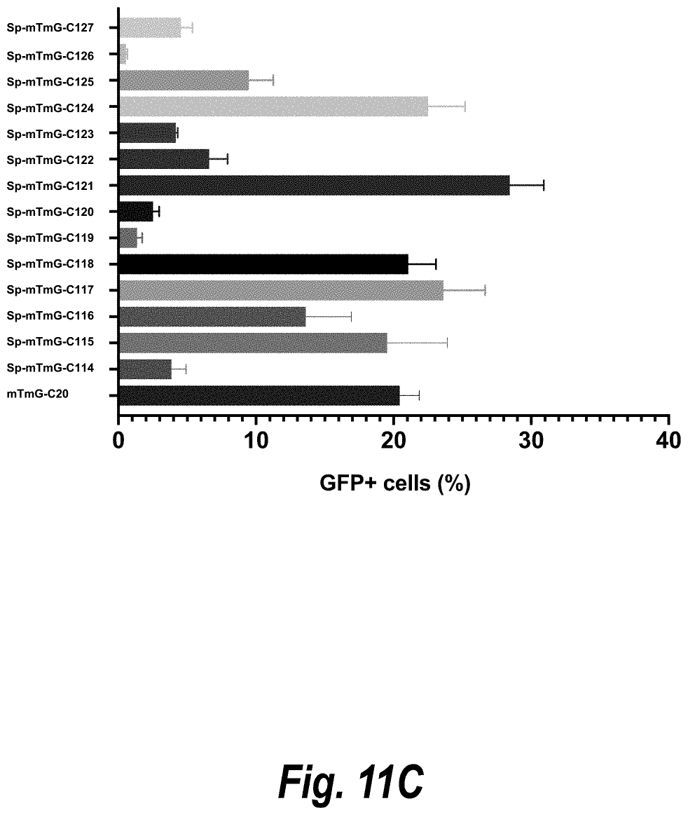

Additional chemically modified crRNAs containing either 2′-amino RNA or 4′-thio RNA (i.e., sugar ring oxygen in ribose sugar is replaced with sulfur) modifications were designed, synthesized, and tested for gene editing efficiency. crRNAs C114-C134 were tested in the TLR assay with the MCV1a target site or MCV1b target site, or in the mTmG reporter system, each of which is described above. As shown in FIG. 11A, crRNAs C116-C118 and C122-C134 was paired with the T2 tracrRNA. 5 pmol of an RNP containing Cas9 with the various crRNAs and the tracrRNA were transfected into the TLR-MCV1 line described above and the % mCherry expression was detected. As shown in FIG. 11B, crRNAs C116-C118 and C122-C134 were used in a modified TLR-MCV1 assay in which an unmodified tracrRNA and SpCas9 were stably expressed as well. 100 pmol of each crRNA was transfected into the cell line and the % mCherry expression was detected. Finally, as shown in FIG. 11C, crRNAs C114-C127 were tested in the mTmG reporter assay described above with 5 pmol of an RNP containing Cas9 with the various crRNAs and the T2 tracrRNA. The crRNA sequences are shown in Table 6. Each crRNA tested either had one or more 2′-amino ribose modifications or one or more 4′-thio RNA modifications. The screen revealed that crRNAs containing one or more 2′-amino ribose modifications or one or more 4′-thio RNA modifications maintain effective gene editing activity, while possessing additional chemical modifications that can improve stability.

### Example 6

### Chemically Modified tracrRNAs Containing 4′-thio RNA Modifications

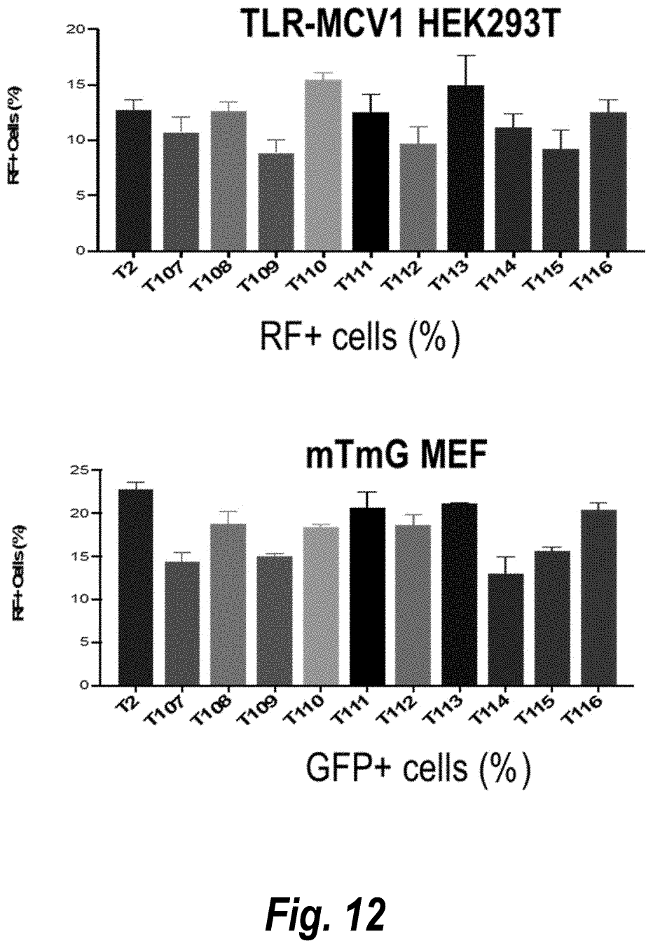

Additional chemically modified tracrRNAs containing 4′-thio RNA modifications were designed, synthesized, and tested for gene editing efficiency. tracrRNAs T107-T116 were tested in the TLR assay or in the mTmG reporter system, each of which is described above. Each of T107-T116 had the same chemical modification pattern as T2, except a 4′-thio RNA modification was introduced at one or more of the unmodified residues. 5 pmol of an RNP containing Cas9 with the various tracrRNAs and the C20 crRNA were transfected into the TLR-MCV1 line or mTmG line and the fluorescence was detected. The tracrRNA sequences are shown in Table 2. As shown in FIG. 12, all of the tracrRNAs tested retained effective gene editing activity. The inclusion of 4′-thio RNA modifications at previously unmodified positions provides tracrRNAs that are closer to being 100% chemically modified. T107 for example, has a modification at all but 5 nucleotides.

### Example 7

### In Vivo Gene Editing

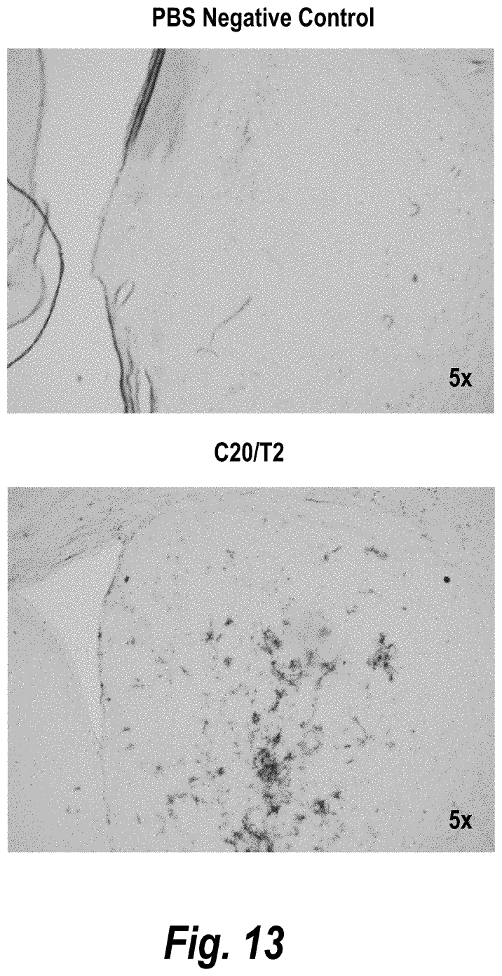

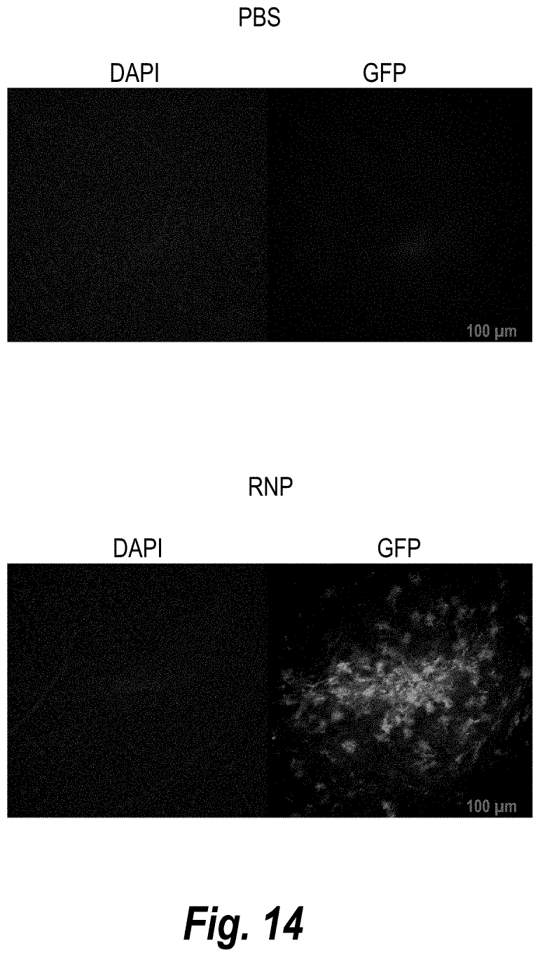

The various chemically modified guide RNAs have displayed substantial gene editing activity in vitro while possessing enhanced stability (e.g., serum stability). The in vivo activity of select chemically modified guide RNAs was next determined in the mTmG transgenic mouse. RNPs made up of select crRNAs and tracrRNAs, along with Cas9, were intrastriatally (IS) injected into the mouse at a dose of 150-200 pmol. Six days following injection, mouse brain tissue was stained to detect GFP expression. The guide RNA crRNA/tracrRNA pairs were used: C20/T2, C29/T2, C20/T41, and C29/T41. As shown in FIG. 13, GFP was expressed in brain tissue from mice receiving a C20/T2 containing RNP. As shown in FIG. 14, GFP was expressed in brain tissue from mice receiving a C20/T41 containing RNP. The data shows that the chemically modified guide RNAs are capable of gene editing activity in vivo.

1. A chemically modified guide RNA comprising:
(a) a crRNA portion comprising (i) a guide sequence capable of hybridizing to a target polynucleotide sequence, and (ii) a repeat sequence; and
(b) a tracrRNA portion comprising an anti-repeat nucleotide sequence that is complementary to the repeat sequence,
wherein the crRNA portion comprises at least 50% modified nucleotides; and
wherein the crRNA portion comprises between one and ten 2′-deoxy modified ribose groups.

2. The chemically modified guide RNA of claim 1, wherein the modified nucleotides each independently comprise a modification of a ribose group, a phosphate group, a nucleobase, or a combination thereof, optionally wherein:
each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC});
at least 80%, at least 90%, or 100% of the ribose groups are chemically modified;
each modification of the phosphate group is independently selected from the group consisting of a phosphorothioate, phosphonoacetate (PACE), thiophosphonoacetate (thioPACE), amide, triazole, phosphonate, and phosphotriester modification;
each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups;
the guide RNA comprises at least 90% or 100% modified nucleotide;
the crRNA portion comprises between 1 and 20 phosphorothioate modifications; and/or
the chemically modified guide RNA comprises at least about 50% activity relative to an unmodified guide RNA.

3-10. (canceled)

11. The chemically modified guide RNA of claim 1, wherein at least one nucleotide of the crRNA portion comprises each of a 2′-deoxy chemical modification and a phosphorothioate chemical modification, optionally wherein
one or more of the nucleotides at positions 4, 5, 6, 12, 15, 16, 19, 22, 23, and 24 from the 5′ end of the crRNA portion comprise a 2′-deoxy chemical modification;
the nucleotides at positions 4, 5, and 6 from the 5′ end of the crRNA portion comprise each of a 2′-deoxy chemical modification and a phosphorothioate chemical modification;
the nucleotide at position 12 from the 5′ end of the crRNA portion comprises each of a 2′-deoxy chemical modification and a phosphorothioate chemical modification;
the nucleotides at positions 15, 16, and 19 from the 5′ end of the crRNA portion comprise each of a 2′-deoxy chemical modification and a phosphorothioate chemical modification; or
the nucleotides at positions 22, 23, and 24 from the 5′ end of the crRNA portion comprise each of a 2′-deoxy chemical modification and a phosphorothioate chemical modification.

12-16. (canceled)

17. The chemically modified guide RNA of claim 1, comprising a crRNA portion modification pattern selected from the group consisting of:
(crRNA 38)
(SEQ ID NO: 5)
mN#mN#mN#dN#dN#dN#mNmNmNmNfNfNfNfNrN#rN#fNfNrN#mNmGr
U#rU#rU#mUmAmGmAmGmCmUmAmU#mG#mC#mU;
(crRNA 40)
(SEQ ID NO: 6)
mN#mN#mN#dN#dN#dN#mNmNmNmNfNfNfNfNfNfNfNfNfNmNmGfUfU
fUfUfAmGmAmGmCmUmAmU#mG#mC#mU;
(crRNA 41)
(SEQ ID NO: 7)
mN#mN#mN#mNmNmNmNmNmNmNfNfNfNfNdN#dN#fNfNdN#mNmGrU#r
U#rU#fUfAmGmAmGmCmUmAmU#mG#mC#mU;
(crRNA 42)
(SEQ ID NO: 8)
mN#mN#mN#mNmNmNmNmNmNmNfNfNfNfNrN#rN#fNfNrN#mNmGdU#d
U#dU#fUfAmGmAmGmCmUmAmU#mG#mC#mU;
and
(crRNA 44)
(SEQ ID NO: 9)
mN#mN#mN#mNmNmNmNmNmNmNfNdN#fNfNrN#rN#fNfNrN#mNmGrU#
rU#rU#fUfAmGmAmGmCmUmAmU#mG#mC#mU,
wherein rN = RNA, mN = 2′-O-methyl RNA, fN = 2′-fluoro RNA, dN = 2′ -deoxy RNA, N#N = phosphorothioate linkage, and N = any nucleotide.

18. The chemically modified guide RNA of claim 1, comprising a tracrRNA portion modification pattern selected any of tracrRNAs 2-116 of Table 2.

19. A chemically modified guide RNA comprising:
(a) a crRNA portion comprising (i) a guide sequence capable of hybridizing to a target polynucleotide sequence, and (ii) a repeat sequence; and
(b) a tracrRNA portion comprising an anti-repeat nucleotide sequence that is complementary to the repeat sequence,
wherein the nucleotides at positions 4, 5, and 6 from the 5′ end of the crRNA portion comprise a 2′-fluoro chemical modification or a phosphorothioate chemical modification.

20. The chemically modified guide RNA of claim 19, comprising one or more additional chemical modifications, selected from a modification of a ribose group, a phosphate group, a nucleobase, or a combination thereof, optionally wherein:
each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC});
at least 80%, at least 90%, or 100% of the ribose groups are chemically modified;
each modification of the phosphate group is independently selected from the group consisting of a phosphorothioate, phosphonoacetate (PACE), thiophosphonoacetate (thioPACE), amide, triazole, phosphonate, and phosphotriester modification;
each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups; and/or
the guide RNA comprises at least 90% or 100% modified nucleotide.

21-28. (canceled)

29. The chemically modified guide RNA of claim 19, wherein the nucleotides at positions 4, 5, and 6 from the 5′ end of the crRNA portion comprise a 2′-fluoro chemical modification, optionally wherein:
the chemically modified guide RNA further comprises a 2′-fluoro chemical modification at one or more of positions 15, 16, 19, 22, 23, or 24 from the 5′ end of the crRNA portion;
the chemically modified guide RNA further comprises a 2′-fluoro chemical modification at positions 15, 16, 19, 22, 23, and 24 from the 5′ end of the crRNA portion.

30-31. (canceled)

32. The chemically modified guide RNA of claim 19, wherein the nucleotides at positions 4, 5, and 6 from the 5′ end of the crRNA portion comprise phosphorothioate chemical modification, optionally wherein:
the chemically modified guide RNA further comprises a 2′-fluoro chemical modification at one or more of positions 15, 16, 19, 22, 23, or 24 from the 5′ end of the crRNA portion; or
the chemically modified guide RNA further comprises a 2′-fluoro chemical modification at positions 15, 16, 19, 22, 23, and 24 from the 5′ end of the crRNA portion.

33-34. (canceled)

35. The chemically modified guide RNA of claim 19, comprising a crRNA portion modification pattern selected from the group consisting of:
(crRNA 33)
(SEQ ID NO: 121)
mN#mN#mN#rN#rN#rN#mNmNmNmNrN#rN#rN#rN#rN#rN#rN#rN#rNm
NmGrU#rU#rU#rU#rA#mGmAmGmCmUmAmU#mG#mC#mU;
(crRNA 34)
(SEQ ID NO: 122)
mN#mN#mN#rN#rN#rN#mNmNmNmNrN#rN#rN#rN#rN#rN#rN#rN#rNm
NmGrUrUrUrUrAmGmAmGmCmUmAmU#mG#mC#mU;
(crRNA 36)
(SEQ ID NO: 123)
mN#mN#mN#rN#rN#rN#mNmNmNmNrN#rN#rN#rN#rN#rN#rN#rN#rNm
NmGrUrUrUmUmAmGmAmGmCmUmAmU#mG#mC#mU;
(crRNA 37)
(SEQ ID NO: 124)
mN#mN#mN#rN#rN#rN#mNmNmNmNfNfNfNfNrN#rN#fNfNrN#mNmGr
U#rU#rU#mUmAmGmAmGmCmUmAmU#mG#mC#mU;
(crRNA 39)
(SEQ ID NO: 125)
mN#mN#mN#rN#rN#rN#mNmNmNmNfNfNfNfNfNfNfNfNfNmNmGfUfU
fUfUfAmGmAmGmCmUmAmU#mG#mC#mU;
and
(crRNA 45)
(SEQ ID NO: 126)
mN#mN#mN#fNfNfNmNmNmNmNfNfNfNfNfNfNfNfNfNmNmGfUfUfUf
UfAmGmAmGmCmUmAmU#mG#mC#mU,
wherein rN = RNA, mN = 2′-O-methyl RNA, fN = 2′-fluoro RNA, dN = 2′ -deoxy RNA, N#N = phosphorothioate linkage, and N = any nucleotide.

36. The chemically modified guide RNA of claim 19, comprising a tracrRNA portion modification pattern selected from any of tracrRNA 2-116 of Table 2.

37-50. (canceled)

51. A chemically modified guide RNA comprising:
(a) a crRNA portion comprising (i) a guide sequence capable of hybridizing to a target polynucleotide sequence, and (ii) a repeat sequence; and
(b) a tracrRNA portion comprising an anti-repeat nucleotide sequence that is complementary to the repeat sequence,
wherein:
the crRNA portion and the tracrRNA portion each independently comprise at least one chemically modified nucleotide; and
the tracrRNA portion comprises at least one 2′-deoxy modified ribose group.

52. The chemically modified guide RNA of claim 51, wherein:
the chemically modified guide RNA comprises a crRNA portion modification pattern selected any one of crRNAs 1-134 of Table 1; and/or
the modified nucleotides each independently comprise a modification of a ribose group, a phosphate group, a nucleobase, or a combination thereof, optionally wherein:
each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC});
at least 80%, at least 90%, or 100% of the ribose groups are chemically modified;
each modification of the phosphate group is independently selected from the group consisting of a phosphorothioate, phosphonoacetate (PACE), thiophosphonoacetate (thioPACE), amide, triazole, phosphonate, and phosphotriester modification;
each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups; and/or
the guide RNA comprises at least 90% or 100% modified nucleotide.

53-60. (canceled)

61. The chemically modified guide RNA of claim 51, comprising a tracrRNA portion modification pattern selected from the group consisting of:
(tracrRNA 74)
(SEQ ID NO: 80)
mA#mG#mC#mAmUmAmGmCmAmAmGdUdUmArAmAmArUmAmAmGmGrCrU
mArGrUrCmCrGrUrUmAmUmCmAmAmCmUmUmGmAmAmAmAmAmGmUmGm
GmCmAmCmCmGmAmGmUmCmGmGmUmGmC#mU#mU#mU;
(tracrRNA 75)
(SEQ ID NO: 81)
mA#mG#mC#mAmUmAmGmCmAmAmGrUrUmAdAmAmAdUmAmAmGmGrCrU
mArGrUrCmCrGrUrUmAmUmCmAmAmCmUmUmGmAmAmAmAmAmGmUmGm
GmCmAmCmCmGmAmGmUmCmGmGmUmGmC#mU#mU#mU;
(tracrRNA 76)
(SEQ ID NO: 82)
mA#mG#mC#mAmUmAmGmCmAmAmGdUdUmAdAmAmAdUmAmAmGmGrCrU
mArGrUrCmCrGrUrUmAmUmCmAmAmCmUmUmGmAmAmAmAmAmGmUmGm
GmCmAmCmCmGmAmGmUmCmGmGmUmGmC#mU#mU#mU;
(tracrRNA 77)
(SEQ ID NO: 83)
mA#mG#mC#mAmUmAmGmCmAmAmGrUrUmArAmAmArUmAmAmGmGdCdU
mArGrUrCmCrGrUrUmAmUmCmAmAmCmUmUmGmAmAmAmAmAmGmUmGm
GmCmAmCmCmGmAmGmUmCmGmGmUmGmC#mU#mU#mU;
(tracrRNA 78)
(SEQ ID NO: 84)
mA#mG#mC#mAmUmAmGmCmAmAmGrUrUmArAmAmArUmAmAmGmGrCrU
mAdGdUdCmCrGrUrUmAmUmCmAmAmCmUmUmGmAmAmAmAmAmGmUmGm
GmCmAmCmCmGmAmGmUmCmGmGmUmGmC#mU#mU#mU;
(tracrRNA 79)
(SEQ ID NO: 85)
mA#mG#mC#mAmUmAmGmCmAmAmGrUrUmArAmAmArUmAmAmGmGrCrU
mArGrUrCmCdGdUdUmAmUmCmAmAmCmUmUmGmAmAmAmAmAmGmUmGm
GmCmAmCmCmGmAmGmUmCmGmGmUmGmC#mU#mU#mU;
(tracrRNA 80)
(SEQ ID NO: 86)
mA#mG#mC#mAmUmAmGmCmAmAmGrUrUmArAmAmArUmAmAmGmGdCdU
mArGrUrCmCdGdUdUmAmUmCmAmAmCmUmUmGmAmAmAmAmAmGmUmGm
GmCmAmCmCmGmAmGmUmCmGmGmUmGmC#mU#mU#mU;
(tracrRNA 81)
(SEQ ID NO: 87)
mA#mG#mC#mAmUmAmGmCmAmAmGrUrUmArAmAmArUmAmAmGmGdCdU
mAdGdUdCmCrGrUrUmAmUmCmAmAmCmUmUmGmAmAmAmAmAmGmUmGm
GmCmAmCmCmGmAmGmUmCmGmGmUmGmC#mU#mU#mU;
(tracrRNA 82)
(SEQ ID NO: 88)
mA#mG#mC#mAmUmAmGmCmAmAmGrUrUmArAmAmArUmAmAmGmGrCrU
mAdGdUdCmCdGdUdUmAmUmCmAmAmCmUmUmGmAmAmAmAmAmGmUmGm
GmCmAmCmCmGmAmGmUmCmGmGmUmGmC#mU#mU#mU;
(tracrRNA 83)
(SEQ ID NO: 89)
mA#mG#mC#mAmUmAmGmCmAmAmGrUrUmArAmAmArUmAmAmGmGrCrU
mAdGrUdCmCrGrUrUmAmUmCmAmAmCmUmUmGmAmAmAmAmAmGmUmGm
GmCmAmCmCmGmAmGmUmCmGmGmUmGmC#mU#mU#mU;
(tracrRNA 84)
(SEQ ID NO: 90)
mA#mG#mC#mAmUmAmGmCmAmAmGrUrUmArAmAmArUmAmAmGmGrCrU
mArGrUrCmCdGrUdUmAmUmCmAmAmCmUmUmGmAmAmAmAmAmGmUmGm
GmCmAmCmCmGmAmGmUmCmGmGmUmGmC#mU#mU#mU;
(tracrRNA 85)
(SEQ ID NO: 91)
mA#mG#mC#mAmUmAmGmCmAmAmGrUrUmArAmAmArUmAmAmGmGdCdU
mAdGrUdCmCdGrUdUmAmUmCmAmAmCmUmUmGmAmAmAmAmAmGmUmGm
GmCmAmCmCmGmAmGmUmCmGmGmUmGmC#mU#mU#mU;
(tracrRNA 86)
(SEQ ID NO: 92)
mA#mG#mC#mAmUmAmGmCmAmAmGrUrUmArAmAmArUmAmAmGmGrCrU
mAdGrUdCmCdGrUdUmAmUmCmAmAmCmUmUmGmAmAmAmAmAmGmUmGm
GmCmAmCmCmGmAmGmUmCmGmGmUmGmC#mU#mU#mU;
(tracrRNA 87)
(SEQ ID NO: 93)
mA#mG#mC#mAmUmAmGmCmAmAmGrUrUmArAmAmArUmAmAmGmGdCrU
mAdGrUdCmCdGrUdUmAmUmCmAmAmCmUmUmGmAmAmAmAmAmGmUmGm
GmCmAmCmCmGmAmGmUmCmGmGmUmGmC#mU#mU#mU;
(tracrRNA 104)
(SEQ ID NO: 108)
mA#mG#mC#mAmUmAmGmCmAmAmGrUrUmArAmAmAdUmAmAmGmGdCdU
mArGdUdCmCrGrUrUmAmUmCmAmAmCmUmUmGmAmAmAmAmAmGmUmGm
GmCmAmCmCmGmAmGmUmCmGmGmUmGmC#mU#mU#mU;
(tracrRNA 105)
(SEQ ID NO: 109)
mA#mG#mC#mAmUmAmGmCmAmAmGrUdUmArAmAmAdUmAmAmGmGdCdU
mAdGdUdCmCrGrUrUmAmUmCmAmAmCmUmUmGmAmAmAmAmAmGmUmGm
GmCmAmCmCmGmAmGmUmCmGmGmUmGmC#mU#mU#mU;
and
(tracrRNA 106)
(SEQ ID NO: 110)
mA#mG#mC#mAmUmAmGmCmAmAmGdUdUmAdAmAmAdUmAmAmGmGdCdU
mAdGdUdCmCdGdUdUmAmUmCmAmAmCmUmUmGmAmAmAmAmAmGmUmGm
GmCmAmCmCmGmAmGmUmCmGmGmUmGmC#mU#mU#mU,
wherein rN = RNA, mN = 2′-O-methyl RNA, fN = 2′-fluoro RNA, dN = 2′ -deoxy RNA, N#N = phosphorothioate linkage, and N = any nucleotide.

62-75. (canceled)

76. A chemically modified guide RNA comprising:
(a) a crRNA portion comprising (i) a guide sequence capable of hybridizing to a target polynucleotide sequence, and (ii) a repeat sequence; and
(b) a tracrRNA portion comprising an anti-repeat nucleotide sequence that is complementary to the repeat sequence,
wherein the crRNA portion comprises at least one 2′-NH₂ (2′-amino RNA) modification.

77. The chemically modified guide RNA of claim 76, wherein:
a pyrimidine nucleotide comprises the 2′-NH₂modification and/or a purine nucleotide comprises the 2′-NH₂modification;
the crRNA portion comprises a 2′-NH₂ (2′-amino RNA) modification at one of more positions 16, 19, 22, 23, and 24 from the 5′ end of the crRNA portion;
the crRNA portion comprises a 2′-NH₂ (2′-amino) modification at positions 22, 23, and 24 from the 5′ end of the crRNA portion;
the crRNA portion comprises a 2′-NH₂ (2′-amino) modification at positions 19, 22, 23, and 24 from the 5′ end of the crRNA portion; and/or
the crRNA portion comprises a 2′-NH₂ (2′-amino) modification at positions 16 and 19 from the 5′ end of the crRNA portion.

78-87. (canceled)

88. The chemically modified guide RNA of claim 76, wherein:
the tracrRNA portion comprises a modification pattern selected from the group consisting of: tracrRNA 1 through tracrRNA 116 of Table 2;
the crRNA portion further comprises one or more additional modified nucleotides, each independently selected from a modification of a ribose group, a phosphate group, a nucleobase, or a combination thereof and/or
tracrRNA portion comprises one or more additional modified nucleotides, each independently selected from a modification of a ribose group, a phosphate group, a nucleobase, or a combination thereof,
optionally wherein:
each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC});
at least 80%, at least 90%, or 100% of the ribose groups are chemically modified;
each modification of the phosphate group is independently selected from the group consisting of a phosphorothioate, phosphonoacetate (PACE), thiophosphonoacetate (thioPACE), amide, triazole, phosphonate, and phosphotriester modification;
each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups; and/or
the guide RNA comprises at least 90% or 100% modified nucleotide.

89-95. (canceled)

96. The chemically modified guide RNA of claim 76, comprising a crRNA portion modification pattern selected from the group consisting of:
(crRNA 114)
(SEQ ID NO: 227)
mN#mN#mN#mNmNmNmNmNmNmNfNfNfNfNrN#rN#fNfNaNmNmGaUaU
aUfUfAmGmAmGmCmUmAmU#mG#mC#mU;
(crRNA 115)
(SEQ ID NO: 228)
mN#mN#mN#mNmNmNmNmNmNmNfNfNfNfNrN#rN#fNfNaNmNmGrU#r
U#rUffUfAmGmAmGmCmUmAmU#mG#mC#mU;
(crRNA 116)
(SEQ ID NO: 229)
mN#mN#mN#mNmNmNmNmNmNmNfNfNfNfNrN#rN#fNfNrN#mNmGaUr
U#rUffUfAmGmAmGmCmUmAmU#mG#mC#mU;
(crRNA 117)
(SEQ ID NO: 230)
mN#mN#mN#mNmNmNmNmNmNmNfNfNfNfNrN#rN#fNfNrN#mNmGrU#
aUrU#fUfAmGmAmGmCmUmAmU#mG#mC#mU;
(crRNA 118)
(SEQ ID NO: 231)
mN#mN#mN#mNmNmNmNmNmNmNfNfNfNfNrN#rN#fNfNrN#mNmGrU#
rU#aUfUfAmGmAmGmCmUmAmU#mG#mC#mU;
and
(crRNA 128)
(SEQ ID NO: 241)
mN#mN#mN#mNmNmNmNmNmNmNfNfNfNfNrN#rN#fNfNrN#mNmGaUa
UaUfUfAmGmAmGmCmUmAmU#mG#mC#mU,
wherein rN = RNA, mN = 2′-O-methyl RNA, fN = 2′-fluoro RNA, dN = 2′-deoxy RNA, aN = 2′-NH2 (2′-amino RNA), N#N = phosphorothioate linkage, and N = any nucleotide.

97-105. (canceled)

106. A chemically modified guide RNA comprising:
(a) a crRNA portion comprising (i) a guide sequence capable of hybridizing to a target polynucleotide sequence, and (ii) a repeat sequence; and
(b) a tracrRNA portion comprising an anti-repeat nucleotide sequence that is complementary to the repeat sequence,
wherein one or both of the crRNA portion and tracrRNA portion comprises at least one 4′-thio RNA modification.

107. The chemically modified guide RNA of claim 106, wherein the crRNA portion comprises a 4′-thio RNA modification at one of more positions 19, 22, 23, and 24 from the 5′ end of the crRNA portion, optionally wherein:
the crRNA portion comprises a 4′-thio RNA modification at position 19 from the 5′ end of the crRNA portion;
the crRNA portion comprises a 4′-thio RNA modification at position 22 from the 5′ end of the crRNA portion;
the crRNA portion comprises a 4′-thio RNA modification at position 23 from the 5′ end of the crRNA portion;
the crRNA portion comprises a 4′-thio RNA modification at position 24 from the 5′ end of the crRNA portion;
the crRNA portion comprises a 4′-thio RNA modification at positions 22 and 23 from the 5′ end of the crRNA portion;
the crRNA portion comprises a 4′-thio RNA modification at positions 22 and 24 from the 5′ end of the crRNA portion;
the crRNA portion comprises a 4′-thio RNA modification at positions 23 and 24 from the 5′ end of the crRNA portion; or
the crRNA portion comprises a 4′-thio RNA modification at positions 19, 22, 23, and 24 from the 5′ end of the crRNA portion.

108-115. (canceled)

116. The chemically modified guide RNA of claim 106, wherein the tracrRNA portion comprises a 4′-thio RNA modification at one of more positions 12, 13, 18, 24, 27, 31, and 32 from the 5′ end of the tracrRNA portion, optionally wherein:
the tracrRNA portion comprises a 4′-thio RNA modification at position 12 from the 5′ end of the tracrRNA portion;
the tracrRNA portion comprises a 4′-thio RNA modification at position 13 from the 5′ end of the tracrRNA portion;
the tracrRNA portion comprises a 4′-thio RNA modification at position 18 from the 5′ end of the tracrRNA portion;
the tracrRNA portion comprises a 4′-thio RNA modification at position 24 from the 5′ end of the tracrRNA portion;
the tracrRNA portion comprises a 4′-thio RNA modification at position 27 from the 5′ end of the tracrRNA portion;
the tracrRNA portion comprises a 4′-thio RNA modification at position 31 from the 5′ end of the tracrRNA portion;
the tracrRNA portion comprises a 4′-thio RNA modification at position 32 from the 5′ end of the tracrRNA portion;
the tracrRNA portion comprises a 4′-thio RNA modification at positions 12, 13, and 18 from the 5′ end of the tracrRNA portion;
the tracrRNA portion comprises a 4′-thio RNA modification at positions 24, 27, 31, and 32 from the 5′ end of the tracrRNA portion; or
the tracrRNA portion comprises a 4′-thio RNA modification at positions 12, 13, 18, 24, 27, 31, and 32 from the 5′ end of the tracrRNA portion.

117-126. (canceled)

127. The chemically modified guide RNA of claim 106, wherein the crRNA portion and/or the tracrRNA portion further comprise one or more additional modified nucleotides, each independently selected from a modification of a ribose group, a phosphate group, a nucleobase, or a combination thereof, optionally wherein:
each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC});
at least 80%, at least 90%, or 100% of the ribose groups are chemically modified;
each modification of the phosphate group is independently selected from the group consisting of a phosphorothioate, phosphonoacetate (PACE), thiophosphonoacetate (thioPACE), amide, triazole, phosphonate, and phosphotriester modification;
each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups; and/or
the guide RNA comprises at least 90% or 100% modified nucleotide.

128-134. (canceled)

135. The chemically modified guide RNA of claim 106, comprising a crRNA portion modification pattern selected from the group consisting of:
(crRNA 119)
(SEQ ID NO: 232)
mN#mN#mN#mNmNmNmNmNmNmNfNfNfNfNrN#rN#fNfNsN#mNmGsU#s
U#sU#fUfAmGmAmGmCmUmAmU#mG#mC#mU;
(crRNA 120)
(SEQ ID NO: 233)
mN#mN#mN#mNmNmNmNmNmNmNfNfNfNfNrN#rN#fNfNsNmNmGsUsUs
UfUfAmGmAmGmCmUmAmU#mG#mC#mU;
(crRNA 121)
(SEQ ID NO: 234)
mN#mN#mN#mNmNmNmNmNmNmNfNfNfNfNrN#rN#fNfNsNmNmGrU#rU
rU#fUfAmGmAmGmCmUmAmU#mG#mC#mU;
(crRNA 122)
(SEQ ID NO: 235)
mN#mN#mN#mNmNmNmNmNmNmNfNfNfNfNrN#rN#fNfNrN#mNmGsUrUr
U#fUfAmGmAmGmCmUmAmU#mG#mC#mU;
(crRNA 123)
(SEQ ID NO: 236)
mN#mN#mN#mNmNmNmNmNmNmNfNfNfNfNrN#rN#fNfNrN#mNmGrU#s
UrU#fUfAmGmAmGmCmUmAmU#mG#mC#mU;
(crRNA 124)
(SEQ ID NO: 237)
mN#mN#mN#mNmNmNmNmNmNmNfNfNfNfNrN#rN#fNfNrN#mNmGrU#r
U#sU#fUfAmGmAmGmCmUmAmU#mG#mC#mU;
(crRNA 125)
(SEQ ID NO: 238)
mN#mN#mN#mNmNmNmNmNmNmNfNfNfNfNrN#rN#fNfNrN#mNmGsUrU
sUfUfAmGmAmGmCmUmAmU#mG#mC#mU;
(crRNA 126)
(SEQ ID NO: 239)
mN#mN#mN#mNmNmNmNmNmNmNfNfNfNfNrN#rN#fNfNrN#mNmGsUsU
rU#fUfAmGmAmGmCmUmAmU#mG#mC#mU;
(crRNA 127)
(SEQ ID NO: 240)
mN#mN#mN#mNmNmNmNmNmNmNfNfNfNfNrN#rN#fNfNrN#mNmGrU#s
UsUfUfAmGmAmGmCmUmAmU#mG#mC#mU;
(crRNA 129)
(SEQ ID NO: 242)
mN#mN#mN#mNmNmNmNmNmNmNfNfNfNfNrN#rN#fNfNrN#mNmGsU#s
U#sU#fUfAmGmAmGmCmUmAmU#mG#mC#mU;
and
(crRNA 130)
(SEQ ID NO: 243)
mN#mN#mN#mNmNmNmNmNmNmNfNfNfNfNrN#rN#fNfNrN#mNmGsUsU
sUfUfAmGmAmGmCmUmAmU#mG#mC#mU,
wherein rN = RNA, mN = 2′-O-methyl RNA, fN = 2′-fluoro RNA, sN = 4′-thio RNA, N#N = phosphorothioate linkage, and N = any nucleotide, optionally wherein the tracrRNA portion comprises a modification pattern selected from the group consisting of: tracrRNA 1 through tracrRNA 116 of Table 2.

136. (canceled)

137. The chemically modified guide RNA of claim 106, comprising a tracrRNA portion modification pattern selected from the group consisting of:
(tracrRNA 107)
(SEQ ID NO: 111)
mA#mG#mC#mAmUmAmGmCmAmAmGsUsUmArAmAmAsUmAmAmGmGrCsU
mArGsUrCmCrGsUsUmAmUmCmAmAmCmUmUmGmAmAmAmAmAmGmUmGm
GmCmAmCmCmGmAmGmUmCmGmGmUmGmC#mU#mU#mU;
(tracrRNA 108)
(SEQ ID NO: 112)
mA#mG#mC#mAmUmAmGmCmAmAmGsUsUmArAmAmAsUmAmAmGmGrCrU
mArGrUrCmCrGrUrUmAmUmCmAmAmCmUmUmGmAmAmAmAmAmGmUmGm
GmCmAmCmCmGmAmGmUmCmGmGmUmGmC#mU#mU#mU;
(tracrRNA 109)
(SEQ ID NO: 113)
mA#mG#mC#mAmUmAmGmCmAmAmGrUrUmArAmAmArUmAmAmGmGrCsU
mArGsUrCmCrGsUsUmAmUmCmAmAmCmUmUmGmAmAmAmAmAmGmUmGm
GmCmAmCmCmGmAmGmUmCmGmGmUmGmC#mU#mU#mU;
(tracrRNA 110)
(SEQ ID NO: 114)
mA#mG#mC#mAmUmAmGmCmAmAmGsUrUmArAmAmArUmAmAmGmGrCrU
mArGrUrCmCrGrUrUmAmUmCmAmAmCmUmUmGmAmAmAmAmAmGmUmGm
GmCmAmCmCmGmAmGmUmCmGmGmUmGmC#mU#mU#mU;
(tracrRNA 111)
(SEQ ID NO: 115)
mA#mG#mC#mAmUmAmGmCmAmAmGrUsUmArAmAmArUmAmAmGmGrCrU
mArGrUrCmCrGrUrUmAmUmCmAmAmCmUmUmGmAmAmAmAmAmGmUmGm
GmCmAmCmCmGmAmGmUmCmGmGmUmGmC#mU#mU#mU;
(tracrRNA 112)
(SEQ ID NO: 116)
mA#mG#mC#mAmUmAmGmCmAmAmGrUrUmArAmAmAsUmAmAmGmGrCrU
mArGrUrCmCrGrUrUmAmUmCmAmAmCmUmUmGmAmAmAmAmAmGmUmGm
GmCmAmCmCmGmAmGmUmCmGmGmUmGmC#mU#mU#mU;
(tracrRNA 113)
(SEQ ID NO: 117)
mA#mG#mC#mAmUmAmGmCmAmAmGrUrUmArAmAmArUmAmAmGmGrCsU
mArGrUrCmCrGrUrUmAmUmCmAmAmCmUmUmGmAmAmAmAmAmGmUmGm
GmCmAmCmCmGmAmGmUmCmGmGmUmGmC#mU#mU#mU;
(tracrRNA 114)
(SEQ ID NO: 118)
mA#mG#mC#mAmUmAmGmCmAmAmGrUrUmArAmAmArUmAmAmGmGrCrU
mArGsUrCmCrGrUrUmAmUmCmAmAmCmUmUmGmAmAmAmAmAmGmUmGm
GmCmAmCmCmGmAmGmUmCmGmGmUmGmC#mU#mU#mU;
(tracrRNA 115)
(SEQ ID NO: 119)
mA#mG#mC#mAmUmAmGmCmAmAmGrUrUmArAmAmArUmAmAmGmGrCrU
mArGrUrCmCrGsUrUmAmUmCmAmAmCmUmUmGmAmAmAmAmAmGmUmGm
GmCmAmCmCmGmAmGmUmCmGmGmUmGmC#mU#mU#mU;
and
(tracrRNA 116)
(SEQ ID NO: 120)
mA#mG#mC#mAmUmAmGmCmAmAmGrUrUmArAmAmArUmAmAmGmGrCrU
mArGrUrCmCrGrUsUmAmUmCmAmAmCmUmUmGmAmAmAmAmAmGmUmGm
GmCmAmCmCmGmAmGmUmCmGmGmUmGmC#mU#mU#mU,
wherein rN = RNA, mN = 2′-O-methyl RNA, fN = 2′-fluoro RNA, sN = 4′-thio RNA, N#N = phosphorothioate linkage, and N = any nucleotide, optionally wherein the crRNA portion comprises a modification pattern selected from the group consisting of: crRNA 1 through crRNA 134 of Table 1.

138. (canceled)

139. The chemically modified guide RNA of claim 1, further comprising at least one moiety conjugated to the guide RNA, optionally wherein:
the at least one moiety is conjugated to at least one of the 5′ end of the crRNA portion, the 3′ end of the crRNA portion, the 5′ end of the tracrRNA portion, and the 3′ end of the tracrRNA portion;
the at least one moiety increases cellular uptake of the guide RNA;
the at least one moiety promotes specific tissue distribution of the guide RNA;
the at least one moiety is selected from the group consisting of fatty acids, steroids, secosteroids, lipids, gangliosides analogs, nucleoside analogs, endocannabinoids, vitamins, receptor ligands, peptides, aptamers, and alkyl chains;
the at least one moiety is selected from the group consisting of cholesterol, docosahexaenoic acid (DHA), docosanoic acid (DCA), lithocholic acid (LA), GalNAc, amphiphilic block copolymer (ABC), hydrophilic block copolymer (HBC), poloxamer, Cy5, and Cy3;
the at least one moiety is a modified lipid, optionally wherein:
the modified lipid is a branched lipid;
the modified lipid is a branched lipid of Formula I,
X-MC(═Y)M-Z-[L-MC(═Y)M-R]n, Formula I:
where X is a moiety that links the lipid to the guide RNA, each Y is independently oxygen or sulfur, each M is independently CH₂, NH, O or S, Z is a branching group which allows two or three (“n”) chains to be joined to a chemically modified guide RNA, L is an optional linker moiety, and each R is independently a saturated, monounsaturated or polyunsaturated linear or branched moiety from 2 to 30 atoms in length, a sterol, or other hydrophobic group; or
the modified lipid is a headgroup-modified lipid;
the modified lipid is a headgroup-modified lipid of Formula II,
X-MC(═Y)M-Z-[L-MC(═Y)M-R]n-L-K-J, Formula II:
where X is a moiety that links the lipid to the guide RNA, each Y is independently oxygen or sulfur, each M is independently CH₂, NH, N-alkyl, O or S, Z is a branching group which allows two or three (“n”) chains to be joined to chemically modified guide RNA, each L is independently an optional linker moiety, and R is a saturated, monounsaturated or polyunsaturated linear or branched moiety from 2 to 30 atoms in length, a sterol, or other hydrophobic group, K is a phosphate, sulfate, or amide and J is an aminoalkane or quaternary aminoalkane group; and/or
the at least one moiety is conjugated to the guide RNA via a linker, optionally wherein the linker is selected from the group consisting of an ethylene glycol chain, an alkyl chain, a polypeptide, a polysaccharide, and a block copolymer.

140-151. (canceled)

152. The chemically modified guide RNA of claim 1, wherein the guide RNA binds to a Cas9 nuclease selected from the group consisting of S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9), optionally wherein:
the Cas9 is a variant Cas9 with altered activity, optionally wherein the variant Cas9 is selected from the group consisting of a Cas9 nickase (nCas9), a catalytically dead Cas9 (dCas9), a hyper accurate Cas9 (HypaCas9), a high fidelity Cas9 (Cas9-HF), an enhanced specificity Cas9 (eCas9), and an expanded PAM Cas9 (xCas9);
the Cas9 off-target activity is reduced relative to an unmodified guide RNA; and/or
the Cas9 on-target activity is increased relative to an unmodified guide RNA.

153-156. (canceled)

157. The chemically modified guide RNA of claim 1, further comprising a nucleotide or non-nucleotide loop or linker linking the 3′ end of the crRNA portion to the 5′ end of the tracrRNA portion, optionally wherein:
the non-nucleotide linker comprises an ethylene glycol oligomer linker;
the nucleotide loop is chemically modified; and/or
the nucleotide loop comprises the nucleotide sequence of GAAA.

158-162. (canceled)

163. A method of altering expression of a target gene in a cell, comprising administering to said cell a genome editing system comprising:
the chemically modified guide RNA of claim 1; and
an RNA-guided nuclease or a polynucleotide encoding an RNA-guided nuclease, optionally wherein:
the target gene is in a cell in an organism;
expression of the target gene is knocked out or knocked down;
the sequence of the target gene is modified, edited, corrected or enhanced;
the guide RNA and the RNA-guided nuclease comprise a ribonucleoprotein (RNP) complex;
the RNA-guided nuclease is selected from the group consisting of S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9);
the polynucleotide encoding an RNA-guided nuclease comprises a vector, optionally wherein the vector is a viral vector, optionally wherein the viral vector is an adeno-associated virus (AAV) vector or a lentivirus (LV) vector;
the polynucleotide encoding an RNA-guided nuclease comprises a synthetic mRNA; and/or
expression of the target gene is reduced by at least about 20%.

164-175. (canceled)

176. A CRISPR genome editing system comprising:
a chemically modified guide RNA of claim 1; and
an RNA-guided nuclease or a polynucleotide encoding an RNA-guided nuclease, optionally wherein:
wherein the RNA-guided nuclease is selected from the group consisting of S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9);
the Cas9 is a variant Cas9 with altered activity, optionally wherein the variant Cas9 is selected from the group consisting of a Cas9 nickase (nCas9), a catalytically dead Cas9 (dCas9), a hyper accurate Cas9 (HypaCas9), a high fidelity Cas9 (Cas9-HF), an enhanced specificity Cas9 (eCas9), and an expanded PAM Cas9 (xCas9);
the Cas9 off-target activity is reduced relative to an unmodified guide RNA; and/or
the Cas9 on-target activity is increased relative to an unmodified guide RNA.

177-181. (canceled)
